SEQUENCE LISTING (761 sequences)

SEQ ID NO: 1 - DNA, 34 bp, Artificial Sequence
  Description of Artificial Sequence Synthetic oligonucleotide
gggacggtcggtagattctagaattgtgcttccc

SEQ ID NO: 2 - DNA, 19 bp, Artificial Sequence
  Description of Artificial Sequence Synthetic oligonucleotide
tgtccagtgttaggaaaaa

SEQ ID NO: 3 - DNA, 37 bp, Artificial Sequence
  Description of Artificial Sequence Synthetic oligonucleotide
gacgatgccttcagcacagatgtggcttctgtatgag

SEQ ID NO: 4 - DNA, 34 bp, Artificial Sequence
  Description of Artificial Sequence Synthetic oligonucleotide
gctggctcggtcaagatctagaattgtgcttcct

SEQ ID NO: 5 - DNA, 34 bp, Artificial Sequence
  Description of Artificial Sequence Synthetic oligonucleotide
gggacggtcggtagatcatcggtcagtgtcccca

SEQ ID NO: 6 - DNA, 20 bp, Artificial Sequence
  Description of Artificial Sequence Synthetic oligonucleotide
gatgtctgtctccttgatgt

SEQ ID NO: 7 - DNA, 36 bp, Artificial Sequence
  Description of Artificial Sequence Synthetic oligonucleotide
gacgatgccttcagcacaatgtgggggttttatttt

SEQ ID NO: 8 - DNA, 34 bp, Artificial Sequence
  Description of Artificial Sequence Synthetic oligonucleotide
gctggctcggtcaagacatcggtcagtgtccccg

SEQ ID NO: 9 - DNA, 34 bp, Artificial Sequence
  Description of Artificial Sequence Synthetic oligonucleotide
gggacggtcggtagattattttataatgcaaaag

SEQ ID NO: 10 - DNA, 37 bp, Artificial Sequence
  Description of Artificial Sequence Synthetic oligonucleotide
gacgatgccttcagcacagtgaattgccagattagtg

SEQ ID NO: 11 - DNA, 18 bp, Artificial Sequence
  Description of Artificial Sequence Synthetic oligonucleotide
tctaaagtgctgggattg

SEQ ID NO: 12 - DNA, 34 bp, Artificial Sequence
  Description of Artificial Sequence Synthetic oligonucleotide
gctggctcggtcaagatattttataatgcaaaac

SEQ ID NO: 13 - DNA, 34 bp, Artificial Sequence
  Description of Artificial Sequence Synthetic oligonucleotide
gggacggtcggtagataaggtctttgtacgtgta

SEQ ID NO: 14 - DNA, 19 bp, Artificial Sequence
  Description of Artificial Sequence Synthetic oligonucleotide
ccaggtactgccttacaaa

SEQ ID NO: 15 - DNA, 38 bp, Artificial Sequence
  Description of Artificial Sequence Synthetic oligonucleotide
gacgatgccttcagcacagctcccaaaataaatcactc

SEQ ID NO: 16 - DNA, 34 bp, Artificial Sequence
  Description of Artificial Sequence Synthetic oligonucleotide
gctggctcggtcaagaaaggtctttgtacgtgtg

SEQ ID NO: 17 - DNA, 34 bp, Artificial Sequence
  Description of Artificial Sequence Synthetic oligonucleotide
gggacggtcggtagattggagtcgggggagtcat

SEQ ID NO: 18 - DNA, 36 bp, Artificial Sequence
  Description of Artificial Sequence Synthetic oligonucleotide
gacgatgccttcagcacatagttcaagggtaaagga

SEQ ID NO: 19 - DNA, 18 bp, Artificial Sequence
  Description of Artificial Sequence Synthetic oligonucleotide
gaggacgagatgtaagag

SEQ ID NO: 20 - DNA, 34 bp, Artificial Sequence
  Description of Artificial Sequence Synthetic oligonucleotide
gctggctcggtcaagatggagtcgggggagtcac

SEQ ID NO: 21 - DNA, 34 bp, Artificial Sequence
  Description of Artificial Sequence Synthetic oligonucleotide
gggacggtcggtagatcagcgcatcctgaaccac

SEQ ID NO: 22 - DNA, 19 bp, Artificial Sequence
  Description of Artificial Sequence Synthetic oligonucleotide
gctggaacgagttcatcct

SEQ ID NO: 23 - DNA, 36 bp, Artificial Sequence
  Description of Artificial Sequence Synthetic oligonucleotide
gacgatgccttcagcacaggaccccacctttcttgt

SEQ ID NO: 24 - DNA, 34 bp, Artificial Sequence
  Description of Artificial Sequence Synthetic oligonucleotide
gctggctcggtcaagacagcgcatcctgaaccat

SEQ ID NO: 25 - DNA, 34 bp, Artificial Sequence
  Description of Artificial Sequence Synthetic oligonucleotide
gggacggtcggtagattcctgctcttttctctag

SEQ ID NO: 26 - DNA, 38 bp, Artificial Sequence
  Description of Artificial Sequence Synthetic oligonucleotide
gacgatgccttcagcacacactgactgcttactctacc

SEQ ID NO: 27 - DNA, 18 bp, Artificial Sequence
  Description of Artificial Sequence Synthetic oligonucleotide
tactgtgtctcagctcca

SEQ ID NO: 28 - DNA, 34 bp, Artificial Sequence
  Description of Artificial Sequence Synthetic oligonucleotide
gctggctcggtcaagatcctgctcttttctctaa

SEQ ID NO: 29 - DNA, 34 bp, Artificial Sequence
  Description of Artificial Sequence Synthetic oligonucleotide
gggacggtcggtagatgtgaatcccaatacgaag

SEQ ID NO: 30 - DNA, 40 bp, Artificial Sequence
  Description of Artificial Sequence Synthetic oligonucleotide
gacgatgccttcagcacataaaaaataaccaggtactcca

SEQ ID NO: 31 - DNA, 24 bp, Artificial Sequence
  Description of Artificial Sequence Synthetic oligonucleotide
gatgagtccttcaccaaacataca

SEQ ID NO: 32 - DNA, 34 bp, Artificial Sequence
  Description of Artificial Sequence Synthetic oligonucleotide
gctggctcggtcaagagtgaatcccaatacgaaa

SEQ ID NO: 33 - DNA, 34 bp, Artificial Sequence
  Description of Artificial Sequence Synthetic oligonucleotide
gggacggtcggtagatggtgggaggttccagcca

SEQ ID NO: 34 - DNA, 18 bp, Artificial Sequence
  Description of Artificial Sequence Synthetic oligonucleotide
gcaggaagaaagctagaa

SEQ ID NO: 35 - DNA, 36 bp, Artificial Sequence
  Description of Artificial Sequence Synthetic oligonucleotide
gacgatgccttcagcacaaggcaggataatgacaac

SEQ ID NO: 36 - DNA, 34 bp, Artificial Sequence
  Description of Artificial Sequence Synthetic oligonucleotide
gctggctcggtcaagaggtgggaggttccagccg

SEQ ID NO: 37 - DNA, 34 bp, Artificial Sequence
  Description of Artificial Sequence Synthetic oligonucleotide
gggacggtcggtagatcatttccacctcaccaaa

SEQ ID NO: 38 - DNA, 18 bp, Artificial Sequence
  Description of Artificial Sequence Synthetic oligonucleotide
aggtattcccggcgtttc

SEQ ID NO: 39 - DNA, 36 bp, Artificial Sequence
  Description of Artificial Sequence Synthetic oligonucleotide
gacgatgccttcagcacatgttgtgcgtctgcttcc

SEQ ID NO: 40 - DNA, 34 bp, Artificial Sequence
  Description of Artificial Sequence Synthetic oligonucleotide
gctggctcggtcaagacatttccacctcaccaag

SEQ ID NO: 41 - DNA, 34 bp, Artificial Sequence
  Description of Artificial Sequence Synthetic oligonucleotide
gggacggtcggtagattgtgaagaactgttgctc

SEQ ID NO: 42 - DNA, 20 bp, Artificial Sequence
  Description of Artificial Sequence Synthetic oligonucleotide
ctgaagctcatctgccttct

SEQ ID NO: 43 - DNA, 36 bp, Artificial Sequence
  Description of Artificial Sequence Synthetic oligonucleotide
gacgatgccttcagcacatccccttccttcttacct

SEQ ID NO: 44 - DNA, 34 bp, Artificial Sequence
  Description of Artificial Sequence Synthetic oligonucleotide
gctggctcggtcaagatgtgaagaactgttgctg

SEQ ID NO: 45 - DNA, 34 bp, Artificial Sequence
  Description of Artificial Sequence Synthetic oligonucleotide
gggacggtcggtagatgcccgcttttcttcatcg

SEQ ID NO: 46 - DNA, 38 bp, Artificial Sequence
  Description of Artificial Sequence Synthetic oligonucleotide
gacgatgccttcagcacactgtcttcaagggcttacac

SEQ ID NO: 47 - DNA, 18 bp, Artificial Sequence
  Description of Artificial Sequence Synthetic oligonucleotide
tccaacttcaggcaaaac

SEQ ID NO: 48 - DNA, 34 bp, Artificial Sequence
  Description of Artificial Sequence Synthetic oligonucleotide
gctggctcggtcaagagcccgcttttcttcatca

SEQ ID NO: 49 - DNA, 34 bp, Artificial Sequence
  Description of Artificial Sequence Synthetic oligonucleotide
gggacggtcggtagatcccaaggccaacagggag

SEQ ID NO: 50 - DNA, 38 bp, Artificial Sequence
  Description of Artificial Sequence Synthetic oligonucleotide
gacgatgccttcagcacagcattcttatgccagtgttc

SEQ ID NO: 51 - DNA, 18 bp, Artificial Sequence
  Description of Artificial Sequence Synthetic oligonucleotide
atccatcccatcctgtgt

SEQ ID NO: 52 - DNA, 34 bp, Artificial Sequence
  Description of Artificial Sequence Synthetic oligonucleotide
gctggctcggtcaagacccaaggccaacagggaa

SEQ ID NO: 53 - DNA, 34 bp, Artificial Sequence
  Description of Artificial Sequence Synthetic oligonucleotide
gggacggtcggtagatgagtgggtgctgttcccg

SEQ ID NO: 54 - DNA, 36 bp, Artificial Sequence
  Description of Artificial Sequence Synthetic oligonucleotide
gacgatgccttcagcacagttactgcctctctgacc

SEQ ID NO: 55 - DNA, 18 bp, Artificial Sequence
  Description of Artificial Sequence Synthetic oligonucleotide
agtgtgacctgctctctt

SEQ ID NO: 56 - DNA, 34 bp, Artificial Sequence
  Description of Artificial Sequence Synthetic oligonucleotide
gctggctcggtcaagagagtgggtgctgttccca

SEQ ID NO: 57 - DNA, 36 bp, Artificial Sequence
  Description of Artificial Sequence Synthetic oligonucleotide
gacgatgccttcagcacaaacacattccccctctac

SEQ ID NO: 58 - DNA, 19 bp, Artificial Sequence
  Description of Artificial Sequence Synthetic oligonucleotide
gtctctattccaagccaag

SEQ ID NO: 59 - DNA, 34 bp, Artificial Sequence
  Description of Artificial Sequence Synthetic oligonucleotide
gggacggtcggtagatccccgctccagctcctca

SEQ ID NO: 60 - DNA, 34 bp, Artificial Sequence
  Description of Artificial Sequence Synthetic oligonucleotide
gctggctcggtcaagaccccgctccagctcctct

SEQ ID NO: 61 - DNA, 34 bp, Artificial Sequence
  Description of Artificial Sequence Synthetic oligonucleotide
gggacggtcggtagatccgcttctgcttctgctg

SEQ ID NO: 62 - DNA, 36 bp, Artificial Sequence
  Description of Artificial Sequence Synthetic oligonucleotide
gacgatgccttcagcacaaggagaagagggaggaga

SEQ ID NO: 63 - DNA, 18 bp, Artificial Sequence
  Description of Artificial Sequence Synthetic oligonucleotide
ggagcacgtaaggagaaa

SEQ ID NO: 64 - DNA, 34 bp, Artificial Sequence
  Description of Artificial Sequence Synthetic oligonucleotide
gctggctcggtcaagaccgcttctgcttctgctc

SEQ ID NO: 65 - DNA, 34 bp, Artificial Sequence
  Description of Artificial Sequence Synthetic oligonucleotide
gggacggtcggtagatggccaggggctggagggc

SEQ ID NO: 66 - DNA, 20 bp, Artificial Sequence
  Description of Artificial Sequence Synthetic oligonucleotide
tcttcagttctctcagcttc

SEQ ID NO: 67 - DNA, 36 bp, Artificial Sequence
  Description of Artificial Sequence Synthetic oligonucleotide
gacgatgccttcagcacatcactaggggctcttacc

SEQ ID NO: 68 - DNA, 34 bp, Artificial Sequence
  Description of Artificial Sequence Synthetic oligonucleotide
gctggctcggtcaagaggccaggggctggagggt

SEQ ID NO: 69 - DNA, 34 bp, Artificial Sequence
  Description of Artificial Sequence Synthetic oligonucleotide
gggacggtcggtagattcctcccgctgcttcagt

SEQ ID NO: 70 - DNA, 44 bp, Artificial Sequence
  Description of Artificial Sequence Synthetic oligonucleotide
gacgatgccttcagcacatcacttacccatcatacttctttttc

SEQ ID NO: 71 - DNA, 18 bp, Artificial Sequence
  Description of Artificial Sequence Synthetic oligonucleotide
aatcctgcctcccacctt

SEQ ID NO: 72 - DNA, 34 bp, Artificial Sequence
  Description of Artificial Sequence Synthetic oligonucleotide
gctggctcggtcaagatcctcccgctgcttcagc

SEQ ID NO: 73 - DNA, 34 bp, Artificial Sequence
  Description of Artificial Sequence Synthetic oligonucleotide
gggacggtcggtagatagaaattccctcccaact

SEQ ID NO: 74 - DNA, 36 bp, Artificial Sequence
  Description of Artificial Sequence Synthetic oligonucleotide
gacgatgccttcagcacatgattgagccagttgttt

SEQ ID NO: 75 - DNA, 19 bp, Artificial Sequence
  Description of Artificial Sequence Synthetic oligonucleotide
ggggtgtattttgagagtg

SEQ ID NO: 76 - DNA, 34 bp, Artificial Sequence
  Description of Artificial Sequence Synthetic oligonucleotide
gctggctcggtcaagaagaaattccctcccaacc

SEQ ID NO: 77 - DNA, 34 bp, Artificial Sequence
  Description of Artificial Sequence Synthetic oligonucleotide
gggacggtcggtagatgctggtttgactggacgg

SEQ ID NO: 78 - DNA, 38 bp, Artificial Sequence
  Description of Artificial Sequence Synthetic oligonucleotide
gacgatgccttcagcacaaccttggtataatcctttcc

SEQ ID NO: 79 - DNA, 18 bp, Artificial Sequence
  Description of Artificial Sequence Synthetic oligonucleotide
aggcaacctaatccactt

SEQ ID NO: 80 - DNA, 34 bp, Artificial Sequence
  Description of Artificial Sequence Synthetic oligonucleotide
gctggctcggtcaagagctggtttgactggacgc

SEQ ID NO: 81 - DNA, 34 bp, Artificial Sequence
  Description of Artificial Sequence Synthetic oligonucleotide
gggacggtcggtagatagtgctgtgatacctgga

SEQ ID NO: 82 - DNA, 34 bp, Artificial Sequence
  Description of Artificial Sequence Synthetic oligonucleotide
gctggctcggtcaagaagtgctgtgatacctggc

SEQ ID NO: 83 - DNA, 18 bp, Artificial Sequence
  Description of Artificial Sequence Synthetic oligonucleotide
acacccacaaaacaagaa

SEQ ID NO: 84 - DNA, 38 bp, Artificial Sequence
  Description of Artificial Sequence Synthetic oligonucleotide
gacgatgccttcagcacaggaacaaggacataaaagag

SEQ ID NO: 85 - DNA, 34 bp, Artificial Sequence
  Description of Artificial Sequence Synthetic oligonucleotide
gggacggtcggtagataggaaatgttagccctgt

SEQ ID NO: 86 - DNA, 37 bp, Artificial Sequence
  Description of Artificial Sequence Synthetic oligonucleotide
gacgatgccttcagcacactccacttctctatgcctc

SEQ ID NO: 87 - DNA, 19 bp, Artificial Sequence
  Description of Artificial Sequence Synthetic oligonucleotide
gtccccagctatgtattgt

SEQ ID NO: 88 - DNA, 34 bp, Artificial Sequence
  Description of Artificial Sequence Synthetic oligonucleotide
gctggctcggtcaagaaggaaatgttagccctgc

SEQ ID NO: 89 - DNA, 34 bp, Artificial Sequence
  Description of Artificial Sequence Synthetic oligonucleotide
gggacggtcggtagatctcagggagggagagaga

SEQ ID NO: 90 - DNA, 18 bp, Artificial Sequence
  Description of Artificial Sequence Synthetic oligonucleotide
gggacagacagacagaca

SEQ ID NO: 91 - DNA, 36 bp, Artificial Sequence
  Description of Artificial Sequence Synthetic oligonucleotide
gacgatgccttcagcacacaactccttcttcagcac

SEQ ID NO: 92 - DNA, 34 bp, Artificial Sequence
  Description of Artificial Sequence Synthetic oligonucleotide
gctggctcggtcaagactcagggagggagagagt

SEQ ID NO: 93 - DNA, 34 bp, Artificial Sequence
  Description of Artificial Sequence Synthetic oligonucleotide
gggacggtcggtagatgcggctgccccgtcctgt

SEQ ID NO: 94 - DNA, 39 bp, Artificial Sequence
  Description of Artificial Sequence Synthetic oligonucleotide
gacgatgccttcagcacagtgtgtctatgtgtctgtgtg

SEQ ID NO: 95 - DNA, 18 bp, Artificial Sequence
  Description of Artificial Sequence Synthetic oligonucleotide
cggacttctccttcttgt

SEQ ID NO: 96 - DNA, 34 bp, Artificial Sequence
  Description of Artificial Sequence Synthetic oligonucleotide
gctggctcggtcaagagcggctgccccgtcctgc

SEQ ID NO: 97 - DNA, 18 bp, Artificial Sequence
  Description of Artificial Sequence Synthetic oligonucleotide
tagggtaagcagcaagag

SEQ ID NO: 98 - DNA, 19 bp, Artificial Sequence
  Description of Artificial Sequence Synthetic oligonucleotide
cacaaggcaagagataaca

SEQ ID NO: 99 - DNA, 34 bp, Artificial Sequence
  Description of Artificial Sequence Synthetic oligonucleotide
gggacggtcggtagatcaggcaagatagacagca

SEQ ID NO: 100 - DNA, 34 bp, Artificial Sequence
  Description of Artificial Sequence Synthetic oligonucleotide
gctggctcggtcaagacaggcaagatagacagcg

SEQ ID NO: 101 - DNA, 21 bp, Artificial Sequence
  Description of Artificial Sequence Synthetic oligonucleotide
gtgcccatacgaacagaatag

SEQ ID NO: 102 - DNA, 18 bp, Artificial Sequence
  Description of Artificial Sequence Synthetic oligonucleotide
tgccaagtaccccaagag

SEQ ID NO: 103 - DNA, 34 bp, Artificial Sequence
  Description of Artificial Sequence Synthetic oligonucleotide
gggacggtcggtagatccattcctccccagacat

SEQ ID NO: 104 - DNA, 34 bp, Artificial Sequence
  Description of Artificial Sequence Synthetic oligonucleotide
gctggctcggtcaagaccattcctccccagacac

SEQ ID NO: 105 - DNA, 34 bp, Artificial Sequence
  Description of Artificial Sequence Synthetic oligonucleotide
gggacggtcggtagatcgtgcgagcagcgaaagc

SEQ ID NO: 106 - DNA, 23 bp, Artificial Sequence
  Description of Artificial Sequence Synthetic oligonucleotide
ccagagagaagtcgaggaagaga

SEQ ID NO: 107 - DNA, 36 bp, Artificial Sequence
  Description of Artificial Sequence Synthetic oligonucleotide
gacgatgccttcagcacagtcacccccaaaagcagg

SEQ ID NO: 108 - DNA, 34 bp, Artificial Sequence
  Description of Artificial Sequence Synthetic oligonucleotide
gctggctcggtcaagacgtgcgagcagcgaaagg

SEQ ID NO: 109 - DNA, 36 bp, Artificial Sequence
  Description of Artificial Sequence Synthetic oligonucleotide
gacgatgccttcagcacacttttcctcctagcccac

SEQ ID NO: 110 - DNA, 21 bp, Artificial Sequence
  Description of Artificial Sequence Synthetic oligonucleotide
aagtgatgtaaccctcctctc

SEQ ID NO: 111 - DNA, 34 bp, Artificial Sequence
  Description of Artificial Sequence Synthetic oligonucleotide
gggacggtcggtagattcagctataaatagggcc

SEQ ID NO: 112 - DNA, 34 bp, Artificial Sequence
  Description of Artificial Sequence Synthetic oligonucleotide
gctggctcggtcaagatcagctataaatagggca

SEQ ID NO: 113 - DNA, 34 bp, Artificial Sequence
  Description of Artificial Sequence Synthetic oligonucleotide
gggacggtcggtagattgatggcgggtgccaagg

SEQ ID NO: 114 - DNA, 36 bp, Artificial Sequence
  Description of Artificial Sequence Synthetic oligonucleotide
gacgatgccttcagcacagctctttcctttgcttcc

SEQ ID NO: 115 - DNA, 18 bp, Artificial Sequence
  Description of Artificial Sequence Synthetic oligonucleotide
cactgggggtcctcttac

SEQ ID NO: 116 - DNA, 34 bp, Artificial Sequence
  Description of Artificial Sequence Synthetic oligonucleotide
gctggctcggtcaagatgatggcgggtgccaaga

SEQ ID NO: 117 - DNA, 34 bp, Artificial Sequence
  Description of Artificial Sequence Synthetic oligonucleotide
gggacggtcggtagatcaagggcactcacattat

SEQ ID NO: 118 - DNA, 36 bp, Artificial Sequence
  Description of Artificial Sequence Synthetic oligonucleotide
gacgatgccttcagcacagctcttgcgtctgtttcc

SEQ ID NO: 119 - DNA, 18 bp, Artificial Sequence
  Description of Artificial Sequence Synthetic oligonucleotide
tttcccttctgtcccctt

SEQ ID NO: 120 - DNA, 34 bp, Artificial Sequence
  Description of Artificial Sequence Synthetic oligonucleotide
gctggctcggtcaagacaagggcactcacattac

SEQ ID NO: 121 - DNA, 34 bp, Artificial Sequence
  Description of Artificial Sequence Synthetic oligonucleotide
gggacggtcggtagatgtgacttttggttcccac

SEQ ID NO: 122 - DNA, 19 bp, Artificial Sequence
  Description of Artificial Sequence Synthetic oligonucleotide
aactcggggtcactggtct

SEQ ID NO: 123 - DNA, 36 bp, Artificial Sequence
  Description of Artificial Sequence Synthetic oligonucleotide
gacgatgccttcagcacacagcgggtatggaggatg

SEQ ID NO: 124 - DNA, 34 bp, Artificial Sequence
  Description of Artificial Sequence Synthetic oligonucleotide
gctggctcggtcaagagtgacttttggttcccat

SEQ ID NO: 125 - DNA, 18 bp, Artificial Sequence
  Description of Artificial Sequence Synthetic oligonucleotide
acaccagttctccctcct

SEQ ID NO: 126 - DNA, 36 bp, Artificial Sequence
  Description of Artificial Sequence Synthetic oligonucleotide
gacgatgccttcagcacacccacctttcctaatcct

SEQ ID NO: 127 - DNA, 34 bp, Artificial Sequence
  Description of Artificial Sequence Synthetic oligonucleotide
gggacggtcggtagatttgggactctgcgtcaag

SEQ ID NO: 128 - DNA, 34 bp, Artificial Sequence
  Description of Artificial Sequence Synthetic oligonucleotide
gctggctcggtcaagattgggactctgcgtcaat

SEQ ID NO: 129 - DNA, 34 bp, Artificial Sequence
  Description of Artificial Sequence Synthetic oligonucleotide
gggacggtcggtagatctctcaaagcccacacaa

SEQ ID NO: 130 - DNA, 34 bp, Artificial Sequence
  Description of Artificial Sequence Synthetic oligonucleotide
gctggctcggtcaagactctcaaagcccacacac

SEQ ID NO: 131 - DNA, 21 bp, Artificial Sequence
  Description of Artificial Sequence Synthetic oligonucleotide
agaaaaagaaaaggaaaaaga

SEQ ID NO: 132 - DNA, 38 bp, Artificial Sequence
  Description of Artificial Sequence Synthetic oligonucleotide
gacgatgccttcagcacaggaaagttacaaggctatga

SEQ ID NO: 133 - DNA, 34 bp, Artificial Sequence
  Description of Artificial Sequence Synthetic oligonucleotide
gggacggtcggtagatacctgcctctaaggtctg

SEQ ID NO: 134 - DNA, 37 bp, Artificial Sequence
  Description of Artificial Sequence Synthetic oligonucleotide
gacgatgccttcagcacaaggagaagacagttcaagg

SEQ ID NO: 135 - DNA, 18 bp, Artificial Sequence
  Description of Artificial Sequence Synthetic oligonucleotide
acagttgccagagaaaag

SEQ ID NO: 136 - DNA, 34 bp, Artificial Sequence
  Description of Artificial Sequence Synthetic oligonucleotide
gctggctcggtcaagaacctgcctctaaggtctc

SEQ ID NO: 137 - DNA, 18 bp, Artificial Sequence
  Description of Artificial Sequence Synthetic oligonucleotide
tcacttgcctctactcca

SEQ ID NO: 138 - DNA, 21 bp, Artificial Sequence
  Description of Artificial Sequence Synthetic oligonucleotide
ataccagaaagactaagctcc

SEQ ID NO: 139 - DNA, 34 bp, Artificial Sequence
  Description of Artificial Sequence Synthetic oligonucleotide
gggacggtcggtagatgggtgagctctgtgggca

SEQ ID NO: 140 - DNA, 34 bp, Artificial Sequence
  Description of Artificial Sequence Synthetic oligonucleotide
gctggctcggtcaagagggtgagctctgtgggcc

SEQ ID NO: 141 - DNA, 34 bp, Artificial Sequence
  Description of Artificial Sequence Synthetic oligonucleotide
gggacggtcggtagatccaagggttatggcaggg

SEQ ID NO: 142 - DNA, 38 bp, Artificial Sequence
  Description of Artificial Sequence Synthetic oligonucleotide
gacgatgccttcagcacacctgactatttggggttgtg

SEQ ID NO: 143 - DNA, 19 bp, Artificial Sequence
  Description of Artificial Sequence Synthetic oligonucleotide
atcgctctctgcttctgct

SEQ ID NO: 144 - DNA, 34 bp, Artificial Sequence
  Description of Artificial Sequence Synthetic oligonucleotide
gctggctcggtcaagaccaagggttatggcagga

SEQ ID NO: 145 - DNA, 34 bp, Artificial Sequence
  Description of Artificial Sequence Synthetic oligonucleotide
gggacggtcggtagatccaaaaccccagcgctgt

SEQ ID NO: 146 - DNA, 38 bp, Artificial Sequence
  Description of Artificial Sequence Synthetic oligonucleotide
gacgatgccttcagcacactctttatcctgcttatggt

SEQ ID NO: 147 - DNA, 18 bp, Artificial Sequence
  Description of Artificial Sequence Synthetic oligonucleotide
ccaagctcactctgtagg

SEQ ID NO: 148 - DNA, 34 bp, Artificial Sequence
  Description of Artificial Sequence Synthetic oligonucleotide
gctggctcggtcaagaccaaaaccccagcgctgc

SEQ ID NO: 149 - DNA, 18 bp, Artificial Sequence
  Description of Artificial Sequence Synthetic oligonucleotide
aatacaatggaagccaag

SEQ ID NO: 150 - DNA, 18 bp, Artificial Sequence
  Description of Artificial Sequence Synthetic oligonucleotide
cctaatcgaacagaaagg

SEQ ID NO: 151 - DNA, 34 bp, Artificial Sequence
  Description of Artificial Sequence Synthetic oligonucleotide
gggacggtcggtagatccagtctccatccacttc

SEQ ID NO: 152 - DNA, 34 bp, Artificial Sequence
  Description of Artificial Sequence Synthetic oligonucleotide
gctggctcggtcaagaccagtctccatccacttt

SEQ ID NO: 153 - DNA, 34 bp, Artificial Sequence
  Description of Artificial Sequence Synthetic oligonucleotide
gggacggtcggtagattgaacggcatgacgggga

SEQ ID NO: 154 - DNA, 19 bp, Artificial Sequence
  Description of Artificial Sequence Synthetic oligonucleotide
aagtgtttctgctgtgcct

SEQ ID NO: 155 - DNA, 37 bp, Artificial Sequence
  Description of Artificial Sequence Synthetic oligonucleotide
gacgatgccttcagcacacaagtcctggttttccatc

SEQ ID NO: 156 - DNA, 34 bp, Artificial Sequence
  Description of Artificial Sequence Synthetic oligonucleotide
gctggctcggtcaagatgaacggcatgacggggg

SEQ ID NO: 157 - DNA, 34 bp, Artificial Sequence
  Description of Artificial Sequence Synthetic oligonucleotide
gggacggtcggtagataccccaggatgcccacac

SEQ ID NO: 158 - DNA, 19 bp, Artificial Sequence
  Description of Artificial Sequence Synthetic oligonucleotide
gtttatcctcctcatgtcc

SEQ ID NO: 159 - DNA, 37 bp, Artificial Sequence
  Description of Artificial Sequence Synthetic oligonucleotide
gacgatgccttcagcacagttaccttttccacctctc

SEQ ID NO: 160 - DNA, 34 bp, Artificial Sequence
  Description of Artificial Sequence Synthetic oligonucleotide
gctggctcggtcaagaaccccaggatgcccacat

SEQ ID NO: 161 - DNA, 34 bp, Artificial Sequence
  Description of Artificial Sequence Synthetic oligonucleotide
gggacggtcggtagatggaaacaaaccaaaatga

SEQ ID NO: 162 - DNA, 18 bp, Artificial Sequence
  Description of Artificial Sequence Synthetic oligonucleotide
ccagcacccaaaataaga

SEQ ID NO: 163 - DNA, 36 bp, Artificial Sequence
  Description of Artificial Sequence Synthetic oligonucleotide
gacgatgccttcagcacaataagttgaagccctccc

SEQ ID NO: 164 - DNA, 34 bp, Artificial Sequence
  Description of Artificial Sequence Synthetic oligonucleotide
gctggctcggtcaagaggaaacaaaccaaaatgg

SEQ ID NO: 165 - DNA, 34 bp, Artificial Sequence
  Description of Artificial Sequence Synthetic oligonucleotide
gggacggtcggtagatggcttcacggaggaagaa

SEQ ID NO: 166 - DNA, 19 bp, Artificial Sequence
  Description of Artificial Sequence Synthetic oligonucleotide
ttaggagctgtgaggtatg

SEQ ID NO: 167 - DNA, 36 bp, Artificial Sequence
  Description of Artificial Sequence Synthetic oligonucleotide
gacgatgccttcagcacataagatggagcagggtag

SEQ ID NO: 168 - DNA, 34 bp, Artificial Sequence
  Description of Artificial Sequence Synthetic oligonucleotide
gctggctcggtcaagaggcttcacggaggaagag

SEQ ID NO: 169 - DNA, 34 bp, Artificial Sequence
  Description of Artificial Sequence Synthetic oligonucleotide
gggacggtcggtagataaagggctcccaacacca

SEQ ID NO: 170 - DNA, 20 bp, Artificial Sequence
  Description of Artificial Sequence Synthetic oligonucleotide
tgagcacaagatcagagagg

SEQ ID NO: 171 - DNA, 37 bp, Artificial Sequence
  Description of Artificial Sequence Synthetic oligonucleotide
gacgatgccttcagcacaagacagagacgcaggaatg

SEQ ID NO: 172 - DNA, 34 bp, Artificial Sequence
  Description of Artificial Sequence Synthetic oligonucleotide
gctggctcggtcaagaaaagggctcccaacaccg

SEQ ID NO: 173 - DNA, 34 bp, Artificial Sequence
  Description of Artificial Sequence Synthetic oligonucleotide
gggacggtcggtagatagggacaaccaaagtgac

SEQ ID NO: 174 - DNA, 19 bp, Artificial Sequence
  Description of Artificial Sequence Synthetic oligonucleotide
atcatcagaacagccctac

SEQ ID NO: 175 - DNA, 37 bp, Artificial Sequence
  Description of Artificial Sequence Synthetic oligonucleotide
gacgatgccttcagcacacaagcccacctacttactc

SEQ ID NO: 176 - DNA, 34 bp, Artificial Sequence
  Description of Artificial Sequence Synthetic oligonucleotide
gctggctcggtcaagaagggacaaccaaagtgat

SEQ ID NO: 177 - DNA, 34 bp, Artificial Sequence
  Description of Artificial Sequence Synthetic oligonucleotide
gggacggtcggtagattgggcgtcctggtgggca

SEQ ID NO: 178 - DNA, 18 bp, Artificial Sequence
  Description of Artificial Sequence Synthetic oligonucleotide
tcttcgggctaactcttt

SEQ ID NO: 179 - DNA, 37 bp, Artificial Sequence
  Description of Artificial Sequence Synthetic oligonucleotide
gacgatgccttcagcacactgtcactccaaaccttct

SEQ ID NO: 180 - DNA, 34 bp, Artificial Sequence
  Description of Artificial Sequence Synthetic oligonucleotide
gctggctcggtcaagatgggcgtcctggtgggcg

SEQ ID NO: 181 - DNA, 34 bp, Artificial Sequence
  Description of Artificial Sequence Synthetic oligonucleotide
gggacggtcggtagatctcagcttcatgcagggc

SEQ ID NO: 182 - DNA, 19 bp, Artificial Sequence
  Description of Artificial Sequence Synthetic oligonucleotide
cccactcagccctgctctt

SEQ ID NO: 183 - DNA, 36 bp, Artificial Sequence
  Description of Artificial Sequence Synthetic oligonucleotide
gacgatgccttcagcacagcatccttggcggtcttg

SEQ ID NO: 184 - DNA, 34 bp, Artificial Sequence
  Description of Artificial Sequence Synthetic oligonucleotide
gctggctcggtcaagactcagcttcatgcagggt

SEQ ID NO: 185 - DNA, 34 bp, Artificial Sequence
  Description of Artificial Sequence Synthetic oligonucleotide
gggacggtcggtagatctcctcattgcctccttc

SEQ ID NO: 186 - DNA, 21 bp, Artificial Sequence
  Description of Artificial Sequence Synthetic oligonucleotide
cacctcttttctccttctctt

SEQ ID NO: 187 - DNA, 36 bp, Artificial Sequence
  Description of Artificial Sequence Synthetic oligonucleotide
gacgatgccttcagcacacccaccccctctatctac

SEQ ID NO: 188 - DNA, 34 bp, Artificial Sequence
  Description of Artificial Sequence Synthetic oligonucleotide
gctggctcggtcaagactcctcattgcctccttt

SEQ ID NO: 189 - DNA, 34 bp, Artificial Sequence
  Description of Artificial Sequence Synthetic oligonucleotide
gggacggtcggtagatgtcccccacaagtcctcg

SEQ ID NO: 190 - DNA, 36 bp, Artificial Sequence
  Description of Artificial Sequence Synthetic oligonucleotide
gacgatgccttcagcacagacctgtaccctttaccc

SEQ ID NO: 191 - DNA, 18 bp, Artificial Sequence
  Description of Artificial Sequence Synthetic oligonucleotide
tgtttccctgtctgtttc

SEQ ID NO: 192 - DNA, 34 bp, Artificial Sequence
  Description of Artificial Sequence Synthetic oligonucleotide
gctggctcggtcaagagtcccccacaagtcctca

SEQ ID NO: 193 - DNA, 34 bp, Artificial Sequence
  Description of Artificial Sequence Synthetic oligonucleotide
gggacggtcggtagatgtgactcggtcctatacc

SEQ ID NO: 194 - DNA, 18 bp, Artificial Sequence
  Description of Artificial Sequence Synthetic oligonucleotide
gtgggctgtgattgtgtt

SEQ ID NO: 195 - DNA, 39 bp, Artificial Sequence
  Description of Artificial Sequence Synthetic oligonucleotide
gacgatgccttcagcacatctcgtcgtcgtagtagttgt

SEQ ID NO: 196 - DNA, 34 bp, Artificial Sequence
  Description of Artificial Sequence Synthetic oligonucleotide
gctggctcggtcaagagtgactcggtcctatact

SEQ ID NO: 197 - DNA, 34 bp, Artificial Sequence
  Description of Artificial Sequence Synthetic oligonucleotide
gggacggtcggtagatagtatggtaattaggaag

SEQ ID NO: 198 - DNA, 36 bp, Artificial Sequence
  Description of Artificial Sequence Synthetic oligonucleotide
gacgatgccttcagcacactgacactgagccacaac

SEQ ID NO: 199 - DNA, 19 bp, Artificial Sequence
  Description of Artificial Sequence Synthetic oligonucleotide
aactgatgagcaagaagga

SEQ ID NO: 200 - DNA, 34 bp, Artificial Sequence
  Description of Artificial Sequence Synthetic oligonucleotide
gctggctcggtcaagaagtatggtaattaggaaa

SEQ ID NO: 201 - DNA, 34 bp, Artificial Sequence
  Description of Artificial Sequence Synthetic oligonucleotide
gggacggtccgtagataaaattgtttcctgtgat

SEQ ID NO: 202 - DNA, 39 bp, Artificial Sequence
  Description of Artificial Sequence Synthetic oligonucleotide
gacgatgccttcagcacacattgctattctcaggctata

SEQ ID NO: 203 - DNA, 20 bp, Artificial Sequence
  Description of Artificial Sequence Synthetic oligonucleotide
cccattctctgcttgacagt

SEQ ID NO: 204 - DNA, 34 bp, Artificial Sequence
  Description of Artificial Sequence Synthetic oligonucleotide
gctggctcggtcaagaaaaattgtttcctgtgac

SEQ ID NO: 205 - DNA, 18 bp, Artificial Sequence
  Description of Artificial Sequence Synthetic oligonucleotide
ccagagaggggataaaga

SEQ ID NO: 206 - DNA, 37 bp, Artificial Sequence
  Description of Artificial Sequence Synthetic oligonucleotide
gacgatgccttcagcacagagtgtcaagaggaacagg

SEQ ID NO: 207 - DNA, 34 bp, Artificial Sequence
  Description of Artificial Sequence Synthetic oligonucleotide
gggacggtcggtagattggctgctgaggtctgag

SEQ ID NO: 208 - DNA, 34 bp, Artificial Sequence
  Description of Artificial Sequence Synthetic oligonucleotide
gctggctcggtcaagatggctgctgaggtctgat

SEQ ID NO: 209 - DNA, 21 bp, Artificial Sequence
  Description of Artificial Sequence Synthetic oligonucleotide
gctttttcttttcattacatc

SEQ ID NO: 210 - DNA, 39 bp, Artificial Sequence
  Description of Artificial Sequence Synthetic oligonucleotide
gacgatgccttcagcacacctcttttagaatcagagaca

SEQ ID NO: 211 - DNA, 34 bp, Artificial Sequence
  Description of Artificial Sequence Synthetic oligonucleotide
gggacggtcggtagatggtagtgttaccagaaag

SEQ ID NO: 212 - DNA, 34 bp, Artificial Sequence
  Description of Artificial Sequence Synthetic oligonucleotide
gctggctcggtcaagaggtagtgttaccagaaat

SEQ ID NO: 213 - DNA, 34 bp, Artificial Sequence
  Description of Artificial Sequence Synthetic oligonucleotide
gggacggtcggtagattgtgcaccgggatatttt

SEQ ID NO: 214 - DNA, 36 bp, Artificial Sequence
  Description of Artificial Sequence Synthetic oligonucleotide
gacgatgccttcagcacaatgtgtgcttgggttctt

SEQ ID NO: 215 - DNA, 19 bp, Artificial Sequence
  Description of Artificial Sequence Synthetic oligonucleotide
ggtgtttctcctcctctct

SEQ ID NO: 216 - DNA, 34 bp, Artificial Sequence
  Description of Artificial Sequence Synthetic oligonucleotide
gctggctcggtcaagatgtgcaccgggatatttc

SEQ ID NO: 217 - DNA, 34 bp, Artificial Sequence
  Description of Artificial Sequence Synthetic oligonucleotide
gggacggtcggtagatgtgggcaccaaacgctat

SEQ ID NO: 218 - DNA, 36 bp, Artificial Sequence
  Description of Artificial Sequence Synthetic oligonucleotide
gacgatgccttcagcacagatgtagggctggaagtg

SEQ ID NO: 219 - DNA, 19 bp, Artificial Sequence
  Description of Artificial Sequence Synthetic oligonucleotide
tcaagaaaaatgggagttg

SEQ ID NO: 220 - DNA, 34 bp, Artificial Sequence
  Description of Artificial Sequence Synthetic oligonucleotide
gctggctcggtcaagagtgggcaccaaacgctac

SEQ ID NO: 221 - DNA, 18 bp, Artificial Sequence
  Description of Artificial Sequence Synthetic oligonucleotide
tgtagcatcggtaggttc

SEQ ID NO: 222 - DNA, 21 bp, Artificial Sequence
  Description of Artificial Sequence Synthetic oligonucleotide
caacatcagactttctttttc

SEQ ID NO: 223 - DNA, 34 bp, Artificial Sequence
  Description of Artificial Sequence Synthetic oligonucleotide
gggacggtcggtagattggtacagggctagtttt

SEQ ID NO: 224 - DNA, 34 bp, Artificial Sequence
  Description of Artificial Sequence Synthetic oligonucleotide
gctggctcggtcaagatggtacagggctagtttc

SEQ ID NO: 225 - DNA, 34 bp, Artificial Sequence
  Description of Artificial Sequence Synthetic oligonucleotide
gggacggtcggtagataggcgggccaagggtgaa

SEQ ID NO: 226 - DNA, 18 bp, Artificial Sequence
  Description of Artificial Sequence Synthetic oligonucleotide
ttctctctccccttctgt

SEQ ID NO: 227 - DNA, 39 bp, Artificial Sequence
  Description of Artificial Sequence Synthetic oligonucleotide
gacgatgccttcagcacataaatgttcactcttcttgct

SEQ ID NO: 228 - DNA, 34 bp, Artificial Sequence
  Description of Artificial Sequence Synthetic oligonucleotide
gctggctcggtcaagaaggcgggccaagggtgag

SEQ ID NO: 229 - DNA, 21 bp, Artificial Sequence
  Description of Artificial Sequence Synthetic oligonucleotide
gggttgttccagggcgctatt

SEQ ID NO: 230 - DNA, 36 bp, Artificial Sequence
  Description of Artificial Sequence Synthetic oligonucleotide
gacgatgccttcagcacatgtggagaggccgggtgc

SEQ ID NO: 231 - DNA, 34 bp, Artificial Sequence
  Description of Artificial Sequence Synthetic oligonucleotide
gggacggtcggtagatgaaccagccccctggaag

SEQ ID NO: 232 - DNA, 34 bp, Artificial Sequence
  Description of Artificial Sequence Synthetic oligonucleotide
gctggctcggtcaagagaaccagccccctggaat

SEQ ID NO: 233 - DNA, 34 bp, Artificial Sequence
  Description of Artificial Sequence Synthetic oligonucleotide
gggacggtcggtagatcaggcttggagacctggt

SEQ ID NO: 234 - DNA, 34 bp, Artificial Sequence
  Description of Artificial Sequence Synthetic oligonucleotide
gctggctcggtcaagacaggcttggagacctggg

SEQ ID NO: 235 - DNA, 36 bp, Artificial Sequence
  Description of Artificial Sequence Synthetic oligonucleotide
gacgatgccttcagcacagggtattcagttggaagg

SEQ ID NO: 236 - DNA, 19 bp, Artificial Sequence
  Description of Artificial Sequence Synthetic oligonucleotide
aaggcaaggttcttagttg

SEQ ID NO: 237 - DNA, 34 bp, Artificial Sequence
  Description of Artificial Sequence Synthetic oligonucleotide
gggacggtcggtagattctaaaagcacttgaaat

SEQ ID NO: 238 - DNA, 34 bp, Artificial Sequence
  Description of Artificial Sequence Synthetic oligonucleotide
gctggctcggtcaagatctaaaagcacttgaaag

SEQ ID NO: 239 - DNA, 36 bp, Artificial Sequence
  Description of Artificial Sequence Synthetic oligonucleotide
gacgatgccttcagcacacctgctagtgttttctgg

SEQ ID NO: 240 - DNA, 19 bp, Artificial Sequence
  Description of Artificial Sequence Synthetic oligonucleotide
tgtaactgataggtggtgg

SEQ ID NO: 241 - DNA, 34 bp, Artificial Sequence
  Description of Artificial Sequence Synthetic oligonucleotide
gggacggtccgtagattgaagattctgctcagcg

SEQ ID NO: 242 - DNA, 34 bp, Artificial Sequence
  Description of Artificial Sequence Synthetic oligonucleotide
gacgatgccttcagcacaagggcccgggactcat

SEQ ID NO: 243 - DNA, 18 bp, Artificial Sequence
  Description of Artificial Sequence Synthetic oligonucleotide
tttggggtcctgcggatg

SEQ ID NO: 244 - DNA, 34 bp, Artificial Sequence
  Description of Artificial Sequence Synthetic oligonucleotide
gctggctcggtcaagatgaagattctgctcagca

SEQ ID NO: 245 - DNA, 34 bp, Artificial Sequence
  Description of Artificial Sequence Synthetic oligonucleotide
gggacggtcggtagatcaaagaagacgaaaatga

SEQ ID NO: 246 - DNA, 23 bp, Artificial Sequence
  Description of Artificial Sequence Synthetic oligonucleotide
ctcaagtttgttactgatttctc

SEQ ID NO: 247 - DNA, 36 bp, Artificial Sequence
  Description of Artificial Sequence Synthetic oligonucleotide
gacgatgccttcagcacagggttacgtctgctcttc

SEQ ID NO: 248 - DNA, 34 bp, Artificial Sequence
  Description of Artificial Sequence Synthetic oligonucleotide
gctggctcggtcaagacaaagaagacgaaaatgg

SEQ ID NO: 249 - DNA, 36 bp, Artificial Sequence
  Description of Artificial Sequence Synthetic oligonucleotide
gacgatgccttcagcacactgctccgaaacacggtc

SEQ ID NO: 250 - DNA, 23 bp, Artificial Sequence
  Description of Artificial Sequence Synthetic oligonucleotide
gcatcttcagcccttcttactct

SEQ ID NO: 251 - DNA, 34 bp, Artificial Sequence
  Description of Artificial Sequence Synthetic oligonucleotide
gggacggtcggtagatctcctgggcaccacgggc

SEQ ID NO: 252 - DNA, 34 bp, Artificial Sequence
  Description of Artificial Sequence Synthetic oligonucleotide
gctggctcggtcaagactcctgggcaccacggga

SEQ ID NO: 253 - DNA, 36 bp, Artificial Sequence Sequence
  Description of Artificial Sequence Synthetic oligonucleotide
gacgatgccttcagcacatgggattagacacgagag

SEQ ID NO: 254 - DNA, 19 bp, Artificial Sequence Sequence
  Description of Artificial Sequence Synthetic oligonucleotide
aaagaactggaagaaggaa

SEQ ID NO: 255 - DNA, 34 bp, Artificial Sequence Sequence
  Description of Artificial Sequence Synthetic oligonucleotide
gggacggtcggtagatgtcacctcctttccacta

SEQ ID NO: 256 - DNA, 34 bp, Artificial Sequence Sequence
  Description of Artificial Sequence Synthetic oligonucleotide
gctggctcggtcaagagtcacctcctttccactc

SEQ ID NO: 257 - DNA, 38 bp, Artificial Sequence Sequence
  Description of Artificial Sequence Synthetic oligonucleotide
gacgatgccttcagcacaagaaaaatgagagggaaaac

SEQ ID NO: 258 - DNA, 18 bp, Artificial Sequence Sequence
  Description of Artificial Sequence Synthetic oligonucleotide
gatgaagggaaatggaac

SEQ ID NO: 259 - DNA, 34 bp, Artificial Sequence
  Description of Artificial Sequence Synthetic oligonucleotide
gggacggtcggtagatccaactatataggagccg

SEQ ID NO: 260 - DNA, 34 bp, Artificial Sequence
  Description of Artificial Sequence Synthetic oligonucleotide
gctggctcggtcaagaccaactatataggagcct

SEQ ID NO: 261 - DNA, 19 bp, Artificial Sequence
  Description of Artificial Sequence Synthetic oligonucleotide
ctgaaccgaggagattttt

SEQ ID NO: 262 - DNA, 19 bp, Artificial Sequence
  Description of Artificial Sequence Synthetic oligonucleotide
tgatgcttacagaactggg

SEQ ID NO: 263 - DNA, 34 bp, Artificial Sequence
  Description of Artificial Sequence Synthetic oligonucleotide
gggacggtcggtagatgtgtagtgggcagggtta

SEQ ID NO: 264 - DNA, 34 bp, Artificial Sequence
  Description of Artificial Sequence Synthetic oligonucleotide
gctggctcggtcaagagtgtagtgggcagggttg

SEQ ID NO: 265 - DNA, 36 bp, Artificial Sequence
  Description of Artificial Sequence Synthetic oligonucleotide
gacgatgccttcagcacaaaaagaaccctggtgaag

SEQ ID NO: 266 - DNA, 19 bp, Artificial Sequence
  Description of Artificial Sequence Synthetic oligonucleotide
ccctgataaaagagatgga

SEQ ID NO: 267 - DNA, 34 bp, Artificial Sequence
  Description of Artificial Sequence Synthetic oligonucleotide
gggacggtcggtagatcgcatgggagtcagggat

SEQ ID NO: 268 - DNA, 34 bp, Artificial Sequence
  Description of Artificial Sequence Synthetic oligonucleotide
gctggctcggtcaagacgcatgggagtcagggag

SEQ ID NO: 269 - DNA, 36 bp, Artificial Sequence
  Description of Artificial Sequence Synthetic oligonucleotide
gacgatgccttcagcacaatgagggagcaagacaag

SEQ ID NO: 270 - DNA, 21 bp, Artificial Sequence
  Description of Artificial Sequence Synthetic oligonucleotide
tgataaaagagatggaaggag

SEQ ID NO: 271 - DNA, 34 bp, Artificial Sequence
  Description of Artificial Sequence Synthetic oligonucleotide
gggacggtcggtagatgtcactgtttgtcactgt

SEQ ID NO: 272 - DNA, 34 bp, Artificial Sequence
  Description of Artificial Sequence Synthetic oligonucleotide
gctggctcggtcaagagtcactgtttgtcactgc

SEQ ID NO: 273 - DNA, 36 bp, Artificial Sequence
  Description of Artificial Sequence Synthetic oligonucleotide
gacgatgccttcagcacactttttagccaagtggag

SEQ ID NO: 274 - DNA, 18 bp, Artificial Sequence
  Description of Artificial Sequence Synthetic oligonucleotide
ggatctgaggaatctgtg

SEQ ID NO: 275 - DNA, 34 bp, Artificial Sequence
  Description of Artificial Sequence Synthetic oligonucleotide
gggacggtcggtagataccaggcagagagaaaat

SEQ ID NO: 276 - DNA, 34 bp, Artificial Sequence
  Description of Artificial Sequence Synthetic oligonucleotide
gctggctcggtcaagaaccaggcagagagaaaaa

SEQ ID NO: 277 - DNA, 21 bp, Artificial Sequence
  Description of Artificial Sequence Synthetic oligonucleotide
cttcactgagcgtccgcagag

SEQ ID NO: 278 - DNA, 16 bp, Artificial Sequence
  Description of Artificial Sequence Synthetic oligonucleotide
ccgtcggcccgattca

SEQ ID NO: 279 - DNA, 18 bp, Artificial Sequence
  Description of Artificial Sequence Synthetic oligonucleotide
caggcgagcctcagccct

SEQ ID NO: 280 - DNA, 18 bp, Artificial Sequence
  Description of Artificial Sequence Synthetic oligonucleotide
caggcgagcctcagcccc

SEQ ID NO: 281 - DNA, 20 bp, Artificial Sequence
  Description of Artificial Sequence Synthetic oligonucleotide
tcatttcccaatttacctcc

SEQ ID NO: 282 - DNA, 18 bp, Artificial Sequence
  Description of Artificial Sequence Synthetic oligonucleotide
cctctttcccatctccct

SEQ ID NO: 283 - DNA, 34 bp, Artificial Sequence
  Description of Artificial Sequence Synthetic oligonucleotide
gggacggtcggtagatagccaggagcctgcgtca

SEQ ID NO: 284 - DNA, 34 bp, Artificial Sequence
  Description of Artificial Sequence Synthetic oligonucleotide
gctggctcggtcaagaagccaggagcctgcgtcc

SEQ ID NO: 285 - DNA, 19 bp, Artificial Sequence
  Description of Artificial Sequence Synthetic oligonucleotide
cattgctcttcctctctgt

SEQ ID NO: 286 - DNA, 20 bp, Artificial Sequence
  Description of Artificial Sequence Synthetic oligonucleotide
gtgtcatcattcctttcttg

SEQ ID NO: 287 - DNA, 34 bp, Artificial Sequence
  Description of Artificial Sequence Synthetic oligonucleotide
gggacggtcggtagattcagagacatgagtccat

SEQ ID NO: 288 - DNA, 34 bp, Artificial Sequence
  Description of Artificial Sequence Synthetic oligonucleotide
gctggctcggtcaagatcagagacatgagtccac

SEQ ID NO: 289 - DNA, 37 bp, Artificial Sequence
  Description of Artificial Sequence Synthetic oligonucleotide
gacgatgccttcagcacacacctgtcccaccctattt

SEQ ID NO: 290 - DNA, 18 bp, Artificial Sequence
  Description of Artificial Sequence Synthetic oligonucleotide
gtcctgaacccccattct

SEQ ID NO: 291 - DNA, 34 bp, Artificial Sequence
  Description of Artificial Sequence Synthetic oligonucleotide
gggacggtcggtagatgcctgcactgcgttccta

SEQ ID NO: 292 - DNA, 34 bp, Artificial Sequence
  Description of Artificial Sequence Synthetic oligonucleotide
gctggctcggtcaagagcctgcactgcgttcctg

SEQ ID NO: 293 - DNA, 19 bp, Artificial Sequence
  Description of Artificial Sequence Synthetic oligonucleotide
gctcctctgccttctgctt

SEQ ID NO: 294 - DNA, 18 bp, Artificial Sequence
  Description of Artificial Sequence Synthetic oligonucleotide
atttgcccactgcccttc

SEQ ID NO: 295 - DNA, 34 bp, Artificial Sequence
  Description of Artificial Sequence Synthetic oligonucleotide
gggacggtcggtagattggctgcaggtgcgtcca

SEQ ID NO: 296 - DNA, 34 bp, Artificial Sequence
  Description of Artificial Sequence Synthetic oligonucleotide
gctggctcggtcaagatggctgcaggtgcgtccg

SEQ ID NO: 297 - DNA, 18 bp, Artificial Sequence
  Description of Artificial Sequence Synthetic oligonucleotide
gccacacacaccttaaca

SEQ ID NO: 298 - DNA, 18 bp, Artificial Sequence
  Description of Artificial Sequence Synthetic oligonucleotide
aaagttctctgcctccaa

SEQ ID NO: 299 - DNA, 34 bp, Artificial Sequence
  Description of Artificial Sequence Synthetic oligonucleotide
gggacggtcggtagatagctctcagctggggtgc

SEQ ID NO: 300 - DNA, 34 bp, Artificial Sequence
  Description of Artificial Sequence Synthetic oligonucleotide
gctggctcggtcaagaagctctcagctggggtgt

SEQ ID NO: 301 - DNA, 18 bp, Artificial Sequence
  Description of Artificial Sequence Synthetic oligonucleotide
agcagaatgggcaataga

SEQ ID NO: 302 - DNA, 18 bp, Artificial Sequence
  Description of Artificial Sequence Synthetic oligonucleotide
agagatgtgggcagagaa

SEQ ID NO: 303 - DNA, 34 bp, Artificial Sequence
  Description of Artificial Sequence Synthetic oligonucleotide
gggacggtcggtagatggaaagcctactttctta

SEQ ID NO: 304 - DNA, 34 bp, Artificial Sequence
  Description of Artificial Sequence Synthetic oligonucleotide
gctggctcggtcaagaggaaagcctactttcttg

SEQ ID NO: 305 - DNA, 18 bp, Artificial Sequence
  Description of Artificial Sequence Synthetic oligonucleotide
acagccataacaggagtg

SEQ ID NO: 306 - DNA, 20 bp, Artificial Sequence
  Description of Artificial Sequence Synthetic oligonucleotide
gggttactcaacctaagaga

SEQ ID NO: 307 - DNA, 34 bp, Artificial Sequence
  Description of Artificial Sequence Synthetic oligonucleotide
gggacggtcggtagatgttctctttgggaaaacg

SEQ ID NO: 308 - DNA, 34 bp, Artificial Sequence
  Description of Artificial Sequence Synthetic oligonucleotide
gctggctcggtcaagagttctctttgggaaaaca

SEQ ID NO: 309 - DNA, 37 bp, Artificial Sequence
  Description of Artificial Sequence Synthetic oligonucleotide
gacgatgccttcagcacagaacagaaaccacagaacc

SEQ ID NO: 310 - DNA, 18 bp, Artificial Sequence
  Description of Artificial Sequence Synthetic oligonucleotide
gtcccaccctattttgag

SEQ ID NO: 311 - DNA, 34 bp, Artificial Sequence
  Description of Artificial Sequence Synthetic oligonucleotide
gggacggtcggtagatcactggcccacctccctt

SEQ ID NO: 312 - DNA, 34 bp, Artificial Sequence
  Description of Artificial Sequence Synthetic oligonucleotide
gctggctcggtcaagacactggcccacctccctc

SEQ ID NO: 313 - DNA, 36 bp, Artificial Sequence
  Description of Artificial Sequence Synthetic oligonucleotide
gacgatgccttcagcacaaccatgcctgacttaacc

SEQ ID NO: 314 - DNA, 19 bp, Artificial Sequence
  Description of Artificial Sequence Synthetic oligonucleotide
ttgtttcctgtcctctttc

SEQ ID NO: 315 - DNA, 34 bp, Artificial Sequence
  Description of Artificial Sequence Synthetic oligonucleotide
gggacggtcggtagatgttaagaggctgggcagt

SEQ ID NO: 316 - DNA, 34 bp, Artificial Sequence
  Description of Artificial Sequence Synthetic oligonucleotide
gctggctcggtcaagagttaagaggctgggcagc

SEQ ID NO: 317 - DNA, 37 bp, Artificial Sequence
  Description of Artificial Sequence Synthetic oligonucleotide
gacgatgccttcagcacaagtgttgttaggagcaaag

SEQ ID NO: 318 - DNA, 18 bp, Artificial Sequence
  Description of Artificial Sequence Synthetic oligonucleotide
cttaggaaactgaggtgg

SEQ ID NO: 319 - DNA, 34 bp, Artificial Sequence
  Description of Artificial Sequence Synthetic oligonucleotide
gggacggtcggtagatctgcagcctgggcaacag

SEQ ID NO: 320 - DNA, 34 bp, Artificial Sequence
  Description of Artificial Sequence Synthetic oligonucleotide
gctggctcggtcaagactgcagcctgggcaacac

SEQ ID NO: 321 - DNA, 36 bp, Artificial Sequence
  Description of Artificial Sequence Synthetic oligonucleotide
gacgatgccttcagcacatggacacatttgagcttt

SEQ ID NO: 322 - DNA, 19 bp, Artificial Sequence
  Description of Artificial Sequence Synthetic oligonucleotide
cttccccagagatgactac

SEQ ID NO: 323 - DNA, 34 bp, Artificial Sequence
  Description of Artificial Sequence Synthetic oligonucleotide
gggacggtcggtagatccccatcctactcagcac

SEQ ID NO: 324 - DNA, 34 bp, Artificial Sequence
  Description of Artificial Sequence Synthetic oligonucleotide
gctggctcggtcaagaccccatcctactcagcat

SEQ ID NO: 325 - DNA, 36 bp, Artificial Sequence
  Description of Artificial Sequence Synthetic oligonucleotide
gacgatgccttcagcacaggttacagtgagccaaga

SEQ ID NO: 326 - DNA, 20 bp, Artificial Sequence
  Description of Artificial Sequence Synthetic oligonucleotide
aggtgaagaaagcaaaatac

SEQ ID NO: 327 - DNA, 34 bp, Artificial Sequence
  Description of Artificial Sequence Synthetic oligonucleotide
gggacggtcggtagattggtgtgtgttttgtttc

SEQ ID NO: 328 - DNA, 34 bp, Artificial Sequence
  Description of Artificial Sequence Synthetic oligonucleotide
gctggctcggtcaagatggtgtgtgttttgtttt

SEQ ID NO: 329 - DNA, 19 bp, Artificial Sequence
  Description of Artificial Sequence Synthetic oligonucleotide
ttgagaccctacagagcca

SEQ ID NO: 330 - DNA, 18 bp, Artificial Sequence
  Description of Artificial Sequence Synthetic oligonucleotide
ggcaagctgaggtgaaag

SEQ ID NO: 331 - DNA, 34 bp, Artificial Sequence
  Description of Artificial Sequence Synthetic oligonucleotide
gggacggtcggtagataataaggtaagaaatgag

SEQ ID NO: 332 - DNA, 34 bp, Artificial Sequence
  Description of Artificial Sequence Synthetic oligonucleotide
gctggctcggtcaagaaataaggtaagaaatgac

SEQ ID NO: 333 - DNA, 19 bp, Artificial Sequence
  Description of Artificial Sequence Synthetic oligonucleotide
taatttctaatggccttcc

SEQ ID NO: 334 - DNA, 20 bp, Artificial Sequence
  Description of Artificial Sequence Synthetic oligonucleotide
tcacttactccctgatgtct

SEQ ID NO: 335 - DNA, 34 bp, Artificial Sequence
  Description of Artificial Sequence Synthetic oligonucleotide
gggacggtcggtagatcattgggttttccctcat

SEQ ID NO: 336 - DNA, 34 bp, Artificial Sequence
  Description of Artificial Sequence Synthetic oligonucleotide
gctggctcggtcaagacattgggttttccctcac

SEQ ID NO: 337 - DNA, 38 bp, Artificial Sequence
  Description of Artificial Sequence Synthetic oligonucleotide
gacgatgccttcagcacaacatttagtgccaacatcac

SEQ ID NO: 338 - DNA, 18 bp, Artificial Sequence
  Description of Artificial Sequence Synthetic oligonucleotide
ctcttccctgagacacca

SEQ ID NO: 339 - DNA, 34 bp, Artificial Sequence
  Description of Artificial Sequence Synthetic oligonucleotide
gggacggtcggtagatgaaggtgaaggccagagc

SEQ ID NO: 340 - DNA, 34 bp, Artificial Sequence
  Description of Artificial Sequence Synthetic oligonucleotide
gctggctcggtcaagagaaggtgaaggccagagt

SEQ ID NO: 341 - DNA, 18 bp, Artificial Sequence
  Description of Artificial Sequence Synthetic oligonucleotide
gaggctgagacagaagaa

SEQ ID NO: 342 - DNA, 22 bp, Artificial Sequence
  Description of Artificial Sequence Synthetic oligonucleotide
gtttgacattaaagaaaatgag

SEQ ID NO: 343 - DNA, 34 bp, Artificial Sequence
  Description of Artificial Sequence Synthetic oligonucleotide
gggacggtcggtagatggctggagtgcagtgatt

SEQ ID NO: 344 - DNA, 34 bp, Artificial Sequence
  Description of Artificial Sequence Synthetic oligonucleotide
gctggctcggtcaagaggctggagtgcagtgatg

SEQ ID NO: 345 - DNA, 16 bp, Artificial Sequence
  Description of Artificial Sequence Synthetic oligonucleotide
cacgctgttgagtggg

SEQ ID NO: 346 - DNA, 16 bp, Artificial Sequence
  Description of Artificial Sequence Synthetic oligonucleotide
cgcaggtctacggtca

SEQ ID NO: 347 - DNA, 34 bp, Artificial Sequence
  Description of Artificial Sequence Synthetic oligonucleotide
gggacggtcggtagatcccgggtctgaggctgcg

SEQ ID NO: 348 - DNA, 34 bp, Artificial Sequence
  Description of Artificial Sequence Synthetic oligonucleotide
gctggctcggtcaagacccgggtctgaggctgcc

SEQ ID NO: 349 - DNA, 19 bp, Artificial Sequence
  Description of Artificial Sequence Synthetic oligonucleotide
cacacacacacacacacac

SEQ ID NO: 350 - DNA, 19 bp, Artificial Sequence
  Description of Artificial Sequence Synthetic oligonucleotide
ggtcccttactttcctctt

SEQ ID NO: 351 - DNA, 34 bp, Artificial Sequence
  Description of Artificial Sequence Synthetic oligonucleotide
gggacggtcggtagatcctatccctacttcccct

SEQ ID NO: 352 - DNA, 34 bp, Artificial Sequence
  Description of Artificial Sequence Synthetic oligonucleotide
gctggctcggtcaagacctatccctacttccccc

SEQ ID NO: 353 - DNA, 18 bp, Artificial Sequence
  Description of Artificial Sequence Synthetic oligonucleotide
gtgaccccaaaagagaga

SEQ ID NO: 354 - DNA, 19 bp, Artificial Sequence
  Description of Artificial Sequence Synthetic oligonucleotide
ctagcttgttactgcctcc

SEQ ID NO: 355 - DNA, 34 bp, Artificial Sequence
  Description of Artificial Sequence Synthetic oligonucleotide
gggacggtcggtagatggcacgaccccgccccca

SEQ ID NO: 356 - DNA, 34 bp, Artificial Sequence
  Description of Artificial Sequence Synthetic oligonucleotide
gctggctcggtcaagaggcacgaccccgcccccg

SEQ ID NO: 357 - DNA, 19 bp, Artificial Sequence
  Description of Artificial Sequence Synthetic oligonucleotide
tccacaacctcaaaaccac

SEQ ID NO: 358 - DNA, 18 bp, Artificial Sequence
  Description of Artificial Sequence Synthetic oligonucleotide
cacagtcctgcaagctca

SEQ ID NO: 359 - DNA, 34 bp, Artificial Sequence
  Description of Artificial Sequence Synthetic oligonucleotide
gggacggtcggtagatccgtggccgtggctcact

SEQ ID NO: 360 - DNA, 34 bp, Artificial Sequence
  Description of Artificial Sequence Synthetic oligonucleotide
gctggctcggtcaagaccgtggccgtggctcacc

SEQ ID NO: 361 - DNA, 34 bp, Artificial Sequence
  Description of Artificial Sequence Synthetic oligonucleotide
gacgatgccttcagcacagttcggggctccactt

SEQ ID NO: 362 - DNA, 16 bp, Artificial Sequence
  Description of Artificial Sequence Synthetic oligonucleotide
tagcgggacagcgctg

SEQ ID NO: 363 - DNA, 34 bp, Artificial Sequence
  Description of Artificial Sequence Synthetic oligonucleotide
gggacggtcggtagatcccggcgcgcctcggaga

SEQ ID NO: 364 - DNA, 34 bp, Artificial Sequence
  Description of Artificial Sequence Synthetic oligonucleotide
gctggctcggtcaagacccggcgcgcctcggagt

SEQ ID NO: 365 - DNA, 36 bp, Artificial Sequence
  Description of Artificial Sequence Synthetic oligonucleotide
gacgatgccttcagcacaaatacactgggtcctgct

SEQ ID NO: 366 - DNA, 18 bp, Artificial Sequence
  Description of Artificial Sequence Synthetic oligonucleotide
atactgctggcctttctc

SEQ ID NO: 367 - DNA, 34 bp, Artificial Sequence
  Description of Artificial Sequence Synthetic oligonucleotide
gggacggtcggtagatggtcaggggagcccagag

SEQ ID NO: 368 - DNA, 34 bp, Artificial Sequence
  Description of Artificial Sequence Synthetic oligonucleotide
gctggctcggtcaagaggtcaggggagcccagaa

SEQ ID NO: 369 - DNA, 18 bp, Artificial Sequence
  Description of Artificial Sequence Synthetic oligonucleotide
cctggcaactaacctctt

SEQ ID NO: 370 - DNA, 21 bp, Artificial Sequence
  Description of Artificial Sequence Synthetic oligonucleotide
aggcagtctctctgtctactc

SEQ ID NO: 371 - DNA, 34 bp, Artificial Sequence
  Description of Artificial Sequence Synthetic oligonucleotide
gggacggtcggtagatattggccctgctcaggat

SEQ ID NO: 372 - DNA, 34 bp, Artificial Sequence
  Description of Artificial Sequence Synthetic oligonucleotide
gctggctcggtcaagaattggccctgctcaggac

SEQ ID NO: 373 - DNA, 17 bp, Artificial Sequence
  Description of Artificial Sequence Synthetic oligonucleotide
ccagccctaaacctaaa

SEQ ID NO: 374 - DNA, 20 bp, Artificial Sequence
  Description of Artificial Sequence Synthetic oligonucleotide
aacctctcaagatcagacac

SEQ ID NO: 375 - DNA, 34 bp, Artificial Sequence
  Description of Artificial Sequence Synthetic oligonucleotide
gggacggtcggtagatttagcacttaataagtac

SEQ ID NO: 376 - DNA, 34 bp, Artificial Sequence
  Description of Artificial Sequence Synthetic oligonucleotide
gctggctcggtcaagattagcacttaataagtat

SEQ ID NO: 377 - DNA, 18 bp, Artificial Sequence
  Description of Artificial Sequence Synthetic oligonucleotide
ccccacaacaaagaaaga

SEQ ID NO: 378 - DNA, 18 bp, Artificial Sequence
  Description of Artificial Sequence Synthetic oligonucleotide
gaagccaactctccaaca

SEQ ID NO: 379 - DNA, 34 bp, Artificial Sequence
  Description of Artificial Sequence Synthetic oligonucleotide
gggacggtcggtagatcaaggatttcaaaaacca

SEQ ID NO: 380 - DNA, 34 bp, Artificial Sequence
  Description of Artificial Sequence Synthetic oligonucleotide
gctggctcggtcaagacaaggatttcaaaaaccg

SEQ ID NO: 381 - DNA, 36 bp, Artificial Sequence
  Description of Artificial Sequence Synthetic oligonucleotide
gacgatgccttcagcacaccagggaagagcagaacc

SEQ ID NO: 382 - DNA, 18 bp, Artificial Sequence
  Description of Artificial Sequence Synthetic oligonucleotide
tgtacgggaagaggcaga

SEQ ID NO: 383 - DNA, 34 bp, Artificial Sequence
  Description of Artificial Sequence Synthetic oligonucleotide
gggacggtcggtagatagggtgacacaggccacg

SEQ ID NO: 384 - DNA, 34 bp, Artificial Sequence
  Description of Artificial Sequence Synthetic oligonucleotide
gctggctcggtcaagaagggtgacacaggccacc

SEQ ID NO: 385 - DNA, 18 bp, Artificial Sequence
  Description of Artificial Sequence Synthetic oligonucleotide
atcccatcccaacacaca

SEQ ID NO: 386 - DNA, 20 bp, Artificial Sequence
  Description of Artificial Sequence Synthetic oligonucleotide
ccgagaccaaactcattcac

SEQ ID NO: 387 - DNA, 34 bp, Artificial Sequence
  Description of Artificial Sequence Synthetic oligonucleotide
gggacggtcggtagatggcagagcctgagtcact

SEQ ID NO: 388 - DNA, 34 bp, Artificial Sequence
  Description of Artificial Sequence Synthetic oligonucleotide
gctggctcggtcaagaggcagagcctgagtcacc

SEQ ID NO: 389 - DNA, 19 bp, Artificial Sequence
  Description of Artificial Sequence Synthetic oligonucleotide
cctgtttctcaaccttctc

SEQ ID NO: 390 - DNA, 21 bp, Artificial Sequence
  Description of Artificial Sequence Synthetic oligonucleotide
atggtctatggaacctaatct

SEQ ID NO: 391 - DNA, 34 bp, Artificial Sequence
  Description of Artificial Sequence Synthetic oligonucleotide
gggacggtcggtagatgcactgattctgcttcca

SEQ ID NO: 392 - DNA, 34 bp, Artificial Sequence
  Description of Artificial Sequence Synthetic oligonucleotide
gctggctcggtcaagagcactgattctgcttccc

SEQ ID NO: 393 - DNA, 19 bp, Artificial Sequence
  Description of Artificial Sequence Synthetic oligonucleotide
aaggacagggtcaggaaag

SEQ ID NO: 394 - DNA, 18 bp, Artificial Sequence
  Description of Artificial Sequence Synthetic oligonucleotide
cagagggaggaaggaggt

SEQ ID NO: 395 - DNA, 34 bp, Artificial Sequence
  Description of Artificial Sequence Synthetic oligonucleotide
gggacggtcggtagatatggaggagggtgtctgg

SEQ ID NO: 396 - DNA, 34 bp, Artificial Sequence
  Description of Artificial Sequence Synthetic oligonucleotide
gctggctcggtcaagaatggaggagggtgtctgt

SEQ ID NO: 397 - DNA, 34 bp, Artificial Sequence
  Description of Artificial Sequence Synthetic oligonucleotide
gacgatgccttcagcacattcccaaagacccaca

SEQ ID NO: 398 - DNA, 16 bp, Artificial Sequence
  Description of Artificial Sequence Synthetic oligonucleotide
cctccaccgctatcac

SEQ ID NO: 399 - DNA, 34 bp, Artificial Sequence
  Description of Artificial Sequence Synthetic oligonucleotide
gggacggtcggtagattggctgcaggacgtccag

SEQ ID NO: 400 - DNA, 34 bp, Artificial Sequence
  Description of Artificial Sequence Synthetic oligonucleotide
gctggctcggtcaagatggctgcaggacgtccaa

SEQ ID NO: 401 - DNA, 16 bp, Artificial Sequence
  Description of Artificial Sequence Synthetic oligonucleotide
ttcccaaagacccaca

SEQ ID NO: 402 - DNA, 16 bp, Artificial Sequence
  Description of Artificial Sequence Synthetic oligonucleotide
cctccaccgctatcac

SEQ ID NO: 403 - DNA, 34 bp, Artificial Sequence; identical to SEQ ID NO: 399
  Description of Artificial Sequence Synthetic oligonucleotide

SEQ ID NO: 404 - DNA, 34 bp, Artificial Sequence; identical to SEQ ID NO: 400
  Description of Artificial Sequence Synthetic oligonucleotide

SEQ ID NO: 405 - DNA, 16 bp, Artificial Sequence
  Description of Artificial Sequence Synthetic oligonucleotide
cccaaccacccgttcc

SEQ ID NO: 406 - DNA, 16 bp, Artificial Sequence
  Description of Artificial Sequence Synthetic oligonucleotide
gcgcgggagctagaga

SEQ ID NO: 407 - DNA, 34 bp, Artificial Sequence
  Description of Artificial Sequence Synthetic oligonucleotide
gggacggtcggtagatgaagctgcgggccggacc

SEQ ID NO: 408 - DNA, 34 bp, Artificial Sequence
  Description of Artificial Sequence Synthetic oligonucleotide
gctggctcggtcaagagaagctgcgggccggacg

SEQ ID NO: 409 - DNA, 20 bp, Artificial Sequence
  Description of Artificial Sequence Synthetic oligonucleotide
cgagtgggaagaaaagtaga

SEQ ID NO: 410 - DNA, 18 bp, Artificial Sequence
  Description of Artificial Sequence Synthetic oligonucleotide
atgactgcctgcctagaa

SEQ ID NO: 411 - DNA, 34 bp, Artificial Sequence
  Description of Artificial Sequence Synthetic oligonucleotide
gggacggtcggtagataagatagggtagaggccg

SEQ ID NO: 412 - DNA, 34 bp, Artificial Sequence
  Description of Artificial Sequence Synthetic oligonucleotide
gctggctcggtcaagaaagatagggtagaggcca

SEQ ID NO: 413 - DNA, 20 bp, Artificial Sequence
  Description of Artificial Sequence Synthetic oligonucleotide
gaggagtgagggaaagtaag

SEQ ID NO: 414 - DNA, 18 bp, Artificial Sequence
  Description of Artificial Sequence Synthetic oligonucleotide
aaatggagagagatggga

SEQ ID NO: 415 - DNA, 34 bp, Artificial Sequence
  Description of Artificial Sequence Synthetic oligonucleotide
gggacggtcggtagatccaggaaatgacatgatc

SEQ ID NO: 416 - DNA, 34 bp, Artificial Sequence
  Description of Artificial Sequence Synthetic oligonucleotide
gctggctcggtcaagaccaggaaatgacatgatt

SEQ ID NO: 417 - DNA, 19 bp, Artificial Sequence
  Description of Artificial Sequence Synthetic oligonucleotide
tgagttgaacagcacttgg

SEQ ID NO: 418 - DNA, 18 bp, Artificial Sequence
  Description of Artificial Sequence Synthetic oligonucleotide
agggtaagggagggaaaa

SEQ ID NO: 419 - DNA, 34 bp, Artificial Sequence
  Description of Artificial Sequence Synthetic oligonucleotide
gggacggtcggtagattgattctttcgcttggcg

SEQ ID NO: 420 - DNA, 34 bp, Artificial Sequence
  Description of Artificial Sequence Synthetic oligonucleotide
gctggctcggtcaagatgattctttcgcttggca

SEQ ID NO: 421 - DNA, 20 bp, Artificial Sequence
  Description of Artificial Sequence Synthetic oligonucleotide
tagaaaagaagaaaaatcaa

SEQ ID NO: 422 - DNA, 18 bp, Artificial Sequence
  Description of Artificial Sequence Synthetic oligonucleotide
acacacacacacacacac

SEQ ID NO: 423 - DNA, 34 bp, Artificial Sequence
  Description of Artificial Sequence Synthetic oligonucleotide
gggacggtcggtagatcatcaccttttagtttct

SEQ ID NO: 424 - DNA, 34 bp, Artificial Sequence
  Description of Artificial Sequence Synthetic oligonucleotide
gctggctcggtcaagacatcaccttttagtttcc

SEQ ID NO: 425 - DNA, 21 bp, Artificial Sequence
  Description of Artificial Sequence Synthetic oligonucleotide
acagaagaacaacaacaaaac

SEQ ID NO: 426 - DNA, 19 bp, Artificial Sequence
  Description of Artificial Sequence Synthetic oligonucleotide
tgcgtatgaggtaaagaga

SEQ ID NO: 427 - DNA, 34 bp, Artificial Sequence
  Description of Artificial Sequence Synthetic oligonucleotide
gggacggtcggtagatatgagtgaagcctgtctc

SEQ ID NO: 428 - DNA, 34 bp, Artificial Sequence
  Description of Artificial Sequence Synthetic oligonucleotide
gctggctcggtcaagaatgagtgaagcctgtctg

SEQ ID NO: 429 - DNA, 21 bp, Artificial Sequence
  Description of Artificial Sequence Synthetic oligonucleotide
acagaagaacaacaacaaaac

SEQ ID NO: 430 - DNA, 19 bp, Artificial Sequence
  Description of Artificial Sequence Synthetic oligonucleotide
tgcgtatgaggtaaagaga

SEQ ID NO: 431 - DNA, 34 bp, Artificial Sequence
  Description of Artificial Sequence Synthetic oligonucleotide
gggacggtcggtagatggaccataatcttgaagt

SEQ ID NO: 432 - DNA, 34 bp, Artificial Sequence
  Description of Artificial Sequence Synthetic oligonucleotide
gctggctcggtcaagaggaccataatcttgaaga

SEQ ID NO: 433 - DNA, 21 bp, Artificial Sequence
  Description of Artificial Sequence Synthetic oligonucleotide
gcttgtcttgtctgataggtg

SEQ ID NO: 434 - DNA, 21 bp, Artificial Sequence
  Description of Artificial Sequence Synthetic oligonucleotide
caacgtgagaatttccaaaat

SEQ ID NO: 435 - DNA, 34 bp, Artificial Sequence
  Description of Artificial Sequence Synthetic oligonucleotide
gggacggtcggtagattgagaatttccaaaatag

SEQ ID NO: 436 - DNA, 34 bp, Artificial Sequence
  Description of Artificial Sequence Synthetic oligonucleotide
gctggctcggtcaagatgagaatttccaaaataa

SEQ ID NO: 437 - DNA, 19 bp, Artificial Sequence
  Description of Artificial Sequence Synthetic oligonucleotide
tacattcaaggcaagaaaa

SEQ ID NO: 438 - DNA, 27 bp, Artificial Sequence
  Description of Artificial Sequence Synthetic oligonucleotide
tgattagttacaattacctctagtatc

SEQ ID NO: 439 - DNA, 34 bp, Artificial Sequence
  Description of Artificial Sequence Synthetic oligonucleotide
gggacggtcggtagatagtttgtcagtaaatgta

SEQ ID NO: 440 - DNA, 34 bp, Artificial Sequence
  Description of Artificial Sequence Synthetic oligonucleotide
gctggctcggtcaagaagtttgtcagtaaatgtt

SEQ ID NO: 441 - DNA, 38 bp, Artificial Sequence
  Description of Artificial Sequence Synthetic oligonucleotide
gacgatgccttcagcacaagagagcagctagactgaga

SEQ ID NO: 442 - DNA, 18 bp, Artificial Sequence
  Description of Artificial Sequence Synthetic oligonucleotide
ttcctgcaaacagttgag

SEQ ID NO: 443 - DNA, 34 bp, Artificial Sequence
  Description of Artificial Sequence Synthetic oligonucleotide
gggacggtcggtagatagttgagggctcaggatt

SEQ ID NO: 444 - DNA, 34 bp, Artificial Sequence
  Description of Artificial Sequence Synthetic oligonucleotide
gctggctcggtcaagaagttgagggctcaggata

SEQ ID NO: 445 - DNA, 38 bp, Artificial Sequence; identical to SEQ ID NO: 441
  Description of Artificial Sequence Synthetic oligonucleotide

SEQ ID NO: 446 - DNA, 20 bp, Artificial Sequence
  Description of Artificial Sequence Synthetic oligonucleotide
gtaaataaaatgggatggtg

SEQ ID NO: 447 - DNA, 34 bp, Artificial Sequence
  Description of Artificial Sequence Synthetic oligonucleotide
gggacggtcggtagatgccccagcaagctgcatg

SEQ ID NO: 448 - DNA, 34 bp, Artificial Sequence
  Description of Artificial Sequence Synthetic oligonucleotide
gctggctcggtcaagagccccagcaagctgcatc

SEQ ID NO: 449 - DNA, 20 bp, Artificial Sequence
  Description of Artificial Sequence Synthetic oligonucleotide
ccttttgtgttttgttttgt

SEQ ID NO: 450 - DNA, 18 bp, Artificial Sequence
  Description of Artificial Sequence Synthetic oligonucleotide
cttctccaccttccattc

SEQ ID NO: 451 - DNA, 34 bp, Artificial Sequence
  Description of Artificial Sequence Synthetic oligonucleotide
gggacggtcggtagatgggaactcctaaatcaaa

SEQ ID NO: 452 - DNA, 34 bp, Artificial Sequence
  Description of Artificial Sequence Synthetic oligonucleotide
gctggctcggtcaagagggaactcctaaatcaag

SEQ ID NO: 453 - DNA, 36 bp, Artificial Sequence
  Description of Artificial Sequence Synthetic oligonucleotide
gacgatgccttcagcacaacgatggggtcagagtca

SEQ ID NO: 454 - DNA, 21 bp, Artificial Sequence
  Description of Artificial Sequence Synthetic oligonucleotide
cctacatttcacacacgaaca

SEQ ID NO: 455 - DNA, 34 bp, Artificial Sequence
  Description of Artificial Sequence Synthetic oligonucleotide
gggacggtcggtagatacacactcctctctcaag

SEQ ID NO: 456 - DNA, 34 bp, Artificial Sequence
  Description of Artificial Sequence Synthetic oligonucleotide
gctggctcggtcaagaacacactcctctctcaaa

SEQ ID NO: 457 - DNA, 16 bp, Artificial Sequence
  Description of Artificial Sequence Synthetic oligonucleotide
gccatcgtctttccct

SEQ ID NO: 458 - DNA, 18 bp, Artificial Sequence
  Description of Artificial Sequence Synthetic oligonucleotide
tcctccctccttctctct

SEQ ID NO: 459 - DNA, 34 bp, Artificial Sequence
  Description of Artificial Sequence Synthetic oligonucleotide
gggacggtcggtagatcctccacccaccagggcc

SEQ ID NO: 460 - DNA, 34 bp, Artificial Sequence
  Description of Artificial Sequence Synthetic oligonucleotide
gctggctcggtcaagacctccacccaccagggca

SEQ ID NO: 461 - DNA, 19 bp, Artificial Sequence
  Description of Artificial Sequence Synthetic oligonucleotide
cctctttctcctcctcttc

SEQ ID NO: 462 - DNA, 20 bp, Artificial Sequence
  Description of Artificial Sequence Synthetic oligonucleotide
ctcttcctgtcttctcctct

SEQ ID NO: 463 - DNA, 34 bp, Artificial Sequence
  Description of Artificial Sequence Synthetic oligonucleotide
gggacggtcggtagatagatggacctctacagga

SEQ ID NO: 464 - DNA, 34 bp, Artificial Sequence
  Description of Artificial Sequence Synthetic oligonucleotide
gctggctcggtcaagaagatggacctctacaggg

SEQ ID NO: 465 - DNA, 18 bp, Artificial Sequence
  Description of Artificial Sequence Synthetic oligonucleotide
ctcctccaactcctttac

SEQ ID NO: 466 - DNA, 19 bp, Artificial Sequence
  Description of Artificial Sequence Synthetic oligonucleotide
atacttctcactgcatcct

SEQ ID NO: 467 - DNA, 34 bp, Artificial Sequence
  Description of Artificial Sequence Synthetic oligonucleotide
gggacggtcggtagatcctgtcccctccctagtt

SEQ ID NO: 468 - DNA, 34 bp, Artificial Sequence
  Description of Artificial Sequence Synthetic oligonucleotide
gctggctcggtcaagacctgtcccctccctagtc

SEQ ID NO: 469 - DNA, 19 bp, Artificial Sequence
  Description of Artificial Sequence Synthetic oligonucleotide
caccttcctgaactcactc

SEQ ID NO: 470 - DNA, 18 bp, Artificial Sequence
  Description of Artificial Sequence Synthetic oligonucleotide
tgatgtctgtgctgtcct

SEQ ID NO: 471 - DNA, 34 bp, Artificial Sequence
  Description of Artificial Sequence Synthetic oligonucleotide
gggacggtcggtagattctggtccactcaaggag

SEQ ID NO: 472 - DNA, 34 bp, Artificial Sequence
  Description of Artificial Sequence Synthetic oligonucleotide
gctggctcggtcaagatctggtccactcaaggaa

SEQ ID NO: 473 - DNA, 18 bp, Artificial Sequence
  Description of Artificial Sequence Synthetic oligonucleotide
tcgggaggtgtaagtaag

SEQ ID NO: 474 - DNA, 19 bp, Artificial Sequence
  Description of Artificial Sequence Synthetic oligonucleotide
ccacagtcagaagagacaa

SEQ ID NO: 475 - DNA, 34 bp, Artificial Sequence
  Description of Artificial Sequence Synthetic oligonucleotide
gggacggtcggtagatagagaccctggtcccaag

SEQ ID NO: 476 - DNA, 34 bp, Artificial Sequence
  Description of Artificial Sequence Synthetic oligonucleotide
gctggctcggtcaagaagagaccctggtcccaaa

SEQ ID NO: 477 - DNA, 22 bp, Artificial Sequence
  Description of Artificial Sequence Synthetic oligonucleotide
tttatcactacaccccctactc

SEQ ID NO: 478 - DNA, 18 bp, Artificial Sequence
  Description of Artificial Sequence Synthetic oligonucleotide
gacagaccgaccaatcac

SEQ ID NO: 479 - DNA, 34 bp, Artificial Sequence
  Description of Artificial Sequence Synthetic oligonucleotide
gggacggtcggtagatccctgggaaggttgagag

SEQ ID NO: 480 - DNA, 34 bp, Artificial Sequence
  Description of Artificial Sequence Synthetic oligonucleotide
gctggctcggtcaagaccctgggaaggttgagaa

SEQ ID NO: 481 - DNA, 18 bp, Artificial Sequence
  Description of Artificial Sequence Synthetic oligonucleotide
ctgtctgtttgggtcttc

SEQ ID NO: 482 - DNA, 18 bp, Artificial Sequence
  Description of Artificial Sequence Synthetic oligonucleotide
cgttgttctctgtccact

SEQ ID NO: 483 - DNA, 34 bp, Artificial Sequence
  Description of Artificial Sequence Synthetic oligonucleotide
gggacggtcggtagatggccaaatgtctaaaagt

SEQ ID NO: 484 - DNA, 34 bp, Artificial Sequence
  Description of Artificial Sequence Synthetic oligonucleotide
gctggctcggtcaagaggccaaatgtctaaaagc

SEQ ID NO: 485 - DNA, 19 bp, Artificial Sequence
  Description of Artificial Sequence Synthetic oligonucleotide
cgtatctcttgcctttctt

SEQ ID NO: 486 - DNA, 18 bp, Artificial Sequence
  Description of Artificial Sequence Synthetic oligonucleotide
cttctcttatgccttccc

SEQ ID NO: 487 - DNA, 34 bp, Artificial Sequence
  Description of Artificial Sequence Synthetic oligonucleotide
gggacggtcggtagatttacttgaaaggacacca

SEQ ID NO: 488 - DNA, 34 bp, Artificial Sequence
  Description of Artificial Sequence Synthetic oligonucleotide
gctggctcggtcaagattacttgaaaggacaccg

SEQ ID NO: 489 - DNA, 19 bp, Artificial Sequence
  Description of Artificial Sequence Synthetic oligonucleotide
cgtatctcttgcctttctt

SEQ ID NO: 490 - DNA, 18 bp, Artificial Sequence
  Description of Artificial Sequence Synthetic oligonucleotide
cttctcttatgccttccc

SEQ ID NO: 491 - DNA, 34 bp, Artificial Sequence
  Description of Artificial Sequence Synthetic oligonucleotide
gggacggtcggtagatttctgcactaaagctgta

SEQ ID NO: 492 - DNA, 34 bp, Artificial Sequence
  Description of Artificial Sequence Synthetic oligonucleotide
gctggctcggtcaagattctgcactaaagctgtc

SEQ ID NO: 493 - DNA, 18 bp, Artificial Sequence
  Description of Artificial Sequence Synthetic oligonucleotide
tgggaagaaaaagagaag

SEQ ID NO: 494 - DNA, 18 bp, Artificial Sequence
  Description of Artificial Sequence Synthetic oligonucleotide
gttgaaacactgcacaag

SEQ ID NO: 495 - DNA, 34 bp, Artificial Sequence
  Description of Artificial Sequence Synthetic oligonucleotide
gggacggtcggtagatcagggctgttgggtgaag

SEQ ID NO: 496 - DNA, 34 bp, Artificial Sequence
  Description of Artificial Sequence Synthetic oligonucleotide
gctggctcggtcaagacagggctgttgggtgaaa

SEQ ID NO: 497 - DNA, 36 bp, Artificial Sequence
  Description of Artificial Sequence Synthetic oligonucleotide
gacgatgccttcagcacatgaatagacagggacgaa

SEQ ID NO: 498 - DNA, 19 bp, Artificial Sequence
  Description of Artificial Sequence Synthetic oligonucleotide
gaccttggaaataatggag

SEQ ID NO: 499 - DNA, 34 bp, Artificial Sequence
  Description of Artificial Sequence Synthetic oligonucleotide
gggacggtcggtagatcaacccagcaaaaatgga

SEQ ID NO: 500 - DNA, 34 bp, Artificial Sequence
  Description of Artificial Sequence Synthetic oligonucleotide
gctggctcggtcaagacaacccagcaaaaatggg

SEQ ID NO: 501 - DNA, 40 bp, Artificial Sequence
  Description of Artificial Sequence Synthetic oligonucleotide
gacgatgccttcagcacattggaagtgagataagataggt

SEQ ID NO: 502 - DNA, 18 bp, Artificial Sequence
  Description of Artificial Sequence Synthetic oligonucleotide
acggtgagaatgagaggt

SEQ ID NO: 503 - DNA, 34 bp, Artificial Sequence
  Description of Artificial Sequence Synthetic oligonucleotide
gggacggtcggtagataaaacagacatcagaggt

SEQ ID NO: 504 - DNA, 34 bp, Artificial Sequence
  Description of Artificial Sequence Synthetic oligonucleotide
gctggctcggtcaagaaaaacagacatcagagga

SEQ ID NO: 505 - DNA, 38 bp, Artificial Sequence
  Description of Artificial Sequence Synthetic oligonucleotide
gacgatgccttcagcacagatgaaaccctgtctctact

SEQ ID NO: 506 - DNA, 20 bp, Artificial Sequence
  Description of Artificial Sequence Synthetic oligonucleotide
ttatcaaccttagtctccct

SEQ ID NO: 507 - DNA, 34 bp, Artificial Sequence
  Description of Artificial Sequence Synthetic oligonucleotide
gggacggtcggtagatacctgccaccacacccaa

SEQ ID NO: 508 - DNA, 34 bp, Artificial Sequence
  Description of Artificial Sequence Synthetic oligonucleotide
gctggctcggtcaagaacctgccaccacacccag

SEQ ID NO: 509 - DNA, 35 bp, Artificial Sequence
  Description of Artificial Sequence Synthetic oligonucleotide
gacgatgccttcagcacagctgatgtggttgtgag

SEQ ID NO: 510 - DNA, 19 bp, Artificial Sequence
  Description of Artificial Sequence Synthetic oligonucleotide
gttcctgtagctcgtgtag

SEQ ID NO: 511 - DNA, 34 bp, Artificial Sequence
  Description of Artificial Sequence Synthetic oligonucleotide
gggacggtcggtagatctccccgccctgcagcaa

SEQ ID NO: 512 - DNA, 34 bp, Artificial Sequence
  Description of Artificial Sequence Synthetic oligonucleotide
gctggctcggtcaagactccccgccctgcagcag

SEQ ID NO: 513 - DNA, 38 bp, Artificial Sequence
  Description of Artificial Sequence Synthetic oligonucleotide
gacgatgccttcagcacatggctggactttgactgata

SEQ ID NO: 514 - DNA, 20 bp, Artificial Sequence
  Description of Artificial Sequence Synthetic oligonucleotide
tcttgtttgtgtcacagtgc

SEQ ID NO: 515 - DNA, 34 bp, Artificial Sequence
  Description of Artificial Sequence Synthetic oligonucleotide
gggacggtcggtagattgtgtcacagtgctctga

SEQ ID NO: 516 - DNA, 34 bp, Artificial Sequence
  Description of Artificial Sequence Synthetic oligonucleotide
gctggctcggtcaagatgtgtcacagtgctctgg

SEQ ID NO: 517 - DNA, 39 bp, Artificial Sequence
  Description of Artificial Sequence Synthetic oligonucleotide
gacgatgccttcagcacatttaagtaacatgacaaactc

SEQ ID NO: 518 - DNA, 18 bp, Artificial Sequence
  Description of Artificial Sequence Synthetic oligonucleotide
atctgataactgagcagg

SEQ ID NO: 519 - DNA, 34 bp, Artificial Sequence
  Description of Artificial Sequence Synthetic oligonucleotide
gggacggtcggtagatctattaagtaactggtgt

SEQ ID NO: 520 - DNA, 34 bp, Artificial Sequence
  Description of Artificial Sequence Synthetic oligonucleotide
gctggctcggtcaagactattaagtaactggtgg

SEQ ID NO: 521 - DNA, 37 bp, Artificial Sequence
  Description of Artificial Sequence Synthetic oligonucleotide
gacgatgccttcagcacaattctcccatttctcctgt

SEQ ID NO: 522 - DNA, 18 bp, Artificial Sequence
  Description of Artificial Sequence Synthetic oligonucleotide
tgcctcttctcctcattc

SEQ ID NO: 523 - DNA, 35 bp, Artificial Sequence
  Description of Artificial Sequence Synthetic oligonucleotide
gggacggtcggtagatccctaatgtcttcctctga

SEQ ID NO: 524 - DNA, 35 bp, Artificial Sequence
  Description of Artificial Sequence Synthetic oligonucleotide
gctggctcggtcaagaccctaatgtcttcctctgg

SEQ ID NO: 525 - DNA, 18 bp, Artificial Sequence
  Description of Artificial Sequence Synthetic oligonucleotide
atctcctgatccaagtcc

SEQ ID NO: 526 - DNA, 18 bp, Artificial Sequence
  Description of Artificial Sequence Synthetic oligonucleotide
cacactgtgcccatctac

SEQ ID NO: 527 - DNA, 34 bp, Artificial Sequence
  Description of Artificial Sequence Synthetic oligonucleotide
gggacggtcggtagatctgactgattacctcatg

SEQ ID NO: 528 - DNA, 34 bp, Artificial Sequence
  Description of Artificial Sequence Synthetic oligonucleotide
gctggctcggtcaagactgactgattacctcata

SEQ ID NO: 529 - DNA, 22 bp, Artificial Sequence
  Description of Artificial Sequence Synthetic oligonucleotide
cataggtaaagatctgtaggtg

SEQ ID NO: 530 - DNA, 20 bp, Artificial Sequence
  Description of Artificial Sequence Synthetic oligonucleotide
ccaccttggaagttggcaaa

SEQ ID NO: 531 - DNA, 34 bp, Artificial Sequence
  Description of Artificial Sequence Synthetic oligonucleotide
gggacggtcggtagatattaaatcgcctctctct

SEQ ID NO: 532 - DNA, 34 bp, Artificial Sequence
  Description of Artificial Sequence Synthetic oligonucleotide
gctggctcggtcaagaattaaatcgcctctctcc

SEQ ID NO: 533 - DNA, 36 bp, Artificial Sequence
  Description of Artificial Sequence Synthetic oligonucleotide
gacgatgccttcagcacaagggctttttcaggtaga

SEQ ID NO: 534 - DNA, 18 bp, Artificial Sequence
  Description of Artificial Sequence Synthetic oligonucleotide
gacctttcctgggtagaa

SEQ ID NO: 535 - DNA, 34 bp, Artificial Sequence
  Description of Artificial Sequence Synthetic oligonucleotide
gggacggtcggtagatactctgaacctgggggac

SEQ ID NO: 536 - DNA, 34 bp, Artificial Sequence
  Description of Artificial Sequence Synthetic oligonucleotide
gctggctcggtcaagaactctgaacctgggggat

SEQ ID NO: 537 - DNA, 34 bp, Artificial Sequence
  Description of Artificial Sequence Synthetic oligonucleotide
gggacggtcggtagatgatcaacacaatcttcaa

SEQ ID NO: 538 - DNA, 24 bp, Artificial Sequence
  Description of Artificial Sequence Synthetic oligonucleotide
cagctgaaagagatgaaatttact

SEQ ID NO: 539 - DNA, 42 bp, Artificial Sequence
  Description of Artificial Sequence Synthetic oligonucleotide
gacgatgccttcagcacaaacttatgaagattaaggcatagg

SEQ ID NO: 540 - DNA, 34 bp, Artificial Sequence
  Description of Artificial Sequence Synthetic oligonucleotide
gctggctcggtcaagagatcaacacaatcttcag

SEQ ID NO: 541 - DNA, 34 bp, Artificial Sequence
  Description of Artificial Sequence Synthetic oligonucleotide
gctggctcggtcaagagggctgggctgctaggga

SEQ ID NO: 542 - DNA, 20 bp, Artificial Sequence
  Description of Artificial Sequence Synthetic oligonucleotide
agacgagttcaaggtgagtg

SEQ ID NO: 543 - DNA, 36 bp, Artificial Sequence
  Description of Artificial Sequence Synthetic oligonucleotide
gacgatgccttcagcacaccaagtttccgagtttcc

SEQ ID NO: 544 - DNA, 34 bp, Artificial Sequence
  Description of Artificial Sequence Synthetic oligonucleotide
gggacggtcggtagatgggctgggctgctagggg

SEQ ID NO: 545 - DNA, 34 bp, Artificial Sequence
  Description of Artificial Sequence Synthetic oligonucleotide
gggacggtcggtagatgtaccaatacatcctgca

SEQ ID NO: 546 - DNA, 34 bp, Artificial Sequence
  Description of Artificial Sequence Synthetic oligonucleotide
gctggctcggtcaagagtaccaatacatcctgcc

SEQ ID NO: 547 - DNA, 18 bp, Artificial Sequence
  Description of Artificial Sequence Synthetic oligonucleotide
ctgctgatgtctctgttg

SEQ ID NO: 548 - DNA, 38 bp, Artificial Sequence
  Description of Artificial Sequence Synthetic oligonucleotide
gacgatgccttcagcacagacttactttgctcacactt

SEQ ID NO: 549 - DNA, 34 bp, Artificial Sequence
  Description of Artificial Sequence Synthetic oligonucleotide
gggacggtcggtagatcctcacttcctcaacgcc

SEQ ID NO: 550 - DNA, 21 bp, Artificial Sequence
  Description of Artificial Sequence Synthetic oligonucleotide
cctctctgtctggttatcttg

SEQ ID NO: 551 - DNA, 36 bp, Artificial Sequence
  Description of Artificial Sequence Synthetic oligonucleotide
gacgatgccttcagcacaagtgtgcctcctggttag

SEQ ID NO: 552 - DNA, 34 bp, Artificial Sequence
  Description of Artificial Sequence Synthetic oligonucleotide
gctggctcggtcaagacctcacttcctcaacgct

SEQ ID NO: 553 - DNA, 22 bp, Artificial Sequence
  Description of Artificial Sequence Synthetic oligonucleotide
gccaagactaggaagtaagtgt

SEQ ID NO: 554 - DNA, 22 bp, Artificial Sequence
  Description of Artificial Sequence Synthetic oligonucleotide
cccagaaccacaaagctagtaa

SEQ ID NO: 555 - DNA, 21 bp, Artificial Sequence
  Description of Artificial Sequence Synthetic oligonucleotide
tgccctggtcacctcctttcc

SEQ ID NO: 556 - DNA, 23 bp, Artificial Sequence
  Description of Artificial Sequence Synthetic oligonucleotide
ctgaccctgaccttcatactcaa

SEQ ID NO: 557 - DNA, 25 bp, Artificial Sequence
  Description of Artificial Sequence Synthetic oligonucleotide
agaagaaagaagcctctctacagtt

SEQ ID NO: 558 - DNA, 17 bp, Artificial Sequence
  Description of Artificial Sequence Synthetic oligonucleotide
aacagatcaggttggtg

SEQ ID NO: 559 - DNA, 23 bp, Artificial Sequence
  Description of Artificial Sequence Synthetic oligonucleotide
caaagatgaccttatggctctga

SEQ ID NO: 560 - DNA, 22 bp, Artificial Sequence
  Description of Artificial Sequence Synthetic oligonucleotide
gtctcggaacatgacctttagt

SEQ ID NO: 561 - DNA, 24 bp, Artificial Sequence
  Description of Artificial Sequence Synthetic oligonucleotide
tgactaagaatgtaatggggaaga

SEQ ID NO: 562 - DNA, 23 bp, Artificial Sequence
  Description of Artificial Sequence Synthetic oligonucleotide
ctttgtggatctttctgcggtgt

SEQ ID NO: 563 - DNA, 23 bp, Artificial Sequence
  Description of Artificial Sequence Synthetic oligonucleotide
ccatgttgaggagcccagagtga

SEQ ID NO: 564 - DNA, 21 bp, Artificial Sequence
  Description of Artificial Sequence Synthetic oligonucleotide
attacagttgtgagattgtgc

SEQ ID NO: 565 - DNA, 20 bp, Artificial Sequence
  Description of Artificial Sequence Synthetic oligonucleotide
ggccttctatgtactaggcg

SEQ ID NO: 566 - DNA, 21 bp, Artificial Sequence
  Description of Artificial Sequence Synthetic oligonucleotide
ctctttctggaggcatcaatc

SEQ ID NO: 567 - DNA, 14 bp, Artificial Sequence
  Description of Artificial Sequence Synthetic oligonucleotide
cacagggagacccc

SEQ ID NO: 568 - DNA, 22 bp, Artificial Sequence
  Description of Artificial Sequence Synthetic oligonucleotide
gccttattttccactcccacct

SEQ ID NO: 569 - DNA, 21 bp, Artificial Sequence
  Description of Artificial Sequence Synthetic oligonucleotide
tacctttccccatcccaactg

SEQ ID NO: 570 - DNA, 18 bp, Artificial Sequence
  Description of Artificial Sequence Synthetic oligonucleotide
tcagcatatgtttggatt

SEQ ID NO: 571 - DNA, 19 bp, Artificial Sequence
  Description of Artificial Sequence Synthetic oligonucleotide
atttgagagaaggtagggt

SEQ ID NO: 572 - DNA, 18 bp, Artificial Sequence
  Description of Artificial Sequence Synthetic oligonucleotide
tttgttactctgtagcca

SEQ ID NO: 573 - DNA, 20 bp, Artificial Sequence
  Description of Artificial Sequence Synthetic oligonucleotide
aaatattcagtaacttgttt

SEQ ID NO: 574 - DNA, 20 bp, Artificial Sequence
  Description of Artificial Sequence Synthetic oligonucleotide
aagcagcaatcgaatccctt

SEQ ID NO: 575 - DNA, 21 bp, Artificial Sequence
  Description of Artificial Sequence Synthetic oligonucleotide
tgttgttgtttggctagctcc

SEQ ID NO: 576 - DNA, 21 bp, Artificial Sequence
  Description of Artificial Sequence Synthetic oligonucleotide
cctgccttactgagagccaaa

SEQ ID NO: 577 - DNA, 21 bp, Artificial Sequence
  Description of Artificial Sequence Synthetic oligonucleotide
cacgccaattcccaccatcct

SEQ ID NO: 578 - DNA, 23 bp, Artificial Sequence
  Description of Artificial Sequence Synthetic oligonucleotide
gtccgtcgagggggaatgtgttt

SEQ ID NO: 579 - DNA, 18 bp, Artificial Sequence
  Description of Artificial Sequence Synthetic oligonucleotide
aatgtgtttcttgggggt

SEQ ID NO: 580 - DNA, 25 bp, Artificial Sequence
  Description of Artificial Sequence Synthetic oligonucleotide
ctaaccatcttccaaatgcttaatc

SEQ ID NO: 581 - DNA, 20 bp, Artificial Sequence
  Description of Artificial Sequence Synthetic oligonucleotide
tccttgagtctgagtttccc

SEQ ID NO: 582 - DNA, 17 bp, Artificial Sequence
  Description of Artificial Sequence Synthetic oligonucleotide
cacaagaaaccctgaaa

SEQ ID NO: 583 - DNA, 20 bp, Artificial Sequence
  Description of Artificial Sequence Synthetic oligonucleotide
ctcggcgtgcttggtaataa

SEQ ID NO: 584 - DNA, 24 bp, Artificial Sequence
  Description of Artificial Sequence Synthetic oligonucleotide
cggagccgaactctggaggaatct

SEQ ID NO: 585 - DNA, 24 bp, Artificial Sequence
  Description of Artificial Sequence Synthetic oligonucleotide
ggctggcaagttgttccatcccac

SEQ ID NO: 586 - DNA, 15 bp, Artificial Sequence
  Description of Artificial Sequence Synthetic oligonucleotide
tgagcagcgcatcct

SEQ ID NO: 587 - DNA, 18 bp, Artificial Sequence
  Description of Artificial Sequence Synthetic oligonucleotide
tgcagcccactgactcaa

SEQ ID NO: 588 - DNA, 18 bp, Artificial Sequence
  Description of Artificial Sequence Synthetic oligonucleotide
gctgttactcagtatgat

SEQ ID NO: 589 - DNA, 16 bp, Artificial Sequence
  Description of Artificial Sequence Synthetic oligonucleotide
ccgaagaccaagacgc

SEQ ID NO: 590 - DNA, 21 bp, Artificial Sequence
  Description of Artificial Sequence Synthetic oligonucleotide
tcttccataaaaacaaggctc

SEQ ID NO: 591 - DNA, 19 bp, Artificial Sequence
  Description of Artificial Sequence Synthetic oligonucleotide
aaacaagaaattctgttta

SEQ ID NO: 592 - DNA, 18 bp, Artificial Sequence
  Description of Artificial Sequence Synthetic oligonucleotide
tgacagctcccattggaa

SEQ ID NO: 593 - DNA, 17 bp, Artificial Sequence
  Description of Artificial Sequence Synthetic oligonucleotide
aattaatgcgatccctc

SEQ ID NO: 594 - DNA, 18 bp, Artificial Sequence
  Description of Artificial Sequence Synthetic oligonucleotide
gacagctcccattggaag

SEQ ID NO: 595 - DNA, 23 bp, Artificial Sequence
  Description of Artificial Sequence Synthetic oligonucleotide
attgggcagggataagagaaaag

SEQ ID NO: 596 - DNA, 22 bp, Artificial Sequence
  Description of Artificial Sequence Synthetic oligonucleotide
gatgaatcacagaatgcggtat

SEQ ID NO: 597 - DNA, 18 bp, Artificial Sequence
  Description of Artificial Sequence Synthetic oligonucleotide
cacacagcagttcacgca

SEQ ID NO: 598 - DNA, 22 bp, Artificial Sequence
  Description of Artificial Sequence Synthetic oligonucleotide
gccaagactaggaagtaagtgt

SEQ ID NO: 599 - DNA, 22 bp, Artificial Sequence
  Description of Artificial Sequence Synthetic oligonucleotide
cccagaaccacaaagctagtaa

SEQ ID NO: 600 - DNA, 21 bp, Artificial Sequence
  Description of Artificial Sequence Synthetic oligonucleotide
tgccctggtcacctcctttcc

SEQ ID NO: 601 - DNA, 19 bp, Artificial Sequence
  Description of Artificial Sequence Synthetic oligonucleotide
agtggctcacttgctaacg

SEQ ID NO: 602 - DNA, 21 bp, Artificial Sequence
  Description of Artificial Sequence Synthetic oligonucleotide
ctggggaagaaaataaatgaa

SEQ ID NO: 603 - DNA, 20 bp, Artificial Sequence
  Description of Artificial Sequence Synthetic oligonucleotide
cttgctcttaggatacacgt

SEQ ID NO: 604 - DNA, 19 bp, Artificial Sequence
  Description of Artificial Sequence Synthetic oligonucleotide
agcgtcttcaccatctgct

SEQ ID NO: 605 - DNA, 20 bp, Artificial Sequence
  Description of Artificial Sequence Synthetic oligonucleotide
gggaaggaggaagccaaaca

SEQ ID NO: 606 - DNA, 20 bp, Artificial Sequence
  Description of Artificial Sequence Synthetic oligonucleotide
acatgtctgatgatacctgg

SEQ ID NO: 607 - DNA, 17 bp, Artificial Sequence
  Description of Artificial Sequence Synthetic oligonucleotide
gccatgcacgatttccc

SEQ ID NO: 608 - DNA, 19 bp, Artificial Sequence
  Description of Artificial Sequence Synthetic oligonucleotide
cactgtgcccatctacgag

SEQ ID NO: 609 - DNA, 18 bp, Artificial Sequence
  Description of Artificial Sequence Synthetic oligonucleotide
ggacctgactgattacct

SEQ ID NO: 610 - DNA, 24 bp, Artificial Sequence
  Description of Artificial Sequence Synthetic oligonucleotide
gagtagctaggatcacaggtgcgt

SEQ ID NO: 611 - DNA, 24 bp, Artificial Sequence
  Description of Artificial Sequence Synthetic oligonucleotide
tgttcgagatttaagaaagttggc

SEQ ID NO: 612 - DNA, 21 bp, Artificial Sequence
  Description of Artificial Sequence Synthetic oligonucleotide
caggtgcgtgccaccatgccc

SEQ ID NO: 613 - DNA, 18 bp, Artificial Sequence
  Description of Artificial Sequence Synthetic oligonucleotide
cacacaattttccactta

SEQ ID NO: 614 - DNA, 18 bp, Artificial Sequence
  Description of Artificial Sequence Synthetic oligonucleotide
gactccagttttctatca

SEQ ID NO: 615 - DNA, 18 bp, Artificial Sequence
  Description of Artificial Sequence Synthetic oligonucleotide
atgttgatgtaatctact

SEQ ID NO: 616 - DNA, 19 bp, Artificial Sequence
  Description of Artificial Sequence Synthetic oligonucleotide
tggggcaagcaacagtggt

SEQ ID NO: 617 - DNA, 21 bp, Artificial Sequence
  Description of Artificial Sequence Synthetic oligonucleotide
taggcagggcaagggattagg

SEQ ID NO: 618 - DNA, 21 bp, Artificial Sequence
  Description of Artificial Sequence Synthetic oligonucleotide
tttaaattctctgacagagac

SEQ ID NO: 619 - DNA, 25 bp, Artificial Sequence
  Description of Artificial Sequence Synthetic oligonucleotide
gccaccagcccacactctgaacctg

SEQ ID NO: 620 - DNA, 24 bp, Artificial Sequence
  Description of Artificial Sequence Synthetic oligonucleotide
ccatcagccttcacccacgtgcca

SEQ ID NO: 621 - DNA, 15 bp, Artificial Sequence
  Description of Artificial Sequence Synthetic oligonucleotide
gcctcagcttgacct

SEQ ID NO: 622 - DNA, 24 bp, Artificial Sequence
  Description of Artificial Sequence Synthetic oligonucleotide
ggtaagtgcgtgcctgggagatgc

SEQ ID NO: 623 - DNA, 20 bp, Artificial Sequence
  Description of Artificial Sequence Synthetic oligonucleotide
cggggtggggaggacagagc

SEQ ID NO: 624 - DNA, 19 bp, Artificial Sequence
  Description of Artificial Sequence Synthetic oligonucleotide
gaggacagagcaaaaggat

SEQ ID NO: 625 - DNA, 20 bp, Artificial Sequence
  Description of Artificial Sequence Synthetic oligonucleotide
tgccttacaatatacaatgg

SEQ ID NO: 626 - DNA, 21 bp, Artificial Sequence
  Description of Artificial Sequence Synthetic oligonucleotide
caatgggtaaggagtaaagtt

SEQ ID NO: 627 - DNA, 15 bp, Artificial Sequence
  Description of Artificial Sequence Synthetic oligonucleotide
ttccagctgctttta

SEQ ID NO: 628 - DNA, 22 bp, Artificial Sequence
  Description of Artificial Sequence Synthetic oligonucleotide
gaacaaacctccgagatgctac

SEQ ID NO: 629 - DNA, 24 bp, Artificial Sequence
  Description of Artificial Sequence Synthetic oligonucleotide
gtcttatgttactgggctttcacc

SEQ ID NO: 630 - DNA, 31 bp, Artificial Sequence
  Description of Artificial Sequence Synthetic oligonucleotide
caccctctagaattcactattaattttcaac

SEQ ID NO: 631 - DNA, 21 bp, Artificial Sequence
  Description of Artificial Sequence Synthetic oligonucleotide
tttcgctccatcaaccaagtc

SEQ ID NO: 632 - DNA, 26 bp, Artificial Sequence
  Description of Artificial Sequence Synthetic oligonucleotide
gcccagttatacctcagtgttgtaac

SEQ ID NO: 633 - DNA, 21 bp, Artificial Sequence
  Description of Artificial Sequence Synthetic oligonucleotide
tgtatgcacgtgcgtgatctg

SEQ ID NO: 634 - DNA, 25 bp, Artificial Sequence
  Description of Artificial Sequence Synthetic oligonucleotide
ctgtaagcatctggaattgtcatga

SEQ ID NO: 635 - DNA, 21 bp, Artificial Sequence
  Description of Artificial Sequence Synthetic oligonucleotide
ggaccctaagaaccccaggat

SEQ ID NO: 636 - DNA, 17 bp, Artificial Sequence
  Description of Artificial Sequence Synthetic oligonucleotide
acagggctggcagccac

SEQ ID NO: 637 - DNA, 21 bp, Artificial Sequence
  Description of Artificial Sequence Synthetic oligonucleotide
ggagctgtgaggtatgggctt

SEQ ID NO: 638 - DNA, 21 bp, Artificial Sequence
  Description of Artificial Sequence Synthetic oligonucleotide
tttggtgggttgtcattgaca

SEQ ID NO: 639 - DNA, 20 bp, Artificial Sequence
  Description of Artificial Sequence Synthetic oligonucleotide
cactcagccctgctctttcc

SEQ ID NO: 640 - DNA, 19 bp, Artificial Sequence
  Description of Artificial Sequence Synthetic oligonucleotide
ctggctcctgacccttgct

SEQ ID NO: 641 - DNA, 19 bp, Artificial Sequence
  Description of Artificial Sequence Synthetic oligonucleotide
ccgtggcttcatggtgact

SEQ ID NO: 642 - DNA, 29 bp, Artificial Sequence
  Description of Artificial Sequence Synthetic oligonucleotide
ttctcactgtgatatataaactcagaccc

SEQ ID NO: 643 - DNA, 29 bp, Artificial Sequence
  Description of Artificial Sequence Synthetic oligonucleotide
tcattacatcaggtatattgcactgtaaa

SEQ ID NO: 644 - DNA, 22 bp, Artificial Sequence
  Description of Artificial Sequence Synthetic oligonucleotide
gctgcattggagaggactgatc

SEQ ID NO: 645 - DNA, 25 bp, Artificial Sequence
  Description of Artificial Sequence Synthetic oligonucleotide
tgctagtgttttctggttgcatatt

SEQ ID NO: 646 - DNA, 19 bp, Artificial Sequence
  Description of Artificial Sequence Synthetic oligonucleotide
gcgaagtgtcggacaccaa

SEQ ID NO: 647 - DNA, 25 bp, Artificial Sequence
  Description of Artificial Sequence Synthetic oligonucleotide
aagatgaccttatggctctgagatg

SEQ ID NO: 648 - DNA, 28 bp, Artificial Sequence
  Description of Artificial Sequence Synthetic oligonucleotide
ggccttgaagaagattttatattgagaa

SEQ ID NO: 649 - DNA, 20 bp, Artificial Sequence
  Description of Artificial Sequence Synthetic oligonucleotide
gatgggtgatcagccgaatc

SEQ ID NO: 650 - DNA, 26 bp, Artificial Sequence
  Description of Artificial Sequence Synthetic oligonucleotide
aggtcagtacagagggtatcatgaga

SEQ ID NO: 651 - DNA, 17 bp, Artificial Sequence
  Description of Artificial Sequence Synthetic oligonucleotide
cgccctcggcactcttg

SEQ ID NO: 652 - DNA, 25 bp, Artificial Sequence
  Description of Artificial Sequence Synthetic oligonucleotide
ggctcagtctttgatctttagcaag

SEQ ID NO: 653 - DNA, 23 bp, Artificial Sequence
  Description of Artificial Sequence Synthetic oligonucleotide
atgggctaacacaggagatgatg

SEQ ID NO: 654 - DNA, 18 bp, Artificial Sequence
  Description of Artificial Sequence Synthetic oligonucleotide
agcctctgccctcctcca

SEQ ID NO: 655 - DNA, 22 bp, Artificial Sequence
  Description of Artificial Sequence Synthetic oligonucleotide
tgtcaagatgcagctgaaggtc

SEQ ID NO: 656 - DNA, 20 bp, Artificial Sequence
  Description of Artificial Sequence Synthetic oligonucleotide
tggacatatgggcggactct

SEQ ID NO: 657 - DNA, 19 bp, Artificial Sequence
  Description of Artificial Sequence Synthetic oligonucleotide
catccttggcggtcttggt

SEQ ID NO: 658 - DNA, 20 bp, Artificial Sequence
  Description of Artificial Sequence Synthetic oligonucleotide
ggaggatgccatctcgaaca

SEQ ID NO: 659 - DNA, 21 bp, Artificial Sequence
  Description of Artificial Sequence Synthetic oligonucleotide
ctacctgtccggtgcatcatc

SEQ ID NO: 660 - DNA, 24 bp, Artificial Sequence
  Description of Artificial Sequence Synthetic oligonucleotide
cgatgaacagttggaataggttgt

SEQ ID NO: 661 - DNA, 29 bp, Artificial Sequence
  Description of Artificial Sequence Synthetic oligonucleotide
tcagagacactgaagaacttaaagaaatc

SEQ ID NO: 662 - DNA, 28 bp, Artificial Sequence
  Description of Artificial Sequence Synthetic oligonucleotide
cggttaacttataaagaaacggatgttc

SEQ ID NO: 663 - DNA, 24 bp, Artificial Sequence
  Description of Artificial Sequence Synthetic oligonucleotide
ggcaccgtgtagacttgatctaaa

SEQ ID NO: 664 - DNA, 23 bp, Artificial Sequence
  Description of Artificial Sequence Synthetic oligonucleotide
ggttacgtctgctcttcgatcct

SEQ ID NO: 665 - DNA, 25 bp, Artificial Sequence
  Description of Artificial Sequence Synthetic oligonucleotide
tctcggaacatgacctttagtctgt

SEQ ID NO: 666 - DNA, 16 bp, Artificial Sequence
  Description of Artificial Sequence Synthetic oligonucleotide
ctatgcatacttttgc

SEQ ID NO: 667 - DNA, 17 bp, Artificial Sequence
  Description of Artificial Sequence Synthetic oligonucleotide
caattggagttgggagg

SEQ ID NO: 668 - DNA, 17 bp, Artificial Sequence
  Description of Artificial Sequence Synthetic oligonucleotide
tgtgatacctggaacag

SEQ ID NO: 669 - DNA, 16 bp, Artificial Sequence
  Description of Artificial Sequence Synthetic oligonucleotide
ccaaacaacaggacgg

SEQ ID NO: 670 - DNA, 18 bp, Artificial Sequence
  Description of Artificial Sequence Synthetic oligonucleotide
cactcacattataattag

SEQ ID NO: 671 - DNA, 13 bp, Artificial Sequence
  Description of Artificial Sequence Synthetic oligonucleotide
tggcctggcggtg

SEQ ID NO: 672 - DNA, 18 bp, Artificial Sequence
  Description of Artificial Sequence Synthetic oligonucleotide
aaccaaaatgaaggagag

SEQ ID NO: 673 - DNA, 14 bp, Artificial Sequence
  Description of Artificial Sequence Synthetic oligonucleotide
acggaggaagaggt

SEQ ID NO: 674 - DNA, 17 bp, Artificial Sequence
  Description of Artificial Sequence Synthetic oligonucleotide
agtgtgatcatcacttt

SEQ ID NO: 675 - DNA, 15 bp, Artificial Sequence
  Description of Artificial Sequence Synthetic oligonucleotide
tgcagggctacatga

SEQ ID NO: 676 - DNA, 16 bp, Artificial Sequence
  Description of Artificial Sequence Synthetic oligonucleotide
tgcctccttctcacac

SEQ ID NO: 677 - DNA, 17 bp, Artificial Sequence
  Description of Artificial Sequence Synthetic oligonucleotide
tcctataccgtgggtgt

SEQ ID NO: 678 - DNA, 19 bp, Artificial Sequence
  Description of Artificial Sequence Synthetic oligonucleotide
tactcatcttcctaattac

SEQ ID NO: 679 - DNA, 16 bp, Artificial Sequence
  Description of Artificial Sequence Synthetic oligonucleotide
tgttaccagaaagaaa

SEQ ID NO: 680 - DNA, 15 bp, Artificial Sequence
  Description of Artificial Sequence Synthetic oligonucleotide
cataccacaaaacca

SEQ ID NO: 681 - DNA, 14 bp, Artificial Sequence
  Description of Artificial Sequence Synthetic oligonucleotide
tcatgggcatttca

SEQ ID NO: 682 - DNA, 16 bp, Artificial Sequence
  Description of Artificial Sequence Synthetic oligonucleotide
aagacgaaaatgaatc

SEQ ID NO: 683 - DNA, 16 bp, Artificial Sequence
  Description of Artificial Sequence Synthetic oligonucleotide
aagaattgccctgcct

SEQ ID NO: 684 - DNA, 19 bp, Artificial Sequence
  Description of Artificial Sequence Synthetic oligonucleotide
atgcatagttttgcattat

SEQ ID NO: 685 - DNA, 16 bp, Artificial Sequence
  Description of Artificial Sequence Synthetic oligonucleotide
aattggggttgggagg

SEQ ID NO: 686 - DNA, 17 bp, Artificial Sequence
  Description of Artificial Sequence Synthetic oligonucleotide
ctgtgatacctggcaca

SEQ ID NO: 687 - DNA, 15 bp, Artificial Sequence
  Description of Artificial Sequence Synthetic oligonucleotide
aaacagcaggacggg

SEQ ID NO: 688 - DNA, 18 bp, Artificial Sequence
  Description of Artificial Sequence Synthetic oligonucleotide
actcacattacaattagt

SEQ ID NO: 689 - DNA, 14 bp, Artificial Sequence
  Description of Artificial Sequence Synthetic oligonucleotide
tggcctggcgatgt

SEQ ID NO: 690 - DNA, 17 bp, Artificial Sequence
  Description of Artificial Sequence Synthetic oligonucleotide
accaaaatggaggagag

SEQ ID NO: 691 - DNA, 14 bp, Artificial Sequence
  Description of Artificial Sequence Synthetic oligonucleotide
acggaggaagaagt

SEQ ID NO: 692 - DNA, 16 bp, Artificial Sequence
  Description of Artificial Sequence Synthetic oligonucleotide
cagtgtgatcgtcact

SEQ ID NO: 693 - DNA, 16 bp, Artificial Sequence
  Description of Artificial Sequence Synthetic oligonucleotide
tcatgcagggttacat

SEQ ID NO: 694 - DNA, 17 bp, Artificial Sequence
  Description of Artificial Sequence Synthetic oligonucleotide
cctccttttcacaccga

SEQ ID NO: 695 - DNA, 18 bp, Artificial Sequence
  Description of Artificial Sequence Synthetic oligonucleotide
ctatactgtgggtgtcat

SEQ ID NO: 696 - DNA, 19 bp, Artificial Sequence
  Description of Artificial Sequence Synthetic oligonucleotide
caaatatctactcattttc

SEQ ID NO: 697 - DNA, 16 bp, Artificial Sequence
  Description of Artificial Sequence Synthetic oligonucleotide
tgttaccagaaataaa

SEQ ID NO: 698 - DNA, 16 bp, Artificial Sequence
  Description of Artificial Sequence Synthetic oligonucleotide
accacaaacccaggtc

SEQ ID NO: 699 - DNA, 16 bp, Artificial Sequence
  Description of Artificial Sequence Synthetic oligonucleotide
tatcatgggcctttca

SEQ ID NO: 700 - DNA, 16 bp, Artificial Sequence
  Description of Artificial Sequence Synthetic oligonucleotide
aagacgaaaatggatc

SEQ ID NO: 701 - DNA, 15 bp, Artificial Sequence
  Description of Artificial Sequence Synthetic oligonucleotide
aagaactgccctgcc

SEQ ID NO: 702 - DNA, 30 bp, Artificial Sequence
  Description of Artificial Sequence Synthetic oligonucleotide
gggatatatagtagaaaaacaagcctgtct

SEQ ID NO: 703 - DNA, 15 bp, Artificial Sequence
  Description of Artificial Sequence Synthetic oligonucleotide
ggcccgctcctggct

SEQ ID NO: 704 - DNA, 21 bp, Artificial Sequence
  Description of Artificial Sequence Synthetic oligonucleotide
accagaaacaaatgccaacca

SEQ ID NO: 705 - DNA, 29 bp, Artificial Sequence
  Description of Artificial Sequence Synthetic oligonucleotide
catagtttaggataaacaaaagggattca

SEQ ID NO: 706 - DNA, 25 bp, Artificial Sequence
  Description of Artificial Sequence Synthetic oligonucleotide
gctattgaatggatgtgccttattt

SEQ ID NO: 707 - DNA, 18 bp, Artificial Sequence
  Description of Artificial Sequence Synthetic oligonucleotide
cagcccctctgctccaag

SEQ ID NO: 708 - DNA, 19 bp, Artificial Sequence
  Description of Artificial Sequence Synthetic oligonucleotide
ggtgacgtttgcgcatctc

SEQ ID NO: 709 - DNA, 18 bp, Artificial Sequence
  Description of Artificial Sequence Synthetic oligonucleotide
ctgggcccaccgagttac

SEQ ID NO: 710 - DNA, 19 bp, Artificial Sequence
  Description of Artificial Sequence Synthetic oligonucleotide
acattccccttccacgctt

SEQ ID NO: 711 - DNA, 21 bp, Artificial Sequence
  Description of Artificial Sequence Synthetic oligonucleotide
gccatccttgttgaacgtgaa

SEQ ID NO: 712 - DNA, 18 bp, Artificial Sequence
  Description of Artificial Sequence Synthetic oligonucleotide
gagcaacagccgcctgag

SEQ ID NO: 713 - DNA, 20 bp, Artificial Sequence
  Description of Artificial Sequence Synthetic oligonucleotide
gaaagctaactcccctgacg

SEQ ID NO: 714 - DNA, 27 bp, Artificial Sequence
  Description of Artificial Sequence Synthetic oligonucleotide
agtttgttttcctattagaggtttcca

SEQ ID NO: 715 - DNA, 30 bp, Artificial Sequence
  Description of Artificial Sequence Synthetic oligonucleotide
catattcaagaaagattatctccaactctt

SEQ ID NO: 716 - DNA, 23 bp, Artificial Sequence
  Description of Artificial Sequence Synthetic oligonucleotide
gagttggtggcataaaaccctaa

SEQ ID NO: 717 - DNA, 31 bp, Artificial Sequence
  Description of Artificial Sequence Synthetic oligonucleotide
caacttaatcactactactccatgtaaagca

SEQ ID NO: 718 - DNA, 24 bp, Artificial Sequence
  Description of Artificial Sequence Synthetic oligonucleotide
aaccccacaccttcagtctagaaa

SEQ ID NO: 719 - DNA, 22 bp, Artificial Sequence
  Description of Artificial Sequence Synthetic oligonucleotide
cagtgtgaaaccaagggatgtc

SEQ ID NO: 720 - DNA, 20 bp, Artificial Sequence
  Description of Artificial Sequence Synthetic oligonucleotide
tgtcatggaaacgccacaac

SEQ ID NO: 721 - DNA, 21 bp, Artificial Sequence
  Description of Artificial Sequence Synthetic oligonucleotide
tgcatggcatcagcatatgtt

SEQ ID NO: 722 - DNA, 17 bp, Artificial Sequence
  Description of Artificial Sequence Synthetic oligonucleotide
tccccctctgtccaggc

SEQ ID NO: 723 - DNA, 25 bp, Artificial Sequence
  Description of Artificial Sequence Synthetic oligonucleotide
aagttaatcaagccttttcaattgg

SEQ ID NO: 724 - DNA, 23 bp, Artificial Sequence
  Description of Artificial Sequence Synthetic oligonucleotide
gatctctgtgagtgtgcgtctgt

SEQ ID NO: 725 - DNA, 17 bp, Artificial Sequence
  Description of Artificial Sequence Synthetic oligonucleotide
gcagggcagagggagga

SEQ ID NO: 726 - DNA, 19 bp, Artificial Sequence
  Description of Artificial Sequence Synthetic oligonucleotide
acatgaccagggcccactt

SEQ ID NO: 727 - DNA, 20 bp, Artificial Sequence
  Description of Artificial Sequence Synthetic oligonucleotide
gcgggagctagagagcagtg

SEQ ID NO: 728 - DNA, 20 bp, Artificial Sequence
  Description of Artificial Sequence Synthetic oligonucleotide
tgaagggtaagggagggaaa

SEQ ID NO: 729 - DNA, 20 bp, Artificial Sequence
  Description of Artificial Sequence Synthetic oligonucleotide
ctcttatgccttccccacca

SEQ ID NO: 730 - DNA, 27 bp, Artificial Sequence
  Description of Artificial Sequence Synthetic oligonucleotide
tggaaacctctaataggaaaacaaact

SEQ ID NO: 731 - DNA, 22 bp, Artificial Sequence
  Description of Artificial Sequence Synthetic oligonucleotide
cctgtccccaccttctctctct

SEQ ID NO: 732 - DNA, 17 bp, Artificial Sequence
  Description of Artificial Sequence Synthetic oligonucleotide
aaggaaagctggatatg

SEQ ID NO: 733 - DNA, 25 bp, Artificial Sequence
  Description of Artificial Sequence Synthetic oligonucleotide
ccacctcccttctagcctcagttgc

SEQ ID NO: 734 - DNA, 19 bp, Artificial Sequence
  Description of Artificial Sequence Synthetic oligonucleotide
cgaatgtggctgcccagcc

SEQ ID NO: 735 - DNA, 18 bp, Artificial Sequence
  Description of Artificial Sequence Synthetic oligonucleotide
aacagatctggtctacct

SEQ ID NO: 736 - DNA, 14 bp, Artificial Sequence
  Description of Artificial Sequence Synthetic oligonucleotide
cccacctggagaat

SEQ ID NO: 737 - DNA, 17 bp, Artificial Sequence
  Description of Artificial Sequence Synthetic oligonucleotide
tgagaaaaaaggttccg

SEQ ID NO: 738 - DNA, 14 bp, Artificial Sequence
  Description of Artificial Sequence Synthetic oligonucleotide
tgctcaggatagcc

SEQ ID NO: 739 - DNA, 14 bp, Artificial Sequence
  Description of Artificial Sequence Synthetic oligonucleotide
aggaagcgtggcct

SEQ ID NO: 740 - DNA, 16 bp, Artificial Sequence
  Description of Artificial Sequence Synthetic oligonucleotide
cgcccagtaatacaga

SEQ ID NO: 741 - DNA, 16 bp, Artificial Sequence
  Description of Artificial Sequence Synthetic oligonucleotide
tcgttccactggacgt

SEQ ID NO: 742 - DNA, 12 bp, Artificial Sequence
  Description of Artificial Sequence Synthetic oligonucleotide
tcggcgctggtc

SEQ ID NO: 743 - DNA, 14 bp, Artificial Sequence
  Description of Artificial Sequence Synthetic oligonucleotide
cttggcgtcgcgtc

SEQ ID NO: 744 - DNA, 18 bp, Artificial Sequence
  Description of Artificial Sequence Synthetic oligonucleotide
ttgaaaggacaccatatt

SEQ ID NO: 745 - DNA, 19 bp, Artificial Sequence
  Description of Artificial Sequence Synthetic oligonucleotide
cactaaagctgtaatatta

SEQ ID NO: 746 - DNA, 17 bp, Artificial Sequence
  Description of Artificial Sequence Synthetic oligonucleotide
tcttcctctgggtaaca

SEQ ID NO: 747 - DNA, 17 bp, Artificial Sequence
  Description of Artificial Sequence Synthetic oligonucleotide
aggaaagctgggtatgt

SEQ ID NO: 748 - DNA, 24 bp, Artificial Sequence
  Description of Artificial Sequence Synthetic oligonucleotide
cccacctccctcctagcctcagtt

SEQ ID NO: 749 - DNA, 22 bp, Artificial Sequence
  Description of Artificial Sequence Synthetic oligonucleotide
tcgaatgtgactgcccagcctc

SEQ ID NO: 750 - DNA, 16 bp, Artificial Sequence
  Description of Artificial Sequence Synthetic oligonucleotide
agatctggtctgcctc

SEQ ID NO: 751 - DNA, 14 bp, Artificial Sequence
  Description of Artificial Sequence Synthetic oligonucleotide
tcccacctggggaa

SEQ ID NO: 752 - DNA, 16 bp, Artificial Sequence
  Description of Artificial Sequence Synthetic oligonucleotide
ctgagaaaaaagcttc

SEQ ID NO: 753 - DNA, 14 bp, Artificial Sequence
  Description of Artificial Sequence Synthetic oligonucleotide
tgctcaggacagcc

SEQ ID NO: 754 - DNA, 14 bp, Artificial Sequence
  Description of Artificial Sequence Synthetic oligonucleotide
caaggaagggtggc

SEQ ID NO: 755 - DNA, 17 bp, Artificial Sequence
  Description of Artificial Sequence Synthetic oligonucleotide
cccagtaatccagacac

SEQ ID NO: 756 - DNA, 16 bp, Artificial Sequence
  Description of Artificial Sequence Synthetic oligonucleotide
ttccattggacgtcct

SEQ ID NO: 757 - DNA, 13 bp, Artificial Sequence
  Description of Artificial Sequence Synthetic oligonucleotide
tctcggcgctcgt

SEQ ID NO: 758 - DNA, 15 bp, Artificial Sequence
  Description of Artificial Sequence Synthetic oligonucleotide
ttggcatcgcgtcag

SEQ ID NO: 759 - DNA, 16 bp, Artificial Sequence
  Description of Artificial Sequence Synthetic oligonucleotide
acaccgtatttttcac

SEQ ID NO: 760 - DNA, 18 bp, Artificial Sequence
  Description of Artificial Sequence Synthetic oligonucleotide
ctaaagctgtcatattac

SEQ ID NO: 761 - DNA, 16 bp, Artificial Sequence
  Description of Artificial Sequence Synthetic oligonucleotide
tcctctgagtaacaac

(19) United States
(12) Patent Application Publication
(10) Pub. No.: US 2007/0128597 A1
(43) Pub. Date: Jun. 7, 2007

(54) Single nucleotide polymorphisms sensitively predicting adverse drug reactions (adr) and drug efficacy

(76) Inventors: Stephan Schwers, Koln (DE); Harald Kallabis, Koln (DE); Udo Stropp, Haan (DE)

Correspondence Address: MINTZ, LEVIN, COHN, FERRIS, GLOVSKY AND POPEO, P.C, 1400 PAGE MILL ROAD, PALO ALTO, CA, 94304-1124, US

(21) Appl. No.: 10/525,278

(22) PCT Filed: Aug. 18, 2003
(86) PCT No.: PCT/EP03/09126

§ 371 Date: Nov. 21, 2005

(30) Foreign Application Priority Data
Aug. 19, 2002 (EP) 020118158.2

(51) Int. Cl. C12Q 1/68; C07H 21/04; C12P 21/06; C07K 14/705

(52) U.S. Cl. 435/6; 435/69.1; 435/320.1; 435/325; 530/350; 536/23.5

(57) ABSTRACT

Single Nucleotide Polymorphisms sensitively predicting Advserse Drug Reactions (ADR) and Drug Efficacy Abs tract. The invention provides diagnostic methods and kits including oligo and/or polynucleotides or derivatives, including as well antibodies determining whether a human subject is at risk of getting adverse drug reaction after statin therapy or whether the human subject is a high or low responder or a good a or bad metabolizer of statins. The invention provides further diagnostic methods and kits including antibodies determining whether a human subject is at risk for a cardiovascular disease. Still further the invention provides polymorphic sequences and other genes. The present invention further relates to isolated polynucleotides encoding a phenotype associated (PA) gene polypeptide useful in methods to identify therapeutic agents and useful for preparation of a medicament to treat cardiovascular disease or influence drug response, the polynucleotide is selected from the group comprising: SEQ ID 1-168 with allelic variation as indicated in the sequences section contained in a functional surrounding like full length cDNA for PA gene polypeptide and with or without the PA gene promoter sequence.

### TECHNICAL FIELD

This invention relates to genetic polymorphisms useful for assessing cardiovascular risks in humans, including, but not limited to, atherosclerosis, ischemia/reperfusion, hypertension, restenosis, arterial inflammation, myocardial infarction, and stroke. In addition it relates to genetic polymorphisms useful for assessing the response to lipid lowering drug therapy. Specifically, the present invention identifies and describes gene variations which are individually present in humans with cardiovascular disease states, rela to humans with normal, or non-cardiovascular disease states, and/or in response to medications relevant to cardiovascular disease. Further, the present invention provides methods for the identification and therapeutic use of compounds as treatments of cardiovascular disease. Moreover, the present invention provides methods for the diagnostic monitoring of patients undergoing clinical evaluation for the treatment of cardiovascular disease, and for monitoring the efficacy of compounds in clinical trials. Still further, the present invention provides methods to use gene variations to predict personal medication schemes omitting adverse drug reactions and allowing an adjustment of the drug dose to achieve maximum benefit for the patient. Additionally, the present invention describes methods for the diagnostic evaluation and prognosis of various cardiovascular diseases, and for the identification of subjects exhibiting a predisposition to such conditions.

### BACKGROUND OF THE INVENTION

Cardiovascular disease is a major health risk throughout the industrialized world.

Cardiovascular diseases include but are not limited by the following disorders of the heart and the vascular system: congestive heart failure, myocardial infarction, atherosclerosis, ischemic diseases of the heart, coronary heart disease, all kinds of atrial and ventricular arrhythmias, hypertensive vascular diseases and peripheral vascular diseases.

Heart failure is defined as a pathophysiologic state in which an abnormality of cardiac function is responsible for the failure of the heart to pump blood at a rate commensurate with the requirement of the metabolizing tissue. It includes all forms of pumping failure such as high-output and low-output, acute and chronic, right-sided or left-sided, systolic or diastolic, independent of the underlying cause.

Myocardial infarction (MI) is generally caused by an abrupt decrease in coronary blood flow that follows a thrombotic occlusion of a coronary artery previously narrowed by arteriosclerosis. MI prophylaxis (primary and secondary prevention) is included as well as the acute treatment of MI and the prevention of complications.

Ischemic diseases are conditions in which the coronary flow is restricted resulting in an perfusion which is inadequate to meet the myocardial requirement for oxygen. This group of diseases include stable angina, unstable angina and asyrriptomatic ischemia.

Arrhythmias include all forms of atrial and ventricular tachyarrhythmias (atrial tachycardia, atrial flutter, atrial fibrillation, atrio-ventricular reentrant tachycardia, preexitation syndrome, ventricular tachycardia, ventricular flutter, ventricular fibrillation) as well as bradycardic forms of arrhythmias.

Hypertensive vascular diseases include primary as well as all kinds of secondary arterial hypertension (renal, endocrine, neurogenic, others).

Peripheral vascular diseases are defined as vascular diseases in which arterial and/or venous flow is reduced resulting in an imbalance between blood supply and tissue oxygen demand. It includes chronic peripheral arterial occlusive disease (PAOD), acute arterial thrombosis and embolism, inflammatory vascular disorders, Raynaud's phenomenon and venous disorders.

Atherosclerosis, the most prevalent of vascular diseases, is the principal cause of heart attack, stroke, and gangrene of the extremities, and thereby the principal cause of death. Atherosclerosis is a complex disease involving many cell types and molecular factors (for a detailed review, see Ross, 1993, Nature 362: 801-809 and Lusis, A. J., Nature 407, 233-241 (2000)). The process, in normal circumstances a protective response to insults to the endothelium and smooth muscle cells (SMCs) of the wall of the artery, consists of the formation of fibrofatty and fibrous lesions or plaques, preceded and accompanied by inflammation. The advanced lesions of atherosclerosis may occlude the artery concerned, and result from an excessive inflammatory-fibroproliferative response to numerous different forms of insult. For example, shear stresses are thought to be responsible for the frequent occurrence of atherosclerotic plaques in regions of the circulatory system where turbulent blood flow occurs, such as branch points and irregular structures.

The first observable event in the formation of an atherosclerotic plaque occurs when blood-borne monocytes adhere to the vascular endothelial layer and transmigrate through to the sub-endothelial space. Adjacent endothelial cells at the same time produce oxidized low density lipoprotein (LDL). These oxidized LDLs are then taken up in large amounts by the monocytes through scavenger receptors expressed on their surfaces. In contrast to the regulated pathway by which native LDL (nLDL) is taken up by nLDL specific receptors, the scavenger pathway of uptake is not regulated by the monocytes.

These lipid-filled monocytes are called foam cells, and are the major constituent of the fatty streak. Interactions between foam cells and the endothelial and SMCs which surround them lead to a state of chronic local inflammation which can eventually lead to smooth muscle cell proliferation and migration, and the formation of a fibrous plaque. Such plaques occlude the blood vessel concerned and thus restrict the flow of blood, resulting in ischemia.

Ischemia is a condition characterized by a lack of oxygen supply in tissues of organs due to inadequate perfusion. Such inadequate perfusion can have number of natural causes, including atherosclerotic or restenotic lesions, anemia, or stroke, to name a few. Many medical interventions, such as the interruption of the flow of blood during bypass surgery, for example, also lead to ischemia. In addition to sometimes being caused by diseased cardiovascular tissue, ischemia may sometimes affect cardiovascular tissue, such as in ischemic heart disease. Ischemia may occur in any organ, however, that is suffering a lack of oxygen supply.

The most common cause of ischemia in the heart is atherosclerotic disease of epicardial coronary arteries. By reducing the lumen of these vessels, atherosclerosis causes an absolute decrease in myocardial perfusion in the basal state or limits appropriate increases in perfusion when the demand for flow is augmented. Coronary blood flow can also be limited by arterial thrombi, spasm, and, rarely, coronary emboli, as well as by ostial narrowing due to luetic aortitis. Congenital abnormalities, such as anomalous origin of the left anterior descending coronary artery from the pulmonary artery, may cause myocardial ischemia and infarction in infancy, but this cause is very rare in adults. Myocardial ischemia can also occur if myocardial oxygen demands are abnormally increased, as in severe ventricular hypertrophy due to hypertension or aortic stenosis. The latter can be present with angina that is indistinguishable from that caused by coronary atherosclerosis. A reduction in the oxygen-carrying capacity of the blood, as in extremely severe anemia or in the presence of carboxy-hemoglobin, is a rare cause of myocardial ischemia. Not infrequently, two or more causes of ischemia will coexist, such as an increase in oxygen demand due to left ventricular hypertrophy and a reduction in oxygen supply secondary to coronary atherosclerosis.

The foregoing studies are aimed at defining the role of particular gene variations presumed to be involved in the misleading of normal cellular function leading to cardiovascular disease. However, such approaches cannot identify the full panoply of gene variations that are involved in the disease process.

At present, the only available treatments for cardiovascular disorders are pharmaceutical based medications that are not targeted to an individual's actual defect; examples include angiotensin converting enzyme (ACE) inhibitors and diuretics for hypertension, insulin supplementation for non-insulin dependent diabetes mellitus (NDDM), cholesterol reduction strategies for dyslipidaemia, anticoagulants, blockers for cardiovascular disorders and weight reduction strategies for obesity. If targeted treatment strategies were available it might be possible to predict the response to a particular regime of therapy and could markedly increase the effectiveness of such treatment. Although targeted therapy requires accurate diagnostic tests for disease susceptibility, once these tests are developed the opportunity to utilize targeted therapy will become widespread. Such diagnostic tests could initially serve to identify individuals at most risk of hypertension and could allow them to make changes in lifestyle or diet that would serve as preventative measures. The benefits associated by coupling the diagnostic tests with a system of targeted therapy could include the reduction in dosage of administered drugs and thus the amount of unpleasant side effects suffered by an individual. In more severe cases a diagnostic test may suggest that earlier surgical intervention would be useful in preventing a further deterioration in condition.

It is an object of the invention to provide genetic diagnosis of predisposition or susceptibility for cardiovascular diseases. Another related object is to provide treatment to reduce or prevent or delay the onset of disease in those predisposed or susceptible to this disease. A further object is to provide means for carrying out this diagnosis.

Accordingly, a first aspect of the invention provides a method of diagnosis of disease in an individual, said method comprising determining one, various or all genotypes in said individual of the genes listed in the Examples.

In another aspect, the invention provides a method of identifying an individual predisposed or susceptible to a disease, said method comprising determining one, various or all genotypes in said individual of the genes listed in the Examples.

The invention is of advantage in that it enables diagnosis of a disease or of certain disease states via genetic analysis which can yield useable results before onset of disease symptoms, or before onset of severe symptoms. The invention is further of advantage in that it enables diagnosis of predisposition or susceptibility to a disease or of certain disease states via genetic analysis.

The invention may also be of use in confirming or corroborating the results of other diagnostic methods. The diagnosis of the invention may thus suitably be used either as an isolated technique or in combination with other methods and apparatus for diagnosis, in which latter case the invention provides a further test on which a diagnosis may be assessed.

The present invention stems from using allelic association as a method for genotyping individuals; allowing the investigation of the molecular genetic basis for cardiovascular diseases. In a specific embodiment the invention tests for the polymorphisms in the sequences of the listed genes in the Examples. The invention demonstrates a link between this polymorphisms and predispositions to cardio-vascular diseases by showing that allele frequencies significantly differ when individuals with “bad” serum lipids are compared to individuals with “good” serum levels. The meaning of “good and bad” serum lipid levels is defined in Table 1a.

The PROCAM algorithm defines also a risk assessment based on lipids (LDL-cholesterol, HDL-cholesterol, triglycerides) and risk factors like smoking, high blood pressure or diabetes mellitus (Assmann, G., Schulte, H. von Ecjkardstein, A: Am J Cardiol 77 (1996): 1179-1184).

Certain disease states would benefit, that is to say the suffering of the patient may be reduced or prevented or delayed, by administration of treatment or therapy in advance of disease appearance; this can be more reliably carried out if advance diagnosis of predisposition or susceptibility to disease can be diagnosed.

Pharmacogenomics and Adverse Drug Reactions

Adverse drug reactions (ADRs) remain a major clinical problem. A recent meta-analysis suggested that in the USA in 1994, ADRs were responsible for 100 000 deaths, making them between the fourth and sixth commonest cause of death (Lazarou 1998, J. Am. Med. Assoc. 279:1200). Although these figures have been heavily criticized, they emphasize the importance of ADRs. Indeed, there is good evidence that ADRs account for 5% of all hospital admissions and increase the length of stay in hospital by two days at an increased cost of ˜$2500 per patient. ADRs are also one of the commonest causes of drug withdrawal, which has enormous financial implications for the pharmaceutical industry. ADRs, perhaps fortunately, only affect a minority of those taking a particular drug. Although factors that determine susceptibility are unclear in most cases, there is increasing interest in the role of genetic factors. Indeed, the role of inheritable variations in predisposing patients to ADRs has been appreciated since the late 1950s and early 1960s through the discovery of deficiencies in enzymes such as pseudocholinesterase (butyryl-cholinesterase) and glucose-6-phosphate dehydrogenase (G6PD). More recently, with the first draft of the human genome just completed, there has been renewed interest in this area with the introduction of terms such as pharmacogenomics and toxicogenomics. Essentially, the aim of pharmacogenomics is to produce personalized medicines, whereby administration of the drug class and dosage is tailored to an individual genotype. Thus, the term pharmacogenomics embraces both efficacy and toxicity.

The 3-hydroxy-3-methylglutaryl coenzyme A (HMG-CoA) reductase inhibitors (“statins”) specifically inhibit the enzyme HMG-CoA reductase which catalyzes the rate limiting step in cholesterol biosynthesis. These drugs are effective in reducing the primary and secondary risk of coronary -artery disease and coronary events, such as heart attack, in middle-aged and older men and women, in both diabetic and non-diabetic patients, and are often prescribed for patients with hyperlipidemia. Statins used in secondary prevention of coronary artery or heart disease significantly reduce the risk of stroke, total mortality and morbidity and attacks of myocardial ischemia; the use of statins is also associated with improvements in endothelial and fibrinolytic functions and decreased platelet thrombus formation.

The tolerability of these drugs during long term administration is an important issue. Adverse reactions involving skeletal muscle are not uncommon, and sometimes serious adverse reactions involving skeletal muscle such as myopathy and rhabdomyolysis may occur, requiring discontinuation of the drug. In addition an increase in serum creatine kinase (CK) may be a sign of a statin related adverse event. The extend of such adverse events can be read from the extend of the CK level increase (as compared to the upper limit of normal [ULN]).

Occasionally arthralgia, alone or in association with myalgia, has been reported. Also an elevation of liver transaminases has been associated with statin administration.

It was shown that the drug response to statin therapy is a class effects, i.e. all known and presumably also all so far undiscovered statins share the same benefical and harmful effects (Ucar, M. et al., Drug Safety 2000, 22:441). It follows that the discovery of diagnostic tools to predict the drug response to a single statin will also be of aid to guide therapy with other statins.

The present invention provides diagnostic tests to predict the patient's individual response to statin therapy. Such responses include, but are not limited by the extent of adverse drug reactions, the level of lipid lowering or the drug's influence on disease states. Those diagnostic tests may predict the response to statin therapy either alone or in combination with another diagriostic test or another drug regimen.

### DETAILED DESCRIPTION OF THE INVENTION

The present invention is based at least in part on the discovery that a specific allele of a polymorphic region of a so called “candidate gene” (as defined below) is associated with CVD or drug response.

For the present invention the following candidate genes were analyzed:
Genes found to be expressed in-cardiac tissue (Hwang et al., Circulation 1997, 96:4146-4203).
Genes from the following metabolic pathways and their regulatory elements:
   Lipid Metabolism

Numerous studies have shown a connection between serum lipid levels and cardiovascular diseases. Candidate genes falling into this group include but are not limited by genes of the cholesterol pathway, apolipoproteins and their modifiying factors.

Coagulation

Ischemic diseases of the heart and in particular myocardial infarction may be caused by a thrombotic occlusion. Genes falling into this group include all genes of the coagulation cascade and their regulatory elements.

Inflammation

Complications of atherosclerosis are the most common causes of death in Western societies. In broad outline atherosclerosis can be considered to be a form of chronic inflammation resulting from interaction modified lipoproteins, monocyte-derived macrophages, T cells, and the normal cellular elements of the arterial wall. This inflammatory process can ultimately lead to the development of complex lesions, or plaques, that protrude into the arterial lumen. Finally plaque rupture and thrombosis result in the acute clinical complications of myocardial infarction and stroke (Glass et al., Cell 2001, 104:503-516).

It follows that all genes related to inflammatory processes, including but not limited by cytokines, cytokine receptors and cell adhesion molecules are candidate genes for CVD.

Glucose and Energy Metabolism

As glucose and energy metabolism is interdependent with the metabolism of lipids (see above) also the former pathways contain candidate genes. Energy metabolism in general also relates to obesity, which is an independent risk factor for CVD (Melanson et al., Cardiol Rev 2001 9:202-207). In addition high blood glucose levels are associated with many microvascular and macrovascular complications and may therefore affect an individuals disposition to CVD (Duckworth, Curr Atheroscler Rep 2001, 3:383-391).

Hypertension

As hypertension is an independent risk factor for CVD, also genes that are involved in the regulation of systolic and diastolic blood pressure affect an individuals risk for CVD (Safar, Curr Opin Cardiol 2000, 15:258-263). Interestingly hypertension and diabetes (see above) appear to be interdependent, since hypertension is approximately twice as frequent in patients with diabetes compared with patients without the disease. Conversely, recent data suggest that hypertensive persons are more predisposed to the development of diabetes than are normotensive persons (Sowers et al., Hypertension 2001, 37:1053-1059).

Genes Related to Drug Response

Those genes include metabolic pathways involved in the absorption, distribution, metabolism, excretion and toxicity (ADMET) of drugs. Prominent members of this group are the cytochrome P450 proteins which catalyze many reactions involved in drug metabolism.

Unclassified Genes

As stated above, the mechanisms that lead to cardiovascular diseases or define the patient's individual response to drugs are not completely elucidated. Hence also candidate genes were analysed, which could not be assigned to the above listed categories. The present invention is based at least in part on the discovery of polymorphisms, that lie in genomic regions of unknown physiological function.

Results

After conducting an association study, we surprisingly found polymorphic sites in a number of candidate genes which show a strong correlation with the following phenotypes of the patients analysed: “Healthy” as used herein refers to individuals that neither suffer from existing CVD, nor exhibit an increased risk for CVD through their serum lipid level profile. “CVD prone” as used herein refers to individuals with existing CVD and/or a serum lipid profile that confers a high risk to get CVD (see Table 1a for definitions of healthy and CVD prone serum lipid levels). “High responder” as used herein refers to patients who benefit from relatively small amounts of a given drug. “Low responder” as used herein refers to patients who need relatively high doses in order to obtain benefit from the medication. “Tolerant patient” refers to individuals who can tolerate high doses of a medicament without exhibiting adverse drug reactions. “ADR patient” as used herein refers to individuals who suffer from ADR or show clinical symptoms (like creatine kinase elevation in blood) even after receiving only minor doses of a medicament (see Table 1b for a detailed definition of drug response phenotypes).

Polymorphic sites in candidate genes that were found to be significantly associated with either of the above mentioned phenotypes will be referred to as “phenotype associated SNPs” (PA SNPs). The respective genomic loci that harbour PA SNPs will be referred to as “phenotype associated genes” (PA genes), irrespective of the actual function of this gene locus.

In particular we surprisingly found PA SNPs associated with CVD, drug efficacy (EFF) or adverse drug reactions (ADR) in the following genes.

### ABCB11: ATP-binding Cassette, Sub-family B (MDR/TAP), Member 11

The membrane-associated protein encoded by this gene is a member of the superfamily of ATP-binding cassette (ABC) transporters. ABC proteins transport various molecules across extra- and intra-cellular membranes. ABC genes are divided into seven distinct subfamilies (ABCI, MDR/TAP, MRP, ALD, OABP, GCN20, White). This protein is a member of the MDR/TAP subfamily. Members of the MDR/TAP subfamily are involved in multidrug resistance. The protein encoded by this gene is the major canalicular bile salt export pump in man. Mutations in this gene cause a form of progressive familial intrahepatic cholestases which are a group of inherited disorders with severe cholestatic liver disease from early infancy.

### ABCB4: ATP-binding Cassette, Sub-family B (MDR/TAP), Member 4

The membrane-associated protein encoded by this gene is a member of the superfamily of ATP-binding cassette (ABC) transporters. ABC proteins transport various molecules across extra- and intra-cellular membranes. ABC genes are divided into seven distinct subfamilies (ABC1, MDR/TAP, MRP, ALD, OABP, GCN20, White). This protein is a member of the MDR/TAP subfamily. Members of the MDR/TAP subfamily are involved in multidrug resistance as well as antigen presentation. This gene encodes a full transporter and member of the p-glycoprotein family of membrane proteins with phosphatidylcholine as its substrate. The function of this protein has not yet been determined; however, it may involve transport of phospholipids from liver hepatocytes into bile. Alternative splicing of this gene results in several products of undetermined function.

### ABCC1: ATP-binding Cassette, Sub-family C (CFTR/MRP), Member 1

The protein encoded by this gene is a member of the superfamily of ATP-binding cassette (ABC) transporters. ABC proteins transport various molecules across extra- and intra-cellular membranes. ABC genes are divided into seven distinct subfamilies (ABC1, MDR/TAP, MRP, ALD, OABP, GCN20, White). This full transporter is a member of the MRP subfamily which is involved in multi-drug resistance. This protein functions as a multispecific organic anion transporter, with oxidized glutatione, cysteinyl leukotrienes, and activated aflatoxin B1 as substrates. This protein also transports glucuronides and sulfate conjugates of steroid hormones and bile salts. Alternative splicing by exon deletion results in several splice variants but maintains the original open reading frame in all forms.

### ACTB mRNA for Mutant Beta-actin

Beta actin is one of six different actin isoforms which have been identified. ACTB is one of the two nonmuscle cytoskeletal actin. Actins are highly conserved proteins that are involved in cell motility, structure and integrity. Alpha actins are a major constituent of the contractile apparatus.

### Actin, Alpha Skeletal Muscle (Alpha-Actin 1)

Actin alpha 1 which is expressed in skeletal muscle is one of six different actin isoforms which have been identified. Actins are highly conserved proteins that are involved in cell motility, structure and integrity. Alpha actins are a major constituent of the contractile apparatus.

### ADCYAP1: Adenylate Cyclase Activating Polypeptide 1 (Pituitary)

This gene encodes adenylate cyclase activating polypeptide 1. Mediated by adenylate cyclase activating polypeptide 1 receptors, this polypeptide stimulates adenylate cyclase and subsequently increases the cAMP level in target cells. Adenylate cyclase activating polypeptide 1 is not only a hypophysiotropic hormone, but also functions as a neurotransmitter and neuromodulator. In addition, it plays a role in paracrine and autocrine regulation of certain types of cells. This gene is composed of five exons. Exons 1 and 2 encode the 5′UTR and signal peptide, respectively; exon 4 encodes an adenylate cyclase activating polypeptide 1-related peptide; and exon 5 encodes the mature peptide and 3′UTR This gene encodes three different mature peptides, including two isotypes: a shorter form and a longer form.

### ADRB3: Adrenergic, Beta-3-, Receptor

The ADRB3 gene product, beta-3-adrenergic receptor, is located mainly in adipose tissue and is involved in the regulation of lipolysis and thermogenesis. Beta adrenergic receptors are involved in the epenephrine and norepinephrine-induced activation of adenylate cyclase through the action of G proteins.

### AGL: Amylo-1,6-glucosidase, 4-alpha-glucanotransferase (Glycogen Debranching Enzyme, Glycogen Storage Disease Type III)

Glycogen debranching enzyme is involved in glycogen degradation and has two independent catalytic activities: a 4-alpha-glucotransferase activity (EC 2.4.1.25) and a amylo-1,6-glucosidase activity (EC 3.4.1.33). Both activities occur at different sites on the single polypeptide chain. Mutations in this gene cause glycogen storage disease. A wide range of clinical and enzymatic variability occurs in glycogen debrancher deficiency, some of which may be due to tissue-specific alternative splicing. Six splice varients that differ in the 5′ end have been identified in liver and muscle tissue. Variants 1, 5, and 6 are present in both liver and muscle, whereas variants 2, 3, and 4 occur in muscle. Variants 1 through 4 encode identical proteins (isoform 1) that include 27 N-terminal amino acids not found in splice variants 5 and 6. Variants 5 and 6 encode different amino-terminal ends of 10 and 11 amino acids in protein isoforms 2 and 3, respectively, with the remainder of the peptide identical to that of isoforms 1.

### AKAP1: A Kinase (PRKA) Anchor Protein 1

Anchors cAMP-dependent protein kinase near its physiological substrates, interacts with both the type I and type II regulatory subunits.

### Angiotensinogen Gene

The protein encoded by this gene, pre-angiotensinogen or angiotensinogen precursor, is expressed in the liver and is cleaved by the enzyme renin in response to lowered blood pressure. The resulting product, angiotensin I is then cleaved by angiotensin converting enzyme (ACE) to generate the physiologically active enzyme angiotensin II. The protein is involved in maintaining blood pressure and in the pathogenesis of essential hypertension and preeclampsia.

### ANXA6: Annexin A6

Annexin VI belongs to a family of calcium-dependent membrane and phospholipid binding proteins. Although their functions are still not clearly defined, several members of the annexin family have been implicated in membrane-related events along exocytotic and endocytotic pathways. The annexin VI gene is approximately 60 kbp long and contains 26 exons. It encodes a protein of about 68 kDa that consists of eight 68-amino acid repeats separated by linking sequences of variable lengths. It is highly similar to human annexins I and II sequences, each of which contain four such repeats. Exon 21. of annexin VI is alternatively spliced, giving rise to two isoforms that differ by a 6-amino acid insertion at the start of the seventh repeat. Annexin VI has been implicated in mediating the endosome aggregation and vesicle fusion in secreting epithelia during exocytosis.

### AP2B1: Adaptor-Related Protein Complex 2, Beta 1 Subunit

The beta adaptin subunit is part of the clathrin coat assembly complex which links clathrin to receptors in coated pits and vesicles. These vesicles are involved in endocytosis and Golgi processing. The beta 1 subunit is one of the assembly proteins which binds to clathrin and-initiates coat formation.

### APOA1: Apolipoprotein A-I

APOA1 promotes cholesterol efflux from tissues to the liver for excretion. Apolipoprotein A-I is the major protein component of high density lipoprotein (HDL) in the plasma. Synthesized in the liver and small intestine, it consists of two identical chains of 77 amino acids; an 18-amino acid signal peptide is removed co-translationally and a 6-amino acid propeptide is cleaved post-translationally. Variation in the latter step, in addition to modifications leading to so-called isoforms, is responsible for some of the polymorphism observed. APOA1 is a cofactor for lecithin cholesterolacyltransferase (LCAT) which is responsible for the formation of most plasma cholesteryl esters. The APOA1, APOC3 and APOA4 genes are closely linked in both rat and human genomes. The A-I and A-IV genes are transcribed from the same strand, while the C-III gene is transcribed convergently in relation to A-I. Defects in the apolipoprotein A-1 gene are associated with HDL deficiency and Tangier disease.

### APOA4: Apolipoprotein A-IV

Apoliprotein (apo) A-IV gene contains 3 exons separated by two introns. A sequence polymorphism has been identified in the 3′UTR of the third exon. The primary translation product is a 396-residue preprotein which after proteolytic processing is secreted its primary site of synthesis, the intestine, in association with chylomicron particles. Although its precise function is not known, apo A-IV is a potent activator of lecithin-cholesterol acyltransferase in vitro.

### APOB: Apolipoprotein B

Apolipoprotein B (ApoB) is the main apolipoprotein of chylomicrons and low density lipoproteins (LDL). The protein occurs in the plasma in 2 main isoforms, apoB-48 and apoB-100. The first is synthesized exclusively by the gut, the second by the liver. The intestinal (B-48) and hepatic (B-100) forms of apoB are coded by a single gene and by a single mRNA transcript larger than 16 kb. The 2 proteins share a common amino terminal sequence. In the ApoB-100 isoform the precursor has 4,563 amino acids, and the mature apoB-100 has 4,536 amino acid residues. Mature, circulating B-48 is homologous over its entire length (estimated to be between 2,130 and 2,144 amino acid residues) with the amino-terminal portion of B-100 and contains no sequence from the carboxyl end of B-100. From structural studies, it is thought that apoB-48 represents the amino-terminal 47% of apoB-100 and that the carboxyl terminus of apoB48 is in the vicinity of residue 2151 of apoB-100. Apolipoprotein B-48 may be the product of an intestinal mRNA with an in-frame UAA stop codon resulting from a C-to-U change in the codon CAA encoding Gln(2153) in apoB-100 mRNA. Since only the sequence that codes B-100 is present in genomic DNA, this presents the possibility of an organ-specific introduction of a stop codon to an mRNA and the change from CAA to UAA of codon 2153 of the message as a unique RNA editing process.

### APOD: Apolipoprotein D

Apolipoprotein D (Apo-D) is a component of high density lipoprotein that has no marked similarity to other apolipoprotein sequences. It has a high degree of homology to plasma retinol-binding protein and other members of the alpha 2 microglobulin protein superfamily of carrier proteins, also known as lipocalins. It is a glycoprotein of estimated molecular weight 33 KDa. Apo-D is closely associated with the enzyme lecithin:cholesterol acyltransferase—an enzyme involved in lipoprotein metabolism.

### Apolipoprotein B

Apolipoprotein B (ApoB) is the main apolipoprotein of chylomicrons and low density lipoproteins (LDL). The protein occurs in the plasma in 2 main isoforms, apoB-48 and apoB-100. The first is synthesized exclusively by the gut, the second by the liver. The intestinal (B-48) and hepatic (B-100) forms of apoB are coded by a single gene and by a single mRNA transcript larger than 16 kb. The 2 proteins share a common amino terminal sequence. In the ApoB-100 isoform the precursor has 4,563 amino acids, and the mature apoB-100 has 4,536 amino acid residues. Mature, circulating B-48 is homologous over its entire length (estimated to be between 2,130 and 2,144 amino acid residues) with the amino-terminal portion of B-100 and contains no sequence from the carboxyl end of B-100. From structural studies, it is thought that apoB48 represents the amino-terminal 47% of apoB-100 and that the carboxyl terminus of apoB-48 is in the vicinity of residue 2151 of apoB-100. Apolipoprotein B-48 may be the product of an intestinal mRNA with an in-frame UAA stop codon resulting from a C-to-U change in the codon CAA encoding Gln(2153) in apoB-100 mRNA. Since only the sequence that codes B-100 is present in genomic DNA, this presents the possibility of an organ-specific introduction of a stop codon to an mRNA and the change from CAA to UAA of codon 2153 of the message as a unique RNA editing process.

### APXL: Apical Protein-Like (Xenopus laevis)

The protein encoded by this gene shares significant similarities with the apical protein from Xenopus laevis which is implicated in amiloride-sensitive sodium. channel activity. This gene is a strong candidate gene for ocular albinism type 1 syndrome.

### ARF4: ADP-ribosylation Factor 4

ADP-ribosylation factor 4 (ARF4) is a member of the human ARF gene family. These genes encode small guanine nucleotide-binding proteins that stimulate the ADP-ribosyltransferase activity of cholera toxin and play a role in vesicular trafficking and as activators of phospholipase D. The gene products include 6 ARF proteins and 11 ARF-like proteins and constitute 1 family of the RAS superfamily. The ARF proteins are categorized as class I (ARF1, ARF2,and ARF3), class II (ARF4 and ARF5) and class III (ARF6). The members of each class share a common gene organization. The ARF4 gene spans approximately 12 kb and contains six exons and five introns. The ARF4 is the most divergent member of the human ARFs. Conflicting Map positions at 3p14 or 3p21 have been reported for this gene.

### ATP1A2: ATPase, Na+/K+ Transporting, Alpha 2 (+) Polypeptide

Alpha 2 subunit of the sodium- and potassium-transporting ATpase; required for Na+ and K+ gradient maintenance across plasma membrane.

### ATP1B1: ATPase, Na+/K+ Transporting, Beta 1 Polypeptide

Beta 1 subunit of Na+/K+-ATPase.

### ATP1B3: ATPase, Na+/K+ Transporting, Beta 3 Polypeptide

Beta 3 subunit of the Na+/K+-ATPase.

### ATP2A2: ATPase, Ca++ Transporting, Cardiac Muscle, Slow Twitch 2

Slow twitch cardiac muscle Ca2+-ATPase; pumps calcium, may have a role in calcium signaling pathways.

### ATP5G1: ATP Synthase, H+ Transporting, Mitochondrial F0 Complex, Subunit c (Subunit 9), Isoform 1

Isoform 1 (P1) of subunit c, H+-translocating subunit of F0 ATP synthase; catalyzes the synthesis of ATP during oxidative phosphorylation.

### ATP6V1E: ATPase, H+ Transporting, Lysosomal 31 kD, V1 Subunit E

This gene encodes a component of vacuolar ATPase (V-ATPase), a multisubunit enzyme that mediates acidification of eukaryotic intracellular organelles. V-ATPase dependent organelle acidification is necessary for such intracellular processes as protein sorting, zymogen activation, and receptor-mediated endocytosis. V-ATPase is comprised of a cytosolic V1 domain and a transmembrane V0 domain. The V1 domain consists of a hexamer of three A and three B subunits plus the C, D, and E subunits. It contains the ATP catalytic site. The encoded protein is known as the E subunit and is found ubiquitously. Pseudogenes for this gene have been found in the genome.

### ATPase, Ca++ Transporting, Cardiac Muscle, Fast Twitch 1

Fast-twitch skeletal muscle sarcoplasmic reticulum Ca2+-ATPase; pumps calcium.

### AXIN1: Axin

Strongly similar to murine Axin; may regulate embryonic axis formation.

### BMPR1A: Bone Morphogenetic, Protein Receptor, Type IA

The bone morphogenetic protein (BMP) receptors are a family of transmembrane serine/threonine kinases that include the type I receptors BMPR1A and BMPR1B and the type II receptor BMPR2. These receptors are also closely related to the activin receptors, ACVR1 and ACVR2. The ligands of these receptors are members of the TGF-beta superfamily. TGF-betas and activins transduce their signals through the formation of heteromeric complexes with 2 different types of serine (threonine) kinase receptors: type I receptors of about 50-55 kD and type II receptors of about 70-80 kD. Type II receptors bind ligands in the absence of type I receptors, but they require their respective type I receptors for signaling, whereas type I receptors require their respective type II receptors for ligand binding.

### BRD3: Bromodomain Containing 3

This gene was identified based on its homology to the gene encoding the RING3 protein, a serine/threonine kinase. The gene localizes to 9q34, a region which contains several major histocompatibility complex (MHC) genes. The function of the encoded protein is not known.

### CACNA1C: Calcium Channel, Voltage-Dependent, L Type, Alpha 1C Subunit

Alpha 1C subunit of the voltage-dependent calcium channel; channel is of the L type and is expressed in the heart.

### CALB2: Calbindin 2, (29 kD, Calretinin)

Calbindin 2 (calretinin), closely related to calbindin 1, is an intracellular calcium-binding protein belonging to the troponin C superfamily. Calbindin 1 is known to be involved in the vitamin-D-dependent calcium absorption through intestinal and renal epithelia, while the function of neuronal calbindin 1 and calbindin 2 is poorly understood. The sequence of the calbindin 2 cDNA reveals an open reading frame of 271 codons coding for a protein of 31,520 Da, and shares 58% identical residues with human calbindin 1. Calbindin 2 contains five presumably active and one presumably inactive calcium-binding domains. Comparison with the partial sequences available for chick and guinea pig calbindin 2 reveals that the protein is highly conserved in evolution. The calbindin 2 message was detected in the brain, while absent from heart muscle, kidney, liver, lung, spleen, stomach and thyroid gland. There are two additional forms of alternatively spliced calbindin 2 mRNAs encoding C-terminally truncated proteins. Exon 7 can splice to exon 9, resulting in a frame shift and a translational stop at the second codon of exon 9, and encoding calretinin-20k. Exon 7 can also splice to exon 10, resulting in a frame shift and a translational stop at codon 15 of exon 10, and encoding calretinin-22k. The truncated proteins are able to bind calcium.

### Calcium-Transporting ATPase Plasma Membrane, Isoforms 3A/3B (EC 3.6.1.38) (Calcium Pump) (PMCA3)

Plasma membrane Ca2+-ATPase 3; pumps calcium.

### CALM3: Calmodulin 3 (Phosphorylase Kinase, Delta)

Calmodulin 3; binds calcium.

### CAV1: Caveolin 1, Caveolae Protein, 22 kD

The scaffolding protein encoded by this gene is the main component of the caveolae plasma membranes found in most cell types. The protein links integrin subunits to the tyrosine kinase FYN, an initiating step in coupling integrins to the Ras-ERK pathway and promoting cell cycle progression. The gene is a tumor suppressor gene candidate and a negative regulator of the Ras-p42/44 MAP kinase cascade. CAV1 and CAV2 are located next to each other on chromosome 7 and express colocalizing proteins that form a stable hetero-oligomeric complex. By using alternative initiation codons in the same reading frame, two isoforms (alpha and beta) are encoded by a single transcript from this gene.

### CAV3: Caveolin 3

This gene encodes a caveolin family member, which functions as a component of the caveolae plasma membranes found in most cell types. Caveolin proteins are proposed to be scaffolding proteins for organizing and concentrating certain caveolin-interacting molecules. Mutations identified in this gene lead to interference with protein oligomerization or intra-cellular routing, disrupting caveolae formation and resulting in Limb-Girdle muscular dystrophy type-1C (LGMD-1C), hyperCKemia or rippling muscle disease (RMD). Alternative splicing has been identified for this locus, with inclusion or exclusion of a differentially spliced intron. In addition, transcripts utilize multiple polyA sites and contain two potential translation initiation sites.

### CCR2: Chemokine (C-C Motif) Receptor 2

This gene encodes two isoforms of a receptor for monocyte chemoattractant protein-1, a chemokine which specifically mediates monocyte chemotaxis. Monocyte chemoattractant protein-1 is involved in monocyte infiltration in inflammatory diseases such as rheumatoid arthritis as well as in the inflammatory response against tumors. The receptors encoded by this gene mediate agonist-dependent calcium mobilization and inhibition of adenylyl cyclase. This gene is located in the chemokine receptor gene cluster region. Two alternatively spliced transcript variants are expressed by the gene.

### CDH1: Cadherin 1, Type 1, E-cadherin (Epithelial)

This gene is a classical cadherin from the cadherin superfamily. The encoded protein is a calcium dependent cell-cell adhesion glycoprotein comprised of five extracellular cadherin repeats, a transmembrane region and a highly conserved cytoplasmic tail. Mutations in this gene are correlated with gastric, breast, colorectal, thyroid and ovarian cancer. Loss of function is thought to contribute to progression in cancer by increasing proliferation, invasion, and/or metastasis. The ectodomain of this protein mediates bacterial adhesion to mammalian cells and the cytoplasmic domain is required for internalization. Identified transcript variants arise from mutation at consensus splice sites.

### CDH11: Cadherin 11, Type 2, OB-cadherin (Osteoblast)

This gene encodes a type II classical cadherin from the cadherin superfamily, integral membrane proteins that mediate calcium-dependent cell-cell adhesion. Mature cadherin proteins are composed of a large N-terminal extracellular domain, a single membrane-spanning domain, and a small, highly conserved C-terminal cytoplasmic domain. Type II (atypical) cadherins are defined based on their lack of a HAV cell adhesion recognition sequence specific to type I cadherins. Expression of this particular cadherin in osteoblastic cell lines, and its upregulation during differentiation, suggests a specific function in bone development and maintenance. Two splice variants have been identified, one of which encodes an isoform with a truncated cytoplasmic domain.

### CDH13: Cadherin 13, H-cadherin (Heart)

This gene is a member of the cadherin superfamily. The encoded protein is a calcium dependent cell-cell adhesion glycoprotein comprised of five extracellular cadherin repeats, a transmembrane region but, unlike the typical cadherin superfamily member, lacks the highly conserved cytoplasmic region. This particular cadherin is a putative mediator of cell-cell interaction in the heart and may act as a negative regulator of neural cell growth. The gene locus is hypermethylated or deleted in breast, ovarian and lung cancers. Two major mRNA transcripts encoding identical proteins are found, products of alternative polyadenylation sites.

### CENPC1: Centromere Protein C1

Centromere protein C1 is a centromere autoantigen and a component of the inner kinetochore plate. The protein is required for maintaining proper kinetochore size and a timely transition to anaphase. A putative psuedogene exists on chromosome 12.

### Cholesteryl Ester Transfer Protein (CETP)

Cholestery ester transfer protein (CETP) transfers cholesteryl esters between lipoproteins. CETP may effect susceptibility to atherosclerosis.

### CLCN4: Chloride Channel 4

The CLCN family of voltage-dependent chloride channel genes comprises nine members (CLCN1-7, Ka and Kb) which demonstrate quite diverse functional characteristics while sharing significant sequence homology. Chloride channel 4 has an evolutionary conserved CpG island and is conserved in both mouse and hamster. This gene is mapped in close proximity to APXL (Apical protein Xenopus laevis-like) and OA1 (Ocular albinism type I), which are both located on the human X chromosome at band p22.3. The physiological role of chloride channel 4 remains unknown but may contribute to the pathogenesis of neuronal disorders.

### CLCNKA: Chloride Channel Ka

Putative chloride channel; member of the CLC family of voltage-gated chloride channels.

### COL6A3: Collagen, Type VI, Alpha 3

This gene encodes the alpha 3 chain, one of the three alpha chains of type VI collagen, a beaded filament,collagen found in most connective tissues. The alpha 3 chain of type VI collagen is much larger than the alpha 1 and 2 chains. This difference in size is largely due to an increase in the number of subdomains, similar to Von Willebrand Factor type A domains, found in the amino terminal globular domain of all the alpha chains. These domains have been shown to bind extracellular matrix proteins, an interaction that explains the importance of this collagen in organizing matrix components. Mutations in the type VI collagen genes are associated with Bethlem myopathy. In addition to the full length transcript, four transcript variants have been identified that encode proteins with N-terminal globular domains of varying sizes.

### COL7A1: Collagen, Type VII, Alpha I (Epidermolysis Bullosa, Dystrophic, Dominant and Recessive)

This gene encodes the alpha chain of type VII collagen. The type VII collagen fibril, composed of three identical alpha collagen chains, is restricted to the basement zone beneath stratified squamous epithelia. It functions as an anchoring fibril between the external epithelia and the underlying stroma. Mutations in this gene are, associated with all forms of dystrophic epidermolysis bullosa. In the absence of mutations, however, an acquired form of this disease can result from an autoimmune response made to type VII collagen.

### COL9A3: Collagen, Type IX, Alpha 3

This gene encodes one of the three alpha chains of type IX collagen, the major collagen component of hyaline cartilage. Type IX collagen, a heterotrimeric molecule, is usually found in tissues containing type II collagen, a fibrillar collagen. Mutations in this gene are associated with multiple epiphyseal dysplasia.

### COMT: Catechol-O-methyltransferase

Catechol-O-methyltransferase catalyzes the transfer of a methyl group from S-adenosylmethionine to catecholamines, including the neurotransmitters dopamine, epinephrine, and norepinephrine. This O-methylation results in one of the major degradative pathways of the catecholamine transmitters. In addition to its role in the metabolism of endogenous substances, COMT is important in the metabolism of catechol drugs used in the treatment of hypertension, asthma, and Parkinson disease. COMT is found in two forms in tissues, a soluble form (S-COMT) and a membrane-bound form (MB-COMT). The differences between S-COMT and MB-COMT reside within the N-termini. The transcript variants are formed through the use of alternative translation initiation sites and promoters.

### COX10: COX10 Homolog, Cytochrome c Oxidase Assembly Protein, Heme A: Farnesyltransferase (Yeast)

Cytochrome c oxidase (COX), the terminal component of the mitochondrial respiratory chain, catalyzes the electron transfer from reduced cytochrome c to oxygen. This component is a heteromeric complex consisting of 3 catalytic subunits encoded by mitochondrial genes and multiple structural subunits encoded by nuclear genes. The mitochondrially-encoded subunits function in electron transfer, and the nuclear-encoded subunits may function in the regulation and assembly of the complex. This nuclear gene encodes heme A:farnesyltransferase, which is not a structural subunit but required for the expression of functional COX and functions in the maturation of the heme A prosthetic group of COX. This protein is predicted to contain 7-9 transmembrane domains localized in the mitochondrial inner Membrane. A gene mutation, which results in the substitution of a lysine for an asparagine (N204K), is identified to be responsible for cytochrome c oxidase deficiency. In addition, this gene is disrupted in patients with CMT1A (Charcot-Marie-Tooth type 1A) duplication and with HNPP (hereditary neuropathy with liability to pressure palsies) deletion.

### CPB2: Carboxypeptidase B2 (Plasma, Carboxypeptidase U)

Carboxypeptidases are enzymes that hydrolyze C-terminal peptide bonds. The carboxypeptidase family includes metallo-, serine, and cysteine carboxypeptidases. According to their substrate specificity, these enzymes are referred to as carboxypeptidase A (cleaving aliphatic residues) or carboxypeptidase B (cleaving basic amino residues). The protein encoded by this gene is activated by trypsin and acts on carboxypeptidase B substrates. After thrombin activation, the mature protein downregulates fibrinolysis. Polymorphisms have been described for this gene and its promoter region. Available sequence data analyses indicate splice variants that encode different isoforms.

### CPO: Coproporphyrinogen Oxidase (Coproporphyria, Harderoporphyria)

Coproporphyrinogen; catalyzes oxidative decarboxylation in sixth step of heme biosynthesis.

### CRYAB: Crystallin, Alpha B

Crystallins are separated into-two classes: taxon-specific, or enzyme, and ubiquitous. The latter class constitutes the major proteins of vertebrate eye lens and maintains the transparency and refractive index of the lens. Since lens central fiber cells lose their nuclei during development, these crystallins are made and then retained throughout life, making them extremely stable proteins. Mammalian lens crystallins are divided into alpha, beta, and gamma families; beta and gamma crystallins are also considered as a superfamily. Alpha and beta families are further divided into acidic and basic groups. Seven protein regions exist in crystallins: four homologous motifs, a connecting peptide, and N— and C-terminal extensions. Alpha crystallins are composed of two gene products: alpha-A and alpha-B, for acidic and basic, respectively. Alpha crystallins can be induced by heat shock and are members of the small heat shock protein (sHSP also known as the HSP20) family. They act as molecular chaperones although they do not renature proteins and release them in the fashion of a true chaperone; instead they hold them in large soluble aggregates. Post-translational modifications decrease the ability to chaperone. These heterogeneous aggregates consist of 30-40 subunits; the alpha-A and alpha-B subunits have a 3:1 ratio, respectively. Two additional functions of alpha crystallins are an autokinase activity and participation in the intracellular architecture. Alpha-A and alpha-B gene products are differentially expressed; alpha-A is preferentially restricted to the lens and alpha-B is expressed widely in many tissues and organs. Elevated expression of alpha-B crystallin occurs in many neurological diseases; a missense mutation cosegregated in a family with a desmin-related myopathy.

### CSF2RB: Colony Stimulating Factor 2 Receptor, Beta, Low-Affinity (Granulocyte-Macrophage)

CSF2RB is a common beta chain of the high affinity receptor for IL-3, IL-5 and CSF. Defective CSF2RB has been-reported to be associated with protein alveolar proteinosis.

### CUBN: Cubilin (Intrinsic Fact r-cobalamin Receptor)

Cubilin (CUBN) acts as a receptor for intrinsic factor-vitamin B12 complexes. The role of receptor is supported by the presence of 27 CUB domains. Cubulin is located within the epithelium of intestine and kidney. Mutations in CUBN may play a role in autosomal recessive megaloblastic anemia.

### CXorf6: Chromosome X Open Reading Frame 6

### CYP17: Cytochrome P450, Subfamily XVII (Steroid 17-alpha-hydroxylase), Adrenal Hyperplasia

This gene encodes a member of the cytochrome P450 superfamily of enzymes. The cytochrome P450 proteins are monooxygenases which catalyze many reactions involved in drug metabolism and synthesis of cholesterol, steroids and other lipids. This protein localizes to the endoplasmic reticulum. It has both 17alpha-hydroxylase and 17,20-lyase activities and is a key enzyme in the steroidogenic pathway that produces progestins, mineralocorticoids, glucocorticoids, androgens, and estrogens. Mutations in this gene are associated with isolated steroid-17 alpha-hydroxylase deficiency, 17-alpha-hydroxylase/17,20-lyase deficiency, pseudohermaphroditism, and adrenal hyperplasia

### CYP2C8: Cytochrome P450, Subfamily IIC (Mephenytoin 4-hydroxylase), Polypeptide 8

This gene encodes a member of the cytochrome P450 superfamily of enzymes. The cytochrome P450 proteins are monooxygenases which catalyze many reactions involved in drug metabolism and synthesis of cholesterol, steroids and other lipids. This protein localizes to the endoplasmic reticulum and its expression is induced by phenobarbital. The enzyme is known to metabolize many xenobiotics, including the anticonvulsive drug mephenytoin, benzo(a)pyrene, 7-ethyoxycoumarin, and the anti-cancer drug taxol. Two transcript variants for this gene have been described; it is thought that the longer form does not encode an active cytochrome P450 since its protein product lacks the heme binding site. This gene is located within a cluster of cytochrome P450 genes on chromosome 10q24.

### CYP2E: Cytochrome P450, Subfamily HIE (Ethanol-Inducible)

This gene encodes a member of the cytochrome P450 superfamily of enzymes. The cytochrome P450 proteins are monooxygenases which catalyze many reactions involved in drug metabolism and synthesis of cholesterol, steroids and other lipids. This protein localizes to the endoplasmic reticulum and is induced by ethanol, the diabetic state, and starvation. The enzyme metabolizes both endogenous substrates, such as ethanol, acetone, and acetal, as well as exogenous substrates including benzene, carbon tetrachloride, ethylene glycol, and nitrosamines which are premutagens found in cigarette smoke. Due to its many substrates, this enzyme may be involved in such varied processes as gluconeogenesis, hepatic cirrhosis, diabetes; and cancer.

### CYP3A4

This gene, CYP3A4, encodes a member of the cytochrome P450 superfamily of enzymes. The cytochrome P450 proteins are monooxygenases which catalyze many reactions involved in drug metabolism and synthesis of cholesterol, steroids and other lipids. This protein localizes to the endoplasmic reticulum and its expression is induced by glucocorticoids and some pharmacological agents. This enzyme is involved in the metabolism of approximately half the drugs which are are used today, including acetaminophen, codeine, cyclosporin A, diazepam and erythromycin. The enzyme also metabolizes some steroids and carcinogens. This gene is part of a cluster of cytochrome P450 genes on chromosome 7q21.1. Previously another CYP3A gene, CYP3A3, was thought to exist; however, it is now thought that this sequence represents a transcript variant of CYP3A4.

### CYP4F8: Cytochrome P450, Subfamily IVF, Polypeptide 8

This gene, CYP4F8, encodes a member of the cytochrome P450 superfamily of enzymes. The cytochrome P450 proteins are monooxygenases which catalyze many reactions involved in drug metabolism and synthesis of cholesterol, steroids and other lipids. This protein localizes to the endoplasmic reticulum and functions as a 19-hydroxylase of prostaglandins in seminal vesicles. This gene is part of a cluster of cytochrome P450 genes on chromosome 19. Another member of this family, CYP4F3, is approximately 18 kb away.

### CYP8B1: Cytochrome P450, Subfamily VIIIB (Sterol 12-alpha-hydroxylase), Polypeptide 1

This gene encodes a member of the cytochrome P450 superfamily of enzymes. The cytochrome P450 proteins are monooxygenases which catalyze many reactions involved in drug metabolism and synthesis of cholesterol, steroids and other lipids. This endoplasmic reticulum membrane protein catalyzes the conversion of 7 alpha-hydroxy-4-cholesten-3-one into 7-alpha,12-alpha-dihydroxy-4-cholesten-3-one. The balance between these two steroids determines the relative amounts of cholic acid and chenodeoxycholic acid both of which are secreted in the bile and affect the solubility of cholesterol. This gene is unique among the cytochrome P450 genes in that it is intronless.

### DBI: Diazepam Binding Inhibitor (GABA Receptor Modulator, Acyl-Coenzyme A Binding Protein)

Diazepam binding inhibitor (acyl-CoA-binding protein); binds and induces medium-chain acyl-CoA ester synthesis.

### DEFA6: Defensin, Alpha 6, Paneth Cell-Specific

Defensins are a family of microbicidal and cytotoxic peptides thought to be involved in host defense. They are abundant in the granules of neutrophils and also found in the epithelia of mucosal surfaces such as those of the intestine, respiratory tract, urinary tract, and vagina. Members of the defensin family are highly similar in protein sequence and distinguished by a conserved cysteine motif. Several alpha defensin genes appear to be clustered on chromosome 8. The protein encoded by this gene, defensin, alpha 6, is highly expressed-in the secretory granules of Paneth cells of the small intestine, and likely plays a role in host defense of human bowel.

### DEK: DEK Oncogene (DNA Binding)

Site-specific DNA binding protein; involved in transcriptional regulation and signal transduction.

### DFNA5: Deafness, Autosomal Dominant 5

Hearing impairment is a heterogeneous condition with over 40 loci described. The protein encoded by this gene is expressed in fetal cochlea, however, its function is not known. Nonsyndromic hearing impairment is associated with a mutation in this gene.

### DGKD: Diacylglycerol Kinase, Delta (130 kD)

Diacylglycerol kinase delta; phosphorylates the arachidonoyl type of diacylglycerol; contains a pleckstrin homology domain and an EPH domain.

### DOCK1: Dedicator of Cyto-kinesis 1

Dedicator of cyto-kinesis 1 binds to the SH3 domain of CRK protein. It may regulate cell surface extension and may have a role in the cell surface extension of an engulfing cell around a dying cell during apoptosis.

### ECE1: Endothelin Converting Enzyme 1

Endothelin converting enzyme; metalloprotease that regulates a peptide involved in vasocontriction.

### E-Selectin (CD62E)

The endothelial leukocyte adhesion molecule-i is expressed by cytokine-stimulated endothelial cells. It is thought to be responsible for the accumulation of blood leukocytes at sites of inflammation by mediating the adhesion of cells to the vascular lining. It exhibits structural features such as the presence of lectin- and EGF-like domains followed by short consensus repeat (SCR) domains that contain 6 conserved cysteine residues. These proteins are part of the selectin family of cell adhesion molecules. This gene is present in single copy in the human genome and contains 14 exons spanning about 13 kb of DNA. Adhesion molecules participate in the interaction between leukocytes and the endothelium and appear to be involved in the pathogenesis of atherosclerosis.

### ESR1: Estrogen Receptor 1

Estrogen receptor; nuclear receptor transcription factor activated by ligand-binding, involved in hormone-mediated inhibition of gene expression.

### ESR2: Estrogen Receptor 2 (ER Beta)

Estrogen receptor beta 2; transcriptional activator involved in regulation of reproduction; exists in five isoforms.

### F2: Coagulation Factor II (Thrombin)

Coagulation factor II is proteolytically cleaved to form thrombin in the first step of the coagulation cascade which ultimately results in the stemming of blood loss. F2 also plays a role in maintaining vascular integrity during development and postnatal life. Mutations in F2 leads to various forms of thrombosis and dysprothrombinemia.

### F3: Coagulation Factor III (Thromboplastin, Tissue Factor)

This gene encodes coagulation factor III which is a cell surface glycoprotein. This factor enables cells to initiate the blood coagulation cascades, and it functions as the high-affinity receptor for the coagulation factor VII. The resulting complex provides a catalytic event that is responsible for initiation of the coagulation protease cascades by specific limited proteolysis. Unlike the other cofactors of these protease cascades, which circulate as nonfunctional precursors, this factor is a potent initiator that is fully functional when expressed on cell surfaces. There are 3 distinct domains of this factor: extracellular, transmembrane, and cytoplasmic. This protein is the only one in the coagulation pathway for which a congenital deficiency has not been described.

### F5: Coagulation Factor V (Proaccelerin, Labile Factor)

This gene encodes coagulation factor V which is an essential factor of the blood coagulation cascade. This factor circulates in plasma, and is converted to the active form by the release of the activation peptide by thrombin during coagulation. This generates a heavy chain and a light chain which are held together by calcium ions. The active factor V is a cofactor that participates with activated coagulation factor X to activate prothrombin to thrombin. Defects in this gene result in either -an autosomal recessive hemorrhagic diathesis or an autosomal dominant form of thrombophilia, which is known as activated protein C resistance.

### F7: Coagulation Factor VII (Serum Prothrombin Conversion Accelerator)

This gene encodes coagulation factor VII which is a vitamin K-dependent factor essential for hemostasis. This factor circulates in the blood in a zymogen form, and is converted to an active form by either factor IXa, factor Xa, factor XIIa, or thrombin by minor proteolysis. Upon activation of the factor VII, a heavy chain containing a catalytic domain and a light chain containing 2 EGF-like domains are generated, and two chains are held together by a disulfide bond. In the presence of factor III and calcium ions, the activated factor then further activates the coagulation cascade by converting factor IX to factor IXa and/or factor X to factor Xa. Alternative splicing of this gene results in 2 transcripts. Defects in this gene can cause coagulopathy.

### F9: Coagulation Factor IX (Plasma Thromboplastic Component, Christmas Disease, Hemophilia B)

This gene encodes vitamin K-dependent coagulation factor IX that circulates in the blood as an inactive zymogen. This factor is converted to an active form by factor XIa, which excises the activation peptide and thus generates a heavy chain and a light chain held together by one or more disulfide bonds. The role of this activated factor IX in the blood coagulation cascade is to activate factor X to its active form through interactions with Ca+2 ions, membrane phospholipids, and factor VIII. Alterations of this gene, including point mutations, insertions and deletions, cause, factor IX deficiency, which is a recessive X-linked disorder, also called hemophilia B or Christmas disease.

### FABP3: Fatty Acid Binding Protein 3, Muscle and Heart (Mammary-Derived Growth Inhibitor)

The intracellular fatty acid-binding proteins (FABPs) belongs to a multigene family. FABPs are divided into at least three distinct types, namely the hepatic-, intestinal- and cardiac-type. They form 14-15 kDa proteins and are thought to participate in the uptake, intracellular metabolism and/or transport of long-chain fatty acids. They may also be responsible in the modulation of cell growth and proliferation. Fatty acid-binding protein 3 gene contains four exons and its function is to arrest growth of mammary epithelial cells. This gene is a candidate tumor suppressor gene for human breast cancer.

### FACL3: Fatty-Acid-Coenzyme A Ligase, Long-Chain 3

The protein encoded by this gene is an isozyme of the long-chain fatty-acid-coenzyme A ligase family. Although differing in substrate specificity, subcellular localization, and tissue distribution, all isozymes of this family convert free long-chain fatty acids into fatty acyl-CoA esters, and thereby play a key role in lipid biosynthesis and fatty acid degradation. This isozyme is highly expressed in brain, and preferentially utilizes myristate, arachidonate, and eicosapentaenoate as substrates. The amino acid sequence of this isozyme is 92% identical to that of rat homolog.

### FACL4: Fatty-Acid-Coenzyme A Ligase, Long-Chain 4

The protein encoded by this gene is an isozyme of the long-chain fatty-acid-coenzyme A ligase family. Although differing in substrate specificity, subcellular localization, and tissue distribution, all isozymes of this family convert free long-chain fatty acids into fatty acyl-CoA esters, and thereby play a key role in lipid biosynthesis and fatty acid degradation. This isozyme preferentially utilizes arachidonate as substrate. The absence of this enzyme may contribute to the mental retardation or Alport syndrome. Alternative splicing of this gene generates 2 transcript variants.

### FMOL: Flavin Containing Monooxygenase 1

Metabolic N-oxidation of the diet-derived amino-trimethylamine (TMA) is mediated by flavin-containing monooxygenase and is subject to an inherited FMO3 polymorphism in man resulting in a small subpopulation with reduced TMA N-oxidation capacity resulting in fish odor syndrome Trimethylaminuria Three forms of the enzyme, FMO1 found in fetal liver, FMO2 found in adult liver, and FMO3 are encoded by genes clustered in the 1q23-25 region. Flavin-containing monooxygenases are NADPH-dependent flavoenzymes that catalyzes the oxidation of soft nucleophilic heteroatom centers in drugs, pesticides, and xenobiotics.

### GAA: Glucosidase, Alpha; Acid (Pompe Disease, Glycogen Storage Disease Type II)

This gene encodes acid alpha-glucosidase, which is essential for the degradation of glycogen to glucose in lysosomes. Different forms of acid alpha-glucosidase are obtained by proteolytic processing. Defects in this gene are the cause of glycogen storage disease II, also known as Pompe's disease, which is an autosomal recessive disorder with a broad clinical spectrum.

### GAPD: Glyceraldehyde-3-phosphate Dehydrogenase

Glyceraldehyde-3-phosphate dehydrogenase catalyzes an important energy-yielding step in carbohydrate metabolism, the reversible oxidative phosphorylation of glyceraldehyde-3-phosphate in the presence of inorganic phosphate and nicotinamide adenine dinucleotide (NAD). The enzyme exists as a tetramer of identical chains. A GAPD pseudogene has been mapped to Xp21-p11and 15 GAPD-like loci have been identified.

### GARS: Glycyl-tRNA Synthetase

Aminoacyl-tRNA synthetases are a class of enzymes that charge tRNAs with their cognate amino acids. Glycyl-tRNA synthetase is an (alpha)2 dimer which belongs to the class II family of tRNA synthetases. It has been shown to be a target of autoantibodies in the human autoimmune diseases, polymyositis or dermatomyositis.

### GBE1: Glucan (1,4-alpha-), Branching Enzyme 1 (Glycogen Branching Enzyme, Andersen Disease, Glycogen Storage Disease Type IV)

This monomeric enzyme functions in -glycogen synthesis by catalyzing the formation of alpha 1,6-glucosidic linkages. It is most highly expressed in liver and muscle. Deficiency can result in glycogen storage disease TV (Andersen's disease).

### GP6: Glycoprotein VI (Platelet)

Platelet glycoprotein VI; member of the paired Ig-like receptor family.

### GPR-55

Member of the G protein-coupled receptor family.

### GPRC5C: G Protein-Coupled Receptor, Family C, Group 5, Member C

The protein encoded by this gene is a member of the type 3 G protein-coupled receptor family. Members of this superfamily are characterized by a signature 7-transmembrane domain motif. The specific function of this protein is unknown; however, this protein may mediate the cellular effects of retinoic acid on the G protein signal transduction cascade. Alternative splicing in the 5′UTR of this gene results in two transcript variants.

### 3-hydroxy-3-methylglutaryl coenzyme A synthase

3-hydroxy-3-methylglutaryl-Coenzyme A synthase; functions in the first step in ketogenesis.

### HK1: Hexokinase 1

Hexokinases phosphorylate glucose to produce glucose-6-phosphate, thus committing glucose to the glycolytic pathway. This gene encodes a ubiquitous form of hexokinase which localizes to the outer membrane of mitochondria. Mutations in this gene have been associated with hemolytic anemia due to hexokinase deficiency. Alternative splicing of this gene results in five transcript variants which encode different isoforms, some of which are tissue-specific. Each isoform has a distinct N-terminus; the remainder of the protein is identical among all the isoforms. A sixth transcript variant has been described, but due to the presence of several stop codons, it is not thought to encode a protein.

### HLA-B Associated Transcript 3 (BAT3)

A cluster of genes, BAT1-BAT5, has been localized in the vicinity of the genes for TNF alpha and TNF beta. These genes are all within the human major histo-compatibility complex class III region. The protein encoded by this gene is a nuclear protein. It has been implicated in the control of apoptosis and regulating heat shock protein. There are three alternatively spliced transcript variants described for this gene.

### HMGCL: 3-hydroxymethyl-3-methylglutaryl-Coenzyme A lyase (hydroxymethylglutaricaciduria)

3-Hydroxy-3-methylglutaryl coenzyme A lyase; cleaves 3-OH-3-methylglutaryl CoA to acetoacetic acid and acetyl CoA.

### HNF4A: Hepatocyte Nuclear Factor 4, Alpha

Nuclear hormone receptor transcription factor; regulates liver specific gene expression.

### Chromosome 12 BAC RP11-13J12

### Cathepsin B

Cathepsin B;. lysosomal cysteine (thiol).protease that cleaves APP.

### Chromosome 5 clone CTD-2235C13

### Chromosome 7 clone RP11-351B12

### Cytochrome P450 3A Locus

The CYP3A locus includes all the known members of the 3A subfamily of the cyto-chrome P450 superfamily of genes. These genes encode monooxygenases which catalyze many reactions involved in drug metabolism and synthesis of cholesterol, steroids and other lipids. The CYP3A cluster consists of four genes, CYP3A43, CYP3A4, CYP3A7 and CYP3A5. The region also contains two pseudogenes, CYP3A5P1 and CYP3A5P2, as well as several extra exons which may or may not be included in transcripts produced from this region. Previously another CYP3A member, CYP3A3, was thought to exist; however, it is now thought that this sequence represents a transcript variant of CYP3A4.

### ITGB3

The ITGB3 protein product is the integrin beta chain beta 3. Integrins are integral cell-surface proteins composed of an alpha chain and a beta chain. A given chain may combine with multiple partners resulting in different integrins. Integrin beta 3 is found along with the alpha IIb chain in platelets. Integrins are known to participate in cell adhesion as well as cell-surface mediated signalling.

### Methionine Adenosyltransferase Alpha Subunit Gene Fragment

MATIA encodes methionine adenosyltransferase I (alpha isoform). MATIA catalyzes the formation of S-adenosylmethionine from methionine and ATP. Both the beta and alpha isoforms may be encoded by MATIA. Methionine adenosyl-transferase deficiency is known to be caused by recessive as well as dominant mutations, the latter identified in autosomal dominant persistant hyper-methioninemia.

### Homo sapiens PAC Clone RP1-102K2 from 22q12.1-qter

### Homo sapiens Partial ZNF202 Gene for Zinc Finger Protein Homolog, Exon 4

Zinc-finger protein 202 may repress genes involved in lipid metabolism; contains zinc fingers.

### Homo sapiens vHNF1-C mRNA

Hepatocyte Nuclear Factor 1.

### Human 2.5 kb mRNA for Cytoskeletal Tropomyosin TM30(nm)

### Human c-kit Gene

KIT encodes the human homolog of the proto-oncogene c-kit. C-kit was first identified as the cellular homolog of the feline sarcoma viral oncogene v-kit. KIT is a type 3 transmembrane receptor for MGF (mast cell growth factor, also known as stem cell factor). Mutations in KIT are associated with gastrointestinal stromal tumors, mast cell disease, acute myelogenous lukemia, and piebaldism.

### Human Coagulation Factor VII (F7) Gene Exon 1 and Factor X (F10) Gene, Exon 1

This gene encodes coagulation factor VII which is a vitamin K-dependent factor essential for hemostasis. This factor circulates in the blood in a zymogen form, and is converted to an active form by either factor IXa, factor Xa, factor XIIa, or thrombin by minor proteolysis. Upon activation of the factor VII, a heavy chain containing a catalytic domain and a light chain containing 2 EGF-like domains are generated, and two chains are held together by a disulfide bond. In the presence of factor III and calcium ions, the activated factor then further activates the coagulation cascade by converting factor IX to factor IXa and/or factor X to factor Xa Alternative splicing of this gene results in 2 transcripts. Defects in this gene can cause coagulopathy.

### Human Cytochrome P450 (CYP1A2) Gene, Exons 1 and 2

This gene, CYP1A2, encodes a member of the cytochrome P450 superfamily of enzymes. The cytochrome P450 proteins are monooxygenases which catalyze many reactions involved in drug metabolism and synthesis of cholesterol, steroids and other lipids. The protein encoded by this gene localizes to the endoplasmic reticulum and its expression is induced by some polycyclic aromatic hydrocarbons (PAHs), some of which are found in cigarette smoke. The enzyme's endogenous substrate is unknown; however, it is able to metabolize some PAHs to carcinogenic intermediates. Other xenobiotic substrates for this enzyme include caffeine, aflatoxin B1, and acetaminophen. The transcript from this gene contains four Alu sequences flanked by direct repeats in the 3′ untranslated region. A related family member, CYP1A1, is located approximately 25 kb away from CYP1A2 on chromosome.

### Human Multidrug Resistance-Associated Protein mRNA

See ABCC1.

### Human Succinyl CoA:3-oxoacid CoA Transferase Precursor (OXCT) mRNA

The mitochondrial matrix enzyme 3-oxoacid CoA transferase is homodimeric. It is a key enzyme in the extrahepatic utilization of ketone bodies, catalyzing the reversible transfer of coenzyme A from succinyl-CoA to acetoacetate, a necessary step in ketolytic energy production. Deficiencies can result in intermittent ketoacidosis.

### Human T-lymphoma Invasion and Metastasis Inducing TIAM1 Protein (TIAM1) mRNA

Member of the GDP-GTP exchange factor family of proteins; modulates the activity of Rho-like proteins; has a Dbl homology and pleckstrin homology domains.

### IL10: Interleukin 10

Interleukin 10 (cytokine synthesis inhibitory factor); functions as a specific chemotactic factor for CD8+T cells.

### IL17R: Interleukin 17 Receptor

Highly similar to murine II17r; may play a role in T cell activation and induction of IL-2 (I12).

### IL3: Interleukin 3 (Colony-Stimulating Factor, Multiple)

Interleukin-3 (colony-stimulating factor); plays a role in hematopoeisis; member of a family of growth factors.

### IL6: Interleukin 6 (Interferon, Beta 2)

Interleukin 6 (interferon-beta 2); induces the maturation of B cells into immunoglobulin-secreting cells.

### IL8RA: Interleukin 8 Receptor, Alpha

Interleukin 8 receptor alpha; G protein-coupled receptor that mediates neutrophil chemotaxis and binds interleukin 8 (1L8).

### INHBC: Inhibin, Beta C

This gene encodes the beta C chain of inhibin, a member of the TGF-beta superfamily. This subunit forms heterodimers with beta A and beta B subunits. Inhibins and activins, also members of the TGF-beta superfamily, are hormones with opposing actions and are involved in hypothalamic, pituitary, and gonadal hormone secretion, as well as growth and differentiation of various cell types.

### ITGAL: Integrin, Alpha L (Antigen CD11A (p180), Lymphocyte Function-Associated Antigen 1; Alpha Polypeptide)

ITGAL encodes the integrin alpha L chain. Integrins are heterodimeric integral membrane proteins composed of an alpha chain and a beta chain. This I-domain containing alpha integrin combines with the beta 2 chain (ITGB2) to form the integrin lymphocyte function-associated antigen-1 (LFA-1), which is expressed on all leukocytes. LFA-1 plays a central role in leukocyte intercellular adhesion through interactions with its ligands, ICAMs 1-3 (intercellular adhesion molecules 1 through 3), and also functions in lymphocyte costimulatory signaling.

### ITGB2: Integrin, Beta 2 (Antigen CD18 (p95), Lymphocyte Function-Associated Antigen 1; Macrophage Antigen 1 (Mac-1) Beta Subunit)

The ITGB2 protein product is the integrin beta chain beta 2. Integrins are integral cell-surface proteins composed of an alpha chain and a beta chain. A given chain may combine with multiple partners resulting in different integrins. For example, beta 2 combines with the alpha L chain to form the integrin LFA-1, and combines with the alpha M chain to form the integrin Mac-1. Integrins are known to participate in cell adhesion as well as cell-surface mediated signalling.

### KCNQ1: Potassium-Voltage-Gated Channel, KQT-like Subfamily, Member 1

KCNQ1 encodes the K+ channel subunit responsible for the delayed-rectifier K+ current in cardiac myocytes. The delayed-rectifier channel is completed by the protein encoded by KCNE1. Mutations in KCNQ1 cause inherited long-QT syndrome.

### LAMA3: Laminin, Alpha 3 (Nicein (150 kD), Kalinin (165 kD), BM600 (150 kD), Epilegrin)

Laminins are basement membrane components thought to mediate the attachment, migration and organization of cells into tissues during embryonic development by interacting with other extracellular matrix components. The protein encoded by this gene is the alpha-3 chain of laminin 5, which is a complex glycoprotein composed of three subunits (alpha, beta, and gamma). Laminin 5 is thought to be involved in cell adhesion, signal transduction and differentiation of keratinocytes. Mutations in this gene have been identified as the cause of Herlitz type junctional epidermolysis bullosa. Alternative splicing has been observed at this locus but the full-length nature of these variants has not been determined.

### LAMR1: Laminin Receptor 1 (67 kD, Ribosomal Protein SA)

Laminins, a family of extracellular matrix glycoproteins, are the major non-coliagenous constituent of basement membranes. They have been implicated in a wide variety of biological processes including cell adhesion, differentiation, migration, signaling, neurite outgrowth and metastasis. Many of the effects of laminin are mediated through interactions with cell surface receptors. These receptors include members of the integrin family, as well as non-integrin laminin-binding proteins. This gene encodes a high-affinity, non-integrin family, laminin receptor 1. his receptor has been variously called 67 kD laminin receptor, 37 kD laminin receptor precursor (37LRP) and p40 ribosome-associated protein. The amino acid sequence of laminin receptor 1 is highly conserved through evolution, suggesting a key biological function. It has been observed that the level of the laminin receptor transcript is higher in colon carcinoma tissue and lung cancer cell line than their normal counterparts. Also, there is a correlation between the upregulation of this polypeptide in cancer cells and their invasive and metastatic phenotype. Multiple copies of this gene exist, however, most of them are pseudogenes thought to have arisen from retropositional events.

### LDLR: Low Density Lipoprotein Receptor (Familial Hypercholesterolemia)

The low density lipoprotein receptor (LDLR) gene family consists of cell surface proteins involved in receptor-mediated endocytosis of specific ligands. Low density lipoprotein (LDL) is normally bound at the cell membrane and taken into the cell ending up in lysosomes where the protein is degraded and the cholesterol is made available for repression of microsomal enzyme 3-hydroxy-3-methylglutaryl coenzyme A (HMG CoA) reductase, the rate-limiting step in cholesterol synthesis. At the same time, a reciprocal stimulation of cholesterol ester synthesis takes place. Mutations in the LDL receptor (LDLR) gene cause the autosomal dominant disorder, familial hypercholesterolemia.

### LGALS7: Lectin, Galactoside-Binding, Soluble, 7 (Galectin 7)

The galectins are a family of beta-galactoside-binding proteins implicated in modulating cell-cell and cell-matrix interactions. Differential and in situ hybridizations indicate that this lectin is specifically expressed in keratinocytes. It is expressed at all stages of epidermal differentiation (i.e., in basal and suprabasal layers). It is moderately repressed by retinoic acid. The protein was found mainly in stratified squamous epithelium. The antigen localized to basal keratinocytes, although it was also found, albeit at lower levels, in the suprabasal layers where it concentrated to areas of cell-to-cell contact. The cellular localization and its striking down-regulation in cultured keratinocytes imply a role in cell-cell and/or cell-matrix interactions necessary for normal growth control.

### LIMK1: LIM Domain Kinase 1

There are approximately 40 known eukaryotic LIM proteins, so named for the LIM domains they contain. LIM domains are highly conserved cysteine-rich structures containing 2 zinc fingers. Although zinc fingers usually function by binding to DNA or RNA, the LIM motif probably mediates protein-protein interactions. LIM kinase-1 and LIM kinase-2 belong to a small subfamily with a unique combination of 2 N-terminal LIM motifs and a C-terminal protein kinase domain. LIMK1 is likely to be a component of an intracellular signaling pathway and may be involved in brain development. LIMK1 hemizygosity is implicated in the impaired visuospatial constructive cognition of Williams syndrome. Two splice variant have been identified.

### LMNB2: Lamin B2

Lamin B2; member of a family of structural nuclear envelope proteins.

### LPL: Lipoprotein Lipase

LPL encodes lipoprotein lipase, which is expressed in heart, muscle, and adipose tissue. LPL functions as a homodimer, and has the dual functions of triglyceride hydrolase and ligand/bridging factor for receptor-mediated lipoprotein uptake. Severe mutations that cause LPL deficiency result in type I hyperlipoproteinemia, while less extreme mutations in LPL are linked to many disorders of lipoprotein metabolism.

### LRP8: Low Density Lipoprotein Receptor-Related Protein 8, Apolipoprotein e Receptor

This gene encodes an apolipoprotein E receptor, a member of the low density lipoprotein receptor (LDLR) family. Apolipoprotein E is a small lipophilic plasma protein and a component of lipoproteins such as chylomicron remnants, very low density lipoprotein (VLDL), and high density lipoprotein (HDL). The apolipoprotein E receptor is involved in cellular recognition and internalization of these lipoproteins. Alternative splicing generates three transcript variants for this gene; additional variants have been described, but their full length nature has not been determined.

### LSS: Lanosterol Synthase (2,3-oxidosqualene-lanosterol cyclase)

Lanosterol synthase ((S)-2,3-epoxysqualene mutase); catalyzes the cyclization of (S)-2,3-oxidosqualene; forms lanosterdl during sterol biosynthesis.

### LTA: Lymphotoxin Alpha (TNF Superfamily, Member 1)

Lymphotoxin alpha, a member of the tumor necrosis factor family, is a cytokine produced by lymphocytes. LTA is highly inducible, secreted, and exists as homotriieric molecule. LTA forms heterotrimers with lymphotoxin-beta which anchors lymphotoxin-alpha to the cell surface. LTA mediates a large variety of inflammatory, immunostimulatory, and antiviral responses. LTA is also involved in the formation of secondary lymphoid organs during development and plays a role in apoptosis.

### MAOA: Monoamine Oxidase A

MAOA encodes monoamine oxidase A, an enzyme that degrades amine neuro-transmitters, such as dopamine, norepinephrine, and serotonin. Deficiency of this enzyme results in Brunner syndrome.

### MARCKS: Myristoylated Alanine-Rich Protein Kinase C Substrate

The protein encoded by this gene is a substrate for protein kinase C. It is localized to the plasma membrane and is an actin filament crosslinking protein. Phosphorylation by protein kinase C or binding to calcium-calmodulin inhibits its association with actin and with the plasma membrane, leading to its presence in the cytoplasm. The protein is thought to be involved in cell motility, phagocytosis, membrane trafficking and mitogenesis.

### MCL1: Myeloid Cell Leukemia Sequence 1 (BCL2-related)

Similar to BCL2.

### MCP: Membrane Cofactor Protein (CD46, Trophoblast-Lymphocyte Cross-Reactive Antigen)

Membrane cofactor protein; acts as the receptor for the measles virus, may be involved in the regulation of complement activation; contains SCRS.

### METTL1: Methyltransferase-Like 1

This gene is an ortholog of the S. cerevisiae YDL201w gene, which is predicted to encode a methyltransferase. The gene product contains a conserved S-adenosyl-methionine-binding motif, which is typical of a methyltransferase. Alternative splice variants encoding different protein isoforms and transcript variants utilizing alternative polyA sites have been described in the literature.

### MLLT3: Myeloid/Lymphoid or Mixed-Lineage Leukemia (Trithorax Homolog, Drosophila);

Serine and proline rich protein, has a nuclear targeting sequence.

### MTHFD1: Methylenetetrahydrofolate Dehydrogenase (NADP+ Dependent), Methenyltetrahydrofolate Cyclohydrolase, Formyltetrahydrofolate Synthetase

This gene encodes a protein that possesses three distinct enzymatic activities, 5,10-methylenetetrahydrofolate dehydrogenase, 5,10-methenyltetrahydrofolate cyclo-hydrolase and 10-formyltetrahydrofolate synthetase. Each of these activities catalyzes one of three sequential reactions in the interconversion of 1-carbon derivatives of tetrahydrofolate, which are substrates for methionine, thymidylate, and de novo purine syntheses. The trifunctional enzymatic activities are conferred by two major domains, an aminoterminal portion containing the dehydrogenase and cyclohydrolase activities and a larger synthetase domain.

### MTR2 Myotubularin Related Protein 2 (MTMR2)

This gene is a member of the myotubularin family and encodes a putative tyrosine phosphatase. Mutations in this gene are a cause of Charcot-Marie-Tooth disease type 4B, an autosomal recessive demyelinating neuropathy. This gene utilizes multiple polyA signals, only one of which has been determined.

### Muscle Specific Serine Kinase (MSSK1; Serine/Threonine Kinase 23, STK23),

Highly similar to SRPK2; may be protein kinase for SR family of RNA splicing factors; contains a kinase domain.

### MVD: Mevalonate (Diphospho) Decarboxylase

The enzyme mevalonate pyrophosphate decarboxylase catalyzes the conversion of mevalonate pyrophosphate into isopentenyl pyrophosphate in one of the early steps in cholesterol biosynthesis. It decarboxylates and dehydrates its substrate while hydrolyzing ATP.

### MYH11: Myosin, Heavy Polypeptide 11, Smooth Muscle

The protein encoded by this gene is a smooth muscle myosin belonging to the myosin heavy chain family; The gene product is a subunit of a hexameric protein that consists of 2 heavy chain subunits and 2 pairs of non-identical light chain subunits. It functions as a major contractile protein, converting chemical energy into mechanical energy through the hydrolysis of ATP. The gene encoding a human ortholog of rat NUDE1 is transcribed from the reverse strand of MYH11 gene, and its 3′ end overlaps with that of the latter. The pericentric inversion of chromosome 16 [inv(16)(p13q22)] produces a chimeric transcript consisting of the first 165 residues from the N terminus of core-binding factor beta in a fusion with the C-terminal portion of the smooth muscle myosin heavy chain. This chromosomal rearrangement is associated with acute myeloid leukemia of the M4Eo subtype. Alternative splicing generates isoforms that are differentially expressed, with ratios changing during muscle cell maturation. Additional splice variants have been described but their full-length nature has not been determined.

### MYH7: Myosin, Heavy Polypeptide 7, Cardiac Muscle, Beta

MYH7 encodes the cardiac muscle beta (or slow) isoform of myosin. Changes in the relative abundance of MYH7 and MYH6 (the alpha, or fast, isoform of cardiac myosin heavy chain) correlate with the contractile velocity of cardiac muscle. Mutations in MYH7 are associated with familial hypertrophic cardiomyopathy.

### NADH Dehydrogenase (Ubiquinone) 1, Alpha Subcomplex, 4 (9 kD, MLRQ), NDUFA4

Subunit of NADH-ubiquinone Oxidoreductase (Complex I); Transports Electrons From NADH to Ubiquinone.

### NADH-Ubiquinone Oxidoreductase Chain 5 (EC 1.6.5.3)

Subunit of NADH-ubiquinone oxidoreductase (complex I); transports electrons from NADH to ubiquinone.

### NDUFA9: NADH Dehydrogenase (Ubiquinone) 1 Alpha Subcomplex, 9 (39 kD)

### NGFB: Nerve Growth Factor, Beta Polypeptide

Nerve growth factor beta; has roles in neuronal differentiation and survival.

### NGFR: Nerve Growth Factor Receptor (TNFR Superfamily, Member 16)

Nerve growth factor receptor contains an extracellular domain containing four 40-amino acid repeats with 6 cysteine residues at conserved positions followed by a serine/threonine-rich region, a single transmembrane domain, and a 155-amino acid cytoplasmic domain. The cysteine-rich region contains the nerve growth factor binding domain.

### NID2: Nidogen 2

Nidogen-2; basement membrane protein.

### HSU15552: Acidic 82 kDa Protein mRNA

### Nonmuscle Type Myosin Heavy Chain 9 (MH9)

Non-muscle myosin heavy chain 9; motor protein that provides force for muscle contraction, cytokinesis and phagocytosis; contains an ATPase head domain and a rod-like tail domain.

### NPC1: Niemann-Pick Disease, Type C1

NPC1 was identified as the gene that when mutated, results in Niemann-Pick C disease. NPC1 encodes a putative integral membrane protein containing motifs consistent with a role in intracellular transport of cholesterol to post-lysosomal destinations.

### Nth Endonuclease III-like 1 (NTHL1)

Endonuclease; excises damaged pyrimidines.

### NUCB2: Nucleobindin 2

Nucleobindin 2; may bind DNA and calcium; has DNA-binding and EF-hand domains, and a leucine-zipper.

### Nuclear Receptor Subfamily 1, Group I, Member 2 (NR1I2)

The gene product belongs to the nuclear receptor superfamily, members of which are transcription factors characterized by a ligand-binding domain and a DNA-binding domain. The encoded protein is a transcriptional regulator of the cytochrome P450 gene CYP3A4, binding to the response element of the CYP3A4 promoter as a heterodimer with the 9-cis retinoic acid receptor RXR. It is activated by a range of compounds that induce CYP3A4, including dexamethasone and rifampicin. The gene product contains a zinc finger domain. Three alternatively spliced transcripts that encode different isoforms have been described, one of which encodes two products through the use of alternative translation initiation codons. Additional transcript variants derived from alternative promoter usage, alternative splicing, and/or alternative polyadenylation exist, but they have not been fully described.

### OGDH: Oxoglutarate (Alpha-Ketoglutarate) Dehydrogenase (Lipoamide)

Alpha-ketoglutarate or 2-oxoglutarate dehydrogenase; helps convert a-ketoglutarate to succinyl coenzyme A in Krebs cycle.

### OXCT: 3-oxoacid CoA Transferase

The mitochondrial matrix enzyme 3-oxoacid CoA transferase is homodimeric. It is a key enzyme in the extrahepatic utilization of ketone bodies, catalyzing the reversible transfer of coenzyme A from succinyl-CoA to acetoacetate, a necessary step in ketolytic energy production. Deficiencies can result in intermittent ketoacidosis.

### P2RYI: Purinergic Receptor P2Y, G-Protein Coupled, 1

Purinergic receptor P2Y1, a G protein-coupled receptor; mediates responses to ATP and increases inositol phosphate levels.

### PCCA: Propionyl Coenzyme A Carboxylase, Alpha Polypeptide

PCCA encodes the alpha subunit of the heterodimeric mitochondrial enzyme Propionyl-CoA carboxylase. PCCA encodes the biotin-binding region of this enzyme. Mutations in either PCCA or PCCB (encoding the beta subunit) lead to an enzyme deficiency result in propionic acidemia.

### PDGFB: Platelet-Derived Growth Factor Beta Polypeptide (Simian Sarcoma Viral (v-sis) Oncogene Homolog)

The protein encoded by this gene is a member of the platelet-derived growth factor family. The four members of this family are mitogenic factors for cells of mesenchymal origin and are characterized by a motif of eight cysteines. This gene product can exist either as a homodimer or as a heterodimer with the platelet-derived growth factor alpha polypeptide, where the dimers are connected by disulfide bonds. Mutations in this gene are associated with meningioma Reciprocal translocations between chromosomes 22 and 7, at sites where this gene and that for COL1A1 are located, are associated with a particular type of skin tumor called dermatofibro-sarcoma protuberans resulting from unregulated expression of growth factor. Two splice variants have been identified for this gene.

### Period Circadian Protein 2 (AA0347)

This gene is a member of the Period family of genes and is expressed in a circadian pattern in the suprachiasmatic nucleus, the primary circadian pacemaker in the mammalian brain. Genes in this family encode components of the circadian rhythms of locomotor activity, metabolism, and behavior. Circadian expression in the suprachiasmatic nucleus continues in constant darkness, and a shift in the light/dark cycle evokes a proportional shift of gene expression in the suprachiasmatic nucleus. The specific function of this gene is not yet known.

### Peroxisome Proliferative Activated Receptor, Delta (PPARD)

Peroxisome proliferator-activated receptor delta is a member of the steroid hormone receptor superfamily.

### PGM5: Phosphoglucomutase 5

Phosphoglucomutase-related (aciculin) putative structural protein; interacts with the cytoskeletal proteins dystrophin and utrophin.

### PLA2G3: Phospholipase A2, Group III

Group III secreted phospholipase A2; calcium-dependent, displays a preference for phosphatidylglycerol over phosphatidylcholine.

### PLA2G4C: Phospholipase A2, Group IVC (Cytosolic, Calcium-Independent)

Group IVC calcium-independent phospholipase a2; hydrolyzes the phospholipid sn-2 ester bond; member of the phospholipase family.

### PLA2G6: Phospholipase A2, Group VI (Cytosolic, Calcium-Independent)

Cytosolic calcium-independent phospholipase_a2; hydrolyzes the phospholipid sn-2 ester bond; member of the phospholipase family.

### PMVK: Phosphomevalonaie Kinase

Phosphomevalonate kinase; converts mevalonate-5-phosphate to mevalonate-5-diphosphate.

### PNMT: Phenylethanolamine N-methyltransferase

Phenylethanolamine N-methyltransferase; converts norepinephrine to epinephrine.

### PON1: paraoxonase 1

### PON2: paraoxonase 2

Paraoxonase/arylesterase 2; possibly functions in protecting low density lipoprotein against oxidative modification; member of a family that hydrolyzes toxic organo-phosphates.

### PPARA: Peroxisome Proliferative Activated Receptor, Alpha

Peroxisome proliferators are a diverse group of chemicals which include hypolipidemic drugs, herbicides, leukotriene antagonists, and plasticizers, and are so called because they induce an increase in the size and number of peroxisomes. Peroxisomes are subcellular organelles found in plants and animals, and contain enzymes for respiration, cholesterol and lipid metabolism. Infact, the fibrate class of hypolipidemic drugs is used to reduce triglycerides and cholesterol in patients with hyperlipidemia, a major risk factor for coronary heart disease. The action of peroxisome proliferators is thought to be mediated via specific receptors belonging to the steroid hormone receptor superfamily, called PPARs. Thus far, four closely related subtypes, alpha, beta, gamma and delta, have been identified. The subtype PPAR-alpha, encoded by PPARA, is a nuclear transcription factor. Upon activation by peroxisome proliferators, it modulates the expression of target genes involved in lipid metabolism, suggesting a role for PPAR-alpha in lipid homeostasis.

### PPARG: Peroxisome Proliferative Activated Receptor, Gamma

The protein encoded by this gene is a member of the peroxisome proliferator-activated receptor.(PPAR) subfamily of nuclear receptors. PPARs form heterodimers with retinoid X receptors (RXRs) and these heterodimers regulate transcription of various genes. Three subtypes of PPARs are known: PPAR-alpha, PPAR-delta, and PPAR-gamma. The protein encoded by this gene is PPAR-gamma and is a regulator of adipocyte differentiation. Additionally, PPAR-gamma has been implicated in the pathology of numerous diseases including obesity, diabetes, atherosclerosis and cancer. Multiple transcript variants that use alternate promoters and splicing have been identified for this gene. At least three of these variants encode the same isoform.

### PPM1A: Protein Phosphatase 1A (Formerly 2C), Magnesium-Dependent, Alpha Isoform

Magnesium- or manganese-dependent alpha protein phosphatase 1A; regulates cell stress responses.

### Probable G Protein-Coupled Rececptor APJ

### PTPRA: Protein Tyrosine Phosphatase, Receptor Type, A

The protein encoded by this gene is a member of the protein tyrosine phosphatase (PTP) family. PTPs are known to be signaling molecules that regulate a variety of cellular processes including cell growth, differentiation, mitotic cycle, and oncogenic transformation. This PTP contains an extracellular domain, a single transmembrane segment and two tandem intracytoplasmic catalytic domains, and thus represents a receptor-type PTP. This PTP has been shown to dephosphorylate and activate Src family tyrosine kinases, and is implicated in the regulation of integrin signaling, cell adhesion and proliferation. Three alternatively spliced variants of this gene, which encode two distinct isoforms, have been reported.

### PYGM: Phosphorylase, Glycogen; Muscle (McArdle Syndrome, Glycogen Storage Disease Type V)

Muscle glycogen phosphorylase.

### RTN1: Reticulon 1

### RXRA: Retinoid X Receptor, Alpha

Retinoid X receptors (RXks) and retinoic acid receptors (RARs), are nuclear receptors that mediate the biological effects of retinoids by their involvement in retinoic acid-mediated gene activation. These receptors exert their action by binding, as homodimers or heterodimers, to specific sequences in the promoters of target genes and regulating their transcription. The protein encoded by this gene is a member of the steroid and thyroid hormone receptor superfamily of transcriptional regulators.

### RXRB: Retinoid X Receptor, Beta

Retinoid X receptor beta; binds to and serves as transcriptional coactivator for retinoic acid.

### SCA1: Spinocerebellar Ataxia 1 (Olivopontocerebellar Ataxia 1, Autosomal Dominant, Ataxin 1)

The autosomal dominant cerebellar ataxias (ADCA) are a heterogeneous group of neurodegenerative disorders characterized by progressive degeneration of the cerebellum, brain stem and spinal cord. Clinically, ADCA has been divided into three groups: ADCA types I-III. ADCAI is genetically heterogeneous, with five genetic loci, designated spinocerebellar ataxia (SCA) 1, 2, 3, 4 and 6, being assigned to five different chromosomes. ADCAII, which always presents with retinal degeneration (SCA7), and ADCAIII often referred to as the ‘pure’ cerebellar syndrome (SCA5), are most likely homogeneous disorders. Several SCA genes have been cloned and shown to contain CAG repeats in their coding regions. ADCA is caused by the expansion of the CAG repeats, producing an elongated polyglutamine tract in the corresponding protein. The expanded repeats are variable in size and unstable, usually increasing in size when transmitted to successive generations. The function of the ataxins is not known. The SCA1 locus has been mapped to chromosome 6, and it has been determined that the diseased allele contains 41-81 CAG repeats, compared to 6-39 in the normal allele. Several transcript variants of SCA1 in the 5′UTR have been described; however, their full-length nature is not known.

### SDF1: Stromal Cell-Derived Factor 1

Stromal cell-derived factor 1; lymphocyte chemoattractant that signals through the receptor CXCR4.

### SERPINA5: Serine (or Cysteine) Proteinase Inhibitor, Clade A (Alpha-1 Antiproteinase, Antitrypsin), Member 5

Protein C inhibitor (plasminogen activator inhibitor III); may be a serine protease inhibitor; member of the serpin family of serine protease inhibitors.

### SERPINH1: Serine (or Cysteine) Proteinase Inhibitor, Clade H (Heat Shock Protein 47), Member 1, (Collagen Binding Protein 1)

Colligin; collagen-binding protein; Similar to HSPs and to serpin family serine protease inhibitors.

### SLC21A6: Solute Carrier Family 21 (Organic Anion Transporter), Member 6

Organic anion transporter.

### SLC27A1: Solute Carrier Family 27 (Fatty Acid Transporter), Member 1

### SULT1A2: Sulfotransferase Family, Cytosolic, 1A, Phenol-Preferring, member 2 Phenol-metabolizing sulfotransferase 2; sulfonates simple planar phenols.

### THBS3: Thrombospondin 3

Thrombospondin 3 binds heparin and calcium; similar to murine Thbs3

### TBP: TATA Box Binding Protein TATA box binding protein, component of the TFID complex; functions in the initiation of mRNA synthesis and basal transcription.

### TBXA2R: Thromboxane A2 Receptor

Thromboxane A2 receptor (prostaglandin H2 receptor); G protein-coupled receptor, activates Ca2+-activated chloride channels, stimulates platelet aggregation and smooth muscle constriction.

### TCF2: Transcription Factor 2, Hepatic; LF-B3; Variant Hepatic Nuclear Factor

TCF2 encodes transcription factor 2, a liver-specific factor of the homeobox-containing basic helix-turn-helix family. The TCF2 protein is believed to form heterodimers with another liver-specific member of this transcription factor family, TCF1; depending on the TCF2 isoform, the result may be to activate or inhibit transcription of target genes. Mutation of TCF2 that disrupts normal function has been identified as the cause of MODY5 (Maturity-Onset of Diabetes, Type 5). A third human transcript variant is believed to exist based on such a variant in the rat: however, to date such an mRNA species has not been isolated.

### TETRAN: Tetracycline Transporter-Like Protein

Similar to E. coli tetracycline resistance efflux protein.

### TGFB1: Transforming Growth Factor, Beta 1 (Camurati-Engelmann Disease)

Transforming growth factor-beta 1; regulates cell proliferation, differentiation, and apoptosis.

### TGFB2: Transforming Growth Factor, Beta 2

Transforming growth factor-beta 2 (glioblastoma-derived T cell suppressor factor); suppresses IL2-dependent growth of T cells; member of a family of cytokines that transmits signals through transmembrane serine/threonine kinases.

### TGFB3: Transforming Growth Factor, Beta 3

Transforming growth factor-beta 3; transmits signals through tramsmembrane serine/threonine kinases, may be required for normal development of the lung and palate; member of family of cytokines, very strongly similar to murine Tgfb3.

### THPO: Thrombopoietin (Myeloproliferative Leukemia Virus Oncogene Ligand, Megakaryocyte Growth and Development Factor)

Thrombopoietin; binds to c-Mpl receptor and regulates megakaryocyte development.

### TNFAIP2: Tumor Necrosis Factor, Alpha-Induced Protein 2

Secreted by vascular endothelium, expression is induced by tumor necrosis factor alpha, interleukin-1 beta, and lipopolysaccharide.

### TRAP1: Heat Shock Protein 75

Heat shock protein 75; binds and refolds denatured RB1 during M phase and after heat shock; member of the HSP90 family of molecular chaperones.

### TRIP10: Thyroid Hormone Receptor Interactor 10

Similar to the non-kinase domains of FER and Fes/Fps tyrosine kinases; binds to activated Cdc42 and may regulate actin cytoskeleton; contains an SH3 domain.

### TXN: Thioredoxin

Thioredoxin; has dithiol-disulfide oxidoreductase activity.

### USP6: Ubiquitin Specific Protease 6 (Tre-2 Oncogene)

Ubiquitin specific protease 6 (Tre-2 oncogene); cleaves ubiquitin from proteins, has predicted nucleic acid-binding properties.

### UTRN: Utrophin (Homologous to Dystrophin)

This gene shares both structural and functional similarities with the dystrophin gene. It contains an actin-binding N-terminus, a triple coiled-coil repeat central region, and a C-terminus that consists of protein-protein interaction motifs which interact with dystroglycan protein components. The protein encoded by this gene is located at the neuromuscular synapse and myotendinous junctions, where it participates in post-synaptic membrane maintenance and acetylcholine receptor clustering. Mouse studies suggest that this gene may serve as a functional substitute for the dystrophin gene and therefore, may serve as a potential therapeutic alternative to muscular dystrophy which caused by mutations in the dystrophin gene. Alternative splicing of the utrophin gene has been described; however, the full-length nature of these variants has not yet been determined.

### VEGF: Vascular Endothelial Growth Factor

Vascular endothelial growth factor; induces endothelial cell proliferation and vascular permeability.

### VEGFB: Vascular Endothelial Growth Factor B

Vascular endothelial growth factor B; involved in angiogenesis and endothelial cell growth.

### WISP1: WNT1 Inducible Signaling Pathway Protein 1

This gene encodes a member of the WNT1 inducible signaling pathway (WISP) protein subfamily, which belongs to the connective tissue growth factor (CTGF) family. WNT1 is a member of a family of cysteine-rich, glycosylated signaling proteins that mediate diverse developmental processes. The CTGF family members are characterized by four conserved cysteine-rich domains: insulin-like growth factor-binding domain, von Willebrand factor type C module, thrombospondin domain and C-terminal cystine knot-like domain. This gene may be downstream in the WNT1 signaling pathway that is relevant to malignant transformation. It is expressed at a high level in fibroblast cells, and overexpressed in colon tumors. The encoded protein binds to decorin and biglycan, two members of a family of small leucine-rich proteoglycans present in the extracellular matrix of connective tissue, and possibly prevents the inhibitory activity of decorin and biglycan in tumor cell proliferation. It also attenuates p53-mediated apoptosis in response to DNA damage through activation of the Akt kinase. It is 83% identical to the mouse protein at the amino acid level. Alternative splicing of this gene generates 2 transcript variants.

### XDH: Xanthene Dehydrogenase

Xanthine dehydrogenase belongs to the group of molybdenum-containing hydroxylases involved in the oxidative metabolism of purines. The enzyme is a homodimer. Xanthine dehydrogenase can be converted to xanthine oxidase by reversible sulfhydryl oxidation or by irreversible proteolytic modification. Defects in xanthine dehydrogenase cause xanthinuria, may contribute to adult respiratory stress syndrome, and may potentiate influenza infection through an oxygen metabolite-dependent mechanism.

### YAP1: Yes-Associated Protein 1, 65 kD

Yes-associated protein; binds to the proto-oncoprotein Yes; has a WW domain.

### PROCR: Protein C Receptor, Endothelial (EPCR)

Endothelial Protein C receptor; binds protein C in a calcium-dependent manner; member of the CD1/major histocompatibility complex superfamily.

### STX1A: Syntaxin 1A (Brain)

Syntaxin 1A (brain); involved in intracellular transport and neurotransmitter release.

As SNPs are linked to other SNPs in neighboring genes on a chromosome (Linkage Disequilibrium) those SNPs could also be used as marker SNPs. In a recent publication it was shown that SNPs are linked over 100 kb in some cases more than 150 kb (Reich D. E. et al. Nature 411, 199-204, 2001). Hence SNPs lying in regions neighbouring PA SNPs could be linked to the latter and by this being a diagnostic marker. These associations could be performed as described for the gene polymorphism in methods.

### DEFINITIONS

For convenience, the meaning of certain terms and phrases employed in the specification, examples, and appended claims are provided below. Moreover, the definitions by itself are intended to explain a further background of the invention.

The term “allele”, which is used interchangeably herein with “allelic variant” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene. Alleles of a specific gene can differ from each other in a single nucleotide, or several nucleotides, and can include substitutions, deletions, and insertions of nucleotides. An allele of a gene can also be a form of a gene containing a mutation.

The term “allelic variant of a polymorphic region of a gene” refers to a region of a gene having one of several nucleotide sequences found in that region of the gene in other individuals.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present invention.

The term “a homologue of a nucleic acid” refers to a nucleic acid having a nucleotide sequence having a certain degree of homology with the nucleotide sequence of the nucleic acid or complement thereof. A homologue of a double stranded nucleic acid having SEQ ID NO. X is intended to include nucleic acids having a nucleotide sequence which has a certain degree of homology with SEQ ID NO. X or with the complement thereof. Preferred homologous of nucleic acids are capable of hybridizing to the nucleic acid or complement thereof.

The term “interact” as used herein is meant to include detectable interactions between molecules, such as can be detected using, for example, a hybridization assay.

The term interact is also meant to include “binding” interactions between molecules. Interactions may be, for example, protein-protein, protein-nucleic acid, protein-small molecule or small molecule-nucleic acid in nature.

The term “intronic sequence” or “intronic nucleotide sequence” refers to the nucleotide sequence of an intron or portion thereof.

The term “isolated” as used herein with respect to nucleic acids, such as DNA or RNA, refers to molecules separated from other DNAs or RNAs, respectively, that are present in the natural source of the macromolecule. The term isolated as used herein also refers to a nucleic acid or peptide that is substantially free of cellular material, viral material, or culture medium when produced by recombinant DNA techniques, or chemical precursors or other chemicals when chemically synthesized.

Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

The term “lipid” shall refer to a fat or fat-like substance that is insoluble in polar solvents such as water. The term “lipid” is intended to include true fats (e.g. esters of fatty acids and glycerol); lipids (phospholipids, cerebrosides, waxes); sterols (cholesterol, ergosterol) and lipoproteins (e.g. HDL, LDL and VLDL).

The term “locus” refers to a specific position in a chromosome. For example, a locus of a gene refers to the chromosomal position of the gene.

The term “modulation” as used herein refers to both up-regulation, (i.e., activation or stimulation), for example by agonizing, and down-regulation (i.e. inhibition or suppression), for example by antagonizing of a bioactivity (e.g. expression of a gene).

The term “molecular structure” of a gene or a portion thereof refers to the structure as defined by the nucleotide content (including deletions, substitutions, additions of one or more nucleotides), the nucleotide sequence, the state of methylation, and/or any other modification of the gene or portion thereof.

The term “mutated gene” refers to an allelic form of a gene, which is capable of altering the phenotype of a subject having the mutated gene relative to a subject which does not have the mutated gene. If a subject must be homozygous for this mutation to have an altered phenotype, the mutation is said to be recessive. If one copy of the mutated gene is sufficient to alter the genotype of the subject, the mutation is said to be dominant. If a subject has one copy of the mutated gene and has a phenotype that is intermediate between that of a homozygous and that of a heterozygous (for that gene) subject, the mutation is said to be co-dominant.

As used herein, the term “nucleic acid” refers to polynucleotides such as deoxy-ribonucleic acid (DNA), and, where appropriate, ribonucleic acid (RNA). The term should also be understood to include, as equivalents, derivatives, variants and analogs of either RNA or DNA made from nucleotide analogs, including peptide nucleic acids (PNA), morpholino oligonucleotides (J. Summerton and D. Weller, Antisense and Nucleic Acid Drug Development 7:187 (1997)) and, as applicable to the embodiment being described, single (sense or antisense) and double-stranded polynucleotides. Deoxyribonucleotides include deoxyadenosine, deoxycytidine, deoxyguanosine, and deoxythymidine. For purposes of clarity, when referring herein to a nucleotide of a nucleic acid, which can be DNA or an RNA, the term “adenosine”, “cytidine”, “guanosine”, and “thymidine” are used. It is understood that if the nucleic acid is RNA, a nucleotide having a uracil base is uridine.

The term “nucleotide sequence complementary to the nucleotide sequence set forth in SEQ ID NO. x” refers to the nucleotide sequence of the complementary strand of a nucleic acid strand having SEQ ID NO. x. The term “complementary strand” is used herein interchangeably with the term “complement”. The complement of a nucleic acid strand can be the complement of a coding strand or the complement of a non-coding strand. When referring to double stranded nucleic acids, the complement of a nucleic acid having SEQ D NO. x refers to the complementary strand of the strand having SEQ ID NO. x or to any nucleic acid having the nucleotide sequence of the complementary strand of SEQ ID NO. x. When referring to a single stranded nucleic acid having the nucleotide sequence SEQ ID NO. x, the complement of this nucleic acid is a nucleic acid having a nucleotide sequence which is complementary to that of SEQ ID NO. x. The nucleotide sequences and complementary sequences thereof are always given in the 5′ to 3′ direction. The term “complement” and “reverse complement” are used interchangeably herein.

The term “operably linked” is intended to mean that the promoter is associated with the nucleic acid in such a manner as to facilitate transcription of the nucleic acid.

The term “polymorphism” refers to the coexistence of more than one form of a gene or portion thereof. A portion of a gene of which there are at least two different forms, i.e., two different nucleotide sequences, is referred to as a “polymorphic region of a gene”. A polymorphic region can be a single nucleotide, the identity of which differs in different alleles. A polymorphic region can also be several nucleotides long.

A “polymorphic gene” refers to a gene having at least one polymorphic region.

To describe a “polymorphic site” in a nucleotide sequence often there is used an “ambiguity code” that stands for the possible variations of nucleotides in one site. The list of ambiguity codes is summarized in the following table:

Ambiguity Codes
(IUPAC Nomenclature)

| | |
|---|---|
| B | c/g/t |
| D | a/g/t |
| H | a/c/t |
| K | g/t |
| M | a/c |
| N | a/c/g/t |
| R | a/g |
| S | c/g |
| V | a/c/g |
| W | a/t |
| Y | c/t |

So, for example, a “R” in a nucleotide sequence means that either an “a” or a “g” could be at that position.

The terms “protein”, “polypeptide” and “peptide” are used interchangeably herein when referring to a gene product.

A “regulatory element”, also termed herein “regulatory sequence is intended to include elements which are capable of modulating transcription from a basic promoter and include elements such as enhancers and silencers. The term “enhancer”, also referred to herein as “enhancer element”, is intended to include regulatory elements capable of increasing, stimulating, or enhancing transcription from a basic promoter. The term “silencer”, also referred to herein as “silencer element” is intended to include regulatory elements capable of decreasing, inhibiting, or repressing transcription from a basic promoter. Regulatory elements are typically present in 5′ flanking regions of genes. However, regulatory elements have also been shown to be present in other regions of a gene, in particular in introns. Thus, it is possible that genes have regulatory elements located in introns, exons, coding regions, and 3′ flanking sequences. Such regulatory elements are also intended to be encompassed by the present invention and can be identified by any of the assays that can be used to identify regulatory elements in 5′ flanking regions of genes.

The term “regulatory element” further encompasses “tissue specific” regulatory elements, i.e., regulatory elements which effect expression of the selected DNA sequence preferentially in specific cells (e.g., cells of a specific tissue). gene expression occurs preferentially in a specific cell if expression in this cell type is significantly higher than expression in other cell types. The term “regulatory element” also encompasses non-tissue specific regulatory elements, i.e., regulatory elements which are active in most cell types. Furthermore, a regulatory element can be a constitutive regulatory element, i.e., a regulatory element which constitutively regulates transcription, as opposed to a regulatory element which is inducible, i.e., a regulatory element which is active primarily in response to a stimulus. A stimulus can be, e.g., a molecule, such as a hormone, cytokine, heavy metal, phorbol ester, cyclic AMP (cAMP), or retinoic acid.

Regulatory elements are typically bound by proteins, e.g., transcription factors. The term “transcription factor” is intended to include proteins or modified forms thereof, which interact preferentially with specific nucleic acid sequences, i.e., regulatory elements, and which in appropriate conditions stimulate or repress transcription. Some transcription factors are active when they are in the form of a monomer. Alternatively, other transcription factors are active in the form of a dimer consisting of two identical proteins or different proteins (heterodimer). Modified forms of transcription factors are intended to refer to transcription factors having a post-translational modification, such as the attachment of a phosphate group. The activity of a transcription factor is frequently modulated by a post-translational modification. For example, certain transcription factors are active only if they are phosphorylated on specific residues. Alternatively, transcription factors can be active in the absence of phosphorylated residues and become inactivated by phosphorylation. A list of known transcription factors and their DNA binding site can be found, e.g., in public databases, e.g., TFMATRIX Transcription Factor Binding-Site Profile database.

As used herein, the term “specifically hybridizes” or “specifically detects” refers to the ability of a nucleic acid molecule of the invention to hybridize to at least approximately 6, 12, 20, 30, 40, 50, 60, 70, 80, 90, 100, 110, 120, 130 or 140 consecutive nucleotides of either strand of a gene.

The term “wild-type allele” refers to an allele of a gene which, when present in two copies in a subject results in a wild-type phenotype. There can be several different wild-type alleles of a specific gene, since certain nucleotide changes in a gene may not affect the phenotype of a subject having two copies of the gene with the nucleotide changes.

“Adverse drug reaction” (ADR) as used herein refers to an appreciably harmful or unpleasant reaction, resulting from an intervention related to the use of a medicinal product, which predicts hazard from future administration and warrants prevention or specific treatment, or alteration of the dosage regimen, or withdrawal of the product. In it's most severe form an ADR might lead to the death of an individual.

The term “Drug Response” is intended to mean any response that a patient exhibits upon drug administration. Specifically drug response includes beneficial, i.e. desired drug effects, ADR or no detectable reaction at all. More specifically the term drug response could also have a qualitative meaning, i.e. it embraces low or high beneficial effects, respectively and mild or severe ADR, respectively. The term “Statin Response” as used herein refers to drug response after statin administration. An individual drug response includes also a good or bad metabolizing of the drug, meaning that “bad metabolizers” accumulate the drug in the body and by this could show side effects of the drug due to accumulative overdoses.

“Candidate gene” as used herein includes genes that can be assigned to either normal cardiovascular function or to metabolic pathways that are related to onset and/or progression of cardiovascular diseases.

With regard to drug response the term “candidate gene” includes genes that can be assigned to distinct phenotypes regarding the patient's response to drug administration. Those phenotypes may include patients who benefit from relatively small amounts of a given drug (high responders) or patients who need relatively high doses in order to obtain the same benefit (low responders). In addition those phenotypes may include patients who can tolerate high doses of a medicament without exhibiting ADR, or patients who suffer from ADR even after receiving only low doses of a medicament.

As neither the development of cardiovascular diseases nor the patient's response to drug administration is completely understood, the term “candidate gene” may also comprise genes with presently unknown function.

“PA SNP” (phenotype associated SNP) refers to a polymorphic site which shows a significant association with a patients phenotype (healthy, diseased, low or high responder, drug tolerant, ADR prone, etc.)

“PA gene” (phenotype associated gene) refers to a genomic locus harbouring a PA SNP, irrespective of the actual function of this gene locus.

PA gene polypeptide refers to a polypeptide encoded at least in part by a PA gene.

The term “Haplotype” as used herein refers to a group of two or more SNPs that are functionally and/or spatially linked. I.e. haplotypes define groups of SNPs that lie inside genes belonging to identical (or related metabolic) pathways and/or lie on the same chromosome. Haplotypes are expected to give better predictive/diagnostic information than a single SNP

The term “statin” is intended to embrace all inhibitors of the enzyme 3-hydroxy-3-methylglutaryl coenzyme A (HMG-CoA) reductase. Statins specifically inhibit the enzyme HMG-CoA reductase which catalyzes the rate limiting step in cholesterol biosynthesis. Known statins are Atorvastatin, Cerivastatin, Fluvastatin, Lovastatin, Pravastatin and Simvastatin.

Methods for Assessing Cardiovascular Status

The present invention provides diagnostic methods for assessing cardiovascular status in a human individual. Cardiovascular status as used herein refers to the physiological status of an individual's cardiovascular system as reflected in one or more markers or indicators. Status markets include without limitation clinical measurements such as, e.g., blood pressure, electrocardiographic profile, and is differentiated blood flow analysis as well as measurements of LDL- and HDL-Cholesterol levels, other lipids and other well established clinical parameters that are standard in the art. Status markers according to the invention include diagnoses of one or more cardiovascular syndromes, such as, e.g., hypertension, acute myocardial infarction, silent myocardial infarction, stroke, and atherosclerosis. It will be understood that a diagnosis of a cardiovascular syndrome made by a medical practitioner encompasses clinical measurements and medical judgement. Status markers according to the invention are assessed using conventional methods well known in the art. Also included in the evaluation of cardiovascular status are quantitative or qualitative changes in status markers with time, such as would be used, e.g., in the determination of an individual's response to a particular therapeutic regimen.

The methods are carried out by the steps of:
(i) determining the sequence of one or more polymorphic positions within one, several or all of the genes listed in Examples or other genes mentioned in this file in the individual to establish a polymorphic pattern for the individual; and
(ii) comparing the polymorphic pattern established in (i) with the polymorphic patterns of humans exhibiting different markers of cardiovascular status. The polymorphic pattern of the individual is, preferably, highly similar and, most preferably, identical to the polymorphic pattern of individuals who exhibit particular status markers, cardiovascular syndromes, and/or particular patterns of response to therapeutic interventions. Polymorphic patterns may also include polymorphic positions in other genes which are shown, in combination with one or more polymorphic positions in the genes listed in the Examples, to correlate with the presence of particular status markers. In one embodiment, the method involves comparing an individual's polymorphic pattern with polymorphic patterns of individuals who have been shown to respond positively or negatively to a particular therapeutic regimen. Therapeutic regimen as used herein refers to treatments aimed at the elimination or amelioration of symptoms and events associated cardiovascular disease. Such treatments include without limitation one or more of alteration in diet, lifestyle, and exercise regimen; invasive and noninvasive surgical techniques such as atherectomy, angioplasty, and coronary bypass surgery; and pharmaceutical interventions, such as administration of ACE inhibitors, angiotensin II receptor antagonists, diuretics, alpha-adrenoreceptor antagonists, cardiac glycosides, phosphodiesterase inhibitors, beta-adrenoreceptor antagonists, calcium channel blockers, HMG-CoA reductase inhibitors, imidazoline receptor blockers, endothelin receptor blockers, organic nitrites, and modulators of protein function of genes listed in the Examples. Interventions with pharmaceutical agents not yet known whose activity correlates with particular polymorphic patterns associated with cardiovascular disease are also encompassed. It is contemplated, for example, that patients who are candidates for a particular therapeutic regimen will be screened for polymorphic patterns that correlate with responsivity to that particular regimen.

In a preferred embodiment, the method involves comparing an individual's polymorphic pattern with polymorphic patterns of individuals who exhibit or have exhibited one or more markers of cardiovascular disease, such as, e.g., elevated LDL-Cholesterol levels, high blood pressure, abnormal electrocardiographic profile, myocardial infarction, stroke, or atherosclerosis.

In another embodiment, the method involves comparing an individual's polymorphic pattern with polymorphic patterns of individuals who exhibit or have exhibited one or more drug related phenotypes, such as, e.g., low or high drug response, or adverse drug reactions.

In practicing the methods of the invention, an individual's polymorphic pattern can be established by obtaining DNA from the individual and determining the sequence at predetermined polymorphic positions in the genes such as those described in this file.

The DNA may be obtained from any cell source. Non-limiting examples of cell sources available in clinical practice include blood cells, buccal cells, cervicovaginal cells, epithelial cells from urine, fetal cells, or any cells present in tissue obtained by biopsy. Cells may also be obtained from body fluids, including without limitation blood, saliva, sweat, urine, cerebrospinal fluid, feces, and tissue exudates at the site of infection or inflammation. DNA is extracted from the cell source or body fluid using any of the numerous methods that are standard in the art. It will be understood that the particular method used to extract DNA will depend on the nature of the source.

Diagnostic and Prognostic Assays

The present invention provides methods for determining the molecular structure of at least one polymorphic region of a gene, specific allelic variants of said polymorphic region being associated with cardiovascular disease. In one embodiment, determining the molecular structure of a polymorphic region of a gene comprises determining the identity of the allelic variant. A polymorphic region of a gene, of which specific alleles are associated with cardiovascular disease can be located in an exon, an intron, at an intron/exon border, or in the promoter of the gene.

The invention provides methods for determining whether a subject has, or is at risk, of developing a cardiovascular disease. Such disorders can be associated with an aberrant gene activity, e.g., abnormal binding to a form of a lipid, or an aberrant gene protein level. An aberrant gene protein level can result from an aberrant transcription or post-transcriptional regulation. Thus, allelic differences in specific regions of a gene can result in differences of gene protein due to differences in regulation of expression. In particular, some of the identified polymorphisms in the human gene may be associated with differences in the level of transcription, RNA maturation, splicing, or translation of the gene or transcription product.

In preferred embodiments, the methods of the invention can be characterized as comprising detecting, in a sample of cells from′ the subject, the presence or absence of a specific allelic variant of one or more polymorphic regions of a gene. The allelic differences can be: (i) a difference in the identity of at least one nucleotide or (ii) a difference in the number of nucleotides, which difference can be a single nucleotide or several nucleotides.

A preferred detection method is allele specific hybridization using probes over-lapping the polymorphic site and having about 5, 10, 20, 25, or 30 nucleotides around the polymorphic region. Examples of probes for detecting specific allelic variants of the polymorphic region located in intron X are probes comprising a nucleotide sequence set forth in any of SEQ ID NO. X. In a preferred embodiment of the invention, several probes capable of hybridizing specifically to allelic variants are attached to a solid phase support, e.g., a “chip”. Oligonucleotides can be bound to a solid support by a variety of processes, including lithography. For example a chip can hold up to 250,000 oligonucleotides (GeneChip, Affymetrix). Mutation detection analysis using these chips comprising oligonucleotides, also termed “DNA probe arrays” is described e.g., in Cronin et al. (1996) Human Mutation 7:244 and in Kozal et al. (1996) Nature Medicine 2:753. In one embodiment,a chip comprises all the allelic variants of at least one polymorphic region of a gene. The solid phase support is then contacted with a test nucleic acid and hybridization to the specific probes is detected. Accordingly, the identity of numerous allelic variants of one or more genes can be identified in a simple hybridization experiment. For example, the identity of the allelic variant of the nucleotide polymorphism of nucleotide A or G at position 33 of Seq ID 1 (baySNP179) and that of other possible polymorphic regions can be determined in a single hybridization experiment.

In other detection methods, it is necessary to first amplify at least a portion of a gene prior to identifying the allelic variant. Amplification can be performed, e.g., by PCR and/or LCR, according to methods known in the art. In one embodiment, genomic DNA of a cell is exposed to two PCR primers and amplification for a number of cycles sufficient to produce the required amount of amplified DNA. In preferred embodiments, the primers are located between 40 and 350 base pairs apart. Preferred primers for amplifying gene fragments of genes of this file are listed in Table 2 in the Examples.

Alternative amplification methods include: self sustained sequence replication (Guatelli, J. C. et al., 1990, Proc. Natl. Acad. Sci. U.S.A. 87:1874-1878), transcriptional amplification system (woh, D. Y. et al., 1989, Proc. Natl. Acad. Sci. U.S.A. 86:1173-1177), Q-Beta Replicase (Lizardi, P. M. et al., 1988, Bio/Technology 6:1197), or any other nucleic acid amplification method, followed by the detection of the amplified molecules using techniques well known to those of skill in the art.

These detection schemes are especially useful for the detection of nucleic acid molecules if such molecules are present in very low numbers.

In one embodiment, any of a variety of sequencing reactions known in the art can be used to directly sequence at least a portion of a gene and detect allelic variants, e.g., mutations, by comparing the sequence of the sample sequence with the corresponding wild-type (control) sequence. Exemplary sequencing reactions include those based on techniques developed by Maxam and Gilbert (Proc. Natl Acad Sci USA (1977) 74:560) or Sanger (Sanger et al (1977) Proc. Nat. Acad. Sci 74:5463). It is also contemplated that any of a variety of automated sequencing procedures may be utilized when performing the subject assays (Biotechniques (1995) 19:448), including sequencing by mass spectrometry (see, for example, U.S. Pat. No. 5,547,835 and international patent application Publication Number WO 94/16101, entitled DNA Sequencing by Mass Spectrometry by H. Koster; U.S. Pat. No. 5,547,835 and international patent application Publication Number WO 94/21822 entitled “DNA Sequencing by Mass Spectrometry Via Exonuclease Degradation” by H. Koster), and U.S. Pat. No. 5,605,798 and International Patent Application No. PCT/US96/03651 entitled DNA Diagnostics Based on Mass Spectrometry by H. Koster; Cohen et al. (1996) Adv Chromatogr 36:127-162; and Griffin et al. (1993) Appl Biochem Biotechnol 38:147-159). It will be evident to one skilled in the art that, for certain embodiments, the occurrence of only one, two or three of the nucleic acid bases need be determined in the sequencing reaction. For instance, A-track or the like, e.g., where only one nucleotide is detected, can be carried out.

Yet other sequencing methods are disclosed, e.g., in U.S. Pat. No. 5,580,732 entitled “Method of DNA sequencing employing a mixed DNA-polymer chain probe” and U.S. Pat. No. 5,571,676 entitled “Method for mismatch-directed in vitro DNA sequencing”.

In some cases, the presence of a specific allele of a gene in DNA from a subject can be shown by restriction enzyme analysis. For example, a specific nucleotide poly-morphism can result in a nucleotide sequence comprising a restriction site which is absent from the nucleotide sequence of another allelic variant.

In other embodiments, alterations in electrophoretic mobility is used to identify the type of gene allelic variant. For example, single strand conformation polymorphism (SSCP) may be used to detect differences in electrophoretic mobility between mutant and wild type nucleic acids (Orita et al. (1989) Proc. Natl. Acad. Sci USA 86:2766, see also Cotton (1993) Mutat Res 285:125-144; and Hayashi (1992) Genet Anal Tech Appl 9:73-79). Single-stranded DNA fragments of sample and control nucleic acids are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In another preferred embodiment, the subject method utilizes heteroduplex analysis to separate double stranded heteroduplex molecules on the basis of changes in electrophoretic mobility (Keen et al. (1991) Trends Genet 7:5).

In yet another embodiment, the identity of an allelic variant of a polymorphic region is obtained by analyzing the movement of a nucleic acid comprising the polymorphic region in polyacrylamide gels containing a gradient of denaturant is assayed using denaturing gradient gel electrophoresis (DGGE) (Myers et al (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:1275).

Examples of techniques for detecting differences of at least one nucleotide between 2 nucleic acids include, but are not limited to, selective oligonucleotide hybridization, selective amplification, or selective primer extension. For example, oligonucleotide probes may be prepared in which the known polymorphic nucleotide is placed centrally (allele-specific probes) and then hybridized to target DNA under conditions which permit hybridization only if a perfect match is found (Saiki et al. (1986) Nature 324:163); Saiki et al (1989) Proc. Natl Acad. Sci USA 86:6230; and Wallace et al. (1979) Nucl. Acids Res. 6:3543). Such allele specific oligonucleotide hybridization techniques may be used for the simultaneous detection of several nucleotide changes in different polymorphic regions of gene. For example, oligonucleotides having nucleotide sequences of specific allelic variants are attached to a hybridizing membrane and this membrane is then hybridized with labeled sample nucleic acid. Analysis of the hybridization signal will then reveal the identity of the nucleotides of the sample nucleic acid.

Alternatively, allele specific amplification technology which depends on selective PCR amplification may be used. Oligonucleotides used as primers for specific amplification may carry the allelic variant of interest in the center of the molecule (so that amplification depends on differential hybridization) (Gibbs et al (1989) Nucleic Acids Res. 17:2437-2448) or at the extreme 3′ end of one primer where, under appropriate conditions, mismatch can prevent, or reduce polymerase extension (Prossner (1993) Tibtech 11:238; Newton et al. (1989) Nucl. Acids Res. 17:2503). This technique is also termed “PROBE” for Probe Oligo Base Extension. In addition it may be, desirable to introduce a novel restriction site in the region of the mutation to create cleavage-based detection (Gasparini et al (1992) Mol. Cell Probes 6:1).

In another embodiment, identification of the allelic variant is carried out using an oligonucleotide ligation assay (OLA), as described, e.g., in U.S. Pat. No. 4,998,617 and in Landegren, U. et al., Science 241:1077-1080 (1988). The OLA protocol uses two oligonucleotides which are designed to be capable of hybridizing to abutting sequences of a single strand of a target. One of the oligonucleotides is linked to a separation marker, e.g,. biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al., Proc. Natl. Acad. Sci. (U.S.A.) 87:8923-8927 (1990). In this method, PCR is used to achieve the exponential amplification of target DNA, which is then detected using OLA.

Several techniques based on this OLA method have been developed and can be used to detect specific allelic variants of a polymorphic region of a gene. For example, U.S. Pat. No. 5,593,826 discloses an OLA using an oligonucleotide having 3′-amino group and a 5′-phosphorylated oligonucleotide to form a conjugate having a phosphoraridate linkage. In another variation of OLA described in Tobe et al. ((1996) Nucleic Acids Res 24: 3728), OLA combined with PCR permits typing of two alleles in a single microtiter well. By marking each of the allele-specific primers with a unique hapten, i.e. digoxigenin and fluorescein, each LA reaction can be detected by using hapten specific antibodies that are labeled with different enzyme reporters, alkaline phosphatase or horseradish peroxidase. This system permits the detection of the two alleles using a high throughput format that leads to the production of two different colors.

The invention further provides methods for detecting single nucleotide poly-morphisms in a gene. Because single nucleotide polymorphisms constitute sites of variation flanked by regions of invariant sequence, their analysis requires no more than the determination of the identity of the single nucleotide present at the site of variation and it is unnecessary to determine a complete gene sequence for each patient. Several methods have been developed to facilitate the analysis of such single nucleotide polymorphisms.

In one embodiment, the single base polymorphism can be detected by using a specialized exonuclease-resistant-nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data

In another embodiment of the invention, a solution-based method is used for determining the identity of the nucleotide of a polymorphic site. Cohen, D. et al. (French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA TM is described by Goelet, P. et al. (PCT Appln. No. 92/15712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet, P. et al. is preferably a heterogeneous phase assay, in which the prumer or the target molecule is immobilized to a solid phase.

Recently, several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvanen, A. -C., et al., Genomics 8:684-692.(1990), Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoli, L. et al., GATA 9:107-112 (1992); Nyren, P. et, al., Anal. Biochem. 208:171-175 (1993)). These methods differ from GBA TM in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C., et al., Amer. J. Hum. Genet. 52:46-59 (1993)).

For determining the identity of the allelic variant of a polymorphic region located in the coding region of a gene, yet other methods than those described above can be used. For example, identification of an allelic variant which encodes a mutated gene protein can be performed by using an antibody specifically recognizing the mutant protein in, e.g., immunohistochemistry or imnunoprecipitation. Antibodies to wild-type gene protein are described, e.g., in Acton et al. (1999) Science 271:518 (anti-mouse gene antibody cross-reactive with human gene). Other antibodies to wild-type gene or mutated forms of gene proteins can be prepared according to methods known in the art. Alternatively, one can also measure an activity of an gene protein, such as binding to a lipid or lipoprotein. Binding assays are known in the art and involve, e.g., obtaining cells from a subject, and performing binding experiments with a labeled lipid, to determine whether binding to the mutated form of the receptor differs from binding to the wild-type of the receptor.

If a polymorphic region is located in an exon, either in a coding or non-coding region of the gene, the identity of the allelic variant can be determined by determining the molecular structure of the mRNA, pre-mRNA, or cDNA. The molecular structure can be determined using any of the above described methods for determining the molecular structure of the genomic DNA, e.g., sequencing and SSCP.

The methods described herein may be performed, for example, by utilizing pre-packaged diagnostic kits, such as those described above, comprising at least one probe or primer nucleic acid described herein, which may be conveniently used, e.g., to determine whether a subject has or is at risk of developing a disease associated with a specific gene allelic variant.

Sample nucleic acid for using in the above-described diagnostic and prognostic methods can be obtained from any cell type or tissue of a subject. For example, a subject's bodily fluid (e.g. blood) can be obtained by known techniques (e.g. venipuncture) or from human tissues like heart (biopsies, transplanted organs). Alternatively, nucleic acid tests can be performed on dry samples (e.g. hair or skin). Fetal nucleic acid samples for prenatal diagnostics can be obtained from maternal blood as described in International Patent Application No. WO91/07660 to Bianchi. Alternatively, amniocytes or chorionic villi may be obtained for performing prenatal testing.

Diagnostic procedures may also be performed in situ directly upon tissue sections (fixed and/or frozen) of patient tissue obtained from biopsies or resections, such that no nucleic acid purification is necessary. Nucleic acid reagents may be used as probes and/or primers for such in situ procedures (see, for example, Nuovo, G. J., 1992, PCR in situ hybridization: protocols and applications, Raven Press, New York).

In addition to methods which focus primarily on the detection of one nucleic acid sequence, profiles may also be assessed in such detection schemes. Fingerprint profiles may be generated, for example, by utilizing a differential display procedure, Northern analysis and/or RT-PCR.

In practicing the present invention, the distribution of polymorphic patterns in a large number of individuals exhibiting particular markers of cardiovascular status or drug response is determined by any of the methods described above, and compared with the distribution of polymorphic patterns in patients that have been matched for age, ethnic origin, and/or any other statistically or medically relevant parameters, who exhibit quantitatively or qualitatively different status markers. Correlations are achieved using any method known in the art, including nominal logistic regression, chi square tests or standard least squares regression analysis. In this manner, it is possible to establish statistically significant correlations between particular poly-morphic patterns and particular cardiovascular statuses (given in p values). It is further possible to establish statistically significant correlations between particular polymorphic patterns and changes in cardiovascular status or drug response such as, would result, e.g., from particular treatment regimens. In this manner, it is possible to correlate polymorphic patterns with responsivity to particular treatments.

In another embodiment of the present invention two or more polymorphic regions are combined to define so called ‘haplotypes’. Haplotypes are groups of two or more SNPs that are functionally and/or spatially linked. It is possible to combine SNPs that are disclosed in the present invention either with each other or with additional polymorphic regions to form a haplotype. Haplotypes are expected to give better predictive/diagnostic information than a single SNP.

In a preferred embodiment of the present invention a panel of SNPs/haplotypes is defined that predicts the risk for CVD or drug response. This predictive panel is then used for genotyping of patients on a platform that can genotype multiple SNPs at the same time (Multiplexing). Preferred platforms are e.g. gene chips (Affymetrix) or the Luminex LabMAP reader. The subsequent identification and evaluation of a patient's haplotype can then help to guide specific and individualized therapy.

For example the present invention can identify patients exhibiting genetic polymorphisms or haplotypes which indicate an increased risk for adverse drug reactions. In that case the drug dose should be lowered in a way that the risk for ADR is diminished. Also if the patient's response to drug administration is particularly high (or the patient is badly metabolizing the drug), the drug dose should be lowered to avoid the risk of ADR.

In turn if the patient's response to drug administration is low (or the patient is a particularly high metabolizer of the drug), and there is no evident risk of ADR, the drug dose should be raised to an efficacious level.

It is self evident that the ability to predict a patient's individual drug response should affect the formulation of a drug, i.e. drug formulations should be tailored in a way that they suit the different patient classes (low/high responder, poor/good metabolizer, ADR prone patients); Those different drug formulations may encompass different doses of the drug, i.e. the medicinal products contains low or high amounts of the active substance. In another embodiment of the invention the drug formulation may contain additional substances that facilitate the beneficial effects and/or diminish the risk for ADR (Folkers et al. 1991, U.S. Pat. No. 5,316,765).

Isolated Polymorphic Nucleic Acids, Probes, and Vectors

The present invention provides isolated nucleic acids comprising the polymorphic positions described herein for human genes; vectors comprising the nucleic acids; and transformed host cells comprising the vectors. The invention also provides probes which are useful for detecting these polymorphisms.

In practicing the present invention, many conventional techniques in molecular biology, microbiology, and recombinant DNA, are used. Such techniques are well known and are explained fully in, for example, Sambrook et al., 1989, Molecular Cloning: A Laboratory Manual, Second Edition, Cold Spring Harbor Laboratory Press, Cold Spring Harbor, N.Y.; DNA Cloning: A Practical Approach, Volumes I and II, 1985 (D. N. Glover ed.); Oligonucleotide Synthesis, 1984, (M. L. Gait ed.); Nucleic Acid Hybridization, 1985, (Hames and Higgins); Ausubel et al., Current Protocols in Molecular Biology, 1997, (John Wiley and Sons); and Methods in Enzymology Vol. 154 and Vol. 155 (Wu and Grossman, and Wu, eds., respectively).

Insertion of nucleic acids (typically DNAs) comprising the sequences in a functional surrounding like full length cDNA of the present invention into a vector is easily accomplished when the termini of both the DNAs and the vector comprise compatible restriction sites. If this cannot be done, it may be necessary to modify the termini of the DNAs and/or vector by digesting back single-stranded DNA overhangs generated by restriction endonuclease cleavage to produce blunt ends, or to achieve the same result by filling in the single-stranded termini with an appropriate DNA polymerase.

Alternatively, any site desired may be produced, e.g., by ligating nucleotide sequences (linkers) onto the termini. Such linkers may comprise specific oligo-nucleotide-sequences that define desired restriction sites;. Restriction sites can also be generated by the use of the polymerase chain reaction (PCR). See, e.g., Saiki et al., 1988, Science 239:48. The cleaved vector and the DNA fragments may also be modified if required by homopolymeric tailing.

The nucleic acids may be isolated directly from cells or may be chemically synthesized using known methods. Alternatively, the polymerase chain reaction (PCR) method can be used to produce the nucleic acids of the invention, using either chemically synthesized strands or genomic material as templates. Primers used for PCR can be synthesized using the sequence information provided herein and can further be designed to introduce appropriate new restriction sites, if desirable, to facilitate incorporation into a given vector for recombinant expression.

The nucleic acids of the present invention may be flanked by native gene sequences, or may be associated with heterologous sequences, including promoters, enhancers, response elements, signal sequences, polyadenylation sequences, introns, 5′- and 3′-noncoding regions, and the like. The nucleic acids may also be modified by many means known in the art. Non-limiting examples of such modifications include methylation, “caps”, substitution of one or more of the naturally occurring nucleotides with an analog, internucleotide modifications such as, for example, those with uncharged linkages (e.g., methyl phosphonates, phosphotriesters, phosphoro-amidates, carbamates, morpholines etc.) and with charged linkages (e.g., phosphorothioates, phosphorodithioates, etc.). Nucleic acids may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. PNAs are also included. The nucleic acid may be derivatized by formation of a methyl or ethyl phosphotriester or an alkyl phosphoramidate linkage. Furthermore, the nucleic acid sequences of the present invention may also be modified with a label capable of providing a detectable signal, either directly or indirectly. Exemplary labels include radioisotopes, fluorescent molecules, biotin, and the like.

The invention also provides nucleic acid vectors comprising the gene sequences or derivatives or fragments thereof of genes described in the Examples. A large number of vectors, including plasmid and fungal vectors, have been described for replication and/or expression in a variety of eukaryotic and prokaryotic hosts, and may be used for gene therapy as well as for simple cloning or protein expression. Non-limiting examples of suitable vectors include without limitation pUC plasmids, pET plasmids (Novagen, Inc., Madison, Wis.), or pRSET or pREP (Invitrogen, San Diego, Calif.), and many appropriate host cells, using methods disclosed or cited herein or otherwise known to those skilled in the relevant art. The particular choice of vector/host is not critical to the practice of the invention.

Suitable host cells may be transformed/transfected/infected as appropriate by any suitable method including electroporation, CaCl₂ mediated DNA uptake, fungal or viral infection, microinjection, microprojectile, or other established methods. Appropriate host cells included bacteria, archebacteria, fungi, especially yeast, and plant and animal cells, especially mammalian cells. A large number of transcription initiation and termination regulatory regions have been isolated and shown to be effective in the transcription and translation of heterologous proteins in the various hosts. Examples of these regions, methods of isolation, manner of manipulation, etc. are known in the art. Under appropriate expression conditions, host cells can be used as a source of recombinantly produced peptides and polypeptides encoded by genes of the Examples. Nucleic acids encoding peptides or polypeptides from gene sequences of the Examples may also be introduced into cells by recombination events. For example, such a sequence can be introduced into a cell and thereby effect homologous recombination at the site of an endogenous gene or a sequence with substantial identity to the gene. Other recombination-based methods such as non-homologous recombinations or deletion of endogenous genes by homologous recombination may also be used.

In case of proteins that form heterodimers or other multimers, both or all subunits have to be expressed in one system or cell.

The nucleic acids of the present invention find use as probes for the detection of genetic polymorphisms and as templates for the recombinant production of normal or variant peptides or polypeptides encoded by genes listed in the Examples.

Probes in accordance with the present invention comprise without limitation isolated nucleic acids of about 10-100 bp, preferably 15-75 bp and most preferably 17-25 bp in length, which hybridize at high stringency to one or more of the polymorphic sequences disclosed herein or to a sequence immediately adjacent to a polymorphic position. Furthermore, in some embodiments a full-length gene sequence may be used as a probe. In one series of embodiments, the probes span the polymorphic positions in genes disclosed herein. In another series of embodiments, the probes correspond to sequences immediately adjacent to the polymorphic positions.

Polymorphic Polypeptides and Polymorphism-Specific Antibodies

The present invention encompasses isolated peptides and polypeptides encoded by genes listed in the Examples comprising polymorphic positions disclosed herein. In one preferred embodiment, the peptides and polypeptides are useful screening targets to identify cardiovascular drugs. In another preferred embodiments, the peptides and polypeptides are capable of eliciting antibodies in a suitable host animal that react specifically with a polypeptide comprising the polymorphic position and distinguish it from other polypeptides having a different sequence at that position.

Polypeptides according to the invention are preferably at least five or more residues in length, preferably at least fifteen residues. Methods for obtaining these poly-peptides are described below. Many conventional techniques in protein biochemistry and immunology are used. Such techniques are well known and are explained in Immunochemical Methods in Cell and Molecular Biology, 1987 (Mayer and Waler, eds; Academic Press, London); Scopes, 1987, Protein Purification: Principles and Practice, Second Edition (Springer-Verlag, N.Y.) and Handbook of Experimental Immunology, 1986, Volumes I-IV (Weir and Blackwell eds.).

Nucleic acids comprising protein-coding sequences can be used to direct the ITT recombinant expression of polypeptides encoded by genes disclosed herein in intact cells or in cell-free translation systems. The known genetic code, tailored if desired for more efficient expression in a given host organism, can be used to synthesize oligonucleotides encoding the desired amino acid sequences. The polypeptides may be isolated from human cells, or from heterologous organisms or cells (including, but not limited to, bacteria, fungi, insect, plant, and mammalian cells) into which an appropriate protein-coding sequence has been introduced and expressed. Furthermore, the polypeptides may be part of recombinant fusion proteins.

Peptides and polypeptides may be chemically synthesized by commercially available automated procedures, including, without limitation, exclusive solid phase synthesis, partial solid phase methods, fragment condensation or classical solution synthesis. The polypeptides are preferably prepared by solid phase peptide synthesis as described by Merrifield, 1963, J. Am. Chem. Soc. 85:2149.

Methods for polypeptide purification are well-known in the art, including, without limitation, preparative disc-gel electrophoresis, isoelectric focusing, HPLC, reversed-phase HPLC, gel filtration, ion exchange and partition chromatography, and countercurrent distribution. For some purposes, it is preferable to produce the polypeptide in a recombinant system in which the protein contains an additional sequence tag that facilitates purification, such as, but not limited to, a polyhistidine sequence. The polypeptide can then be purified from a crude lysate of the host cell by chromatography on an appropriate solid-phase matrix. Alternatively, antibodies produced against peptides encoded by genes disclosed herein, can be used as purification reagents. Other purification methods are possible.

The present invention also encompasses derivatives and homologues of the poly-peptides. For some purposes, nucleic acid sequences encoding the peptides may be altered by substitutions, additions, or deletions that provide for functionally equivalent molecules, i.e., function-conservative variants. For example, one or more amino acid residues within the sequence can be substituted by another amino acid of similar properties, such as, for example, positively charged amino acids (arginine, lysine, and histidine); negatively charged amino acids (aspartate and glutamate); polar neutral amino acids; and non-polar amino acids.

The isolated polypeptides may be modified by, for example, phosphorylation, sulfation, acylation, or other protein modifications. They may also be modified with a label capable of providing a detectable signal, either directly or indirectly, including, but not limited to, radioisotopes and fluorescent compounds.

The present invention also encompasses antibodies that specifically recognize the polymorphic positions of the invention and distinguish a peptide or polypeptide containing a particular polymorphism from one that contains a different sequence at that position. Such polymorphic position-specific antibodies according to the present invention include polyclonal and monoclonal -antibodies. The antibodies may be elicited in an animal host by immunization with peptides encoded by genes disclosed herein or may be formed by in vitro immunization of immune cells. The immuno-genic components used to elicit the antibodies may be isolated from human cells or produced in recombinant systems. The antibodies may also be produced in recombinant systems programmed with appropriate antibody-encoding DNA. Alternatively, the antibodies may be constructed by biochemical reconstitution of purified heavy and light chains. The antibodies include hybrid antibodies (i.e., containing two sets of heavy chain/light chain combinations, each of which recognizes a different antigen), chimeric antibodies (i.e., in which either the heavy chains, light chains, or both, are fusion proteins), and univalent antibodies (i.e., comprised of a heavy chain/light chain complex bound to the constant region of a second heavy chain). Also included are Fab fragments, including Fab′ and F(ab).sub.2 fragments of antibodies. Methods for the production of all of the above types of antibodies and derivatives are well-known in the art and are discussed in more detail below. For example, techniques for producing and processing polyclonal antisera are disclosed in Mayer and Walker, 1987, immunochemical Methods in Cell and Molecular Biology, (Academic Press, London). The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. See, e.g., Schreier et al., 1980, Hybridoma Techniques; U.S. Pat. Nos. 4,341,761; 4,399,121; 4,427,783; 4,444,887; 4,466,917; 4,472,500; 4,491,632; and 4,493,890. Panels of monoclonal antibodies produced against peptides encoded by genes disclosed herein can be screened for various properties; i.e. for isotype, epitope affinity, etc.

The antibodies of this invention can be purified by standard methods, including but not limited to preparative disc-gel electrophoresis, isoelectric focusing, HPLC, reversed-phase HPLC, gel filtration, ion exchange and partition chromatography, and countercurrent distribution. Purification methods for antibodies are disclosed, e.g., in The Art of Antibody Purification, 1989, Amicon Division, W. R. Grace & Co. General protein purification methods are described in Protein Purification: Principles and Practice, R. K. Scopes, Ed., 1987, Springer-Verlag, New York, N.Y.

Methods for determining the immunogenic capability of the disclosed sequences and the characteristics of the resulting sequence-specific antibodies and immune cells are well-known in the art. For example, antibodies elicited in response to a peptide comprising a particular polymorphic sequence can be tested for their ability to specifically recognize that polymorphic sequence, i.e., to bind differentially to a peptide or polypeptide comprising the polymorphic sequence and thus distinguish it from a similar peptide or polypeptide containing a different sequence at the same position.

Kits

As set forth herein, the invention provides diagnostic methods, e.g., for determining the identity of the allelic variants of polymorphic regions present in the gene loci of genes disclosed herein, wherein specific allelic variants of the polymorphic region are associated with cardiovascular diseases. In a preferred embodiment, the diagnostic kit can be used to determine whether a subject is at risk of developing a cardiovascular disease. This information could then be used, e.g., to optimize treatment of such individuals.

In preferred embodiments, the kit comprises a probe or primer which is capable of hybridizing to a gene and thereby identifying whether the gene contains an allelic variant of a polymorphic region which is associated with a risk for cardiovascular disease. The kit preferably further comprises instructions for use in diagnosing a subject as having, or having a predisposition, towards developing a cardiovascular disease. The probe or primers of the kit can be any of the probes or primers described in this file.

Preferred kits for amplifying a region of a gene comprising a polymorphic region of interest comprise one, two or more primers.

Antibody-Based Diagnostic Methods and Kits

The invention also provides antibody-based methods for detecting polymorphic patterns in a biological sample. The methods comprise the steps of: (i) contacting a sample with one or more antibody preparations, wherein each of the antibody preparations is specific for a particular polymorphic form of the proteins encoded by genes disclosed herein, under conditions in which a stable antigen-antibody complex can form between the antibody and antigenic components in the sample; and (ii) detecting any antigen-antibody complex formed in step (i) using any suitable means known in the art, wherein the detection of a complex indicates the presence of the particular polymorphic form in the sample.

Typically, immunoassays use either a labelled antibody or a labelled antigenic component (e.g., that competes with the antigen in the sample for binding to the antibody). Suitable labels include without limitation enzyme-based, fluorescent, chemiluminescent, radioactive, or dye molecules. Assays that amplify the signals from the probe are also known, such as, for example, those that utilize biotin and avidin, and enzyme-labelled immunoassays, such as ELISA assays.

The present invention also provides kits suitable for antibody-based diagnostic applications. Diagnostic kits typically include one or more of the following components:
(i) Polymorphism-specific antibodies. The antibodies may be pre-labelled; alternatively, the antibody may be unlabelled and the ingredients for labelling may be included in the kit in separate containers, or a secondary, labelled antibody is provided; and
(ii) Reaction components: The kit may also contain other suitably packaged reagents and materials needed for the particular immunoassay protocol, including solid-phase matrices, if applicable, and standards.

The kits referred to above may include instructions for conducting the test. Furthermore, in preferred embodiments, the diagnostic kits are adaptable to high-throughput and/or automated operation.

Drug Tareets and Screening Methods

According to the present invention, nucleotide sequences derived from genes disclosed herein and peptide sequences encoded by genes disclosed herein, particularly those that contain one or more polymorphic sequences, comprise useful targets to identify cardiovascular drugs, i.e., compounds that are effective in treating one or more clinical symptoms of cardiovascular disease. Furthermore, especially when a protein is a multimeric protein that are build of two or more subunits, is a combination of different polymorphic subunits very useful.

Drug targets include without limitation (i) isolated nucleic acids derived from the genes disclosed herein, and (ii) isolated peptides and polypeptides encoded by genes disclosed herein, each of which comprises one or more polymorphic positions.

In Vitro Screening Methods

In one series of embodiments, an isolated nucleic acid comprising one or more polymorphic positions is tested in vitro for its ability to bind test compounds in a sequence-specific manner. The methods comprise:
(i) providing a first nucleic acid containing a particular sequence at a polymorphic position and a second nucleic acid whose sequence is identical to that of the first nucleic acid except for a different sequence at the same polymorphic position;
(ii) contacting the nucleic acids with a multiplicity of test compounds under conditions appropriate for binding; and
(iii) identifying those compounds that bind selectively to either the first or second nucleic acid sequence.

Selective binding as used herein refers to any measurable difference in any parameter of binding, such as, e.g., binding affinity, binding capacity, etc.

In another series of embodiments, an isolated peptide or polypeptide comprising one or more polymorphic positions is tested in vitro for its ability to bind test compounds in a sequence-specific manner. The screening methods involve:
(i) providing a first peptide or polypeptide containing a particular sequence at a polymorphic position and a second peptide or polypeptide whose sequence is identical to the first peptide or polypeptide except for a different sequence at the same polymorphic position;
(ii) contacting the polypeptides with a multiplicity of test compounds under conditions appropriate for binding; and
(iii) identifying those compounds that bind selectively to one of the nucleic acid sequences.

In preferred embodiments, high-throughput screening protocols are used to survey a large number of test compounds for their ability to bind the genes or peptides disclosed above in a sequence-specific manner.

Test compounds are screened from large libraries of synthetic or natural compounds. Numerous means are currently used for random and directed synthesis of saccharide, peptide, and nucleic acid based compounds. Synthetic compound libraries are commercially available from Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Alternatively, libraries of natural compounds in the form of bacterial, fungal, plant and animal extracts are available from e.g. Pan Laboratories (Bothell, Wash.) or MycoSearch (N.C.), or are readily producible. Additionally, natural and synthetically produced libraries and compounds are readily modified through conventional chemical, physical, and biochemical means.

In Vivo Screening Methods

Intact cells or whole animals expressing polymorphic variants of genes disclosed herein can be used in screening methods to identify candidate cardiovascular drugs.

In one series of embodiments, a permanent cell line is established from an individual exhibiting a particular polymorphic pattern. Alternatively, cells (including without limitation mammalian, insect, yeast, or bacterial cells) are programmed to express a gene comprising one or more polymorphic sequences by introduction of appropriate DNA. Identification of candidate compounds can be achieved using any suitable assay, including without limitation (i) assays that measure selective binding of test compounds to particular polymorphic variants of proteins encoded by genes disclosed herein; (ii) assays that measure the ability of a test compound to modify (i.e., inhibit or enhance) a measurable activity or function of proteins encoded by genes disclosed herein; and (iii) assays that measure the ability of a compound to modify (i.e., inhibit or enhance) the transcriptional activity of sequences derived from the promoter (i.e., regulatory) regions of genes disclosed herein.

In another series of embodiments, transgenic animals are created in which (i) one or more human genes disclosed herein, having different sequences at particular polymorphic positions are stably inserted into the genome of the transgenic animal; and/or (ii) the endogenous genes disclosed herein are inactivated and replaced with human genes disclosed herein, having different sequences at particular polymorphic positions. See, e.g., Coffman, Semin. Nephrol. 17:404, 1997; Esther et al., Lab. Invest. 74:953, 1996; Murakami et al., Blood Press. Suppl. 2:36, 1996. Such animals can be treated with candidate compounds and monitored for one or more clinical markers of cardiovascular status.

The following are intended as non-limiting examples of the invention.

Material and Methods

Genotyping of patient DNA with the Pyrosequencing™ Method as described in the patent application WO 9813523:

First a PCR is set up to amplify the flanking regions around a SNP. Therefor 2 ng of genomic DNA (patient sample) are mixed with a primerset (20-40 pmol) producing a 75 to 320 bp PCR fragment with 0.3 to 1 U Qiagens Hot Star Taq Polymerase™ in a total volume of 20 μL. One primer is biotinylated depending on the direction of the sequencing primer. To force the biotinylated primer to be incorporated it is used 0.8 fold.

For primer design, programms like Oligo 6™ (Molecular Biology Insights) or Primer Select™ (DNAStar) are used. PCR setup is performed by a BioRobot 3000™ from Qiagen. PCR takes place in T1 or Tgradient Thermocyclers™ from Biometra.

The whole PCR reaction is transferred into a PSQ plate™ (Pyrosequencing) and prepared using the Sample Prep Tool™ and SNP Reagent Kit™ from Pyro-sequencing according to their instructions.

Preparation of Template for Pyroseguencino™:

Sample preparation using PSQ 96 Sample Prep Tool:
1. Mount the PSQ 96 Sample Prep Tool Cover onto the PSQ 96 Sample Prep Tool as follows: Place the cover on the desk, retract the 4 attachment rods by separating the handle from the magnetic rod holder, fit the magnetic rods into the holes of the cover plate, push the handle downward until a click is heard. The PSQ 96 Sample Prep Tool is now ready for use.
2. To transfer beads from one plate to another, place the covered tool into the PSQ 96 Plate containing the samples and lower the magnetic rods by separating the handle from the magnetic rod holder. Move the tool up and down a few times then wait for 30-60 seconds. Transfer the beads into a new PSQ 96 plate containing the solution of choice.
3. Release the beads by lifting the magnetic rod holder, bringing it together with the handle. Move the tool up and down a few times to make sure that the beads are released.

All steps are performed at room temperature unless otherwise stated.

Immobilization of PCR product:

Biotinylated PCR products are immobilized on streptavidin-coated Dynabeads™ M-280 Streptavidin. Parallel immobilization of several samples are performed in the PSQ 96 Plate.
1. Mix PCR product, 20 μl of a well optimized PCR, with 25 μl 2× BW-buffer II. Add 60-150 μg Dynabeads. It is also possible to add a mix of Dynabeads and 2× BW-buffer II to the PCR product yielding a final BW-buffer II concentration of approximately 1×.
2. Incubate at 65° C. for 15 min agitation-constantly to keep the beads dispersed. For optimal immobilization of fragments longer than 300 bp use 30 min incubation time.

Strand Separation:
4. For strand separation, use the PSQ 96 Sample Prep Tool to transfer the beads with the immobilized sample to a PSQ 96 Plate containing 50 μl 0.50 M NaOH per well. Release the beads.
5. After approximately 1 min, transfer the beads with the immobilized strand to a PSQ 96 Plate containing 99 μl 1× Annealing buffer per well and mix thoroughly.
6. Transfer the beads to a PSQ 96 Plate containing 45 μl of a mix of 1× Annealing buffer and 3-15 pmoles sequencing primer per well.
7. Heat at 80° C. for 2 minutes in the PSQ 96 Sample Prep Thermoplate and move to room temperature.
8. After reaching room temperature, continue with the sequencing reaction.

Sequencing Reaction:
1. Choose the method to be used (“SNP Method”) and enter relevant information in the PSQ 96 Instrument Control software.
2. Place the cartridge and PSQ 96 Plate in the PSQ 96 Instrument.
3. Start the run.
   Genotyping Using the ABI 7700/7900 Instrument (TaqMan)

SNP genotypisation using the TaqMan (Applied Biosystems/Perkin Elmer) was performed according to the manufacturer's instructions. The TaqMan assay is discussed by Lee et al., Nucleic Acids Research 1993, 21: 3761-3766.

Genotyping With a Service Contractor:

Qiagen Genomics, formerly Rapigene, is a service contractor for genotyping SNPs in patient samples. Their method is based on a primer extension method where two complementary primers are designed for each genotype that are labeled with different tags. Depending on the genotype only one primer will be elongated together with a certain tag. This tag can be detected with mass spectrometry and is a measure for the respective genotype. The method is described in the following patent: “Detection and identification of nucleic acid molecules—using tags which may be detected by non-fluorescent spectrometry or potentiometry” (WO 9727325).

### EXAMPLES

To exemplify the present invention and it's utility baySNP 28 will be used in the following:

baySNP 28 is a C to T polymorphism and presumably resides in the gene of the human acidic 82 kDa protein (information taken from table 3). baySNP 28 was genotyped in various patient cohorts using the primers from table 2. As a result the following number of patients carrying different genotypes were found (information combined from tables 3 and 5a):

| | | | | Genotype 12 | Genotype 22 |
|---|---|---|---|---|---|
| baySNP | Cohort | Total | Genotype 11 “CC” | “CT” | “TT” |
| 28 | HELD_FEM_HIRESP | 12 | 1 | 2 | 9 |
| 28 | HELD_FEM_LORESP | 22 | 3 | 12 | 7 |

When comparing the number of female patients exhibiting a high response to statin therapy (HELD_FEM_HBRESP) with the control cohort (HELD_FEM_LORESP) it appears that the number of low responders carrying the CT genotype is increased. This points to a lower statin response among female individuals with the CT genotype. Applying statistical tests on those findings the following p-values were obtained (data taken from table 5b):

| | | GTYPE | GTYPE | GTYPE |
|---|---|---|---|---|
| BAYSNP | COMPARISON | CPVAL | XPVAL | LRPVAL |
| 28 | HELD_FEM_EFF | 0.0506 | 0.0508 | 0.0442 |

As at least one of the GTYPE p values is below 0.05 the association of genotype and statin response phenotype is regarded as statistically-significant. I.e. the analysis of a patient's genotype can predict the response to statin therapy. In more detail one can calculate the relative risk to exhibit a certain statin response phenotype when carrying a certain genotype (data taken from table 6a):

| BAYSNP | COMPARISON | GTYPE1 | GTYPE2 | GTYPE3 | RR1 | RR2 | RR3 |
|---|---|---|---|---|---|---|---|
| 28 | HELD_FEM_EFF | CC | CT | TT | 0.68 | 0.29 | 3.38 |

In case of baySNP 28 the risk to exhibit a high responder phenotype is 3.38 times higher when carrying the TT genotype. This indicates that a TT polymorphism in baySNP 28 is an independent risk factor for high statin response in females. On the other hand carriers of a CT or CC genotype have a reduced risk of being a high responder.

In addition statistical associations can be calculated on the basis on alleles. This calculation would identify risk alleles instead of risk genotypes.

In case of baySNP 28 the following allele counts were obtained (data combined from tables 3 and 5a):

| | | | Allele 1 | Allele 2 |
|---|---|---|---|---|
| baySNP | Cohort | Total | “C” | “T” |
| 28 | HELD_FEM_HIRESP | 12 | 4 | 20 |
| 28 | HELD_FEM_LORESP | 22 | 18 | 26 |

When comparing the number of female patients with high statin response (HELD_FEM_HERESP) with the control cohort (HELD_FEM_LORESP) it appears that the number of high responders carrying the T allele is increased, whereas the number of high responders carrying the C allele is diminished. This points to a higher statin response among female individuals with the T allele. Applying statistical tests on those findings the following p-values were obtained (data taken from table 5b):

| | | ALLELE | ALLELE | ALLELE |
|---|---|---|---|---|
| BAYSNP | COMPARISON | CPVAL | XPVAL | LRPVAL |
| 28 | HELD_FEM_EFF | 0.0411 | 0.0579 | 0.0349 |

As at least one of the ALLELE p values is below 0,05 the association of allele and statin response phenotype is regarded as statistically significant (in this example significant p values were obtained from two statistical tests). I.e. also the analysis of a patient's alleles from baySNP 28 can predict the extend of statin response. In more detail one can calculate the relative risk to exhibit a certain statin response phenotype when carrying a certain allele (data taken from table 6b):

| baySNP | Allele 1 | Allele 2 | COMPARISON | RR1 | RR2 |
|---|---|---|---|---|---|
| 28 | C | T | HELD_FEM_EFF | 0.42 | 2.39 |

In case of baySNP 28 the risk to exhibit a high responder phenotype is 2.39 times higher when carrying the T allele. This indicates that the T allele of baySNP28 is an independent risk factor for a high statin response in females. In other words those patients should receive lower doses of statins in order to avoid ADR. However due to their ‘high responder’ phenotype they will still benefit from the drug. In turn carriers of the C allele should receive higher drug doses in order to experience a benefical therapeutic effect.

Another example is baySNP 29, which is taken to exemplify polymorphisms relevant for adverse drug reactions. baySNP 29 was found significant when comparing male patients with severe ADR to the respective controls (as defined in table 1b).

The relative risk ratios for the genotypes AA, AG and GG were as follows (data taken from table 6a):

| BAYSNP | COMPARISON | GTYPE1 | GTYPE2 | GTYPE3 | RR1 | RR2 | RR3 |
|---|---|---|---|---|---|---|---|
| 29 | HELD_MAL_ADR5ULN | AA | AG | GG | 3.15 | 0.66 | 0.32 |

In this case male patients carrying the AA genotype have a 3.15 times higher risk to suffer from ADR. In other words those patients should either receive lower doses of statins or switch to an alternative therapy in order to avoid ADR. On the other hand male patients with AG or GG genotypes appear to be more resistant to ADR and hence better tolerate statin therapy.

As can be seen from the following tables some of the associations that are disclosed in the present invention are indicative for more than one phenotype. baySNP 1837 is for example linked to ADR, but also to the risk to suffer from CVD (table 6).

TABLE 1a
Definition of “good” and “bad” serum lipid levels

| | “Good” | “Bad” |
|---|---|---|
| LDL-Cholesterol [mg/dL] | 125-150 | 170-200 |
| Cholesterol [mg/dL] | 190-240 | 265-315 |
| HDL-Cholesterol [mg/dL] | 60-105 | 30-55 |
| Triglycerides [mg/dL] | 45-115 | 170-450 |

According to the PROCAM algorithm (Assmann, G., Schulte, H. von Eckardstein, A; Am J. Cardiol 77 (1996); 1179-1184) it is possible to define other cohorts. For example a lipid-based equation would calculate y as follows:
y=−0.0146*LDL+0.0418*1DL−0.3362*In(TRIGLY)

Good or bad cohorts could then be defined in the following way (FEM=female, MAL=male):

FEM GOOD y>=−1.4

FEM BAD y<−1.4

MAL_GOOD y>=−1.7

MAL_BAD y<−1.7

TABLE 1b
Definition of drug response phenotypes

| | |
|---|---|
| Low responder | Decrease of serum LDL of at least 10% and at most 50% |
| | upon administration of 0.8 mg Cerivastatin (female patients) |
| High responder | Decrease of serum LDL of at least 50% upon administration |
| | of 0.4 mg Cerivastatin (female patients) |
| Very low responder | Decrease of serum LDL of at least 10% and at most 35% |
| | upon administration of 0.8 mg Cerivastatin (female patients) |
| Very high responder | Decrease of serum LDL of at least 55% upon administration |
| | of 0.4 mg Cerivastatin (female patients) |
| Ultra low responder | Decrease of serum LDL of at least 10% and at most 25% |
| | upon administration of 0.8 mg Cerivastatin (female patients) |
| Ultra high responder | Decrease of serum LDL of at least 60% upon administration |
| | of 0.4 mg Cerivastatin (female patients) |
| Tolerant patient | No diagnosis of muscle cramps, muscle pain, muscle |
| | weakness, myalgia or myopathy |
| | AND |
| | serum CK levels below 70 mg/dl in women and below 80 mg/dl |
| | in men. |
| ADR patient | Diagnosis of muscle cramps, muscle pain, muscle |
| (CK increase at least | weakness, myalgia or myopathy |
| 2 × ULN) | OR |
| | serum CK levels higher than 140 mg/dl in women and 160 mg/dl |
| | in men. |
| Advanced ADR patient | Serum CK levels higher than 210 mg/dl in women and 240 mg/dl |
| [ADR3] | in men |
| (advanced CK increase, | |
| at least 3 × ULN)* | |
| Severe ADR patient | Serum CK levels higher than 350 mg/dl in women and 400 mg/dl |
| [ADR5] | in men |
| (severe CK increase, at | |
| least 5 × ULN)* | |

*When assembling the cohorts for advanced and severe ADR we focused on the CK serum levels as those provide a more independent measure of statin related ADR.

TABLE 1c
Definition of “high” and “low” serum HDL cholesterol levels

| | Male | Female |
|---|---|---|
| | individuals | individuals |
| ‘High’ HDL-Cholesterol [mg/dL] | >=80 | >=104 |
| ‘Low’ HDL-Cholesterol [mg/dL] | <=35 | <=37 |

An informed consent was signed by the patients and control people. Blood was taken by a physician according to medical standard procedures.

Samples were collected anonymous and labeled with a patient number. DNA was extracted using kits from Qiagen.

TABLE 2a

Oligonucleotide primers used for genotyping using mass
spectrometry
The baySNP number refers to an internal numbering of the PA
SNPs. Primer sequences are listed for preamplification of
the genomic fragments (primers EF and ER) and for subsequent
allele specific PCR of the SNP.

| | | | |
|---|---|---|---|
| baySNP | SNP | Name | Sequence |

| | | | |
|---|---|---|---|
| 28 | C137T | CF | gggacggtcggtagatTCTAGAATTGTGCTTCCC |
| 28 | C137T | EF | TGTCCAGTGTTAGGAAAAA |
| 28 | C137T | ER | GACGATGCCTTCAGCACAGATGTGGCTTCTGTATGAG |
| 28 | C137T | TF | gctggctcggtcaagaTCTAGAATTGTGCTTCCT |
| 29 | A464G | AF | gggacggtcggtagatCATCGGTCAGTGTCCCCA |
| 29 | A464G | EF | GATGTCTGTCTCCTTGATGT |
| 29 | A464G | ER | GACGATGCCTTCAGCACAATGTGGGGGTTTTATTTT |
| 29 | A464G | GF | gctggctcggtcaagaCATCGGTCAGTGTCCCCG |
| 52 | C397G | CR | gggacggtcggtagatTATTTTATAATGCAAAAG |
| 52 | C397G | EF | GACGATGCCTTCAGCACAGTGAATTGCCAGATTAGTG |
| 52 | C397G | ER | TCTAAAGTGCTGGGATTG |
| 52 | C397G | GR | gctggctcggtcaagaTATTTTATAATGCAAAAC |
| 56 | A429G | AF | gggacggtcggtagatAAGGTCTTTGTACGTGTA |
| 56 | A429G | EF | CCAGGTACTGCCTTACAAA |
| 56 | A429G | ER | GACGATGCCTTCAGCACAGCTCCCAAAATAAATCACTC |
| 56 | A429G | GF | gctggctcggtcaagaAAGGTCTTTGTACGTGTG |
| 89 | A159G | AR | gggacggtcggtagatTGGAGTCGGGGGAGTCAT |
| 89 | A159G | EF | GACGATGCCTTCAGCACATAGTTCAAGGGTAAAGGA |
| 89 | A159G | ER | GAGGACGAGATGTAAGAG |
| 89 | A159G | GR | gctggctcggtcaagaTGGAGTCGGGGGAGTCAC |
| 90 | C154T | CF | gggacggtcggtagatCAGCGCATCCTGAACCAC |
| 90 | C154T | EF | GCTGGAACGAGTTCATCCT |
| 90 | C154T | ER | GACGATGCCTTCAGCACAGGACCCCACCTTTCTTGT |
| 90 | C154T | TF | gctggctcggtcaagaCAGCGCATCCTGAACCAT |
| 99 | C58T | CR | gggacggtcggtagatTCCTGCTCTTTTCTCTAG |
| 99 | C58T | EF | GACGATGCCTTCAGCACACACTGACTGCTTACTCTACC |
| 99 | C58T | ER | TACTGTGTCTCAGCTCCA |
| 99 | C58T | TR | gctggctcggtcaagaTCCTGCTCTTTTCTCTAA |
| 140 | C468T | CR | gggacggtcggtagatGTGAATCCCAATACGAAG |
| 140 | C468T | EF | GACGATGCCTTCAGCACATAAAAAATAACCAGGTACTCCA |
| 140 | C468T | ER | GATGAGTCCTTCACCAAACATACA |
| 140 | C468T | TR | gctggctcggtcaagaGTGAATCCCAATACGAAA |
| 152 | A587G | AF | gggacggtcggtagatGGTGGGAGGTTCCAGCCA |
| 152 | A587G | EF | GCAGGAAGAAAGCTAGAA |
| 152 | A587G | ER | GACGATGCCTTCAGCACAAGGCAGGATAATGACAAC |
| 152 | A587G | GF | gctggctcggtcaagaGGTGGGAGGTTCCAGCCG |
| 214 | A209G | AF | gggacggtcggtagatCATTTCCACCTCACCAAA |
| 214 | A209G | EF | AGGTATTCCCGGCGTTTC |
| 214 | A209G | ER | GACGATGCCTTCAGCACATGTTGTGCGTCTGCTTCC |
| 214 | A209G | GF | gctggctcggtcaagaCATTTCCACCTCACCAAG |
| 221 | C339G | CF | gggacggtcggtagatTGTGAAGAACTGTTGCTC |
| 221 | C339G | EF | CTGAAGCTCATCTGCCTTCT |
| 221 | C339G | ER | GACGATGCCTTCAGCACATCCCCTTCCTTCTTACCT |
| 221 | C339G | GF | gctggctcggtcaagaTGTGAAGAACTGTTGCTG |
| 224 | C189T | CR | gggacggtcggtagatGCCCGCTTTTCTTCATCG |
| 224 | C189T | EF | GACGATGCCTTCAGCACACTGTCTTCAAGGGCTTACAC |
| 224 | C189T | ER | TCCAACTTCAGGCAAAAC |
| 224 | C189T | TR | gctggctcggtcaagaGCCCGCTTTTCTTCATCA |
| 294 | C465T | CR | gggacggtcggtagatCCCAAGGCCAACAGGGAG |
| 294 | C465T | EF | GACGATGCCTTCAGCACAGCATTCTTATGCCAGTGTTC |
| 294 | C465T | ER | ATCCATCCCATCCTGTGT |
| 294 | C465T | TR | gctggctcggtcaagaCCCAAGGCCAACAGGGAA |
| 307 | C215T | CR | gggacggtcggtagatGAGTGGGTGCTGTTCCCG |
| 307 | C215T | EF | GACGATGCCTTCAGCACAGTTACTGCCTCTCTGACC |
| 307 | C215T | ER | AGTGTGACCTGCTCTCTT |
| 307 | C215T | TR | gctggctcggtcaagaGAGTGGGTGCTGTTCCCA |
| 411 | A369T | ER | gacgatgccttcagcacaAACACATTCCCCCTCTAC |
| 411 | A369T | EF | GTCTCTATTCCAAGCCAAG |
| 411 | A369T | AF | gggacggtcggtagatCCCCGCTCCAGCTCCTCA |
| 411 | A369T | TF | gctggctcggtcaagaCCCCGCTCCAGCTCCTCT |
| 449 | C323G | CR | gggacggtcggtagatCCGCTTCTGCTTCTGCTG |
| 449 | C323G | EF | GACGATGCCTTCAGCACAAGGAGAAGAGGGAGGAGA |
| 449 | C323G | ER | GGAGCACGTAAGGAGAAA |
| 449 | C323G | GR | gctggctcggtcaagaCCGCTTCTGCTTCTGCTC |
| 466 | C123T | CF | gggacggtcggtagatGGCCAGGGGCTGGAGGGC |
| 466 | C123T | EF | TCTTCAGTTCTCTCAGCTTC |
| 466 | C123T | ER | GACGATGCCTTCAGCACATCACTAGGGGCTCTTACC |
| 466 | C123T | TF | gctggctcggtcaagaGGCCAGGGGCTGGAGGGT |
| 472 | A497G | AR | gggacggtcggtagatTCCTCCCGCTGCTTCAGT |
| 472 | A497G | EF | GACGATGCCTTCAGCACATCACTTACCCATCATACTTCTTTTTC |
| 472 | A497G | ER | AATCCTGCCTCCCACCTT |
| 472 | A497G | GR | gctggctcggtcaagaTCCTCCCGCTGCTTCAGC |
| 542 | A402G | AR | gggacggtcggtagatAGAAATTCCCTCCCAACT |
| 542 | A402G | EF | GACGATGCCTTCAGCACATGATTGAGCCAGTTGTTT |
| 542 | A402G | ER | GGGGTGTATTTTGAGAGTG |
| 542 | A402G | GR | gctggctcggtcaagaAGAAATTCCCTCCCAACC |
| 739 | C87G | CR | gggacggtcggtagatGCTGGTTTGACTGGACGG |
| 739 | C87G | EF | GACGATGCCTTCAGCACAACCTTGGTATAATCCTTTCC |
| 739 | C87G | ER | AGGCAACCTAATCCACTT |
| 739 | C87G | GR | gctggctcggtcaagaGCTGGTTTGACTGGACGC |
| 821 | A140C | AF | gggacggtcggtagatAGTGCTGTGATACCTGGA |
| 821 | A140C | CF | gctggctcggtcaagaAGTGCTGTGATACCTGGC |
| 821 | A140C | EF | ACACCCACAAAACAAGAA |
| 821 | A140C | ER | GACGATGCCTTCAGCACAGGAACAAGGACATAAAAGAG |
| 1005 | A257G | AR | gggacggtcggtagatAGGAAATGTTAGCCCTGT |
| 1005 | A257G | EF | GACGATGCCTTCAGCACACTCCACTTCTCTATGCCTC |
| 1005 | A257G | ER | GTCCCCAGCTATGTATTGT |
| 1005 | A257G | GR | gctggctcggtcaagaAGGAAATGTTAGCCCTGC |
| 1055 | A287T | AF | gggacggtcggtagatCTCAGGGAGGGAGAGAGA |
| 1055 | A287T | EF | GGGACAGACAGACAGACA |
| 1055 | A287T | ER | GACGATGCCTTCAGCACACAACTCCTTCTTCAGCAC |
| 1055 | A287T | TF | gctggctcggtcaagaCTCAGGGAGGGAGAGAGT |
| 1056 | A354G | AR | gggacggtcggtagatGCGGCTGCCCCGTCCTGT |
| 1056 | A354G | EF | GACGATGCCTTCAGCACAGTGTGTCTATGTGTCTGTGTG |
| 1056 | A354G | ER | CGGACTTCTCCTTCTTGT |
| 1056 | A354G | GR | gctggctcggtcaagaGCGGCTGCCCCGTCCTGC |
| 1085 | A251G | EF | TAGGGTAAGCAGCAAGAG |
| 1085 | A251G | ER | CACAAGGCAAGAGATAACA |
| 1085 | A251G | AF | gggacggtcggtagatCAGGCAAGATAGACAGCA |
| 1085 | A251G | GF | gctggctcggtcaagaCAGGCAAGATAGACAGCG |
| 1086 | A104G | EF | GTGCCCATACGAACAGAATAG |
| 1086 | A104G | ER | TGCCAAGTACCCCAAGAG |
| 1086 | A104G | AR | gggacggtcggtagatCCATTCCTCCCCAGACAT |
| 1086 | A104G | GR | gctggctcggtcaagaCCATTCCTCCCCAGACAC |
| 1092 | C1687G | CF | gggacggtcggtagatCGTGCGAGCAGCGAAAGC |
| 1092 | C1687G | EF | CCAGAGAGAAGTCGAGGAAGAGA |
| 1092 | C1687G | ER | GACGATGCCTTCAGCACAGTCACCCCCAAAAGCAGG |
| 1092 | C1687G | GF | gctggctcggtcaagaCGTGCGAGCAGCGAAAGG |
| 1096 | G454T | EF | GACGATGCCTTCAGCACACTTTTCCTCCTAGCCCAC |
| 1096 | G454T | ER | AAGTGATGTAACCCTCCTCTC |
| 1096 | G454T | GR | gggacggtcggtagatTCAGCTATAAATAGGGCC |
| 1096 | G454T | TR | gctggctcggtcaagaTCAGCTATAAATAGGGCA |
| 1101 | C249T | CR | gggacggtcggtagatTGATGGCGGGTGCCAAGG |
| 1101 | C249T | EF | GACGATGCCTTCAGCACAGCTCTTTCCTTTGCTTCC |
| 1101 | C249T | ER | CACTGGGGGTCCTCTTAC |
| 1101 | C249T | TR | gctggctcggtcaagaTGATGGCGGGTGCCAAGA |
| 1204 | A307G | AR | gggacggtcggtagatCAAGGGCACTCACATTAT |
| 1204 | A307G | EF | GACGATGCCTTCAGCACAGCTCTTGCGTCTGTTTCC |
| 1204 | A307G | ER | TTTCCCTTCTGTCCCCTT |
| 1204 | A307G | GR | gctggctcggtcaagaCAAGGGCACTCACATTAC |
| 1504 | C180T | CF | gggacggtcggtagatGTGACTTTTGGTTCCCAC |
| 1504 | C180T | EF | AACTCGGGGTCACTGGTCT |
| 1504 | C180T | ER | GACGATGCCTTCAGCACACAGCGGGTATGGAGGATG |
| 1504 | C180T | TF | gctggctcggtcaagaGTGACTTTTGGTTCCCAT |
| 1511 | G153T | EF | ACACCAGTTCTCCCTCCT |
| 1511 | G153T | ER | GACGATGCCTTCAGCACACCCACCTTTCCTAATCCT |
| 1511 | G153T | GF | gggacggtcggtagatTTGGGACTCTGCGTCAAG |
| 1511 | G153T | TF | gctggctcggtcaagaTTGGGACTCTGCGTCAAT |
| 1524 | A284C | AF | gggacggtcggtagatCTCTCAAAGCCCACACAA |
| 1524 | A284C | CF | gctggctcggtcaagaCTCTCAAAGCCCACACAC |
| 1524 | A284C | EF | AGAAAAAGAAAAGGAAAAAGA |
| 1524 | A284C | ER | GACGATGCCTTCAGCACAGGAAAGTTACAAGGCTATGA |
| 1556 | C367G | CR | gggacggtcggtagatACCTGCCTCTAAGGTCTG |
| 1556 | C367G | EF | GACGATGCCTTCAGCACAAGGAGAAGACAGTTCAAGG |
| 1556 | C367G | ER | ACAGTTGCCAGAGAAAAG |
| 1556 | C367G | GR | gctggctcggtcaagaACCTGCCTCTAAGGTCTC |
| 1561 | A251C | EF | TCACTTGCCTCTACTCCA |
| 1561 | A251C | ER | ATACCAGAAAGACTAAGCTCC |
| 1561 | A251C | AF | gggacggtcggtagatGGGTGAGCTCTGTGGGCA |
| 1561 | A251C | CF | gctggctcggtcaagaGGGTGAGCTCTGTGGGCC |
| 1582 | C389T | CR | gggacggtcggtagatCCAAGGGTTATGGCAGGG |
| 1582 | C389T | EF | GACGATGCCTTCAGCACACCTGACTATTTGGGGTTGTG |
| 1582 | C389T | ER | ATCGCTCTCTGCTTCTGCT |
| 1582 | C389T | TR | gctggctcggtcaagaCCAAGGGTTATGGCAGGA |
| 1638 | A443G | AR | gggacggtcggtagatCCAAAACCCCAGCGCTGT |
| 1638 | A443G | EF | GACGATGCCTTCAGCACACTCTTTATCCTGCTTATGGT |
| 1638 | A443G | ER | CCAAGCTCACTCTGTAGG |
| 1638 | A443G | GR | gctggctcggtcaagaCCAAAACCCCAGCGCTGC |
| 1662 | C251T | EF | AATACAATGGAAGCCAAG |
| 1662 | C251T | ER | CCTAATCGAACAGAAAGG |
| 1662 | C251T | CF | gggacggtcggtagatCCAGTCTCCATCCACTTC |
| 1662 | C251T | TF | gctggctcggtcaagaCCAGTCTCCATCCACTTT |
| 1714 | A376G | AF | gggacggtcggtagatTGAACGGCATGACGGGGA |
| 1714 | A376G | EF | AAGTGTTTCTGCTGTGCCT |
| 1714 | A376G | ER | GACGATGCCTTCAGCACACAAGTCCTGGTTTTCCATC |
| 1714 | A376G | GF | gctggctcggtcaagaTGAACGGCATGACGGGGG |
| 1722 | C89T | CF | gggacggtcggtagatACCCCAGGATGCCCACAC |
| 1722 | C89T | EF | GTTTATCCTCCTCATGTCC |
| 1722 | C89T | ER | GACGATGCCTTCAGCACAGTTACCTTTTCCACCTCTC |
| 1722 | C89T | TF | gctggctcggtcaagaACCCCAGGATGCCCACAT |
| 1757 | A210G | AF | gggacggtcggtagatGGAAACAAACCAAAATGA |
| 1757 | A210G | EF | CCAGCACCCAAAATAAGA |
| 1757 | A210G | ER | GACGATGCCTTCAGCACAATAAGTTGAAGCCCTCCC |
| 1757 | A210G | GF | gctggctcggtcaagaGGAAACAAACCAAAATGG |
| 1765 | A240G | AF | gggacggtcggtagatGGCTTCACGGAGGAAGAA |
| 1765 | A240G | EF | TTAGGAGCTGTGAGGTATG |
| 1765 | A240G | ER | GACGATGCCTTCAGCACATAAGATGGAGCAGGGTAG |
| 1765 | A240G | GF | gctggctcggtcaagaGGCTTCACGGAGGAAGAG |
| 1776 | A200G | AF | gggacggtcggtagatAAAGGGCTCCCAACACCA |
| 1776 | A200G | EF | TGAGCACAAGATCAGAGAGG |
| 1776 | A200G | ER | GACGATGCCTTCAGCACAAGACAGAGACGCAGGAATG |
| 1776 | A200G | GF | gctggctcggtcaagaAAAGGGCTCCCAACACCG |
| 1799 | C370T | CF | gggacggtcggtagatAGGGACAACCAAAGTGAC |
| 1799 | C370T | EF | ATCATCAGAACAGCCCTAC |
| 1799 | C370T | ER | GACGATGCCTTCAGCACACAAGCCCACCTACTTACTC |
| 1799 | C370T | TF | gctggctcggtcaagaAGGGACAACCAAAGTGAT |
| 1806 | A201G | AF | gggacggtcggtagatTGGGCGTCCTGGTGGGCA |
| 1806 | A201G | EF | TCTTCGGGCTAACTCTTT |
| 1806 | A201G | ER | GACGATGCCTTCAGCACACTGTCACTCCAAACCTTCT |
| 1806 | A201G | GF | gctggctcggtcaagaTGGGCGTCCTGGTGGGCG |
| 1837 | C413T | CF | gggacggtcggtagatCTCAGCTTCATGCAGGGC |
| 1837 | C413T | EF | CCCACTCAGCCCTGCTCTT |
| 1837 | C413T | ER | GACGATGCCTTCAGCACAGCATCCTTGGCGGTCTTG |
| 1837 | C413T | TF | gctggctcggtcaagaCTCAGCTTCATGCAGGGT |
| 1870 | C323T | CF | gggacggtcggtagatCTCCTCATTGCCTCCTTC |
| 1870 | C323T | EF | CACCTCTTTTCTCCTTCTCTT |
| 1870 | C323T | ER | GACGATGCCTTCAGCACACCCACCCCCTCTATCTAC |
| 1870 | C323T | TF | gctggctcggtcaagaCTCCTCATTGCCTCCTTT |
| 1882 | C115T | CR | gggacggtcggtagatGTCCCCCACAAGTCCTCG |
| 1882 | C115T | EF | GACGATGCCTTCAGCACAGACCTGTACCCTTTACCC |
| 1882 | C115T | ER | TGTTTCCCTGTCTGTTTC |
| 1882 | C115T | TR | gctggctcggtcaagaGTCCCCCACAAGTCCTCA |
| 1988 | C214T | CF | gggacggtcggtagatGTGACTCGGTCCTATACC |
| 1988 | C214T | EF | GTGGGCTGTGATTGTGTT |
| 1988 | C214T | ER | GACGATGCCTTCAGCACATCTCGTCGTCGTAGTAGTTGT |
| 1988 | C214T | TF | gctggctcggtcaagaGTGACTCGGTCCTATACT |
| 2000 | C349T | CR | gggacggtcggtagatAGTATGGTAATTAGGAAG |
| 2000 | C349T | EF | GACGATGCCTTCAGCACACTGACACTGAGCCACAAC |
| 2000 | C349T | ER | AACTGATGAGCAAGAAGGA |
| 2000 | C349T | TR | gctggctcggtcaagaAGTATGGTAATTAGGAAA |
| 2071 | A338G | AR | gggacggtccgtagatAAAATTGTTTCCTGTGAT |
| 2071 | A338G | EF | GACGATGCCTTCAGCACACATTGCTATTCTCAGGCTATA |
| 2071 | A338G | ER | CCCATTCTCTGCTTGACAGT |
| 2071 | A338G | GR | gctggctcggtcaagaAAAATTGTTTCCTGTGAC |
| 2078 | G876T | EF | CCAGAGAGGGGATAAAGA |
| 2078 | G876T | ER | GACGATGCCTTCAGCACAGAGTGTCAAGAGGAACAGG |
| 2078 | G876T | GF | gggacggtcggtagatTGGCTGCTGAGGTCTGAG |
| 2078 | G876T | TF | gctggctcggtcaagaTGGCTGCTGAGGTCTGAT |
| 2085 | G415T | EF | GCTTTTTCTTTTCATTACATC |
| 2085 | G415T | ER | GACGATGCCTTCAGCACACCTCTTTTAGAATCAGAGACA |
| 2085 | G415T | GF | gggacggtcggtagatGGTAGTGTTACCAGAAAG |
| 2085 | G415T | TF | gctggctcggtcaagaGGTAGTGTTACCAGAAAT |
| 2095 | A406G | AR | gggacggtcggtagatTGTGCACCGGGATATTTT |
| 2095 | A406G | EF | GACGATGCCTTCAGCACAATGTGTGCTTGGGTTCTT |
| 2095 | A406G | ER | GGTGTTTCTCCTCCTCTCT |
| 2095 | A406G | GR | gctggctcggtcaagaTGTGCACCGGGATATTTC |
| 2119 | A67G | AR | gggacggtcggtagatGTGGGCACCAAACGCTAT |
| 2119 | A67G | EF | GACGATGCCTTCAGCACAGATGTAGGGCTGGAAGTG |
| 2119 | A67G | ER | TCAAGAAAAATGGGAGTTG |
| 2119 | A67G | GR | gctggctcggtcaagaGTGGGCACCAAACGCTAC |
| 2141 | A176G | EF | TGTAGCATCGGTAGGTTC |
| 2141 | A176G | ER | CAACATCAGACTTTCTTTTTC |
| 2141 | A176G | AR | gggacggtcggtagatTGGTACAGGGCTAGTTTT |
| 2141 | A176G | GR | gctggctcggtcaagaTGGTACAGGGCTAGTTTC |
| 2182 | A318G | AF | gggacggtcggtagatAGGCGGGCCAAGGGTGAA |
| 2182 | A318G | EF | TTCTCTCTCCCCTTCTGT |
| 2182 | A318G | ER | GACGATGCCTTCAGCACATAAATGTTCACTCTTCTTGCT |
| 2182 | A318G | GF | gctggctcggtcaagaAGGCGGGCCAAGGGTGAG |
| 2234 | G296T | EF | GGGTTGTTCCAGGGCGCTATT |
| 2234 | G296T | ER | GACGATGCCTTCAGCACATGTGGAGAGGCCGGGTGC |
| 2234 | G296T | GF | gggacggtcggtagatGAACCAGCCCCCTGGAAG |
| 2234 | G296T | TF | gctggctcggtcaagaGAACCAGCCCCCTGGAAT |
| 2281 | A227C | AR | gggacggtcggtagatCAGGCTTGGAGACCTGGT |
| 2281 | A227C | CR | gctggctcggtcaagaCAGGCTTGGAGACCTGGG |
| 2281 | A227C | EF | GACGATGCCTTCAGCACAGGGTATTCAGTTGGAAGG |
| 2281 | A227C | ER | AAGGCAAGGTTCTTAGTTG |
| 2298 | A77C | AR | gggacggtcggtagatTCTAAAAGCACTTGAAAT |
| 2298 | A77C | CR | gctggctcggtcaagaTCTAAAAGCACTTGAAAG |
| 2298 | A77C | EF | GACGATGCCTTCAGCACACCTGCTAGTGTTTTCTGG |
| 2298 | A77C | ER | TGTAACTGATAGGTGGTGG |
| 2341 | C286T | CR | gggacggtccgtagatTGAAGATTCTGCTCAGCG |
| 2341 | C286T | EF | GACGATGCCTTCAGCACAAGGGCCCGGGACTCAT |
| 2341 | C286T | ER | TTTGGGGTCCTGCGGATG |
| 2341 | C286T | TR | gctggctcggtcaagaTGAAGATTCTGCTCAGCA |
| 2357 | A165G | AF | gggacggtcggtagatCAAAGAAGACGAAAATGA |
| 2357 | A165G | EF | CTCAAGTTTGTTACTGATTTCTC |
| 2357 | A165G | ER | GACGATGCCTTCAGCACAGGGTTACGTCTGCTCTTC |
| 2357 | A165G | GF | gctggctcggtcaagaCAAAGAAGACGAAAATGG |
| 2366 | G50T | EF | GACGATGCCTTCAGCACACTGCTCCGAAACACGGTC |
| 2366 | G50T | ER | GCATCTTCAGCCCTTCTTACTCT |
| 2366 | G50T | GR | gggacggtcggtagatCTCCTGGGCACCACGGGC |
| 2366 | G50T | TR | gctggctcggtcaagaCTCCTGGGCACCACGGGA |
| 2995 | A299C | ER | gacgatgccttcagcacaTGGGATTAGACACGAGAG |
| 2995 | A299C | EF | AAAGAACTGGAAGAAGGAA |
| 2995 | A299C | AF | gggacggtcggtagatGTCACCTCCTTTCCACTA |
| 2995 | A299C | CF | gctggctcggtcaagaGTCACCTCCTTTCCACTC |
| 3360 | G777T | ER | gacgatgccttcagcacaAGAAAAATGAGAGGGAAAAC |
| 3360 | G777T | EF | GATGAAGGGAAATGGAAC |
| 3360 | G777T | GF | gggacggtcggtagatCCAACTATATAGGAGCCG |
| 3360 | G777T | TF | gctggctcggtcaagaCCAACTATATAGGAGCCT |
| 3464 | A110G | EF | CTGAACCGAGGAGATTTTT |
| 3464 | A110G | ER | TGATGCTTACAGAACTGGG |
| 3464 | A110G | AF | gggacggtcggtagatGTGTAGTGGGCAGGGTTA |
| 3464 | A110G | GF | gctggctcggtcaagaGTGTAGTGGGCAGGGTTG |
| 3975 | A65C | EF | gacgatgccttcagcacaAAAAGAACCCTGGTGAAG |
| 3975 | A65C | ER | CCCTGATAAAAGAGATGGA |
| 3975 | A65C | AR | gggacggtcggtagatCGCATGGGAGTCAGGGAT |
| 3975 | A65C | CR | gctggctcggtcaagaCGCATGGGAGTCAGGGAG |
| 3976 | A239G | EF | gacgatgccttcagcacaATGAGGGAGCAAGACAAG |
| 3976 | A239G | ER | TGATAAAAGAGATGGAAGGAG |
| 3976 | A239G | AR | gggacggtcggtagatGTCACTGTTTGTCACTGT |
| 3976 | A239G | GR | gctggctcggtcaagaGTCACTGTTTGTCACTGC |
| 4206 | A304T | EF | gacgatgccttcagcacaCTTTTTAGCCAAGTGGAG |
| 4206 | A304T | ER | GGATCTGAGGAATCTGTG |
| 4206 | A304T | AR | gggacggtcggtagatACCAGGCAGAGAGAAAAT |
| 4206 | A304T | TR | gctggctcggtcaagaACCAGGCAGAGAGAAAAA |
| 4912 | A74G | EF | CTTCACTGAGCGTCCGCAGAG |
| 4912 | A74G | ER | CCGTCGGCCCGATTCA |
| 4912 | A74G | AR | CAGGCGAGCCTCAGCCCT |
| 4912 | A74G | GR | CAGGCGAGCCTCAGCCCC |
| 4925 | A251C | EF | TCATTTCCCAATTTACCTCC |
| 4925 | A251C | ER | CCTCTTTCCCATCTCCCT |
| 4925 | A251C | AF | gggacggtcggtagatAGCCAGGAGCCTGCGTCA |
| 4925 | A251C | CF | gctggctcggtcaagaAGCCAGGAGCCTGCGTCC |
| 4966 | A251G | EF | CATTGCTCTTCCTCTCTGT |
| 4966 | A251G | ER | GTGTCATCATTCCTTTCTTG |
| 4966 | A251G | AR | gggacggtcggtagatTCAGAGACATGAGTCCAT |
| 4966 | A251G | GR | gctggctcggtcaagaTCAGAGACATGAGTCCAC |
| 5014 | A2057G | ER | gacgatgccttcagcacaCACCTGTCCCACCCTATTT |
| 5014 | A2057G | EF | GTCCTGAACCCCCATTCT |
| 5014 | A2057G | AF | gggacggtcggtagatGCCTGCACTCCGTTCCTA |
| 5014 | A2057G | GF | gctggctcggtcaagaGCCTGCACTGCGTTCCTG |
| 5296 | A251G | EF | GCTCCTCTGCCTTCTGCTT |
| 5296 | A251G | ER | ATTTGCCCACTCCCCTTC |
| 5296 | A251G | AF | gggacggtcggtagatTGGCTGCAGGTGCGTCCA |
| 5296 | A251G | GF | gctggctcggtcaagaTGGCTGCAGGTGCGTCCG |
| 5298 | C172T | EF | GCCACACACACCTTAACA |
| 5298 | C172T | ER | AAAGTTCTCTGCCTCCAA |
| 5298 | C172T | CF | gggacggtcggtagatAGCTCTCAGCTGGGGTGC |
| 5298 | C172T | TF | gctggctcggtcaagaAGCTCTCAGCTGGGGTGT |
| 5457 | A134G | EF | AGCAGAATGGGCAATAGA |
| 5457 | A134G | ER | AGAGATGTGGGCAGAGAA |
| 5457 | A134G | AF | gggacggtcggtagatGGAAAGCCTACTTTCTTA |
| 5457 | A134G | GF | gctggctcggtcaagaGGAAAGCCTACTTTCTTG |
| 5704 | C61T | EF | ACAGCCATAACAGGAGTG |
| 5704 | C61T | ER | GGGTTACTCAACCTAAGAGA |
| 5704 | C61T | CR | gggacggtcggtagatGTTCTCTTTGGGAAAACG |
| 5704 | C61T | TR | gctggctcggtcaagaGTTCTCTTTGGGAAAACA |
| 5717 | A1960G | EF | gacgatgccttcagcacaGAACAGAAACCACAGAACC |
| 5717 | A1960G | ER | GTCCCACCCTATTTTGAG |
| 5717 | A1960G | AR | gggacggtcggtagatCACTGGCCCACCTCCCTT |
| 5717 | A1960G | GR | gctggctcggtcaagaCACTGGCCCACCTCCCTC |
| 5959 | A71G | EF | gacgatgccttcagcacaACCATCCCTGACTTAACC |
| 5959 | A71G | ER | TTGTTTCCTCTCCTCTTTC |
| 5959 | A71G | AR | gggacggtcggtagatGTTAAGAGGCTGGGCAGT |
| 5959 | A71G | GR | gctggctcggtcaagaGTTAAGAGGCTGGGCAGC |
| 6162 | C340G | EF | gacgatgccttcagcacaAGTGTTGTTAGGAGCAAAG |
| 6162 | C340G | ER | CTTAGGAAACTGAGGTGG |
| 6162 | C340G | CR | gggacggtcggtagatCTGCAGCCTGGGCAACAG |
| 6162 | C340G | GR | gctggctcggtcaagaCTGCAGCCTGGGCAACAC |
| 6236 | C906T | ER | gacgatgccttcagcacaTGGACACATTTGAGCTTT |
| 6236 | C906T | EF | CTTCCCCAGAGATGACTAC |
| 6236 | C906T | CF | gggacggtcggtagatCCCCATCCTACTCAGCAC |
| 6236 | C906T | TF | gctggctcggtcaagaCCCCATCCTACTCAGCAT |
| 6744 | C348T | ER | gacgatgccttcagcacaGGTTACAGTGAGCCAAGA |
| 6744 | C348T | EF | AGGTGAAGAAAGCAAAATAC |
| 6744 | C348T | CF | gggacggtcggtagatTGGTGTGTGTTTTGTTTC |
| 6744 | C348T | TF | gctggctcggtcaagaTGGTGTGTGTTTTGTTTT |
| 7133 | C63G | EF | TTGAGACCCTACAGAGCCA |
| 7133 | C63G | ER | GGCAAGCTGAGGTGAAAG |
| 7133 | C63G | CR | gggacggtcggtagatAATAAGGTAAGAAATGAG |
| 7133 | C63G | GR | gctggctcggtcaagaAATAAGGTAAGAAATGAC |
| 8210 | A251G | EF | TAATTTCTAATGGCCTTCC |
| 8210 | A251G | ER | TCACTTACTCCCTGATGTCT |
| 8210 | A251G | AR | gggacggtcggtagatCATTGGGTTTTCCCTCAT |
| 8210 | A251G | GR | gctggctcggtcaagaCATTGGGTTTTCCCTCAC |
| 8592 | C46T | ER | gacgatgccttcagcacaACATTTAGTGCCAACATCAC |
| 8592 | C46T | EF | CTCTTCCCTGAGACACCA |
| 8592 | C46T | CF | gggacggtcggtagatGAAGGTGAAGGCCAGAGC |
| 8592 | C46T | TF | gctggctcggtcaagaGAAGGTGAAGGCCAGAGT |
| 8943 | A251C | EF | GAGGCTGAGACAGAAGAA |
| 8943 | A251C | ER | GTTTGACATTAAAGAAAATGAG |
| 8943 | A251C | AR | gggacggtcggtagatGGCTGGAGTGCAGTGATT |
| 8943 | A251C | CR | gctggctcggtcaagaGGCTGGAGTGCAGTGATG |
| 9193 | C88G | EF | CACGCTGTTGAGTGGG |
| 9193 | C88G | ER | CGCAGGTCTACGGTCA |
| 9193 | C88G | CR | gggacggtcggtagatCCCGGGTCTGAGGCTGCG |
| 9193 | C88G | GR | gctggctcggtcaagaCCCGGGTCTGAGGCTGCC |
| 9516 | A187G | EF | CACACACACACACACACAC |
| 9516 | A187G | ER | GGTCCCTTACTTTCCTCTT |
| 9516 | A187G | AR | gggacggtcggtagatCCTATCCCTACTTCCCCT |
| 9516 | A187G | GR | gctggctcggtcaagaCCTATCCCTACTTCCCCC |
| 9698 | A251G | EF | GTGACCCCAAAAGAGAGA |
| 9698 | A251G | ER | CTAGCTTGTTACTGCCTCC |
| 9698 | A251G | AF | gggacggtcggtagatGGCACGACCCCGCCCCCA |
| 9698 | A251G | GF | gctggctcggtcaagaGGCACGACCCCGCCCCCG |
| 9883 | A249G | EF | TCCACAACCTCAAAACCAC |
| 9883 | A249G | ER | CACAGTCCTGCAAGCTCA |
| 9883 | A249G | AR | gggacggtcggtagatCCGTGGCCGTGGCTCACT |
| 9883 | A249G | GR | gctggctcggtcaagaCCGTGGCCGTGGCTCACC |
| 10481 | A107T | ER | gacgatgccttcagcacaGTTCGGGGCTCCACTT |
| 10481 | A107T | EF | TAGCGGGACAGCGCTG |
| 10481 | A107T | AF | gggacggtcggtagatCCCGGCGCGCCTCGGAGA |
| 10481 | A107T | TF | gctggctcggtcaagaCCCGGCGCGCCTCGGAGT |
| 10542 | C367T | EF | gacgatgccttcagcacaAATACACTGGGTCCTGCT |
| 10542 | C367T | ER | ATACTGCTGGCCTTTCTC |
| 10542 | C367T | CR | gggacggtcggtagatGGTCAGGGGAGCCCAGAG |
| 10542 | C367T | TR | gctggctcggtcaagaGGTCAGGGGAGCCCAGAA |
| 10600 | A251G | EF | CCTGGCAACTAACCTCTT |
| 10600 | A251G | ER | AGGCAGTCTCTCTGTCTACTC |
| 10600 | A251G | AR | gggacggtcggtagatATTGGCCCTGCTCAGGAT |
| 10600 | A251G | GR | gctggctcggtcaagaATTGGCCCTGCTCAGGAC |
| 10621 | C402T | EF | CCAGCCCTAAACCTAAA |
| 10621 | C402T | ER | AACCTCTCAAGATCAGACAC |
| 10621 | C402T | CF | gggacggtcggtagatTTAGCACTTAATAAGTAC |
| 10621 | C402T | TF | gctggctcggtcaagaTTAGCACTTAATAAGTAT |
| 10745 | A251G | EF | CCCCACAACAAAGAAAGA |
| 10745 | A251G | ER | GAAGCCAACTCTCCAACA |
| 10745 | A251G | AF | gggacggtcggtagatCAAGGATTTCAAAAACCA |
| 10745 | A251G | GF | gctggctcggtcaagaCAAGGATTTCAAAAACCG |
| 10771 | C64G | EF | gacgatgccttcagcacaCCAGGGAAGAGCAGAACC |
| 10771 | C64G | ER | TGTACGGGAAGAGGCAGA |
| 10771 | C64G | CR | gggacggtcggtagatAGGGTGACACAGGCCACG |
| 10771 | C64G | GR | gctggctcggtcaagaAGGGTGACACAGGCCACC |
| 10870 | A251G | EF | ATCCCATCCCAACACACA |
| 10870 | A251G | ER | CCGAGACCAAACTCATTCAC |
| 10870 | A251G | AR | gggacggtcggtagatGGCAGAGCCTGAGTCACT |
| 10870 | A251G | GR | gctggctcggtcaagaGGCAGAGCCTGAGTCACC |
| 10877 | A251C | EF | CCTGTTTCTCAACCTTCTC |
| 10877 | A251C | ER | ATGGTCTATGGAACCTAATCT |
| 10877 | A251C | AF | gggacggtcggtagatGCACTGATTCTGCTTCCA |
| 10877 | A251C | CF | gctggctcggtcaagaGCACTGATTCTGCTTCCC |
| 10948 | G140T | EF | AAGGACAGGGTCAGGAAAG |
| 10948 | G140T | ER | CAGAGGGAGGAAGGAGGT |
| 10948 | G140T | GF | gggacggtcggtagatATGGAGGAGGGTGTCTGG |
| 10948 | G140T | TF | gctggctcggtcaagaATGGAGGAGGGTGTCTGT |
| 11001 | C286T | EF | gacgatgccttcagcacaTTCCCAAAGACCCACA |
| 11001 | C286T | ER | CCTCCACCGCTATCAC |
| 11001 | C286T | CR | gggacggtcggtagatTGGCTGCAGGACGTCCAG |
| 11001 | C286T | TR | gctggctcggtcaagaTGGCTGCAGGACGTCCAA |
| 11001 | C286T | EF | TTCCCAAAGACCCACA |
| 11001 | C286T | ER | CCTCCACCGCTATCAC |
| 11001 | C286T | CR | gggacggtcggtagatTGGCTGCAGGACGTCCAG |
| 11001 | C286T | TR | gctggctcggtcaagaTGGCTGCAGGACGTCCAA |
| 11073 | C215G | EF | CCCAACCACCCGTTCC |
| 11073 | C215G | ER | GCGCGGGAGCTAGAGA |
| 11073 | C215G | CF | gggacggtcggtagatGAAGCTGCGGGCCGGACC |
| 11073 | C215G | GF | gctggctcggtcaagaGAAGCTGCGGGCCGGACG |
| 11153 | C116T | EF | CGAGTGGGAAGAAAAGTAGA |
| 11153 | C116T | ER | ATGACTGCCTGCCTAGAA |
| 11153 | C116T | CR | gggacggtcggtagatAAGATAGGGTAGAGGCCG |
| 11153 | C116T | TR | gctggctcggtcaagaAAGATAGGGTAGAGGCCA |
| 11210 | C194T | EF | GAGGAGTGAGGGAAAGTAAG |
| 11210 | C194T | ER | AAATGGAGAGAGATGGGA |
| 11210 | C194T | CF | gggacggtcggtagatCCAGGAAATGACATGATC |
| 11210 | C194T | TF | gctggctcggtcaagaCCAGGAAATGACATGATT |
| 11248 | C225T | EF | TGAGTTGAACAGCACTTGG |
| 11248 | C225T | ER | AGGGTAAGGGAGGGAAAA |
| 11248 | C225T | CR | gggacggtcggtagatTGATTCTTTCGCTTGGCG |
| 11248 | C225T | TR | gctggctcggtcaagaTGATTCTTTCGCTTGGCA |
| 11372 | A251G | EF | TAGAAAAGAAGAAAAATCAA |
| 11372 | A251G | ER | ACACACACACACACACAC |
| 11372 | A251G | AR | gggacggtcggtagatCATCACCTTTTAGTTTCT |
| 11372 | A251G | GR | gctggctcggtcaagaCATCACCTTTTAGTTTCC |
| 11449 | C251G | EF | ACAGAAGAACAACAACAAAAC |
| 11449 | C251G | ER | TGCGTATGAGGTAAAGAGA |
| 11449 | C251G | CF | gggacggtcggtagatATGAGTGAAGCCTGTCTC |
| 11449 | C251G | GF | gctggctcggtcaagaATGAGTGAAGCCTGTCTG |
| 11450 | A251T | EF | ACAGAAGAACAACAACAAAAC |
| 11450 | A251T | ER | TGCGTATGAGGTAAAGAGA |
| 11450 | A251T | AR | gggacggtcggtagatGGACCATAATCTTGAAGT |
| 11450 | A251T | TR | gctggctcggtcaagaGGACCATAATCTTGAAGA |
| 11470 | C251T | EF | GCTTGTCTTGTCTGATAGGTG |
| 11470 | C251T | ER | CAACGTGAGAATTTCCAAAAT |
| 11470 | C251T | CR | gggacggtcggtagatTGAGAATTTCCAAAATAG |
| 11470 | C251T | TR | gctggctcggtcaagaTGAGAATTTCCAAAATAA |
| 11472 | A251T | EF | TACATTCAAGGCAAGAAAA |
| 11472 | A251T | ER | TGATTAGTTACAATTACCTCTAGTATC |
| 11472 | A251T | AF | gggacggtcggtagatAGTTTGTCAGTAAATGTA |
| 11472 | A251T | TF | gctggctcggtcaagaAGTTTGTCAGTAAATGTT |
| 11487 | A485T | EF | gacgatgccttcagcacaAGAGAGCAGCTAGACTGAGA |
| 11487 | A485T | ER | TTCCTGCAAACAGTTGAG |
| 11487 | A485T | AR | gggacggtcggtagatAGTTGAGGGCTCAGGATT |
| 11487 | A485T | TR | gctggctcggtcaagaAGTTGAGGGCTCAGGATA |
| 11488 | C533G | EF | gacgatgccttcagcacaAGAGAGCAGCTAGACTGAGA |
| 11488 | C533G | ER | GTAAATAAAATGGGATGGTG |
| 11488 | C533G | CR | gggacggtcggtagatGCCCCAGCAAGCTGCATG |
| 11488 | C533G | GR | gctggctcggtcaagaGCCCCAGCAAGCTGCATC |
| 11493 | A171G | EF | CCTTTTGTGTTTTGTTTTGT |
| 11493 | A171G | ER | CTTCTCCACCTTCCATTC |
| 11493 | A171G | AF | gggacggtcggtagatGGGAACTCCTAAATCAAA |
| 11493 | A171G | GF | gctggctcggtcaagaGGGAACTCCTAAATCAAG |
| 11502 | C455T | EF | gacgatgccttcagcacaACGATGGGGTCAGAGTCA |
| 11502 | C455T | ER | CCTACATTTCACACACGAACA |
| 11502 | C455T | CR | gggacggtcggtagatACACACTCCTCTCTCAAG |
| 11502 | C455T | TR | gctggctcggtcaagaACACACTCCTCTCTCAAA |
| 11534 | G258T | EF | GCCATCGTCTTTCCCT |
| 11534 | G258T | ER | TCCTCCCTCCTTCTCTCT |
| 11534 | G258T | GR | gggacggtcggtagatCCTCCACCCACCAGGGCC |
| 11534 | G258T | TR | gctggctcggtcaagaCCTCCACCCACCAGGGCA |
| 11537 | A251G | EF | CCTCTTTCTCCTCCTCTTC |
| 11537 | A251G | ER | CTCTTCCTGTCTTCTCCTCT |
| 11537 | A251G | AF | gggacggtcggtagatAGATGGACCTCTACAGGA |
| 11537 | A251G | GF | gctggctcggtcaagaAGATGGACCTCTACAGGG |
| 11560 | A185G | EF | CTCCTCCAACTCCTTTAC |
| 11560 | A185G | ER | ATACTTCTCACTGCATCCT |
| 11560 | A185G | AR | gggacggtcggtagatCCTGTCCCCTCCCTAGTT |
| 11560 | A185G | GR | gctggctcggtcaagaCCTGTCCCCTCCCTAGTC |
| 11594 | C251T | EF | CACCTTCCTGAACTCACTC |
| 11594 | C251T | ER | TGATGTCTGTGCTGTCCT |
| 11594 | C251T | CR | gggacggtcggtagatTCTGGTCCACTCAAGGAG |
| 11594 | C251T | TR | gctggctcggtcaagaTCTGGTCCACTCAAGGAA |
| 11624 | C251T | EF | TCGGGAGGTGTAAGTAAG |
| 11624 | C251T | ER | CCACAGTCAGAAGAGACAA |
| 11624 | C251T | CR | gggacggtcggtagatAGAGACCCTGGTCCCAAG |
| 11624 | C251T | TR | gctggctcggtcaagaAGAGACCCTGGTCCCAAA |
| 11627 | C251T | EF | TTTATCACTACACCCCCTACTC |
| 11627 | C251T | ER | GACAGACCGACCAATCAC |
| 11627 | C251T | CR | gggacggtcggtagatCCCTGGGAAGGTTGAGAG |
| 11627 | C251T | TR | gctggctcggtcaagaCCCTGGGAAGGTTGAGAA |
| 11650 | A146G | EF | CTGTCTGTTTGGGTCTTC |
| 11650 | A146G | ER | CGTTGTTCTCTGTCCACT |
| 11650 | A146G | AR | gggacggtcggtagatGGCCAAATGTCTAAAAGT |
| 11650 | A146G | GR | gctggctcggtcaagaGGCCAAATGTCTAAAAGC |
| 11654 | A251G | EF | CGTATCTCTTGCCTTTCTT |
| 11654 | A251G | ER | CTTCTCTTATGCCTTCCC |
| 11654 | A251G | AF | gggacggtcggtagatTTACTTGAAAGGACACCA |
| 11654 | A251G | GF | gctggctcggtcaagaTTACTTGAAAGGACACCG |
| 11655 | A251C | EF | CGTATCTCTTGCCTTTCTT |
| 11655 | A251C | ER | CTTCTCTTATGCCTTCCC |
| 11655 | A251C | AF | gggacggtcggtagatTTCTGCACTAAAGCTGTA |
| 11655 | A251C | CF | gctggctcggtcaagaTTCTGCACTAAAGCTGTC |
| 11656 | C251T | EF | TGGGAAGAAAAAGAGAAG |
| 11656 | C251T | ER | GTTGAAACACTGCACAAG |
| 11656 | C251T | CR | gggacggtcggtagatCAGGGCTGTTGGGTGAAG |
| 11656 | C251T | TR | gctggctcggtcaagaCAGGGCTGTTGGGTGAAA |
| 11825 | A277G | ER | gacgatgccttcagcacaTGAATAGACAGGGACGAA |
| 11825 | A277G | EF | GACCTTGGAAATAATGGAG |
| 11825 | A277G | AF | gggacggtcggtagatCAACCCAGCAAAAATGGA |
| 11825 | A277G | GF | gctggctcggtcaagaCAACCCAGCAAAAATGGG |
| 11914 | A246T | EF | gacgatgccttcagcacaTTGGAAGTGAGATAAGATAGGT |
| 11914 | A246T | ER | ACGGTGAGAATGAGAGGT |
| 11914 | A246T | AR | gggacggtcggtagatAAAACAGACATCAGAGGT |
| 11914 | A246T | TR | gctggctcggtcaagaAAAACAGACATCAGAGGA |
| 12097 | A411G | ER | gacgatgccttcagcacaGATGAAACCCTGTCTCTACT |
| 12097 | A411G | EF | TTATCAACCTTAGTCTCCCT |
| 12097 | A411G | AF | gggacggtcggtagatACCTGCCACCACACCCAA |
| 12097 | A411G | GF | gctggctcggtcaagaACCTGCCACCACACCCAG |
| 12366 | A412G | ER | gacgatgccttcagcacaGCTGATGTGGTTGTGAG |
| 12366 | A412G | EF | GTTCCTGTAGCTCGTGTAG |
| 12366 | A412G | AF | gggacggtcggtagatCTCCCCGCCCTGCAGCAA |
| 12366 | A412G | GF | gctggctcggtcaagaCTCCCCGCCCTGCAGCAG |
| 12619 | A25G | ER | gacgatgccttcagcacaTGGCTGGACTTTGACTGATA |
| 12619 | A25G | EF | TCTTGTTTGTGTCACAGTGC |
| 12619 | A25G | AF | gggacggtcggtagatTGTGTCACAGTGCTCTGA |
| 12619 | A25G | GF | gctggctcggtcaagaTGTGTCACAGTGCTCTGG |
| 13025 | A585C | EF | gacgatgccttcagcacaTTTAAGTAACATGACAAACTC |
| 13025 | A585C | ER | ATCTGATAACTGAGCAGG |
| 13025 | A585C | AR | gggacggtcggtagatCTATTAAGTAACTGGTGT |
| 13025 | A585C | CR | gctggctcggtcaagaCTATTAAGTAACTGGTGG |
| 13191 | A504G | ER | gacgatgccttcagcacaATTCTCCCATTTCTCCTGT |
| 13191 | A504G | EF | TGCCTCTTCTCCTCATTC |
| 13191 | A504G | AF | gggacggtcggtagatCCCTAATGTCTTCCTCTGA |
| 13191 | A504G | GF | gctggctcggtcaagaCCCTAATGTCTTCCTCTGG |
| 900045 | C116T | EF | ATCTCCTGATCCAAGTCC |
| 900045 | C116T | ER | CACACTGTGCCCATCTAC |
| 900045 | C116T | CR | gggacggtcggtagatCTGACTGATTACCTCATG |
| 900045 | C116T | TR | gctggctcggtcaagaCTGACTGATTACCTCATA |
| 900078 | A251G | EF | CATAGGTAAAGATCTGTAGGTG |
| 900078 | A251G | ER | CCACCTTGGAAGTTGGCAAA |
| 900078 | A251G | AR | gggacggtcggtagatattaaatcgcctctctcT |
| 900078 | A251G | GR | gctggctcggtcaagaattaaatcgcctctctcC |
| 900107 | C426T | ER | gacgatgccttcagcacaAGGGCTTTTTCAGGTAGA |
| 900107 | C426T | EF | GACCTTTCCTGGGTAGAA |
| 900107 | C426T | CF | gggacggtcggtagatACTCTGAACCTGGGGGAC |
| 900107 | C426T | TF | gctggctcggtcaagaACTCTGAACCTGGGGGAT |
| 10000002 | A103G | AF | gggacggtcggtagatGATCAACACAATCTTCAA |
| 10000002 | A103G | EF | CAGCTGAAAGAGATGAAATTTACT |
| 10000002 | A103G | ER | GACGATGCCTTCAGCACAAACTTATGAAGATTAAGGCATAGG |
| 10000002 | A103G | GF | gctggctcggtcaagaGATCAACACAATCTTCAG |
| 10000006 | G107A | AF | gctggctcggtcaagaGGGCTGGGCTGCTAGGGA |
| 10000006 | G107A | EF | AGACGAGTTCAAGGTGAGTG |
| 10000006 | G107A | ER | GACGATGCCTTCAGCACACCAAGTTTCCGAGTTTCC |
| 10000006 | G107A | GF | gggacggtcggtagatGGGCTGGGCTGCTAGGGG |
| 10000014 | A153C | AF | gggacggtcggtagatGTACCAATACATCCTGCA |
| 10000014 | A153C | CF | gctggctcggtcaagaGTACCAATACATCCTGCC |
| 10000014 | A153C | EF | CTGCTGATGTCTCTGTTG |
| 10000014 | A153C | ER | GACGATGCCTTCAGCACAGACTTACTTTGCTCACACTT |
| 10000025 | C291T | CF | gggacggtcggtagatCCTCACTTCCTCAACGCC |
| 10000025 | C291T | EF | CCTCTCTGTCTGGTTATCTTG |
| 10000025 | C291T | ER | GACGATGCCTTCAGCACAAGTGTGCCTCCTGGTTAG |
| 10000025 | C291T | TF | gctggctcggtcaagaCCTCACTTCCTCAACGCT |

TABLE 2b

Oligonucleotide primers used for genotyping
using Pyro-sequencing
The baySNP number refers to an internal number-
ing of the PA SNPs. Primer sequences are listed
for preamplification of the genomic fragments
and for sequencing of the SNP using the pyro-
sequencing method. Bio: Biotinylated Oligonu-
cleotide.

| | | |
|---|---|---|
| BaySNP | Name | Sequence |

| | | |
|---|---|---|
| 2995 | Primer F | GCCAAGACTAGGAAGTAAGTGT |
| 2995 | Primer R | Bio-CCCAGAACCACAAAGCTAGTAA |
| 2995 | Seq. | TGCCCTGGTCACCTCCTTTCC |
| 3689 | Primer F | BIO-CTGACCCTGACCTTCATACTCAA |
| 3689 | Primer R | AGAAGAAAGAAGCCTCTCTACAGTT |
| 3689 | Seq. | AACAGATCAGGTTGGTG |
| 4838 | Primer F | Bio-CAAAGATGACCTTATGGCTCTGA |
| 4838 | Primer R | GTCTCGGAACATGACCTTTAGT |
| 4838 | Seq. | TGACTAAGAATGTAATGGGGAAGA |
| 6498 | Primer F | CTTTGTGGATCTTTCTGCGGTGT |
| 6498 | Primer R | Bio-CCATGTTGAGGAGCCCAGAGTGA |
| 6498 | Seq. | ATTACAGTTGTGAGATTGTGC |
| 8021 | Primer F | GGCCTTCTATGTACTAGGCG |
| 8021 | Primer R | Bio-CTCTTTCTGGAGGCATCAATC |
| 8021 | Seq. | CACAGGGAGACCCC |
| 8060 | Primer F | Bio-GCCTTATTTTCCACTCCCACCT |
| 8060 | Primer R | TACCTTTCCCCATCCCAACTG |
| 8060 | Seq. | TCAGCATATGTTTGGATT |
| 8846 | Primer F | ATTTGAGAGAAGGTAGGGT |
| 8846 | Primer R | BIO-TTTGTTACTCTGTAGCCA |
| 8846 | Seq. | AAATATTCAGTAACTTGTTT |
| 9849 | Primer F | AAG CAG CAA TCG AAT CCC TT |
| 9849 | Primer R | TGT TGT TGT TTG GCT AGC TCC |
| 9849 | Seq. | CCT GCC TTA CTG AGA GCC AAA |
| 10079 | Primer F | Bio-CACGCCAATTCCCACCATCCT |
| 10079 | Primer R | GTCCGTCGAGGGGGAATGTGTTT |
| 10079 | Seq. | AATGTGTTTCTTGGGGGT |
| 10747 | Primer F | CTAACCATCTTCCAAATGCTTAATC |
| 10747 | Primer R | BIO-TCCTTGAGTCTGAGTTTCCC |
| 10747 | Seq. | CACAAGAAACCCTGAAA |
| 11578 | Primer F | CTC GGC GTG CTT GGT AAT AA |
| 11578 | Primer R | CGG AGC CGA ACT CTG GAG GAA TCT |
| 11578 | Seq. | GGC TGG CAA GTT GTT CCA TCC CAC |
| 11644 | Primer F | TGA GCA GCG CAT CCT |
| 11644 | Primer R | TGC AGC CCA CTG ACT CAA |
| 11644 | Seq. | GCT GTT ACT CAG TAT GAT |
| 12008 | Primer F | CCGAAGACCAAGACGC |
| 12008 | Primer R | Bio-TCTTCCATAAAAACAAGGCTC |
| 12008 | Seq. | AAACAAGAAATTCTGTTTA |
| 13937 | Primer F | TGA CAG CTC CCA TTG GAA |
| 13937 | Primer R | AAT TAA TGC GAT CCC TC |
| 13937 | Seq. | GAC AGC TCC CAT TGG AAG |
| 900002 | Primer F | ATTGGGCAGGGATAAGAGAAAAG |
| 900002 | Primer R | Bio-GATGAATCACAGAATGCGGTAT |
| 900002 | Seq. | CACACAGCAGTTCACGCA |
| 900013 | Primer F | GCCAAGACTAGGAAGTAAGTGT |
| 900013 | Primer R | Bio-CCCAGAACCACAAAGCTAGTAA |
| 900013 | Seq. | TGCCCTGGTCACCTCCTTTCC |
| 900025 | Primer F | Bio-AGTGGCTCACTTGCTAACG |
| 900025 | Primer R | CTGGGGAAGAAAATAAATGAA |
| 900025 | Seq. | CTTGCTCTTAGGATACACGT |
| 900032 | Primer F | AGCGTCTTCACCATCTGCT |
| 900032 | Primer R | Bio-GGGAAGGAGGAAGCCAAACA |
| 900032 | Seq. | ACATGTCTGATGATACCTGG |
| 900045 | Primer F | BIO-GCCATGCACGATTTCCC |
| 900045 | Primer R | CACTGTGCCCATCTACGAG |
| 900045 | Seq. | GGACCTGACTGATTACCT |
| 900065 | Primer F | GAGTAGCTAGGATCACAGGTGCGT |
| 900065 | Primer R | BIO-TGTTCGAGATTTAAGAAAGTTGGC |
| 900065 | Seq. | CAGGTGCGTGCCACCATGCCC |
| 900082 | Primer F | CAC ACA ATT TTC CAC TTA |
| 900082 | Primer R | GAC TCC AGT TTT CTA TCA |
| 900082 | Seq. | ATG TTG ATG TAA TCT ACT |
| 900096 | Primer F | TGGGGCAAGCAACAGTGGT |
| 900096 | Primer R | Bio-TAGGCAGGGCAAGGGATTAGG |
| 900096 | Seq. | TTTAAATTCTCTGACAGAGAC |
| 900107 | Primer F | BIO-GCCACCAGCCCACACTCTGAACCTG |
| 900107 | Primer R | CCATCAGCCTTCACCCACGTGCCA |
| 900107 | Seq. | GCCTCAGCTTGACCT |
| 900115 | Primer F | Bio-GGTAAGTGCGTGCCTGGGAGATGC |
| 900115 | Primer R | CGGGGTGGGGAGGACAGAGC |
| 900115 | Seq. | GAGGACAGAGCAAAAGGAT |
| 900121 | Primer F | Bio-TGCCTTACAATATACAATGG |
| 900121 | Primer R | CAATGGGTAAGGAGTAAAGTT |
| 900121 | Seq. | TTCCAGCTGCTTTTA |

TABLE 2c

Oligonucleotide primers used for genotyping
using Restriction Fragment Length Polymorphism
(RFLP)
The baySNP number refers to an internal number-
ing of the PA SNPs. Primer sequences are listed
for preamplification of the genomic fragments.
The restriction enzyme used for RFPL is
indicated.

| | | | |
|---|---|---|---|
| baySNP | Name | Sequence | Enzyme |

| | | | |
|---|---|---|---|
| 900173 | Primer F | GAACAAACCTCCGAGATGCTAC | Hind III |
| 900173 | Primer R | GTCTTATGTTACTGGGCTTTCACC | Hind III |

TABLE 2d

Oligonucleotide primers used for genotyping using TaqMan
The baySNP number refers to an internal numbering of the PA SNPs.
Primer sequences are listed for amplification of the genomic
fragments. In addition the respective fluorescent hybridisation
probes are listed. If not otherwise stated, all fluorescent
probes have a ‘minor groove binder’ (MGB) attached
(Kutyavin et al., Nucleic Acids Research 2000, 28: 655-661).

| | | |
|---|---|---|
| baySNP | F-Sequence | R-Sequence |

| | | |
|---|---|---|
| 52 | CACCCTCTAGAATTCACTATTAATTTTCAAC | GGCCTTGAAGAAGATTTTATATTGAGAA |
| 542 | TTTCGCTCCATCAACCAAGTC | GATGGGTGATCAGCCGAATC |
| 821 | GCCCAGTTATACCTCAGTGTTGTAAC | AGGTCAGTACAGAGGGTATCATGAGA |
| 1056 | TGTATGCACGTGCGTGATCTG | CGCCCTCGGCACTCTTG |
| 1204 | CTGTAAGCATCTGGAATTGTCATGA | GGCTCAGTCTTTGATCTTTAGCAAG |
| 1722 | GGACCCTAAGAACCCCAGGAT | ATGGGCTAACACAGGAGATGATG |
| 1757 | ACAGGGCTGGCAGCCAC | AGCCTCTGCCCTCCTCCA |
| 1765 | GGAGCTGTGAGGTATGGGCTT | TGTCAAGATGCAGCTGAAGGTC |
| 1799 | TTTGGTGCGTTGTCATTGACA | TGGACATATGGGCGGACTCT |
| 1837 | CACTCAGCCCTGCTCTTTCC | CATCCTTGGCGGTCTTGGT |
| 1870 | CTGGCTCCTGACCCTTGCT | GGAGGATGCCATCTCGAACA |
| 1988 | CCGTGGCTTCATGGTGACT | CTACCTGTCCGGTGCATCATC |
| 2000 | TTCTCACTGTGATATATAAACTCAGACCC | CGATGAACAGTTGGAATAGGTTGT |
| 2085 | TCATTACATCAGGTATATTGCACTGTAAA | TCAGAGACACTGAAGAACTTAAAGAAATC |
| 2281 | GCTGCATTGGAGAGGACTGATC | CGGTTAACTTATAAAGAAACGGATGTTC |
| 2298 | TGCTAGTGTTTTCTGGTTGCATATT | GGCACCGTGTAGACTTGATCTAAA |
| 2357 | GCGAAGTGTCGGACACCAA | GGTTACGTCTGCTCTTCGATCCT |
| 4838 | AAGATGACCTTATGGCTCTGAGATG | TCTCGGAACATGACCTTTAGTCTGT |
| 5320 | GGGATATATAGTAGAAAAACAAGCCTGTCT | CAACTTAATCACTACTACTCCATGTAAAGCA |
| 5717 | GGCCCGCTCCTGGCT | AACCCCACACCTTCAGTCTAGAAA |
| 5959 | ACCAGAAACAAATGCCAACCA | CAGTGTGAAACCAAGGGATGTC |
| 6482 | CATAGTTTAGGATAAACAAAAGGGATTCA | TGTCATGGAAACGCCACAAC |
| 8060 | GCTATTGAATGGATGTGCCTTATTT | TGCATGGCATCAGCATATGTT |
| 8816 | CAGCCCCTCTGCTCCAAG | TCCCCCTCTGTCCAGGC |
| 10600 | GGTGACGTTTGCGCATCTC | AAGTTAATCAAGCCTTTTCAATTGG |
| 10771 | CTGGGCCCACCGAGTTAC | GATCTCTGTGAGTGTGCGTCTGT |
| 10948 | ACATTCCCCTTCCACGCTT | GCAGGGCAGAGGGAGGA |
| 11001 | GCCATCCTTGTTGAACGTGAA | ACATGACCAGGGCCCACTT |
| 11073 | GAGCAACAGCCGCCTGAG | GCGGGAGCTAGAGAGCAGTG |
| 11248 | GAAAGCTAACTCCCCTGACG | TGAAGGGTAAGGGAGGGAAA |
| 11654 | AGTTTGTTTTCCTATTAGAGGTTTCCA | CTCTTATGCCTTCCCCACCA |
| 11655 | CATATTCAAGAAAGATTATCTCCAACTCTT | TGGAAACCTCTAATAGGAAAACAAACT |
| 13191 | GAGTTGGTGGCATAAAACCCTAA | CCTGTCCCCACCTTCTCTCTCT |

| | | |
|---|---|---|
| baySNP | VIC-MGB | FAM-MGB |

| | | |
|---|---|---|
| 52 | CTATGCATAcTTTTGC | ATCCATAgTTTTGCATTAT |
| 542 | CAATTGGaGTTGGGAGG | AATTGGgGTTGGGAGG |
| 821 | TGTGATACCTGGaACAG | CTGTGATACCTGGcACA |
| 1056 | CCAAACAaCAGGACGG | AAACAgCAGGACGGG |
| 1204 | CACTCACATTAtAATTAG | ACTCACATTAcAATTAGT |
| 1722 | TGGCCTGGCGgTG | TGGCCTGGCGaTGT |
| 1757 | AACCAAAATGaAGGAGAG | ACCAAAATGgAGGAGAG |
| 1765 | ACGGAGGAAGAgGT | ACGGAGGAAGAaGT |
| 1799 | AGTGTGATCaTCACTTT | CAGTGTGATCgTCACT |
| 1837 | TGCAGGGcTACATGA | TCATGCAGGGtTACAT |
| 1870 | TGCCTCCTTcTCACAC | CCTCCTTtTCACACCGA |
| 1988 | TCCTATACcGTGGGTGT | CTATACtGTGGGTGTCAT |
| 2000 | TACTCATcTTCCTAATTAC | CAAATATCTACTCATtTTC |
| 2085 | TGTTACCAGAAAgAAA | TGTTACCAGAAAtAAA |
| 2281 | CATACCACAAAaCCA | ACCACAAAcCCAGGTC |
| 2298 | TCATGGGCaTTTCA | TATCATGGGCcTTTCA |
| 2357 | AAGACGAAAATGaATC | AAGACGAAAATGgATC |
| 4838 | AAGAAtTGCCCTGCCT | AAGAAcTGCCCTGCC |
| 5320 | AAGGAAAGCTGGaTATG | AGGAAAGCTGGgTATGT |
| 5717 | Vic- | Fam- |
| | CCACCTCCCTtCTAGCCTCAGTTGC- | CCCACCTCCCTcCTAGCCTCAGTT- |
| | TAMRA | TAMRA |
| 5959 | Vic-CGAATGTGgCTGCCCAGCC- | Fam-TCGAATGTGaCTGCCCAGCCTC- |
| | TAMRA | TAMRA |
| 6482 | AACAGATCTGGTCTaCCT | AGATCTGGTCTgCCTC |
| 8060 | CCCACCTGGaGAAT | TCCCACCTGGgGAA |
| 8816 | TGAGAAAAAAGgTTCCG | CTGAGAAAAAAGcTTC |
| 10600 | TGCTCAGGAtAGCC | TGCTCAGGAcAGCC |
| 10771 | AGGAAGcGTGGCCT | CAAGGAAGgGTGGC |
| 10948 | CGCCCAGTAATaCAGA | CCCAGTAATcCAGACAC |
| 11001 | TCGTTCCAcTGGACGT | TTCCAtTGGACGTCCT |
| 11073 | TCGGCGCTgGTC | TCTCGGCGCTcGT |
| 11248 | CTTGGCgTCGCGTC | TTGGCaTCGCGTCAG |
| 11654 | TTGAAAGGACACCaTATT | ACACCgTATTTTTCAC |
| 11655 | CACTAAAGCTGTaATATTA | CTAAAGCTGTcATATTAC |
| 13191 | TCTTCCTCTGgGTAACA | TCCTCTGaGTAACAAC |

TABLE 3
PA SNPs, SNP classes and putative PA genes
The baySNP number refers to an internal numbering of the PA SNPs. Listed are the different polymorphisms found in
our association study. Also from the association study we defined SNP classes; with ADR being adverse drug reaction
related, with EFF being drug efficacy related and CVD being cardiovascular disease related. ADR3 and ADR5 relate to
advanced and severe ADR, whereas VEFF and UEFF relate to very high/low and ultra high/low drug efficacy
(see table 1b). Also accession numbers and descriptions of those gene loci are given that are most homologous to
the PA genes as listed in the sequences section (see below). Homologous genes and their accession numbers
could be found by those skilled in the art in the Genbank database.

| BAYSNP | SNP class | GTYPE11 | GTYPE12 | GTYPE22 | NCBI | DESCRIPTION |
|---|---|---|---|---|---|---|
| 28 | EFF | CC | CT | TT | U15552 | Human acidic 82 kDa protein mRNA, complete cds. |
| 29 | CVD | AA | AG | GG | HS162961 | Human T-lymphoma invasion and metastasis inducing TIAM1 |
| | | | | | | protein (TIAM1) mRNA, complete cds. |
| 29 | ADR3 | AA | AG | GG | HS162961 | Human T-lymphoma invasion and metastasis inducing TIAM1 |
| | | | | | | protein (TIAM1) mRNA, complete cds. |
| 29 | ADR5 | AA | AG | GG | HS162961 | Human T-lymphoma invasion and metastasis inducing TIAM1 |
| | | | | | | protein (TIAM1) mRNA, complete cds. |
| 52 | EFF | CC | CG | GG | X69907 | H. sapiens gene for mitochondrial ATP synthase c subunit (P1 form) |
| 56 | EFF | AA | AG | GG | M92357 | Homo sapiens B94 protein mRNA, complete cds. |
| 89 | CVD | AA | AG | null | L23982 | Homo sapiens (clones: CW52-2, CW27-6, CW15-2, CW26-5, 11-67) |
| | | | | | | collagen type VII intergenic region and (COL7A1) gene, complete cds. |
| 90 | CVD | CC | CT | TT | M65212 | Homo sapiens catechol-O-metbyltransferase (COMT) mRNA, |
| | | | | | | complete cds. |
| 99 | CVD | CC | CT | TT | X96698 | H. sapiens mRNA for D1075-like gene |
| 140 | EFF | CC | CT | TT | M14335 | Human coagulation factor V mRNA, complete cds. |
| 152 | EFF | AA | AG | GG | M32670 | Homo sapiens ITGB3 gene, intron 2, fragment C, partial sequence. |
| 214 | CVD | AA | AG | GG | X66957 | H. sapiens hexokinase I (MK-16) |
| 221 | CVD | CC | CG | GG | X76732 | H. sapiens mRNA for NEFA protein |
| 224 | CVD | CC | CT | TT | M14764 | Human nerve growth factor receptor mRNA, complete cds. |
| 294 | CVD | CC | CT | TT | P02568 | ACTIN, ALPHA SKELETAL MUSCLE (ALPHA-ACTIN 1). |
| 307 | CVD | CC | CT | TT | X63546 | H. sapiens mRNA for tre oncogene (clone 210) |
| 411 | CVD | AA | AT | TT | HS34804 | Human thermostable phenol sulfotransferase (STP2) gene, partial cds. |
| 449 | CVD | CC | CG | GG | M36341 | Human ADP-ribosylation factor 4 (ARF4) mRNA, complete cds. |
| 466 | CVD | CC | CT | TT | AF129756 | Homo sapiens MSH55 gene, partial cds; and CLIC1, DDAH, G6b, |
| | | | | | | G6c, G5b, G6d, G6e, G6f, BAT5, G5b, CSK2B, BAT4, G4, Apo M, |
| | | | | | | BAT3, BAT2, AIF-1, 1C7, LST-1, LTB, TNF, and LTA genes, |
| | | | | | | complete cds. |
| 472 | EFF | AA | AG | GG | M57965 | Homo sapiens (clones lambda gMHC 1, 2, 3 and 4) beta-myosin heavy |
| | | | | | | chain (MYH7) gene, complete cds. |
| 542 | CVD | AA | AG | GG | M64082 | Human flavin-containing monooxygenase (FMO1) mRNA, complete |
| | | | | | | cds. |
| 542 | ADR | AA | AG | GG | M64082 | Human flavin-containing monooxygenase (FMO1) mRNA, complete |
| | | | | | | cds. |
| 739 | CVD | CC | CG | GG | L43509 | Homo sapiens methionine adenosyltransferase alpha subunit gene |
| | | | | | | fragment. |
| 821 | CVD | AA | AC | CC | X80507 | H. sapiens YAP65 mRNA |
| 821 | VEFF | AA | AC | CC | X80507 | H. sapiens YAP65 mRNA |
| 1005 | CVD | AA | AG | GG | M81357 | Human coagulation factor VII (F7) gene exon 1 and factor X (F10) |
| | | | | | | gene, exon 1. |
| 1055 | CVD | AA | AT | TT | J02758 | Human apolipoprotein A-IV gene, complete cds. |
| 1056 | EFF | AA | AG | GG | Q16720 | CALCIUM-TRANSPORTING ATPASE PLASMA MEMBRANE, |
| | | | | | | ISOFORMS 3A/3B (EC 3.6.1.38) (CALCIUM PUMP) (PMCA3). |
| 1085 | CVD | AA | AG | GG | M14564 | Human cytochrome P450c17 (steroid 17-alpha-hydroxylase/17,20 |
| | | | | | | lyase) mRNA, complete cds. |
| 1086 | CVD | AA | AG | GG | M14564 | Human cytochrome P450c17 (steroid 17-alpha-hydroxylase/17,20 |
| | | | | | | lyase) mRNA, complete cds. |
| 1092 | CVD | CC | CG | GG | AF022375 | Homo sapiens vascular endothelial growth factor mRNA, complete |
| | | | | | | cds. |
| 1096 | CVD | GG | GT | TT | X15323 | H. sapiens angiotensinogen gene 5′region and exon 1 |
| 1101 | EFF | CC | CT | TT | AL031005 | Homo sapiens DNA sequence from PAC 329E20 on chromosome |
| | | | | | | 1p34.4-36.13. Contains endothelin-converting-enzyme 1 (ECE-1), |
| | | | | | | EST, STS, CA repeat |
| 1204 | CVD | AA | AG | GG | AC004264 | Homo sapiens PAC clone RP1-102K2 from 22q12.1-qter, complete |
| | | | | | | sequence. |
| 1504 | CVD | CC | CT | TT | AC005175 | Homo sapiens chromosome 19, cosmid R31449, complete sequence. |
| 1511 | EFF | GG | GT | TT | AF009674 | Homo sapiens axin (AXIN) mRNA, partial cds. |
| 1524 | ADR3 | AA | AC | CC | AF223404 | Homo sapiens WNT1 inducible signaling pathway protein 1 (WISP1) |
| | | | | | | gene, promoter and partial cds. |
| 1556 | EFF | CC | CG | GG | L34058 | Homo sapiens cadherin-13 mRNA, complete cds. |
| 1561 | CVD | AA | AC | CC | M31664 | Human cytochrome P450 (CYP1A2) gene, exons 1 and 2. |
| 1582 | CVD | CC | CT | TT | AF050163 | Homo sapiens lipoprotein lipase precursor, gene, partial cds. |
| 1638 | CVD | AA | AG | GG | AF090318 | Homo sapiens sterol 12-alpha hydroxylase CYP8B1 (Cyp8b1) |
| | | | | | | mRNA, partial cds. |
| 1653 | CVD | GG | GT | TT | J02846 | Human tissue factor gene, complete cds. |
| 1662 | CVD | CC | CT | TT | K02402 | Human coagulation factor IX gene, complete cds. |
| 1714 | CVD | AA | AG | GG | D50857 | Human DOCK180 protein mRNA, complete cds. |
| 1722 | ADR5 | CC | CT | TT | D73409 | Homo sapiens mRNA for diacylglycerol kinase delta, complete cds. |
| 1757 | EFF | AA | AG | GG | J04046 | Human calmodulin mRNA, complete cds. |
| 1765 | ADR3 | AA | AG | GG | J05096 | Human Na, K-ATPase subunit alpha 2 (ATP1A2) gene, complete cds. |
| 1765 | ADR5 | AA | AG | GG | J05096 | Human Na, K-ATPase subunit alpha 2 (ATP1A2) gene, complete cds. |
| 1776 | CVD | AA | AG | GG | L22569 | Homo sapiens cathepsin B mRNA, 3′ UTR with a stem-loop structure |
| | | | | | | providing mRNA stability. |
| 1799 | CVD | CC | CT | TT | D21255 | Human mRNA for OB-cadherin-2, complete cds. |
| 1806 | EFF | AA | AG | GG | AF106202 | Homo sapiens endothelial cell protein C receptor precursor (EPCR) |
| | | | | | | gene, complete cds. |
| 1837 | CVD | CC | CT | TT | J00098 | Human apolipoprotein A-I and C-III genes, complete cds. |
| 1837 | ADR5 | CC | CT | TT | X00566 | Human mRNA for lipoprotein apoAI Human apolipoprotein A-I and |
| | | | | | | C-III genes, complete cds. |
| 1837 | ADR | CC | CT | TT | J00098 | Human apolipoprotein A-I and C-III genes, complete cds. |
| 1870 | CVD | CC | CT | TT | M84820 | Human retinoid X receptor beta (RXR-beta) mRNA, complete cds. |
| 1882 | CVD | CC | CT | TT | U06643 | Human keratinocyte lectin 14 (HKL-14) mRNA, complete cds. |
| 1988 | CVD | CC | CT | TT | X61598 | H. sapiens mRNA for colligin (a collagen-binding protein) |
| 2000 | CVD | CC | TT | null | P03915 | NADH-UBIQUINONE OXIDOREDUCTASE CHAIN 5 (EC |
| | | | | | | 1.6.5.3). |
| 2000 | ADR | CC | TT | null | P03915 | NADH-UBIQUINONE OXIDOREDUCTASE CHAIN 5 (EC |
| | | | | | | 1.6.5.3). |
| 2071 | CVD | AA | AG | GG | L04143 | Human c-kit gene. |
| 2078 | CVD | GG | GT | TT | X77584 | H. sapiens mRNA for ATL-derived factor/thiredoxin. |
| 2085 | VEFF | GG | GT | TT | X82540 | H. sapiens mRNA for activin beta-C chain |
| 2095 | CVD | AG | GG | null | L34155 | Homo sapiens laminin-related protein (LamA3) mRNA, complete |
| | | | | | | cds. |
| 2119 | CVD | AA | AG | null | Z22535 | H. sapiens ALK-3 mRNA. |
| 2119 | EFF | AA | AG | null | Z22535 | H. sapiens ALK-3 mRNA. |
| 2141 | EFF | AA | AG | GG | AB035073 | Homo sapiens mRNA for platelet glycoprotein VI, complete cds. |
| 2141 | CVD | AA | AG | GG | AB035073 | Homo sapiens mRNA for platelet glycoprotein VI, complete cds. |
| 2182 | EFF | AA | AG | GG | D32046 | Human gene for thrombopoietin, exon1-exon6, complete cds. |
| 2234 | CVD | GG | GT | TT | AC004264 | Homo sapiens PAC clone RP1-10K2 from 22q12.1-qter, complete |
| | | | | | | sequence. |
| 2281 | VEFF | AA | AC | CC | X87872 | H. sapiens mRNA for hepatocyte nuclear factor 4c |
| 2298 | CVD | AA | AC | CC | V01511 | H. sapiens gene for beta-nerve growth factor (beta-NGF) |
| 2341 | CVD | CC | CT | TT | J03280 | Human phenylethanolamine N-methyltransferase gene, complete cds. |
| 2357 | CVD | AA | AG | GG | O15055 | PERIOD CIRCADIAN PROTEIN 2 (KIAA0347). |
| 2366 | CVD | GG | GT | TT | P35414 | PROBABLE G PROTEIN-COUPLED RECEPTOR APJ. |
| 2423 | CVD | AA | AG | GG | AF000571 | Homo sapiens kidney and cardiac voltage dependent K+ channel |
| | | | | | | (KvLQT1) mRNA, complete cds. |
| 2708 | CVD | CC | CT | TT | AL031005 | Homo sapiens DNA sequence from PAC 329E20 on chromosome |
| | | | | | | 1p34.4-36.13. Contains endothelin-converting-enzyme 1 (ECE-1), |
| | | | | | | EST, STS, CA repeat |
| 2995 | ADR5 | AA | AC | CC | ABCC1 | ABCC1: ATP-binding cassette, sub-family C (CFTR/MRP), member 1 |
| 2995 | UEFF | AA | AC | CC | ABCC1 | ABCC1: ATP-binding cassette, sub-family C (CFTR/MRP), member 1 |
| 3360 | ADR5 | GG | GT | TT | ABCB4 | ABCB4: ATP-binding cassette, sub-family B (MDR/TAP), member 4 |
| 3464 | CVD | AA | AG | GG | M34668 | Human protein tyrosine phosphatase (PTPase-alpha) mRNA. |
| 3689 | EFF | CC | CG | GG | M95724 | H. sapiens centromere autoantigen C (CENPC) mRNA, complete cds. |
| 3975 | UEFF | AA | AC | CC | U43368 | Human VEGF related factor isoform VRF186 precursor (VRF) |
| | | | | | | mRNA, complete cds. |
| 3976 | UEFF | AA | AG | GG | U43368 | Human VEGF related factor isoform VRF186 precursor (VRF) |
| | | | | | | mRNA, complete cds. |
| 4206 | ADR3 | AA | AT | TT | BC000006 | Homo sapiens, ATPase, Na+/K+ transporting, beta 1 polypeptide |
| 4838 | VEFF | AA | AG | GG | L08246 | Human myeloid cell differentiation protein (MCL1) mRNA. |
| 4912 | EFF | AA | AG | GG | AF022375 | Homo sapiens vascular endothelial growth factor mRNA, complete |
| | | | | | | cds. |
| 4925 | CVD | AA | AC | CC | AF036365 | Homo sapiens caveolin-3 (CAV3) mRNA, complete cds. |
| 4966 | ADR3 | AA | AG | GG | AF133298 | Homo sapiens cytochrome P450 (CYP4F8) mRNA, complete cds. |
| 5014 | ADR5 | AA | AG | GG | AL008637 | Human DNA sequence from clone CTA-833B7 on chromosome |
| | | | | | | 22q12.3-13.2 Contains the NCF4 gene for cytosolic neutrophil factor |
| | | | | | | 4 (40 kD), the 5′ part of the CSF2RB gene for granulocyte- |
| | | | | | | macrophage low-affinity colony stimulating factor 2 receptor beta, |
| | | | | | | ESTs, STS |
| 5296 | CVD | AA | AG | GG | J02933 | Human blood coagulation factor VII gene, complete cds. |
| 5296 | EFF | AA | AG | GG | J02933 | Human blood coagulation factor VII gene, complete cds. |
| 5298 | EFF | CC | CT | TT | J02933 | Human blood coagulation factor VII gene, complete cds. |
| 5298 | CVD | CC | CT | TT | J02933 | Human blood coagulation factor VII gene, complete cds. |
| 5320 | EFF | AA | AG | GG | J03799 | Human colin carcinoma laminin-binding protein mRNA, complete |
| | | | | | | cds. |
| 5361 | CVD | AA | AC | CC | L02932 | Human peroxisome proliferator activated receptor mRNA, complete |
| | | | | | | cds. |
| 5457 | EFF | AA | AG | GG | L29529 | Homo sapiens (clone HHT-1 variant harboring HH-05) cardiac L-type |
| | | | | | | voltage dependent calcium channel alpha 1 subunit (CACNL1A1) |
| | | | | | | mRNA, complete cds. |
| 5704 | CVD | CC | CT | TT | M58050 | Human membrane cofactor protein (MCP) mRNA, complete cds. |
| 5717 | ADR3 | AA | AG | GG | AL008637 | Human DNA sequence from clone CTA-833B7 on chromosome |
| | | | | | | 22q12.3-13.2 Contains the NCF4 gene for cytosolic neutrophil factor |
| | | | | | | 4 (40 kD), the 5′ part of the CSF2RB gene for granulocyte- |
| | | | | | | macrophage low-affinity colony stimulating factor 2 receptor beta, |
| | | | | | | ESTs, STS |
| 5959 | CVD | AA | AG | GG | HSHMGCOAS | H. sapiens mRNA for 3-hydroxy-3-methylglutaryl coenzyme A |
| | | | | | | synthase |
| 5959 | ADR5 | AA | AG | GG | HSHMGCOAS | H. sapiens mRNA for 3-hydroxy-3-methylglutaryl coenzyme A |
| | | | | | | synthase |
| 5959 | ADR | AA | AG | GG | HSHMGCOAS | H. sapiens mRNA for 3-hydroxy-3-methylglutaryl coenzyme A |
| | | | | | | synthase |
| 6162 | ADR3 | CC | CG | GG | AF005896 | Homo sapiens Na K-ATPase beta-3 subunit (atp1b3) gene, exon 7 |
| | | | | | | and complete cds. |
| 6162 | ADR | CC | CG | GG | AF005896 | Homo sapiens Na K-ATPase beta-3 subunit (atp1b3) gene, exon 7 |
| | | | | | | and complete cds. |
| 6162 | ADR5 | CC | CG | GG | AF005896 | Homo sapiens Na K-ATPase beta-3 subunit (atp1b3) gene, exon 7 |
| | | | | | | and complete cds. |
| 6236 | ADR5 | CC | CT | TT | HSU62961 | Human succinyl CoA:3-oxoacid CoA transferase precursor (OXCT) |
| | | | | | | mRNA, complete cds. |
| 6236 | ADR3 | CC | CT | TT | HSU62961 | Human succinyl CoA:3-oxoacid CoA transferase precursor (OXCT) |
| | | | | | | mRNA, complete cds. |
| 6482 | CVD | AA | AG | GG | X69086 | H. sapiens mRNA for utrophin |
| 6498 | CVD | AA | AG | GG | X71348 | Homo sapiens vHNF1-C mRNA |
| 6744 | ADR5 | CC | CT | TT | AC002310 | Human Chromosome 16 BAC clone CIT987SK-A-635H12, complete |
| | | | | | | sequence. |
| 7133 | CVD | CC | CG | GG | K02402 | Human coagulation factor IX gene, complete cds. |
| 8021 | CVD | AA | AG | GG | Z13009 | H. sapiens mRNA for E-cadherin |
| 8060 | CVD | AA | AG | GG | Z99572 | Human DNA sequence from PAC 86F14 on chromosome 1q23-1q24. |
| | | | | | | Contains coagulation factor V, ESTs and STS. |
| 8210 | EFF | AA | AG | GG | ABCB11 | ABCB11: ATP-binding cassette, sub-family B (MDR/TAP), member |
| | | | | | | 11 |
| 8592 | VEFF | CC | CT | TT | J04038 | Human glyceraldehyde-3-phosphate dehydrogenase (GAPDH) gene, |
| | | | | | | complete cds. |
| 8816 | EFF | CC | CG | GG | L36033 | Human pre-B cell stimulating factor homologue (SDF1b) mRNA, |
| | | | | | | complete cds. |
| 8846 | CVD | AA | AG | GG | L41162 | Homo sapiens collagen alpha 3 type IX (COL9A3) mRNA, complete |
| | | | | | | cds. |
| 8943 | CVD | AA | AC | CC | AF050163 | Homo sapiens lipoprotein lipase precursor, gene, partial cds. |
| 9193 | CVD | CC | CG | GG | M12674 | Human estrogen receptor mRNA, complete cds. |
| 9443 | CVD | CC | CT | TT | U09587 | Human glycyl-tRNA synthetase mRNA, complete cds. |
| 9516 | CVD | AA | AG | GG | U16720 | Human interleukin 10 (IL10) gene, complete cds. |
| 9698 | ADR | AA | AG | GG | HS5211110 | Homo sapiens X28 region near ALD locus containing dual specificity |
| | | | | | | phosphatase 9 (DUSP9), ribosomal protein L18a (RPL18a), |
| | | | | | | Ca2+/Calmodulin-dependent protein kinase I (CAMKI), creatine |
| | | | | | | transporter (CRTR), CDM protein (CDM), adrenoleukodystrophy |
| | | | | | | protein (AL |
| 9698 | ADR3 | AA | AG | GG | HS5211110 | Homo sapiens X28 region near ALD locus containing dual specificity |
| | | | | | | phosphatase 9 (DUSP9), ribosomal protein L18a (RPL18a), |
| | | | | | | Ca2+/Calmodulin-dependent protein kinase I (CAMKI), creatine |
| | | | | | | transporter (CRTR), CDM protein (CDM), adrenoleukodystrophy |
| | | | | | | protein (AL |
| 9698 | EFF | AA | AG | GG | HS5211110 | Homo sapiens X28 region near ALD locus containing dual specificity |
| | | | | | | phosphatase 9 (DUSP9), ribosomal protein L18a (RPL18a), |
| | | | | | | Ca2+/Calmodulin-dependent protein kinase I (CAMKI), creatine |
| | | | | | | transporter (CRTR), CDM protein (CDM), adrenoleukodystrophy |
| | | | | | | protein (AL |
| 9698 | ADR5 | AA | AG | GG | HS5211110 | Homo sapiens X28 region near ALD locus containing dual specificity |
| | | | | | | phosphatase 9 (DUSP9), ribosomal protein L18a (RPL18a), |
| | | | | | | Ca2+/Calmodulin-dependent protein kinase I (CAMKI), creatine |
| | | | | | | transporter (CRTR), CDM protein (CDM), adrenoleukodystrophy |
| | | | | | | protein (AL |
| 9698 | CVD | AA | AG | GG | HS5211110 | Homo sapiens X28 region near ALD locus containing dual specificity |
| | | | | | | phosphatase 9 (DUSP9), ribosomal protein L18a (RPL18a), |
| | | | | | | Ca2+/Calmodulin-dependent protein kinase I (CAMKI), creatine |
| | | | | | | transporter (CRTR), CDM protein (CDM), adrenoleukodystrophy |
| | | | | | | protein (AL |
| 9849 | CVD | CC | CT | null | X04588 | Human 2.5 kb mRNA for cytoskeletal tropomyosin TM30(nm) |
| 9883 | CVD | AA | AG | GG | BC000140 | PCCA: propionyl Coenzyme A carboxylase, alpha polypeptide |
| 10079 | CVD | AA | AG | GG | X77197 | H. sapiens mRNA for chloride channel |
| 10481 | ADR5 | AA | AT | TT | AF023268 | Homo sapiens clk2 kinase (CLK2), propin1, cote1, glucocerebrosidase |
| | | | | | | (GBA), and metaxin genes, complete cds; metaxin pseudogene |
| | | | | | | and glucocerebrosidase pseudogene; and thrombospondin3 (THBS3) |
| | | | | | | gene, partial cds. |
| 10542 | UEFF | CC | CT | TT | AF066859 | Homo sapiens muscle glycogen phosphorylase (PYGM) mRNA, |
| | | | | | | complete cds. |
| 10542 | ADR5 | CC | CT | TT | AF066859 | Homo sapiens muscle glycogen phosphorylase (PYGM) mRNA, |
| | | | | | | complete cds. |
| 10600 | EFF | AA | AG | GG | AF129756 | Homo sapiens MSH55 gene, partial cds; and CLIC1, DDAH, G6b, |
| | | | | | | G6c, G5b, G6d, G6e, G6f, BAT5, G5b, CSK2B, BAT4, G4, Apo M, |
| | | | | | | BAT3, BAT2, AIF-1, 1C7, LST-1, LTB, TNF, and LTA genes, |
| | | | | | | complete cds. |
| 10621 | CVD | CC | CT | TT | AF220490 | Homo sapiens group III secreted phospholipase A2 mRNA, complete |
| | | | | | | cds. |
| 10745 | ADR5 | AA | AG | GG | D11456 | Human mRNA for Xanthine dehydrogenase, complete cds. |
| 10745 | VEFF | AA | AG | GG | D11456 | Human mRNA for Xanthine dehydrogenase, complete cds. |
| 10747 | ADR | CC | CT | TT | D11456 | Human mRNA for Xanthine dehydrogenase, complete cds. |
| 10747 | CVD | CC | CT | TT | D11456 | Human mRNA for Xanthine dehydrogenase, complete cds. |
| 10747 | ADR3 | CC | CT | TT | D11456 | Human mRNA for Xanthine dehydrogenase, complete cds. |
| 10771 | ADR5 | CC | CG | GG | D37932 | Human mRNA for HPC-1, partial cds. |
| 10771 | EFF | CC | CG | GG | D37932 | Human mRNA for HPC-1, partial cds. |
| 10870 | CVD | AA | AG | GG | AH002776 | LDLR: low density lipoprotein receptor (familial hyper- |
| | | | | | | cholesterolemia) |
| 10877 | CVD | AA | AC | CC | AC005832 | Homo sapiens 12p13.3 BAC RPCI11-500M8 (Roswell Park Cancer |
| | | | | | | Institute Human BAC Library) complete sequence. |
| 10948 | CVD | GG | GT | TT | M10065 | Human apolipoprotein E (epsilon-4 allele) gene, complete cds. |
| 11001 | ADR5 | CC | CT | TT | M34424 | Human acid alpha-glucosidase (GAA) mRNA, complete cds. |
| 11073 | ADR5 | CC | CG | GG | AF070670 | Homo sapiens protein phosphatase 2C alpha 2 mRNA, complete cds. |
| 11153 | CVD | CC | CT | TT | U57623 | Human fatty acid binding protein FABP gene, complete cds. |
| 11210 | CVD | CC | CT | TT | AB014460 | Homo sapiens TSC2, NTHL1/NTH1 and SLC9A3R2/E3KARP |
| | | | | | | genes, partial and complete cds. |
| 11210 | ADR3 | CC | CT | TT | AB014460 | Homo sapiens TSC2, NTHL1/NTH1 and SLC9A3R2/E3KARP |
| | | | | | | genes, partial and complete cds. |
| 11210 | ADR | CC | CT | TT | AB014460 | Homo sapiens TSC2, NTHL1/NTH1 and SLC9A3R2/E3KARP |
| | | | | | | genes, partial and complete cds. |
| 11248 | ADR | CC | CT | TT | X60435 | H. sapiens gene PACAP for pituitary adenylate cyclase activating |
| | | | | | | polypeptide |
| 11248 | CVD | CC | CT | TT | X60435 | H. sapiens gene PACAP for pituitary adenylate cyclase activating |
| | | | | | | polypeptide |
| 11372 | CVD | AA | AG | GG | Z82215 | Human DNA sequence from clone RP1-68O2 on chromosome 22 |
| | | | | | | Contains the 5′ end of the APOL2 gene for apolipoprotein L 2, the |
| | | | | | | APOL gene for apolipoprotein L, the MYH9 gene for nonmuscle type |
| | | | | | | myosin heavy chain 9. ESTs, STSs and GSSs. |
| 11449 | CVD | CC | CG | GG | AF050163 | Homo sapiens lipoprotein lipase precursor, gene, partial cds. |
| 11450 | EFF | AA | AT | TT | AF050163 | Homo sapiens lipoprotein lipase precursor, gene, partial cds. |
| 11470 | CVD | CC | CT | null | AJ006945 | Human P2Y1 gene |
| 11472 | CVD | AA | AT | null | AJ006945 | Human P2Y1 gene |
| 11487 | ADR5 | AT | TT | null | M75106 | Human prepro-plasma carboxypeptidase B mRNA, complete cds. |
| 11487 | ADR3 | AT | TT | null | M75106 | Human prepro-plasma carboxypeptidase B mRNA, complete cds. |
| 11488 | ADR5 | CC | CG | GG | M75106 | Human prepro-plasma carboxypeptidase B mRNA, complete cds. |
| 11488 | UEFF | CC | CG | GG | M75106 | Human prepro-plasma carboxypeptidase B mRNA, complete cds. |
| 11488 | ADR3 | CC | CG | GG | M75106 | Human prepro-plasma carboxypeptidase B mRNA, complete cds. |
| 11493 | CVD | AA | AG | GG | U03882 | Human monocyte chemoattractant protein 1 receptor (MCP-1RA) |
| | | | | | | alternatively spliced mRNA, complete cds. |
| 11502 | ADR3 | CC | CT | TT | U58917 | Homo sapiens IL-17 receptor mRNA, complete cds. |
| 11502 | ADR5 | CC | CT | TT | U58917 | Homo sapiens IL-17 receptor mRNA, complete cds. |
| 11534 | CVD | GG | GT | null | AJ276102 | Homo sapiens mRNA for GPRC5C protein |
| 11537 | CVD | AA | AG | GG | AL022721 | Human DNA sequence from clone 109F14 on chromosome 6p21.2-21.3. |
| | | | | | | Contains the alternatively spliced gene for Transcriptional |
| | | | | | | Enhancer Factor TEF-5, the 60S Ribosomal Protein RPL10A gene, a |
| | | | | | | PUTATIVE ZNF127 LIKE gene, and the PPARD for Peroxisome |
| | | | | | | Proliferato |
| 11537 | EFF | AA | AG | GG | AL022721 | Human DNA sequence from clone 109F14 on chromosome 6p21.2-21.3. |
| | | | | | | Contains the alternatively spliced gene for Transcriptional |
| | | | | | | Enhancer Factor TEF-5, the 60S Ribosomal Protein RPL10A gene, a |
| | | | | | | PUTATIVE ZNF127 LIKE gene, and the PPARD for Peroxisome |
| | | | | | | Proliferato |
| 11560 | EFF | AA | AG | GG | AC006312 | Homo sapiens chromosome 9, clone hRPK.401_G_18, complete |
| | | | | | | sequence. |
| 11578 | CYD | CC | CT | null | AC073593 | Homo sapiens 12 BAC RP11-13J12 (Roswell Park Cancer Institute |
| | | | | | | Human BAC Library) complete sequence. |
| 11594 | ADR3 | CC | CT | TT | AF026069 | Homo sapiens phosphomevalonate kinase (HUMPMKI) gene, partial |
| | | | | | | cds. |
| 11594 | ADR5 | CC | CT | TT | AF026069 | Homo sapiens phosphomevalonate kinase (HUMPMKI) gene, partial |
| | | | | | | cds. |
| 11594 | CVD | CC | CT | TT | AF026069 | Homo sapiens phosphomevalonate kinase (HUMPMKI) gene, partial |
| | | | | | | cds. |
| 11594 | ADR | CC | CT | TT | AF026069 | Homo sapiens phosphomevalonate kinase (HUMPMKI) gene, partial |
| | | | | | | cds. |
| 11624 | CVD | CC | CT | TT | AL022721 | Human DNA sequence from clone 109F14 on chromosome 6p21.2-21.3. |
| | | | | | | Contains the alternatively spliced gene for Transcriptional |
| | | | | | | Enhancer Factor TEF-5, the 60S Ribosomal Protein RPL10 gene, a |
| | | | | | | PUTATIVE ZNF127 LIKE gene, and the PPARD for Peroxisome |
| | | | | | | Proliferato |
| 11624 | EFF | CC | CT | TT | AL022721 | Human DNA sequence from clone 109F14 on chromosome 6p21.2-21.3. |
| | | | | | | Contains the alternatively spliced gene for Transcriptional |
| | | | | | | Enhancer Factor TEF-5, the 60S Ribosomal Protein RPL10A gene, a |
| | | | | | | PUTATIVE ZNF127 LIKE gene, and the PPARD for Peroxisome |
| | | | | | | Proliferato |
| 11627 | CVD | CC | CT | TT | AL022721 | Human DNA sequence from clone 109F14 on chromosome 6p21.2-21.3. |
| | | | | | | Contains the alternatively spliced gene for Transcriptional |
| | | | | | | Enhancer Factor TEF-5, the 60S Ribosomal Protein RPL10A gene, a |
| | | | | | | PUTATIVE ZNFL27 LIKE gene, and the PPARD for Peroxisome |
| | | | | | | Proliferator |
| 11627 | EFF | CC | CT | TT | AL022721 | Human DNA sequence from clone 109F14 on chromosome 6p21.2-21.3. |
| | | | | | | Contains the alternatively spliced gene for Transcriptional |
| | | | | | | Enhancer Factor TEF-5, the 60S Ribosomal Protein RPL10A gene, a |
| | | | | | | PUTATIVE ZNF127 LIKE gene, and the PPARD for Peroxisome |
| | | | | | | Proliferato |
| 11644 | ADR5 | AA | AG | GG | D84371 | Homo sapiens mRNA for serum aryldiakylphosphatase, complete cds. |
| 11650 | EFF | AA | AG | GG | X56668 | Human DNA for calretinin exon 1 |
| 11654 | ADR5 | AA | AG | GG | AJ276180 | Homo sapiens partial ZNF202 gene for zinc finger protein homolog, |
| | | | | | | exon 4 |
| 11654 | ADR3 | AA | AG | GG | AJ276180 | Homo sapiens partial ZNF202 gene for zinc finger protein homolog, |
| | | | | | | exon 4 |
| 11655 | ADR5 | AA | AC | CC | AJ276180 | Homo sapiens partial ZNF202 gene for zinc finger protein homolog, |
| | | | | | | exon 4 |
| 11655 | ADR3 | AA | AC | CC | AJ276180 | Homo sapiens partial ZNF202 gene for zinc finger protein homolog, |
| | | | | | | exon 4 |
| 11656 | CVD | CC | CT | TT | NM_001081 | CUBN: cubilin (intrinsic factor-cobalamin receptor) |
| 11656 | EFF | CC | CT | TT | NM_001081 | CUBN: cubilin (intrinsic factor-cobalamin receptor) |
| 11825 | ADR5 | AA | AG | null | AC008897 | Homo sapiens chromosome 5 clone CTD-2235C13, WORKING |
| | | | | | | DRAFT SEQUENCE, 6 ordered pieces. |
| 11914 | ADR5 | AA | AT | TT | AF030555 | Homo sapiens acyl-CoA synthetase 4 (ACS4) mRNA, complete cds. |
| 12008 | EFF | CC | CT | null | AF107885 | Homo sapiens chromosome 14q24.3 clone BAC270M14 |
| | | | | | | transforming growth factor-beta 3 (TGF-beta 3) gene, complete cds; |
| | | | | | | and unknown genes. |
| 12008 | ADR5 | CC | CT | null | AF107885 | Homo sapiens chromosome 14q24.3 clone BAC270M14 |
| | | | | | | transforming growth factor-beta 3 (TGF-beta 3) gene, complete cds; |
| | | | | | | and unknown genes. |
| 12097 | ADR5 | AG | GG | null | AF280107 | Homo sapiens cytochrome P450 polypeptide 43 (CYP3A43) gene, |
| | | | | | | partial cds; cytochrome P450 polypeptide 4 (CYP3A4) and |
| | | | | | | cytochrome P450 polypeptide 7 (CYP3A7) genes, complete cds; and |
| | | | | | | cytochrome P450 polypeptide 5 (CYP3A5) gene, partial cds. |
| 12097 | ADR3 | AG | GG | null | AF280107 | Homo sapiens cytochrome P450 polypeptide 43 (CYP3A43) gene, |
| | | | | | | partial cds; cytochrome P450 polypeptide 4 (CYP3A4) and |
| | | | | | | cytochrome P450 polypeptide 7 (CYP3A7) genes, complete cds; and |
| | | | | | | cytochrome P450 polypeptide 5 (CYP3A5) gene, partial cds. |
| 12366 | UEFF | AA | AG | GG | D63807 | Human mRNA for lanosterol synthase, complete cds. |
| 12366 | ADR5 | AA | AG | GG | D63807 | Human mRNA for lanosterol synthase, complete cds. |
| 12619 | ADR5 | AG | GG | null | L13744 | Human AF-9 mRNA, complete cds. |
| 13025 | ADR5 | AA | AC | CC | M85168 | Human glycogen debranching enzyme mRNA, complete cds. |
| 13191 | CVD | AA | AG | GG | HSHMGCOAS | H. sapiens mRNA for 3-hydroxy-3-methylglutaryl coenzyme A |
| | | | | | | synthase |
| 13937 | ADR5 | AA | AC | CC | M68840 | Human monoamine oxidase A (MAOA) mRNA, complete cds. |
| 900002 | CVD | GG | GT | TT | AF192304 | Homo sapiens vHNF1-C mRNA |
| 900013 | CVD | CC | CG | GG | L05628 | Human multidrug resistance-associated protein mRNA |
| 900025 | CVD | GG | GT | TT | Z22535 | ALK3 |
| 900032 | CVD | CC | CT | TT | af096786 | GPR-55 |
| 900045 | EFF | CC | CT | TT | X63432 | H. sapiens ACTB mRNA for mutant beta-actin |
| 900065 | CVD | AA | AC | CC | AC009245 | Homo sapiens chromosome 7 clone RP11-351B12, complete |
| | | | | | | sequence |
| 900078 | ADR3 | AA | AG | GG | NM_017460 | CYP3A4 |
| 900078 | ADR5 | AA | AG | GG | NM_017460 | CYP3A4 |
| 900082 | ADR3 | AA | AG | GG | NM_002489 | NADH dehydrogenase (ubiquinone) 1, alpha subcomplex, 4 (9 kD, |
| | | | | | | MLRQ), NDUFA4 |
| 900082 | ADR5 | AA | AG | GG | NM_002489 | NADH dehydrogenase (ubiquinone) 1, alpha subcomplex, 4 (9 kD, |
| | | | | | | MLRQ), NDUFA4 |
| 900096 | CVD | AA | AG | GG | NM_003376 | VEGF |
| 900107 | ADR5 | CC | CT | TT | NM_033013 | nuclear receptor subfamily 1, group I, member 2 (NR1I2) |
| 900115 | ADR5 | AA | AG | GG | ATP2A1 | ATPase, Ca++ transporting, cardiac muscle, fast twitch 1 |
| 900115 | EFF | AA | AG | GG | ATP2A1 | ATPase, Ca++ transporting, cardiac muscle, fast twitch 1 |
| 900121 | ADR | GG | GT | TT | NM_016156 | MTMR2 myotubularin related protein 2 (MTMR2) |
| 900173 | CVD | GG | GT | TT | M76722 | LPL: lipoprotein lipase |
| 10000002 | EFF | AA | AG | GG | M32992 | Cholesteryl ester transfer protein (CETP) |
| 10000006 | CVD | AA | AG | GG | NM_000384 | Apolipoprotein B |
| 10000014 | CVD | AA | AC | CC | M61888 | E-Selectin (CD62E) |
| 10000025 | CVD | CC | CT | TT | AC073593 | Scavenger receptor class B type I |

Null: not defined.

TABLE 4
Cohorts
Given are names (as used in table 5) and formations of the various cohorts that were used for genotyping

| COHORT | Definition |
|---|---|
| HELD_ALL_GOOD/BAD | Healthy elderly individuals of both genders with good or bad serum lipid profiles (as defined in table 1a) |
| HELD_FEM_GOOD/BAD | Healthy elderly individuals (female) with good or bad serum lipid profiles (as defined in table 1a) |
| HELD_MAL_GOOD/BAD | Healthy elderly individuals (male) with good or bad serum lipid profiles (as defined in table 1a) |
| CVD_ALL_CASE/CTRL | Individuals with diagnosis of cardiovascular disease and healthy controls (both genders) |
| CVD_FEM_CASE/CTRL | Individuals with diagnosis of cardiovascular disease and healthy controls (female) |
| CVD_MAL_CASE/CTRL | Individuals with diagnosis of cardiovascular disease and healthy controls (male) |
| HELD_FEM_ADRCTRL | Female individuals that tolerate adminstration of cerivastatin without exhibiting signs of ADR (as defined in table 1b) |
| HELD_FEM_ADRCASE | Female individuals that exhibited ADR (as defined in table 1b) upon administration of cerivastatin |
| HELD_MAL_ADRCTRL | Male individuals that tolerate adminstration of cerivastatin without exhibiting signs of ADR (as defined in table 1b) |
| HELD_MAL_ADRCASE | Male individuals that exhibited ADR (as defined in table 1b) upon administration of cerivastatin |
| HELD_ALL_ADRCTRL | Individuals of both genders that tolerate adminstration of cerivastatin without exhibiting signs of ADR |
| | (as defined in table 1b) |
| HELD_ALL_ADRCASE | Individuals of both genders that exhibited ADR (as defined in table 1b) upon administration of cerivastatin |
| HELD_FEM_LORESP | Female individuals with a minor response to cerivastatin administration (as defined in table 1b) |
| HELD_FEM_HIRESP | Female individuals with a high response to to cerivastatin administration (as defined in table 1b) |
| HELD_FEM_HIHDL/LOHDL | Healthy elderly individuals (female) with high or low serum HDL cholesterol levels (as defined in table 1c) |
| HELD_MAL_HIHDL/LOHDL | Healthy elderly individuals (male) with high or low serum HDL cholesterol levels (as defined in table 1c) |
| HELD_ALL_HIHDL/LOHDL | Healthy elderly individuals of both genders with high or low serum HDL cholesterol levels (as defined in table 1c) |
| HELD_FEM_ADR3CASE | Female individuals that exhibited advanced ADR (as defined in table 1b) upon administration of cerivastatin |
| HELD_MAL_ADR3CASE | Male individuals that exhibited advanced ADR (as defined in table 1b) upon administration of cerivastatin |
| HELD_ALL_ADR3CASE | Individuals of both genders that exhibited advanced ADR (as defined in table 1b) upon administration of cerivastatin |
| HELD_FEM_VLORESP | Female individuals with a very low response to cerivastatin administration (as defined in table 1b) |
| HELD_FEM_VHIRESP | Female individuals with a very high response to cerivastatin administration (as defined in table 1b) |
| HELD_FEM_ADR5CASE | Female individuals that exhibited severe ADR (as defined in table 1b) upon administration of cerivastatin |
| HELD_MAL_ADR5CASE | Male individuals that exhibited severe ADR (as defined in table 1b) upon administration of cerivastatin |
| HELD_ALL_ADR5CASE | Individuals of both genders that exhibited severe ADR (as defined in table 1b) upon administration of cerivastatin |
| HELD_FEM_ULORESP | Female individuals with a ultra low response to cerivastatin administration (as defined in table 1b) |
| HELD_FEM_UHIRESP | Female individuals with a ultra high response to to cerivastatin administration (as defined in table 1b) |

Table 5a and 5b Cohort Sizes and p-values of PA SNPs

The baySNP number refers to an internal numbering of the PA SNPs. Cpval denotes the classical Pearson chi-squared test, Xpval denotes the exact version of Pearson's chi-squared test, LRpval denotes the likelihood-ratio chi-squared test, Cpvalue, Xpvalue, and LRpvalue are calculated as described in (SAS/STAT User's Guide of the SAS OnlineDoc, Version 8), (L. D. Fisher and G. van Belle, Biostatistics, Wiley Interscience 1993), and (A. Agresti, Statistical Science 7, 131 (1992)). The GTYPE and Allele p values were obtained through the respective chi square tests when comparing COHORTs A and B. For GTYPE p value the number of patients in cohort A carrying genotypes 11, 12 or 22 (FQ11 A, FQ 12 A, FQ 22 A; genotypes as defined in table 3) were compared with the respective patients in cohort B (FQ11 B, FQ 12 B, FQ 22 B; genotypes as defined in table 3) resulting in the respective chi square test with a 3×2 matrix. For Allele p values we compared the allele count of alleles 1 and 2 (A1 and A2) in cohorts A and B, respectively (chi square test with a 2×2 matrix). SIZE A and B: Number of patients in cohorts A and B, respectively. See table 4 for definition of COHORTs A and B.

TABLE 5a
Cohort sizes and frequency of alleles and genotypes

| baySNP | A1 | A2 | COHORT A | SIZE A | FQ1 A | FQ2 A | FQ11 A | FQ12 A | FQ22 A | COHORT B | SIZE B | FQ1 B | FQ2 B | FQ11 B | FQ12 B | FQ22 B |
|---|---|---|---|---|---|---|---|---|---|---|---|---|---|---|---|---|
| 28 | C | T | HELD_FEM_HIRESP | 12 | 4 | 20 | 1 | 2 | 9 | HELD_FEM_LORESP | 22 | 18 | 26 | 3 | 12 | 7 |
| 29 | A | G | HELD_ALL_LOHDL | 10 | 12 | 8 | 4 | 4 | 2 | HELD_ALL_HIHDL | 15 | 7 | 23 | 0 | 7 | 8 |
| 29 | A | G | HELD_MAL_ADRCASE3ULN | 26 | 33 | 19 | 13 | 7 | 6 | HELD_MAL_ADRCTRL | 72 | 68 | 76 | 18 | 32 | 22 |
| 29 | A | G | HELD_MAL_ADRCASE5ULN | 9 | 13 | 5 | 5 | 3 | 1 | HELD_MAL_ADRCTRL | 72 | 68 | 76 | 18 | 32 | 22 |
| 52 | C | G | HELD_FEM_HIRESP | 18 | 24 | 12 | 7 | 10 | 1 | HELD_FEM_LORESP | 31 | 27 | 35 | 5 | 17 | 9 |
| 56 | A | G | HELD_FEM_HIRESP | 12 | 5 | 19 | 0 | 5 | 7 | HELD_FEM_LORESP | 22 | 2 | 42 | 0 | 2 | 20 |
| 89 | A | G | HELD_ALL_CASE | 45 | 90 | 0 | 45 | 0 | 0 | HELD_ALL_CTRL | 40 | 77 | 3 | 37 | 3 | 0 |
| 90 | C | T | HELD_FEM_CASE | 31 | 29 | 33 | 8 | 13 | 10 | HELD_FEM_CTRL | 22 | 27 | 17 | 6 | 15 | 1 |
| 99 | C | T | HELD_FEM_BAD | 82 | 54 | 110 | 13 | 28 | 41 | HELD_FEM_GOOD | 80 | 51 | 109 | 5 | 41 | 34 |
| 140 | C | T | HELD_FEM_HIRESP | 12 | 24 | 0 | 0 | 0 | 12 | HELD_FEM_LORESP | 21 | 4 | 38 | 1 | 2 | 18 |
| 152 | A | G | HELD_FEM_HIRESP | 12 | 12 | 12 | 3 | 6 | 3 | HELD_FEM_LORESP | 22 | 33 | 11 | 12 | 9 | 1 |
| 214 | A | G | HELD_ALL_BAD | 97 | 156 | 38 | 59 | 38 | 0 | HELD_ALL_GOOD | 113 | 182 | 44 | 73 | 36 | 4 |
| 214 | A | G | HELD_FEM_BAD | 81 | 131 | 31 | 50 | 31 | 0 | HELD_FEM_GOOD | 78 | 122 | 34 | 48 | 26 | 4 |
| 221 | C | G | HELD_ALL_CASE | 45 | 26 | 64 | 7 | 12 | 26 | HELD_ALL_CTRL | 39 | 27 | 51 | 3 | 21 | 15 |
| 221 | C | G | HELD_FEM_CASE | 31 | 17 | 45 | 4 | 9 | 18 | HELD_FEM_CTRL | 22 | 18 | 26 | 2 | 14 | 6 |
| 224 | C | T | HELD_FEM_BAD | 79 | 110 | 48 | 51 | 8 | 20 | HELD_FEM_GOOD | 79 | 125 | 33 | 60 | 5 | 14 |
| 224 | C | T | HELD_MAL_BAD | 20 | 35 | 5 | 17 | 1 | 2 | HELD_MAL_GOOD | 37 | 51 | 23 | 25 | 1 | 11 |
| 294 | C | T | HELD_ALL_CASE | 45 | 56 | 34 | 16 | 24 | 5 | HELD_ALL_CTRL | 40 | 58 | 22 | 18 | 22 | 0 |
| 307 | C | T | CVD_FEM_CASE | 36 | 19 | 53 | 2 | 15 | 19 | CVD_FEM_CTRL | 38 | 38 | 38 | 9 | 20 | 9 |
| 307 | C | T | HELD_ALL_BAD | 102 | 70 | 134 | 0 | 70 | 32 | HELD_ALL_GOOD | 117 | 63 | 171 | 0 | 63 | 54 |
| 411 | A | T | HELD_ALL_LOHDL | 10 | 17 | 3 | 7 | 3 | 0 | HELD_ALL_HIHDL | 15 | 18 | 12 | 5 | 8 | 2 |
| 449 | C | G | HELD_MAL_BAD | 20 | 3 | 37 | 0 | 3 | 17 | HELD_MAL_GOOD | 37 | 16 | 58 | 1 | 14 | 22 |
| 466 | C | T | CVD_FEM_CASE | 35 | 27 | 43 | 6 | 15 | 14 | CVD_FEM_CTRL | 40 | 44 | 36 | 12 | 20 | 8 |
| 472 | A | G | HELD_FEM_HIRESP | 11 | 22 | 0 | 0 | 0 | 11 | HELD_FEM_LORESP | 22 | 12 | 32 | 3 | 6 | 13 |
| 542 | A | G | HELD_MAL_CASE | 14 | 12 | 16 | 2 | 8 | 4 | HELD_MAL_CTRL | 19 | 2 | 36 | 0 | 2 | 17 |
| 542 | A | G | HELD_MAL_LOHDL | 21 | 14 | 28 | 3 | 8 | 10 | HELD_MAL_HIHDL | 27 | 3 | 51 | 0 | 3 | 24 |
| 542 | A | G | HELD_ALL_ADRCASE | 159 | 53 | 265 | 0 | 53 | 106 | HELD_ALL_ADRCTRL | 154 | 37 | 271 | 2 | 33 | 119 |
| 542 | A | G | HELD_FEM_LOHDL | 23 | 2 | 44 | 0 | 2 | 21 | HELD_FEM_HIHDL | 32 | 10 | 54 | 1 | 8 | 23 |
| 739 | C | G | HELD_ALL_CASE | 45 | 39 | 51 | 9 | 21 | 15 | HELD_ALL_CTRL | 40 | 48 | 32 | 14 | 20 | 6 |
| 821 | A | C | HELD_MAL_BAD2 | 309 | 180 | 438 | 32 | 116 | 161 | HELD_MAL_GOOD2 | 349 | 174 | 524 | 18 | 138 | 193 |
| 821 | A | C | HELD_FEM_VHIRESP | 10 | 4 | 16 | 0 | 4 | 6 | HELD_FEM_VLORESP | 14 | 14 | 14 | 4 | 6 | 4 |
| 1005 | A | G | HELD_MAL_CASE | 14 | 26 | 2 | 12 | 2 | 0 | HELD_MAL_CTRL | 18 | 27 | 9 | 11 | 5 | 2 |
| 1055 | A | T | HELD_MAL_CASE | 9 | 3 | 15 | 0 | 3 | 6 | HELD_MAL_CTRL | 12 | 8 | 16 | 4 | 0 | 8 |
| 1056 | A | G | HELD_FEM_HIRESP | 24 | 30 | 18 | 12 | 6 | 6 | HELD_FEM_LORESP | 33 | 41 | 25 | 10 | 21 | 2 |
| 1085 | A | G | HELD_MAL_BAD | 20 | 17 | 23 | 3 | 11 | 6 | HELD_MAL_GOOD | 36 | 46 | 26 | 15 | 16 | 5 |
| 1085 | A | G | CVD_FEM_CASE | 34 | 51 | 17 | 20 | 11 | 3 | CVD_FEM_CTRL | 40 | 47 | 33 | 16 | 15 | 9 |
| 1086 | A | G | HELD_MAL_BAD | 20 | 24 | 16 | 7 | 10 | 3 | HELD_MAL_GOOD | 36 | 28 | 44 | 5 | 18 | 13 |
| 1092 | C | G | HELD_MAL_BAD | 20 | 9 | 31 | 2 | 5 | 13 | HELD_MAL_GOOD | 37 | 29 | 45 | 4 | 21 | 12 |
| 1096 | G | T | HELD_MAL_CASE | 14 | 7 | 21 | 0 | 7 | 7 | HELD_MAL_CTRL | 18 | 3 | 33 | 0 | 3 | 15 |
| 1096 | G | T | CVD_MAL_CASE | 69 | 21 | 117 | 4 | 13 | 52 | CVD_MAL_CTRL | 33 | 12 | 54 | 0 | 12 | 21 |
| 1101 | C | T | HELD_FEM_HIRESP | 12 | 24 | 0 | 12 | 0 | 0 | HELD_FEM_LORESP | 22 | 40 | 4 | 18 | 4 | 0 |
| 1204 | A | G | HELD_MAL_BAD | 19 | 12 | 26 | 2 | 8 | 9 | HELD_MAL_GOOD | 35 | 9 | 61 | 0 | 9 | 26 |
| 1204 | A | G | HELD_ALL_BAD | 99 | 62 | 136 | 12 | 38 | 49 | HELD_ALL_GOOD | 115 | 52 | 178 | 8 | 36 | 71 |
| 1504 | C | T | HELD_ALL_CASE | 44 | 37 | 51 | 5 | 27 | 12 | HELD_ALL_CTRL | 39 | 36 | 42 | 12 | 12 | 15 |
| 1504 | C | T | HELD_MAL_BAD | 19 | 12 | 26 | 0 | 12 | 7 | HELD_MAL_GOOD | 37 | 33 | 41 | 8 | 17 | 12 |
| 1504 | C | T | HELD_MAL_CASE | 14 | 13 | 15 | 2 | 9 | 3 | HELD_MAL_CTRL | 18 | 12 | 24 | 4 | 4 | 10 |
| 1504 | C | T | HELD_FEM_CASE | 30 | 24 | 36 | 3 | 18 | 9 | HELD_FEM_CTRL | 21 | 24 | 18 | 8 | 8 | 5 |
| 1511 | G | T | HELD_FEM_HIRESP | 12 | 15 | 9 | 3 | 9 | 0 | HELD_FEM_LORESP | 22 | 35 | 9 | 14 | 7 | 1 |
| 1524 | A | C | HELD_FEM_ADRCASE3ULN | 38 | 16 | 60 | 0 | 16 | 22 | HELD_FEM_ADRCTRL | 82 | 39 | 125 | 8 | 23 | 51 |
| 1556 | C | G | HELD_FEM_HIRESP | 12 | 7 | 17 | 0 | 7 | 5 | HELD_FEM_LORESP | 22 | 3 | 41 | 0 | 3 | 19 |
| 1561 | A | C | CVD_FEM_CASE | 36 | 58 | 14 | 23 | 12 | 1 | CVD_FEM_CTRL | 40 | 53 | 27 | 17 | 19 | 4 |
| 1582 | C | T | HELD_MAL_BAD | 20 | 5 | 35 | 0 | 5 | 15 | HELD_MAL_GOOD | 37 | 22 | 52 | 5 | 12 | 20 |
| 1638 | A | G | HELD_FEM_CASE | 31 | 10 | 52 | 1 | 8 | 22 | HELD_FEM_CTRL | 22 | 15 | 29 | 2 | 11 | 9 |
| 1653 | G | T | CVD_MAL_CASE | 69 | 70 | 68 | 15 | 40 | 14 | CVD_MAL_CTRL | 33 | 30 | 36 | 10 | 10 | 13 |
| 1662 | C | T | HELD_MAL_CASE | 14 | 8 | 20 | 4 | 0 | 10 | HELD_MAL_CTRL | 18 | 36 | 0 | 0 | 0 | 18 |
| 1714 | A | G | CVD_MAL_CASE | 66 | 32 | 100 | 3 | 26 | 37 | CVD_MAL_CTRL | 34 | 26 | 42 | 6 | 14 | 14 |
| 1722 | C | T | HELD_FEM_ADRCASE5ULN | 18 | 21 | 15 | 8 | 5 | 5 | HELD_FEM_ADRCTRL | 81 | 71 | 91 | 14 | 43 | 24 |
| 1757 | A | G | HELD_FEM_HIRESP | 20 | 15 | 25 | 4 | 7 | 9 | HELD_FEM_LORESP | 32 | 16 | 48 | 0 | 16 | 16 |
| 1765 | A | G | HELD_ALL_ADRCASE3ULN | 63 | 9 | 117 | 1 | 7 | 55 | HELD_ALL_ADRCTRL | 149 | 56 | 242 | 4 | 48 | 97 |
| 1765 | A | G | HELD_ALL_ADRCASE3ULN | 63 | 9 | 117 | 1 | 7 | 55 | HELD_ALL_ADRCTRL | 149 | 56 | 242 | 4 | 48 | 97 |
| 1765 | A | G | HELD_ALL_ADRCASE5ULN | 27 | 3 | 51 | 0 | 3 | 24 | HELD_ALL_ADRCTRL | 149 | 56 | 242 | 4 | 48 | 97 |
| 1765 | A | G | HELD_ALL_ADRCASE5ULN | 27 | 3 | 51 | 0 | 3 | 24 | HELD_ALL_ADRCTRL | 149 | 56 | 242 | 4 | 48 | 97 |
| 1765 | A | G | HELD_MAL_ADRCASE3ULN | 26 | 2 | 50 | 0 | 2 | 24 | HELD_MAL_ADRCTRL | 70 | 25 | 115 | 2 | 21 | 47 |
| 1765 | A | G | HELD_MAL_ADRCASE3ULN | 26 | 2 | 50 | 0 | 2 | 24 | HELD_MAL_ADRCTRL | 70 | 25 | 115 | 2 | 21 | 47 |
| 1765 | A | G | HELD_MAL_ADRCASE5ULN | 10 | 20 | 0 | 0 | 0 | 10 | HELD_MAL_ADRCTRL | 70 | 25 | 115 | 2 | 21 | 47 |
| 1765 | A | G | HELD_MAL_ADRCASE5ULN | 10 | 20 | 0 | 0 | 0 | 10 | HELD_MAL_ADRCTRL | 70 | 25 | 115 | 2 | 21 | 47 |
| 1765 | A | G | HELD_FEM_ADRCASE3ULN | 37 | 7 | 67 | 1 | 5 | 31 | HELD_FEM_ADRCTRL | 79 | 31 | 127 | 2 | 27 | 50 |
| 1765 | A | G | HELD_FEM_ADRCASE3ULN | 37 | 7 | 67 | 1 | 5 | 31 | HELD_FEM_ADRCTRL | 79 | 31 | 127 | 2 | 27 | 50 |
| 1776 | A | G | HELD_ALL_CASE | 45 | 90 | 0 | 45 | 0 | 0 | HELD_ALL_CTRL | 40 | 74 | 6 | 37 | 0 | 3 |
| 1776 | A | G | HELD_FEM_CASE | 31 | 62 | 0 | 31 | 0 | 0 | HELD_FEM_CTRL | 22 | 40 | 4 | 20 | 0 | 2 |
| 1799 | C | T | HELD_FEM_BAD2 | 291 | 365 | 217 | 123 | 119 | 49 | HELD_FEM_GOOD2 | 356 | 468 | 244 | 145 | 178 | 33 |
| 1799 | C | T | HELD_MAL_CASE | 14 | 15 | 13 | 4 | 7 | 3 | HELD_MAL_CTRL | 18 | 28 | 8 | 11 | 6 | 1 |
| 1806 | A | G | HELD_FEM_HIRESP | 12 | 23 | 1 | 11 | 1 | 0 | HELD_FEM_LORESP | 22 | 34 | 10 | 14 | 6 | 2 |
| 1837 | C | T | HELD_FEM_BAD2 | 304 | 436 | 172 | 164 | 108 | 32 | HELD_FEM_GOOD2 | 355 | 499 | 211 | 166 | 167 | 22 |
| 1837 | C | T | HELD_ALL_BAD2 | 607 | 891 | 323 | 334 | 223 | 50 | HELD_ALL_GOOD2 | 682 | 952 | 412 | 322 | 308 | 52 |
| 1837 | C | T | HELD_ALL_ADRCASE5ULN | 28 | 46 | 10 | 20 | 6 | 2 | HELD_ALL_ADRCTRL | 155 | 208 | 102 | 66 | 76 | 13 |
| 1837 | C | T | HELD_MAL_ADRCASE | 77 | 107 | 47 | 37 | 33 | 7 | HELD_MAL_ADRCTRL | 72 | 86 | 58 | 21 | 44 | 7 |
| 1837 | C | T | HELD_MAL_BAD2 | 303 | 455 | 151 | 170 | 115 | 18 | HELD_MAL_GOOD2 | 327 | 453 | 201 | 156 | 141 | 30 |
| 1870 | C | T | HELD_ALL_CASE | 45 | 29 | 61 | 2 | 25 | 18 | HELD_ALL_CTRL | 39 | 16 | 62 | 3 | 10 | 26 |
| 1870 | C | T | HELD_FEM_CASE | 31 | 22 | 40 | 1 | 20 | 10 | HELD_FEM_CTRL | 22 | 9 | 35 | 1 | 7 | 14 |
| 1882 | C | T | CVD_MAL_CASE | 69 | 79 | 59 | 21 | 37 | 11 | CVD_MAL_CTRL | 34 | 43 | 25 | 9 | 25 | 0 |
| 1988 | C | T | HELD_ALL_BAD | 100 | 143 | 57 | 52 | 39 | 9 | HELD_ALL_GOOD | 116 | 144 | 88 | 48 | 48 | 20 |
| 2000 | C | T | CVD_MAL_CASE | 70 | 136 | 4 | 68 | 2 | 0 | CVD_MAL_CTRL | 34 | 58 | 10 | 29 | 5 | 0 |
| 2000 | C | T | CVD_ALL_CASE | 105 | 202 | 8 | 101 | 4 | 0 | CVD_ALL_CTRL | 74 | 130 | 18 | 65 | 9 | 0 |
| 2000 | C | T | HELD_FEM_CASE2 | 46 | 90 | 2 | 45 | 1 | 0 | HELD_FEM_CTRL2 | 42 | 74 | 10 | 37 | 5 | 0 |
| 2000 | C | T | HELD_MAL_LOHDL | 20 | 40 | 0 | 20 | 0 | 0 | HELD_MAL_HIHDL | 22 | 40 | 4 | 20 | 2 | 0 |
| 2000 | C | T | HELD_FEM_ADRCASE | 79 | 154 | 4 | 77 | 2 | 0 | HELD_FEM_ADRCTRL | 82 | 152 | 12 | 76 | 6 | 0 |
| 2000 | C | T | HELD_MAL_CASE | 14 | 22 | 6 | 11 | 3 | 0 | HELD_MAL_CTRL | 19 | 36 | 2 | 18 | 1 | 0 |
| 2071 | A | G | CVD_ALL_CASE | 102 | 80 | 124 | 14 | 52 | 36 | CVD_ALL_CTRL | 74 | 42 | 106 | 4 | 34 | 36 |
| 2078 | G | T | HELD_MAL_BAD | 18 | 13 | 23 | 1 | 11 | 6 | HELD_MAL_GOOD | 35 | 13 | 57 | 0 | 13 | 22 |
| 2085 | G | T | HELD_FEM_VHIRESP | 10 | 16 | 4 | 6 | 4 | 0 | HELD_FEM_VLORESP | 14 | 13 | 15 | 3 | 7 | 4 |
| 2095 | A | G | CVD_ALL_CASE | 105 | 4 | 206 | 4 | 101 | 0 | CVD_ALL_CTRL | 73 | 146 | 0 | 0 | 73 | 0 |
| 2119 | A | G | HELD_MAL_BAD | 20 | 23 | 17 | 3 | 17 | 0 | HELD_MAL_GOOD | 37 | 53 | 21 | 16 | 21 | 0 |
| 2119 | A | G | HELD_ALL_BAD | 102 | 131 | 73 | 29 | 73 | 0 | HELD_ALL_GOOD | 117 | 166 | 68 | 49 | 68 | 0 |
| 2119 | A | G | HELD_FEM_HIRESP | 12 | 15 | 9 | 3 | 9 | 0 | HELD_FEM_LORESP | 22 | 35 | 9 | 13 | 9 | 0 |
| 2141 | A | G | HELD_FEM_HIRESP | 12 | 6 | 18 | 0 | 6 | 6 | HELD_FEM_LORESP | 22 | 6 | 38 | 2 | 2 | 18 |
| 2141 | A | G | HELD_ALL_CASE | 45 | 17 | 73 | 0 | 17 | 28 | HELD_ALL_CTRL | 39 | 15 | 63 | 3 | 9 | 27 |
| 2182 | A | G | HELD_FEM_HIRESP | 12 | 18 | 6 | 6 | 6 | 0 | HELD_FEM_LORESP | 21 | 16 | 26 | 1 | 14 | 6 |
| 2234 | G | T | HELD_MAL_BAD | 20 | 10 | 30 | 0 | 10 | 10 | HELD_MAL_GOOD | 35 | 32 | 38 | 7 | 18 | 10 |
| 2281 | A | C | HELD_FEM_VHIRESP | 9 | 5 | 13 | 0 | 5 | 4 | HELD_FEM_VLORESP | 13 | 15 | 11 | 4 | 7 | 2 |
| 2298 | A | C | CVD_FEM_CASE | 35 | 18 | 52 | 4 | 10 | 21 | CVD_FEM_CTRL | 38 | 20 | 56 | 0 | 20 | 18 |
| 2298 | A | C | HELD_MAL_CASE2 | 29 | 8 | 50 | 0 | 8 | 21 | HELD_MAL_CTRL2 | 28 | 16 | 40 | 2 | 12 | 14 |
| 2341 | C | T | HELD_FEM_CASE | 31 | 6 | 56 | 0 | 6 | 25 | HELD_FEM_CTRL | 22 | 44 | 0 | 0 | 0 | 22 |
| 2357 | A | G | HELD_ALL_CASE2 | 74 | 28 | 120 | 5 | 18 | 51 | HELD_ALL_CTRL2 | 71 | 25 | 117 | 0 | 25 | 46 |
| 2357 | A | G | HELD_ALL_CASE | 45 | 16 | 74 | 4 | 8 | 33 | HELD_ALL_CTRL | 40 | 14 | 66 | 0 | 14 | 26 |
| 2357 | A | G | HELD_MAL_BAD | 20 | 4 | 36 | 0 | 4 | 16 | HELD_MAL_GOOD | 36 | 17 | 55 | 0 | 17 | 19 |
| 2357 | A | G | HELD_FEM_CASE | 31 | 12 | 50 | 4 | 4 | 23 | HELD_FEM_CTRL | 22 | 7 | 37 | 0 | 7 | 15 |
| 2366 | G | T | CVD_FEM_CASE | 33 | 38 | 28 | 12 | 14 | 7 | CVD_FEM_CTRL | 40 | 31 | 49 | 8 | 15 | 17 |
| 2423 | A | G | CVD_FEM_CASE | 33 | 45 | 21 | 16 | 13 | 4 | CVD_FEM_CTRL | 39 | 38 | 40 | 12 | 14 | 13 |
| 2708 | C | T | CVD_FEM_CASE | 29 | 57 | 1 | 28 | 1 | 0 | CVD_FEM_CTRL | 40 | 73 | 7 | 33 | 7 | 0 |
| 2995 | A | C | HELD_FEM_ADRCASE5ULN | 18 | 16 | 20 | 3 | 10 | 5 | HELD_FEM_ADRCTRL | 82 | 45 | 119 | 4 | 37 | 41 |
| 2995 | A | C | HELD_FEM_UHIRESP | 54 | 24 | 84 | 2 | 20 | 32 | HELD_FEM_ULORESP | 75 | 50 | 100 | 5 | 40 | 30 |
| 3360 | G | T | HELD_MAL_ADRCASE5ULN | 10 | 20 | 0 | 10 | 0 | 0 | HELD_MAL_ADRCTRL | 73 | 122 | 24 | 50 | 22 | 1 |
| 3464 | A | G | HELD_ALL_CASE | 45 | 21 | 69 | 3 | 15 | 27 | HELD_ALL_CTRL | 40 | 35 | 45 | 9 | 17 | 14 |
| 3464 | A | G | HELD_FEM_CASE | 31 | 13 | 49 | 3 | 7 | 21 | HELD_FEM_CTRL | 22 | 19 | 25 | 5 | 9 | 8 |
| 3689 | C | G | HELD_FEM_HIRESP | 6 | 9 | 3 | 3 | 3 | 0 | HELD_FEM_LORESP | 14 | 10 | 18 | 1 | 8 | 5 |
| 3975 | A | C | HELD_FEM_UHIRESP | 56 | 28 | 84 | 2 | 24 | 30 | HELD_FEM_ULORESP | 75 | 58 | 92 | 10 | 38 | 27 |
| 3976 | A | G | HELD_FEM_UHIRESP | 56 | 28 | 84 | 2 | 24 | 30 | HELD_FEM_ULORESP | 75 | 57 | 93 | 11 | 35 | 29 |
| 4206 | A | T | HELD_FEM_ADRCASE3ULN | 37 | 36 | 38 | 8 | 20 | 9 | HELD_FEM_ADRCTRL | 83 | 103 | 63 | 31 | 41 | 11 |
| 4838 | A | G | HELD_FEM_VHIRESP | 10 | 16 | 4 | 7 | 2 | 1 | HELD_FEM_VLORESP | 14 | 14 | 14 | 3 | 8 | 3 |
| 4838 | A | G | HELD_FEM_VHIRESP | 10 | 16 | 4 | 7 | 2 | 1 | HELD_FEM_VLORESP | 14 | 14 | 14 | 3 | 8 | 3 |
| 4838 | A | G | HELD_FEM_VHIRESP | 10 | 16 | 4 | 7 | 2 | 1 | HELD_FEM_VLORESP | 14 | 14 | 14 | 3 | 8 | 3 |
| 4912 | A | G | HELD_FEM_HIRESP | 12 | 14 | 10 | 7 | 0 | 5 | HELD_FEM_LORESP | 20 | 12 | 28 | 5 | 2 | 13 |
| 4925 | A | C | HELD_MAL_CASE | 14 | 21 | 7 | 7 | 7 | 0 | HELD_MAL_CTRL | 18 | 33 | 3 | 15 | 3 | 0 |
| 4966 | A | G | HELD_MAL_ADRCASE3ULN | 26 | 22 | 30 | 7 | 8 | 11 | HELD_MAL_ADRCTRL | 72 | 77 | 67 | 18 | 41 | 13 |
| 5014 | A | G | HELD_ALL_ADRCASE5ULN | 28 | 8 | 48 | 3 | 2 | 23 | HELD_ALL_ADRCTRL | 152 | 77 | 227 | 10 | 57 | 85 |
| 5014 | A | G | HELD_FEM_ADRCASE5ULN | 18 | 5 | 31 | 2 | 1 | 15 | HELD_FEM_ADRCTRL | 81 | 37 | 125 | 5 | 27 | 49 |
| 5296 | A | G | CVD_FEM_CASE | 36 | 10 | 62 | 0 | 10 | 26 | CVD_FEM_CTRL | 40 | 4 | 76 | 0 | 4 | 36 |
| 5296 | A | G | HELD_FEM_HIRESP | 12 | 3 | 21 | 1 | 1 | 10 | HELD_FEM_LORESP | 22 | 9 | 35 | 0 | 9 | 13 |
| 5296 | A | G | CVD_ALL_CASE | 104 | 27 | 181 | 1 | 25 | 78 | CVD_ALL_CTRL | 74 | 10 | 138 | 0 | 10 | 64 |
| 5298 | C | T | HELD_FEM_HIRESP | 11 | 3 | 19 | 1 | 1 | 9 | HELD_FEM_LORESP | 22 | 9 | 35 | 0 | 9 | 13 |
| 5298 | C | T | CVD_ALL_CASE | 101 | 28 | 174 | 3 | 22 | 76 | CVD_ALL_CTRL | 74 | 10 | 138 | 0 | 10 | 64 |
| 5298 | C | T | CVD_FEM_CASE | 35 | 10 | 60 | 1 | 8 | 26 | CVD_FEM_CTRL | 40 | 4 | 76 | 0 | 4 | 36 |
| 5320 | A | G | HELD_FEM_HIRESP | 19 | 12 | 26 | 1 | 10 | 8 | HELD_FEM LORESP | 33 | 37 | 29 | 9 | 19 | 5 |
| 5361 | A | C | CVD_MAL_CASE | 64 | 53 | 75 | 24 | 5 | 35 | CVD_MAL_CTRL | 32 | 36 | 28 | 18 | 0 | 14 |
| 5457 | A | G | HELD_FEM_HIRESP | 12 | 2 | 22 | 1 | 0 | 11 | HELD_FEM_LORESP | 21 | 8 | 34 | 1 | 6 | 14 |
| 5704 | C | T | HELD_MAL_BAD | 20 | 10 | 30 | 1 | 8 | 11 | HELD_MAL_GOOD | 37 | 32 | 42 | 3 | 26 | 8 |
| 5704 | C | T | CVD_MAL_CASE | 68 | 40 | 96 | 5 | 30 | 33 | CVD_MAL_CTRL | 33 | 30 | 36 | 6 | 18 | 9 |
| 5717 | A | G | HELD_FEM_ADRCASE3ULN | 38 | 50 | 26 | 17 | 16 | 5 | HELD_FEM_ADRCTRL | 83 | 83 | 83 | 21 | 41 | 21 |
| 5717 | A | G | HELD_ALL_ADRCASE3ULN | 65 | 74 | 56 | 21 | 32 | 12 | HELD_ALL_ADRCTRL | 156 | 144 | 168 | 34 | 76 | 46 |
| 5959 | A | G | HELD_ALL_CASE | 43 | 52 | 34 | 16 | 20 | 7 | HELD_ALL_CTRL | 38 | 29 | 47 | 4 | 21 | 13 |
| 5959 | A | G | CVD_FEM_CASE | 9 | 12 | 6 | 4 | 4 | 1 | CVD_FEM_CTRL | 13 | 7 | 19 | 0 | 7 | 6 |
| 5959 | A | G | HELD_MAL_CASE | 14 | 15 | 13 | 4 | 7 | 3 | HELD_MAL_CTRL | 17 | 10 | 24 | 0 | 10 | 7 |
| 5959 | A | G | HELD_MAL_ADRCASE5ULN | 9 | 6 | 12 | 2 | 2 | 5 | HELD_MAL_ADRCTRL | 67 | 67 | 67 | 13 | 41 | 13 |
| 5959 | A | G | HELD_FEM_ADRCASE | 72 | 71 | 73 | 15 | 41 | 16 | HELD_FEM_ADRCTRL | 68 | 51 | 85 | 11 | 29 | 28 |
| 6162 | C | G | HELD_ALL_ADRCASE3ULN | 64 | 37 | 91 | 1 | 35 | 28 | HELD_ALL ADRCTRL | 151 | 90 | 212 | 19 | 52 | 80 |
| 6162 | C | G | HELD_ALL_ADRCASE | 156 | 88 | 224 | 6 | 76 | 74 | HELD_ALL_ADRCTRL | 151 | 90 | 212 | 19 | 52 | 80 |
| 6162 | C | G | HELD_ALL_ADRCASE5ULN | 27 | 16 | 38 | 0 | 16 | 11 | HELD_ALL_ADRCTRL | 151 | 90 | 212 | 19 | 52 | 80 |
| 6162 | C | G | HELD_MAL_ADRCASE3ULN | 26 | 13 | 39 | 0 | 13 | 13 | HELD_MAL_ADRCTRL | 71 | 43 | 99 | 11 | 21 | 39 |
| 6162 | C | G | HELD_FEM_ADRCASE5ULN | 18 | 13 | 23 | 0 | 13 | 5 | HELD_FEM_ADRCTRL | 80 | 47 | 113 | 8 | 31 | 41 |
| 6162 | C | G | HELD_MAL_ADRCASE | 74 | 40 | 108 | 3 | 34 | 37 | HELD_MAL_ADRCTRL | 71 | 43 | 99 | 11 | 21 | 39 |
| 6236 | C | T | HELD_ALL_ADRCASE5ULN | 27 | 24 | 30 | 6 | 12 | 9 | HELD_ALL_ADRCTRL | 152 | 84 | 220 | 13 | 58 | 81 |
| 6236 | C | T | HELD_MAL_ADRCASE3ULN | 27 | 23 | 31 | 4 | 15 | 8 | HELD_MAL_ADRCTRL | 72 | 38 | 106 | 5 | 28 | 39 |
| 6236 | C | T | HELD_MAL_ADRCASE5ULN | 10 | 10 | 10 | 2 | 6 | 2 | HELD_MAL_ADRCTRL | 72 | 38 | 106 | 5 | 28 | 39 |
| 6236 | C | T | HELD_ALL_ADRCASE3ULN | 63 | 47 | 79 | 10 | 27 | 26 | HELD_ALL_ADRCTRL | 152 | 84 | 220 | 13 | 58 | 81 |
| 6482 | A | G | HELD_MAL_LOHDL | 17 | 18 | 16 | 5 | 8 | 4 | HELD_MAL_HIHDL | 21 | 34 | 8 | 15 | 4 | 2 |
| 6482 | A | G | HELD_ALL_BAD2 | 619 | 918 | 320 | 340 | 238 | 41 | HELD_ALL_GOOD2 | 709 | 1098 | 320 | 436 | 226 | 47 |
| 6482 | A | G | HELD_MAL_CASE2 | 27 | 43 | 11 | 18 | 7 | 2 | HELD_MAL_CTRL2 | 28 | 32 | 24 | 10 | 12 | 6 |
| 6482 | A | G | HELD_MAL_BAD2 | 309 | 461 | 157 | 173 | 115 | 21 | HELD_MAL_GOOD2 | 339 | 539 | 139 | 220 | 99 | 20 |
| 6498 | A | G | CVD_FEM_CASE | 32 | 60 | 4 | 28 | 4 | 0 | CVD_FEM_CTRL | 35 | 57 | 13 | 25 | 7 | 3 |
| 6744 | C | T | HELD_ALL_ADRCASE5ULN | 26 | 21 | 31 | 4 | 13 | 9 | HELD_ALL_ADRCTRL | 149 | 74 | 224 | 9 | 56 | 84 |
| 7133 | C | G | HELD_MAL_CASE | 14 | 20 | 8 | 10 | 0 | 4 | HELD_MAL_CTRL | 18 | 36 | 0 | 18 | 0 | 0 |
| 8021 | A | G | CVD_FEM_CASE | 28 | 35 | 21 | 8 | 19 | 1 | CVD_FEM_CTRL | 36 | 44 | 28 | 15 | 14 | 7 |
| 8060 | A | G | CVD_FEM_CASE | 35 | 65 | 5 | 31 | 3 | 1 | CVD_FEM_CTRL | 40 | 68 | 12 | 28 | 12 | 0 |
| 8060 | A | G | HELD_FEM_LOHDL | 18 | 29 | 7 | 11 | 7 | 0 | HELD_FEM_HIHDL | 23 | 43 | 3 | 20 | 3 | 0 |
| 8210 | A | G | HELD_FEM_HIRESP | 12 | 9 | 15 | 1 | 7 | 4 | HELD_FEM_LORESP | 22 | 22 | 22 | 9 | 4 | 9 |
| 8592 | C | T | HELD_FEM_VHIRESP | 150 | 122 | 178 | 15 | 92 | 43 | HELD_FEM_VLORESP | 143 | 118 | 168 | 25 | 68 | 50 |
| 8816 | C | G | HELD_FEM_HIRESP | 13 | 15 | 11 | 4 | 7 | 2 | HELD_FEM_LORESP | 11 | 5 | 17 | 0 | 5 | 6 |
| 8846 | A | G | HELD_ALL_BAD | 107 | 161 | 53 | 57 | 47 | 3 | HELD_ALL_GOOD | 116 | 166 | 66 | 62 | 42 | 12 |
| 8943 | A | C | HELD_MAL_BAD | 20 | 35 | 5 | 15 | 5 | 0 | HELD_MAL_GOOD | 37 | 52 | 22 | 20 | 12 | 5 |
| 9193 | C | G | HELD_FEM_BAD | 83 | 155 | 11 | 72 | 11 | 0 | HELD_FEM_GOOD | 80 | 140 | 20 | 60 | 20 | 0 |
| 9193 | C | G | CVD_FEM_CASE | 36 | 63 | 9 | 28 | 7 | 1 | CVD_FEM_CTRL | 40 | 77 | 3 | 37 | 3 | 0 |
| 9443 | C | T | CVD_MAL_CASE | 69 | 43 | 95 | 9 | 25 | 35 | CVD_MAL_CTRL | 33 | 12 | 54 | 0 | 12 | 21 |
| 9516 | A | G | HELD_MAL_CASE | 14 | 17 | 11 | 7 | 3 | 4 | HELD_MAL_CTRL | 18 | 12 | 24 | 2 | 8 | 8 |
| 9698 | A | G | HELD_MAL_ADRCASE | 74 | 8 | 140 | 4 | 0 | 70 | HELD_MAL_ADRCTRL | 72 | 30 | 114 | 14 | 2 | 56 |
| 9698 | A | G | HELD_MAL_ADRCASE3ULN | 27 | 54 | 0 | 0 | 0 | 27 | HELD_MAL_ADRCTRL | 72 | 30 | 114 | 14 | 2 | 56 |
| 9698 | A | G | HELD_FEM_HIRESP | 294 | 105 | 483 | 5 | 95 | 194 | HELD_FEM_LORESP | 298 | 123 | 473 | 16 | 91 | 191 |
| 9698 | A | G | HELD_MAL_ADRCASE5ULN | 10 | 20 | 0 | 0 | 0 | 10 | HELD_MAL_ADRCTRL | 72 | 30 | 114 | 14 | 2 | 56 |
| 9698 | A | G | CVD_ALL_CASE | 102 | 46 | 158 | 17 | 12 | 73 | CVD_ALL_CTRL | 72 | 19 | 125 | 6 | 7 | 59 |
| 9849 | C | T | HELD_FEM_CASE | 31 | 62 | 0 | 31 | 0 | 0 | HELD_FEM_CTRL | 21 | 39 | 3 | 18 | 3 | 0 |
| 9849 | C | T | HELD_MAL_BAD | 20 | 35 | 5 | 15 | 5 | 0 | HELD_MAL_GOOD | 37 | 72 | 2 | 35 | 2 | 0 |
| 9883 | A | G | HELD_FEM_CASE | 31 | 23 | 39 | 7 | 9 | 15 | HELD_FEM_CTRL | 22 | 18 | 26 | 1 | 16 | 5 |
| 9883 | A | G | HELD_ALL_CASE | 45 | 33 | 57 | 9 | 15 | 21 | HELD_ALL_CTRL | 39 | 32 | 46 | 4 | 24 | 11 |
| 10079 | A | G | CVD_ALL_CASE | 103 | 8 | 198 | 4 | 0 | 99 | CVD_ALL_CTRL | 73 | 1 | 145 | 0 | 1 | 72 |
| 10079 | A | G | CVD_MAL_CASE | 68 | 8 | 128 | 4 | 0 | 64 | CVD_MAL_CTRL | 34 | 68 | 0 | 0 | 0 | 34 |
| 10481 | A | T | HELD_FEM_ADRCASE5ULN | 17 | 12 | 22 | 3 | 6 | 8 | HELD_FEM_ADRCTRL | 83 | 97 | 69 | 32 | 33 | 18 |
| 10542 | C | T | HELD_FEM_UHIRESP | 54 | 8 | 100 | 1 | 6 | 47 | HELD_FEM_ULORESP | 75 | 21 | 129 | 0 | 21 | 54 |
| 10542 | C | T | HELD_MAL_ADRCASE5ULN | 10 | 20 | 0 | 0 | 0 | 10 | HELD_MAL_ADRCTRL | 69 | 14 | 124 | 0 | 14 | 55 |
| 10600 | A | G | HELD_FEM_HIRESP | 21 | 42 | 0 | 0 | 0 | 21 | HELD_FEM_LORESP | 33 | 4 | 62 | 0 | 4 | 29 |
| 10621 | C | T | HELD_FEM_CASE | 30 | 52 | 8 | 24 | 4 | 2 | HELD_FEM_CTRL | 20 | 32 | 8 | 12 | 8 | 0 |
| 10745 | A | G | HELD_ALL_ADRCASE5ULN | 27 | 20 | 34 | 5 | 10 | 12 | HELD_ALL_ADRCTRL | 148 | 75 | 221 | 7 | 61 | 80 |
| 10745 | A | G | HELD_FEM_VHIRESP | 153 | 90 | 216 | 11 | 68 | 74 | HELD_FEM_VLORESP | 150 | 77 | 223 | 16 | 45 | 89 |
| 10747 | C | T | HELD_MAL_ADRCASE | 76 | 74 | 78 | 14 | 46 | 16 | HELD_MAL_ADRCTRL | 70 | 64 | 76 | 3 | 58 | 9 |
| 10747 | C | T | CVD_ALL_CASE | 62 | 54 | 70 | 15 | 24 | 23 | CVD_ALL_CTRL | 74 | 51 | 97 | 6 | 39 | 29 |
| 10747 | C | T | HELD_MAL_ADRCASE3ULN | 27 | 24 | 30 | 4 | 16 | 7 | HELD_MAL_ADRCTRL | 70 | 64 | 76 | 3 | 58 | 9 |
| 10771 | C | G | HELD_MAL_ADRCASE5ULN | 10 | 12 | 8 | 4 | 4 | 2 | HELD_MAL ADRCTRL | 70 | 48 | 92 | 6 | 36 | 28 |
| 10771 | C | G | HELD_FEM_HIRESP | 284 | 222 | 346 | 52 | 118 | 114 | HELD_FEM_LORESP | 276 | 185 | 367 | 40 | 105 | 131 |
| 10870 | A | G | HELD_MAL_BAD | 20 | 11 | 29 | 0 | 11 | 9 | HELD_MAL_GOOD | 37 | 19 | 55 | 5 | 9 | 23 |
| 10870 | A | G | HELD_FEM_BAD | 82 | 32 | 132 | 7 | 18 | 57 | HELD_FEM_GOOD | 77 | 46 | 108 | 8 | 30 | 39 |
| 10870 | A | G | HELD_MAL_CASE | 14 | 3 | 25 | 0 | 3 | 11 | HELD_MAL_CTRL | 18 | 12 | 24 | 2 | 8 | 8 |
| 10870 | A | G | HELD_ALL_CASE | 45 | 17 | 73 | 2 | 13 | 30 | HELD_ALL_CTRL | 40 | 27 | 53 | 6 | 15 | 19 |
| 10877 | A | C | HELD_ALL_LOHDL | 9 | 18 | 0 | 0 | 0 | 9 | HELD_ALL_HIHDL | 15 | 7 | 23 | 1 | 5 | 9 |
| 10948 | G | T | HELD_FEM_BAD | 84 | 83 | 85 | 16 | 51 | 17 | HELD_FEM_GOOD | 79 | 95 | 63 | 31 | 33 | 15 |
| 10948 | G | T | HELD_ALL_BAD | 104 | 104 | 104 | 22 | 60 | 22 | HELD_ALL_GOOD | 115 | 138 | 92 | 44 | 50 | 21 |
| 10948 | G | T | HELD_FEM_CASE2 | 44 | 46 | 42 | 9 | 28 | 7 | HELD_FEM_CTRL2 | 42 | 50 | 34 | 17 | 16 | 9 |
| 10948 | G | T | CVD_MAL_CASE | 69 | 63 | 75 | 12 | 39 | 18 | CVD_MAL_CTRL | 34 | 41 | 27 | 12 | 17 | 5 |
| 11001 | C | T | HELD_MAL_ADRCASE5ULN | 10 | 9 | 11 | 2 | 5 | 3 | HELD_MAL_ADRCTRL | 75 | 41 | 109 | 2 | 37 | 36 |
| 11073 | C | G | HELD_MAL_ADRCASE5ULN | 9 | 10 | 8 | 3 | 4 | 2 | HELD_MAL_ADRCTRL | 68 | 43 | 93 | 9 | 25 | 34 |
| 11153 | C | T | HELD_FEM_CASE | 31 | 55 | 7 | 24 | 7 | 0 | HELD_FEM_CTRL | 22 | 33 | 11 | 11 | 11 | 0 |
| 11210 | C | T | HELD_MAL_CASE | 14 | 23 | 5 | 9 | 5 | 0 | HELD_MAL_CTRL | 19 | 37 | 1 | 18 | 1 | 0 |
| 11210 | C | T | HELD_ALL_ADRCASE3ULN | 63 | 110 | 16 | 47 | 16 | 0 | HELD_ALL_ADRCTRL | 144 | 267 | 21 | 125 | 17 | 2 |
| 11210 | C | T | HELD_ALL_ADRCASE | 153 | 275 | 31 | 122 | 31 | 0 | HELD_ALL_ADRCTRL | 144 | 267 | 21 | 125 | 17 | 2 |
| 11248 | C | T | HELD_FEM_ADRCASE | 81 | 131 | 31 | 56 | 19 | 6 | HELD_FEM_ADRCTRL | 79 | 112 | 46 | 38 | 36 | 5 |
| 11248 | C | T | HELD_MAL_BAD | 18 | 33 | 3 | 15 | 3 | 0 | HELD_MAL_GOOD | 34 | 53 | 15 | 19 | 15 | 0 |
| 11248 | C | T | HELD_ALL_CASE | 41 | 68 | 14 | 27 | 14 | 0 | HELD_ALL_CTRL | 31 | 44 | 18 | 13 | 18 | 0 |
| 11372 | A | G | HELD_MAL_BAD | 20 | 25 | 15 | 10 | 5 | 5 | HELD_MAL_GOOD | 36 | 31 | 41 | 10 | 11 | 15 |
| 11449 | C | G | HELD_FEM_CASE | 31 | 6 | 56 | 1 | 4 | 26 | HELD_FEM_CTRL | 22 | 10 | 34 | 0 | 10 | 12 |
| 11450 | A | T | HELD_FEM_HIRESP | 289 | 170 | 408 | 28 | 114 | 147 | HELD_FEM_LORESP | 290 | 139 | 441 | 16 | 107 | 167 |
| 11470 | C | T | HELD_MAL_BAD | 20 | 40 | 0 | 20 | 0 | 0 | HELD_MAL_GOOD | 36 | 67 | 5 | 31 | 5 | 0 |
| 11472 | A | T | HELD_MAL_BAD | 20 | 40 | 0 | 20 | 0 | 0 | HELD_MAL_GOOD | 35 | 65 | 5 | 30 | 5 | 0 |
| 11472 | A | T | HELD_FEM_BAD | 83 | 158 | 8 | 75 | 8 | 0 | HELD_FEM_GOOD | 80 | 158 | 2 | 78 | 2 | 0 |
| 11487 | A | T | HELD_MAL_ADRCASE5ULN | 10 | 20 | 0 | 0 | 10 | 0 | HELD_MAL_ADRCTRL | 69 | 34 | 104 | 34 | 35 | 0 |
| 11487 | A | T | HELD_MAL_ADRCASE3ULN | 27 | 6 | 48 | 6 | 21 | 0 | HELD_MAL_ADRCTRL | 69 | 34 | 104 | 34 | 35 | 0 |
| 11488 | C | G | HELD_MAL_ADRCASE5ULN | 10 | 20 | 0 | 10 | 0 | 0 | HELD_MAL_ADRCTRL | 70 | 102 | 38 | 35 | 32 | 3 |
| 11488 | C | G | HELD_FEM_UHIRESP | 54 | 78 | 30 | 29 | 20 | 5 | HELD_FEM_ULORESP | 77 | 126 | 28 | 49 | 28 | 0 |
| 11488 | C | G | HELD_MAL_ADRCASE3ULN | 26 | 44 | 8 | 20 | 4 | 2 | HELD_MAL_ADRCTRL | 70 | 102 | 38 | 35 | 32 | 3 |
| 11493 | A | G | HELD_MAL_CASE | 14 | 6 | 22 | 0 | 6 | 8 | HELD_MAL_CTRL | 18 | 6 | 30 | 2 | 2 | 14 |
| 11502 | C | T | HELD_MAL_ADRCASE3ULN | 27 | 8 | 46 | 0 | 8 | 19 | HELD_MAL_ADRCTRL | 73 | 44 | 102 | 7 | 30 | 36 |
| 11502 | C | T | HELD_MAL_ADRCASE5ULN | 10 | 2 | 18 | 0 | 2 | 8 | HELD_MAL_ADRCTRL | 73 | 44 | 102 | 7 | 30 | 36 |
| 11534 | G | T | HELD_ALL_BAD | 102 | 204 | 0 | 102 | 0 | 0 | HELD_ALL_GOOD | 117 | 231 | 3 | 114 | 3 | 0 |
| 11537 | A | G | CVD_FEM_CASE | 36 | 52 | 20 | 20 | 12 | 4 | CVD_FEM_CTRL | 39 | 68 | 10 | 30 | 8 | 1 |
| 11537 | A | G | HELD_FEM_HIRESP | 12 | 22 | 2 | 10 | 2 | 0 | HELD_FEM_LORESP | 22 | 31 | 13 | 12 | 7 | 3 |
| 11560 | A | G | HELD_FEM_HIRESP | 12 | 2 | 22 | 1 | 0 | 11 | HELD_FEM_LORESP | 22 | 44 | 0 | 0 | 0 | 22 |
| 11578 | C | T | HELD_FEM_BAD | 61 | 121 | 1 | 60 | 1 | 0 | HELD_FEM_GOOD | 65 | 122 | 8 | 57 | 8 | 0 |
| 11578 | C | T | CVD_FEM_CASE | 30 | 57 | 3 | 27 | 3 | 0 | CVD_FEM_CTRL | 39 | 78 | 0 | 39 | 0 | 0 |
| 11594 | C | T | HELD_FEM_ADRCASE3ULN | 37 | 74 | 0 | 0 | 0 | 37 | HELD_FEM_ADRCTRL | 80 | 10 | 150 | 2 | 6 | 72 |
| 11594 | C | T | HELD_ALL_ADRCASE5ULN | 27 | 54 | 0 | 0 | 0 | 27 | HELD_ALL_ADRCTRL | 151 | 20 | 282 | 2 | 16 | 133 |
| 11594 | C | T | HELD_ALL_CASE | 45 | 10 | 80 | 0 | 10 | 35 | HELD_ALL_CTRL | 41 | 3 | 79 | 0 | 3 | 38 |
| 11594 | C | T | HELD_ALL_ADRCASE | 155 | 9 | 301 | 1 | 7 | 147 | HELD_ALL_ADRCTRL | 151 | 20 | 282 | 2 | 16 | 133 |
| 11594 | C | T | HELD_FEM_ADRCASE5ULN | 18 | 36 | 0 | 0 | 0 | 18 | HELD_FEM_ADRCTRL | 80 | 10 | 150 | 2 | 6 | 72 |
| 11624 | C | T | HELD_ALL_CASE | 42 | 57 | 27 | 21 | 15 | 6 | HELD_ALL_CTRL | 40 | 60 | 20 | 20 | 20 | 0 |
| 11624 | C | T | HELD_MAL_CASE | 13 | 18 | 8 | 8 | 2 | 3 | HELD_MAL_CTRL | 18 | 27 | 9 | 9 | 9 | 0 |
| 11624 | C | T | HELD_FEM_HIRESP | 12 | 22 | 2 | 10 | 2 | 0 | HELD_FEM_LORESP | 21 | 30 | 12 | 12 | 6 | 3 |
| 11627 | C | T | HELD_ALL_CASE | 45 | 58 | 32 | 20 | 18 | 7 | HELD_ALL_CTRL | 40 | 61 | 19 | 21 | 19 | 0 |
| 11627 | C | T | HELD_MAL_CASE | 14 | 18 | 10 | 7 | 4 | 3 | HELD_MAL_CTRL | 18 | 27 | 9 | 9 | 9 | 0 |
| 11627 | C | T | HELD_FEM_HIRESP | 12 | 22 | 2 | 10 | 2 | 0 | HELD_FEM_LORESP | 22 | 31 | 13 | 12 | 7 | 3 |
| 11644 | A | G | HELD_MAL_ADRCASE5ULN | 10 | 2 | 18 | 0 | 2 | 8 | HELD_MAL_ADRCTRL | 68 | 40 | 96 | 7 | 26 | 35 |
| 11650 | A | G | HELD_FEM_HIRESP | 291 | 157 | 425 | 26 | 105 | 160 | HELD_FEM_LORESP | 290 | 181 | 399 | 23 | 135 | 132 |
| 11654 | A | G | HELD_ALL_ADRCASE5ULN | 25 | 17 | 33 | 7 | 3 | 15 | HELD_ALL_ADRCTRL | 136 | 84 | 188 | 14 | 56 | 66 |
| 11654 | A | G | HELD_FEM_ADRCASE5ULN | 15 | 11 | 19 | 5 | 1 | 9 | HELD_FEM_ADRCTRL | 71 | 47 | 95 | 8 | 31 | 32 |
| 11654 | A | G | HELD_FEM_ADRCASE3ULN | 32 | 23 | 41 | 8 | 7 | 17 | HELD_FEM_ADRCTRL | 71 | 47 | 95 | 8 | 31 | 32 |
| 11654 | A | G | HELD_ALL_ADRCASE3ULN | 53 | 39 | 67 | 12 | 15 | 26 | HELD_ALL_ADRCTRL | 136 | 84 | 188 | 14 | 56 | 66 |
| 11655 | A | C | HELD_ALL_ADRCASE5ULN | 26 | 35 | 17 | 16 | 3 | 7 | HELD_ALL_ADRCTRL | 148 | 203 | 93 | 72 | 59 | 17 |
| 11655 | A | C | HELD_FEM_ADRCASE5ULN | 17 | 23 | 11 | 11 | 1 | 5 | HELD_FEM_ADRCTRL | 80 | 104 | 56 | 35 | 34 | 11 |
| 11655 | A | C | HELD_FEM_ADRCASE3ULN | 35 | 45 | 25 | 19 | 7 | 9 | HELD_FEM_ADRCTRL | 80 | 104 | 56 | 35 | 34 | 11 |
| 11656 | C | T | HELD_MAL_BAD | 20 | 20 | 20 | 6 | 8 | 6 | HELD_MAL_GOOD | 36 | 53 | 19 | 19 | 15 | 2 |
| 11656 | C | T | HELD_FEM_HIRESP | 12 | 19 | 5 | 7 | 5 | 0 | HELD_FEM_LORESP | 22 | 24 | 20 | 5 | 14 | 3 |
| 11656 | C | T | HELD_ALL_BAD | 102 | 119 | 85 | 35 | 49 | 18 | HELD_ALL_GOOD | 114 | 156 | 72 | 51 | 54 | 9 |
| 11825 | A | G | HELD_MAL_ADRCASE5ULN | 9 | 15 | 3 | 6 | 3 | 0 | HELD_MAL_ADRCTRL | 63 | 121 | 5 | 58 | 5 | 0 |
| 11914 | A | T | HELD_MAL_ADRCASE5ULN | 9 | 2 | 16 | 1 | 0 | 8 | HELD_MAL_ADRCTRL | 69 | 83 | 55 | 41 | 1 | 27 |
| 11914 | A | T | HELD_ALL_ADRCASE5ULN | 27 | 24 | 30 | 6 | 12 | 9 | HELD_ALL_ADRCTRL | 151 | 178 | 124 | 63 | 52 | 36 |
| 12008 | C | T | HELD_FEM_HIRESP | 278 | 529 | 27 | 251 | 27 | 0 | HELD_FEM_LORESP | 277 | 541 | 13 | 264 | 13 | 0 |
| 12008 | C | T | HELD_ALL_ADRCASE5ULN | 24 | 48 | 0 | 24 | 0 | 0 | HELD_ALL_ADRCTRL | 134 | 256 | 12 | 122 | 12 | 0 |
| 12097 | A | G | HELD_ALL_ADRCASE5ULN | 28 | 6 | 50 | 6 | 22 | 0 | HELD_ALL_ADRCTRL | 155 | 11 | 299 | 11 | 144 | 0 |
| 12097 | A | G | HELD_FEM_ADRCASE3ULN | 38 | 7 | 69 | 7 | 31 | 0 | HELD_FEM_ADRCTRL | 83 | 5 | 161 | 5 | 78 | 0 |
| 12097 | A | G | HELD_MAL_ADRCASE5ULN | 10 | 3 | 17 | 3 | 7 | 0 | HELD_MAL_ADRCTRL | 72 | 6 | 138 | 6 | 66 | 0 |
| 12097 | A | G | HELD_ALL_ADRCASE3ULN | 63 | 10 | 116 | 10 | 53 | 0 | HELD_ALL_ADRCTRL | 155 | 11 | 299 | 11 | 144 | 0 |
| 12366 | A | G | HELD_FEM_UHIRESP | 50 | 82 | 18 | 32 | 18 | 0 | HELD_FEM_ULORESP | 74 | 104 | 44 | 39 | 26 | 9 |
| 12366 | A | G | HELD_ALL_ADRCASE5ULN | 25 | 40 | 10 | 18 | 4 | 3 | HELD_ALL_ADRCTRL | 151 | 229 | 73 | 85 | 59 | 7 |
| 12619 | A | G | HELD_MAL_ADRCASE5ULN | 10 | 1 | 19 | 1 | 9 | 0 | HELD_MAL_ADRCTRL | 71 | 142 | 0 | 0 | 71 | 0 |
| 12619 | A | G | HELD_ALL_ADRCASE5ULN | 27 | 2 | 52 | 2 | 25 | 0 | HELD_ALL_ADRCTRL | 151 | 1 | 301 | 1 | 150 | 0 |
| 13025 | A | C | HELD_ALL_ADRCASE5ULN | 28 | 34 | 22 | 13 | 8 | 7 | HELD_ALL_ADRCTRL | 151 | 201 | 101 | 65 | 71 | 15 |
| 13191 | A | G | HELD_FEM_BAD | 83 | 42 | 124 | 6 | 30 | 47 | HELD_FEM_GOOD | 79 | 62 | 96 | 10 | 42 | 27 |
| 13191 | A | G | HELD_MAL_CASE | 14 | 11 | 17 | 2 | 7 | 5 | HELD_MAL_CTRL | 18 | 5 | 31 | 0 | 5 | 13 |
| 13191 | A | G | HELD_ALL_BAD | 101 | 51 | 151 | 6 | 39 | 56 | HELD_ALL_GOOD | 114 | 81 | 147 | 13 | 55 | 46 |
| 13937 | A | C | HELD_FEM_ADRCASE5ULN | 17 | 19 | 15 | 4 | 11 | 2 | HELD_FEM_ADRCTRL | 83 | 122 | 44 | 42 | 38 | 3 |
| 900002 | G | T | CVD_FEM_CASE | 34 | 23 | 45 | 5 | 13 | 16 | CVD_FEM_CTRL | 40 | 15 | 65 | 2 | 11 | 27 |
| 900013 | C | G | CVD_FEM_CASE | 35 | 49 | 21 | 20 | 9 | 6 | CVD_FEM_CTRL | 40 | 49 | 31 | 13 | 23 | 4 |
| 900013 | C | G | CVD_ALL_CASE | 104 | 150 | 58 | 58 | 34 | 12 | CVD_ALL_CTRL | 74 | 97 | 51 | 29 | 39 | 6 |
| 900025 | G | T | CVD_MAL_CASE | 66 | 41 | 91 | 7 | 27 | 32 | CVD_MAL_CTRL | 34 | 31 | 37 | 7 | 17 | 10 |
| 900032 | C | T | CVD_FEM_CASE | 25 | 47 | 3 | 23 | 1 | 1 | CVD_FEM_CTRL | 37 | 65 | 9 | 28 | 9 | 0 |
| 900045 | C | T | HELD_FEM_HIRESP | 12 | 4 | 20 | 1 | 2 | 9 | HELD_FEM_LORESP | 22 | 18 | 26 | 5 | 8 | 9 |
| 900065 | A | C | CVD_FEM_CASE | 32 | 54 | 10 | 22 | 10 | 0 | CVD_FEM_CTRL | 39 | 50 | 28 | 16 | 18 | 5 |
| 900065 | A | C | CVD_MAL_CASE | 59 | 80 | 38 | 25 | 30 | 4 | CVD_MAL_CTRL | 29 | 36 | 22 | 7 | 22 | 0 |
| 900065 | A | C | CVD_ALL_CASE | 91 | 134 | 48 | 47 | 40 | 4 | CVD_ALL_CTRL | 68 | 86 | 50 | 23 | 40 | 5 |
| 900078 | A | G | HELD_ALL_ADRCASE3ULN | 64 | 116 | 12 | 52 | 12 | 0 | HELD_ALL_ADRCTRL | 155 | 297 | 13 | 142 | 13 | 0 |
| 900078 | A | G | HELD_ALL_ADRCASE5ULN | 27 | 48 | 6 | 21 | 6 | 0 | HELD_ALL_ADRCTRL | 155 | 297 | 13 | 142 | 13 | 0 |
| 900078 | A | G | HELD_FEM_ADRCASE3ULN | 38 | 69 | 7 | 31 | 7 | 0 | HELD_FEM_ADRCTRL | 83 | 161 | 5 | 78 | 5 | 0 |
| 900082 | A | G | HELD_FEM_ADRCASE3ULN | 35 | 25 | 45 | 8 | 9 | 18 | HELD_FEM_ADRCTRL | 74 | 70 | 78 | 17 | 36 | 21 |
| 900082 | A | G | HELD_FEM_ADRCASE5ULN | 17 | 10 | 24 | 3 | 4 | 10 | HELD_FEM_ADRCTRL | 74 | 70 | 78 | 17 | 36 | 21 |
| 900096 | A | G | CVD_ALL_CASE | 101 | 157 | 45 | 60 | 37 | 4 | CVD_ALL_CTRL | 72 | 125 | 19 | 55 | 15 | 2 |
| 900107 | C | T | HELD_MAL_ADRCASE5ULN | 10 | 2 | 18 | 0 | 2 | 8 | HELD_MAL_ADRCTRL | 73 | 43 | 103 | 9 | 25 | 39 |
| 900115 | A | G | HELD_MAL_ADRCASE5ULN | 9 | 6 | 12 | 1 | 4 | 4 | HELD_MAL_ADRCTRL | 72 | 91 | 53 | 27 | 37 | 8 |
| 900115 | A | G | HELD_FEM_HIRESP | 40 | 58 | 22 | 22 | 14 | 4 | HELD_FEM_LORESP | 46 | 62 | 30 | 17 | 28 | 1 |
| 900121 | G | T | HELD_MAL_ADRCASE | 66 | 47 | 85 | 5 | 37 | 24 | HELD_MAL_ADRCTRL | 67 | 56 | 78 | 15 | 26 | 26 |
| 900173 | G | T | CVD_ALL_CASE | 23 | 17 | 29 | 5 | 7 | 11 | CVD_ALL_CTRL | 22 | 26 | 18 | 11 | 4 | 7 |
| 10000002 | A | G | HELD_FEM_HIRESP | 12 | 21 | 3 | 9 | 3 | 0 | HELD_FEM_LORESP | 22 | 25 | 19 | 9 | 7 | 6 |
| 10000006 | A | G | HELD_FEM_CASE | 31 | 58 | 4 | 28 | 2 | 1 | HELD_FEM_CTRL | 22 | 31 | 13 | 11 | 9 | 2 |
| 10000006 | A | G | HELD_ALL_CASE | 44 | 82 | 6 | 39 | 4 | 1 | HELD_ALL_CTRL | 38 | 58 | 18 | 23 | 12 | 3 |
| 10000014 | A | C | HELD_ALL_CASE | 45 | 83 | 7 | 40 | 3 | 2 | HELD_ALL_CTRL | 39 | 64 | 14 | 26 | 12 | 1 |
| 10000014 | A | C | HELD_FEM_CASE | 31 | 58 | 4 | 28 | 2 | 1 | HELD_FEM_CTRL | 22 | 37 | 7 | 15 | 7 | 0 |
| 10000025 | C | T | HELD_MAL_BAD | 20 | 29 | 11 | 9 | 11 | 0 | HELD_MAL_GOOD | 36 | 43 | 29 | 14 | 15 | 7 |

TABLE 5b
p-values of PA SNPs
A SNP is considered as associated to cardiovascular disease, adverse statin response or to efficacy
of statin treatment, respectively, when one of the p values is equal or below 0.05.

| | | GTYPE | GTYPE | GTYPE | ALLELE | ALLELE | ALLELE |
|---|---|---|---|---|---|---|---|
| BAYSNP | COMPARISON | CPVAL | XPVAL | LRPVAL | CPVAL | XPVAL | LRPVAL |
| 28 | HELD_FEM_EFF | 0.0506 | 0.0508 | 0.0442 | 0.0411 | 0.0579 | 0.0349 |
| 29 | HELD_ALL_HDL | 0.021 | 0.0227 | 0.0099 | 0.0089 | 0.0164 | 0.0087 |
| 29 | HELD_MAL_ADR3ULN | 0.0602 | 0.0582 | 0.0664 | 0.0446 | 0.0526 | 0.0435 |
| 29 | HELD_MAL_ADR5ULN | 0.1406 | 0.1835 | 0.1554 | 0.0455 | 0.0778 | 0.0422 |
| 52 | HELD_FEM_EFF | 0.0644 | 0.0861 | 0.0488 | 0.0272 | 0.0362 | 0.0261 |
| 56 | HELD_FEM_EFF | 0.0248 | 0.0379 | 0.0273 | 0.0347 | 0.0479 | 0.0393 |
| 89 | HELD_ALL_CC | 0.0614 | 0.1 | 0.0311 | 0.0638 | 0.1021 | 0.0323 |
| 90 | HELD_FEM_CC | 0.0398 | 0.0424 | 0.0242 | 0.1382 | 0.1687 | 0.137 |
| 99 | HELD_FEM_LIP | 0.0363 | 0.0366 | 0.0338 | 0.8397 | 0.9056 | 0.8397 |
| 140 | HELD_FEM_EFF | 0.3895 | 0.6921 | 0.2368 | 0.1188 | 0.288 | 0.0524 |
| 152 | HELD_FEM_EFF | 0.1084 | 0.1216 | 0.1082 | 0.0373 | 0.0595 | 0.0389 |
| 214 | HELD_ALL_LIP | 0.1139 | 0.1152 | 0.0532 | 0.9756 | 1 | 0.9756 |
| 214 | HELD_FEM_LIP | 0.1095 | 0.1196 | 0.0506 | 0.5567 | 0.5803 | 0.5567 |
| 221 | HELD_ALL_CC | 0.0367 | 0.0359 | 0.0353 | 0.4257 | 0.506 | 0.426 |
| 221 | HELD_FEM_CC | 0.0406 | 0.0424 | 0.0384 | 0.1456 | 0.2083 | 0.1469 |
| 224 | HELD_FEM_LIP | 0.2893 | 0.3016 | 0.2874 | 0.0533 | 0.0709 | 0.0527 |
| 224 | HELD_MAL_LIP | 0.2292 | 0.2815 | 0.1975 | 0.0278 | 0.0392 | 0.0221 |
| 294 | HELD_ALL_CC | 0.0851 | 0.1041 | 0.0327 | 0.1547 | 0.1913 | 0.1534 |
| 307 | CVD_FEM | 0.013 | 0.0118 | 0.0104 | 0.0032 | 0.004 | 0.003 |
| 307 | HELD_ALL_LIP | 0.0255 | 0.0273 | 0.0249 | 0.0934 | 0.0968 | 0.0936 |
| 411 | HELD_ALL_HDL | 0.1529 | 0.2195 | 0.1076 | 0.0588 | 0.1136 | 0.0513 |
| 449 | HELD_MAL_LIP | 0.1321 | 0.0942 | 0.1001 | 0.0535 | 0.0667 | 0.0416 |
| 466 | CVD_FEM | 0.133 | 0.1439 | 0.1301 | 0.0444 | 0.0505 | 0.0438 |
| 472 | HELD_FEM_EFF | 0.0453 | 0.0626 | 0.0116 | 0.0068 | 0.0146 | 0.0009 |
| 542 | HELD_MAL_CC | 0.0014 | 0.0009 | 0.0007 | 0.0002 | 0.0003 | 0.0002 |
| 542 | HELD_MAL_HDL | 0.0054 | 0.0028 | 0.0029 | 0.0004 | 0.0005 | 0.0003 |
| 542 | HELD_ALL_ADR | 0.0257 | 0.0152 | 0.0171 | 0.0971 | 0.1108 | 0.0962 |
| 542 | HELD_FEM_HDL | 0.1914 | 0.1661 | 0.1457 | 0.0613 | 0.0709 | 0.0487 |
| 739 | HELD_ALL_CC | 0.0958 | 0.0983 | 0.0902 | 0.03 | 0.0327 | 0.0296 |
| 821 | HELD_MAL_LIP2 | 0.0426 | 0.0436 | 0.0419 | 0.0865 | 0.0927 | 0.0867 |
| 821 | HELD_FEM_VEFF | 0.1193 | 0.1222 | 0.0584 | 0.0343 | 0.0681 | 0.0306 |
| 1005 | HELD_MAL_CC | 0.2376 | 0.3423 | 0.1618 | 0.0603 | 0.0946 | 0.0502 |
| 1055 | HELD_MAL_CC | 0.0302 | 0.0328 | 0.0084 | 0.2241 | 0.2988 | 0.216 |
| 1056 | HELD_FEM_EFF | 0.0094 | 0.0085 | 0.0079 | 0.9671 | 1 | 0.9671 |
| 1085 | HELD_MAL_LIP | 0.0889 | 0.0964 | 0.0773 | 0.0288 | 0.0462 | 0.0288 |
| 1085 | CVD_FEM | 0.1655 | 0.1833 | 0.156 | 0.0373 | 0.0546 | 0.0359 |
| 1086 | HELD_MAL_LIP | 0.0963 | 0.1125 | 0.0928 | 0.0318 | 0.0475 | 0.0315 |
| 1092 | HELD_MAL_LIP | 0.0493 | 0.0492 | 0.046 | 0.0712 | 0.0958 | 0.0663 |
| 1096 | HELD_MAL_CC | 0.0436 | 0.0623 | 0.0423 | 0.0685 | 0.0895 | 0.0679 |
| 1096 | CVD_MAL | 0.0766 | 0.0645 | 0.0452 | 0.5906 | 0.6848 | 0.5936 |
| 1101 | HELD_FEM_EFF | 0.1158 | 0.2728 | 0.0522 | 0.1279 | 0.2891 | 0.0572 |
| 1204 | HELD_MAL_LIP | 0.0471 | 0.0447 | 0.0362 | 0.0189 | 0.0238 | 0.0214 |
| 1204 | HELD_ALL_LIP | 0.1563 | 0.1592 | 0.1558 | 0.0422 | 0.0485 | 0.0424 |
| 1504 | HELD_ALL_CC | 0.0128 | 0.0133 | 0.0115 | 0.5946 | 0.64 | 0.5946 |
| 1504 | HELD_MAL_LIP | 0.0864 | 0.087 | 0.0247 | 0.1834 | 0.2241 | 0.1799 |
| 1504 | HELD_MAL_CC | 0.051 | 0.0757 | 0.0467 | 0.2868 | 0.3134 | 0.2871 |
| 1504 | HELD_FEM_CC | 0.0535 | 0.0663 | 0.0532 | 0.0878 | 0.1084 | 0.0873 |
| 1511 | HELD_FEM_EFF | 0.0513 | 0.0299 | 0.0413 | 0.1279 | 0.1563 | 0.1329 |
| 1524 | HELD_FEM_ADR3ULN | 0.0684 | 0.0673 | 0.0215 | 0.64 | 0.7419 | 0.6382 |
| 1556 | HELD_FEM_EFF | 0.0063 | 0.0151 | 0.0066 | 0.0129 | 0.0269 | 0.015 |
| 1561 | CVD_FEM | 0.1299 | 0.1484 | 0.1216 | 0.0472 | 0.0666 | 0.0456 |
| 1582 | HELD_MAL_LIP | 0.1444 | 0.1408 | 0.0649 | 0.0389 | 0.0633 | 0.0319 |
| 1638 | HELD_FEM_CC | 0.0876 | 0.0903 | 0.0861 | 0.0318 | 0.0385 | 0.0328 |
| 1653 | CVD_MAL | 0.0269 | 0.0234 | 0.0255 | 0.4812 | 0.5499 | 0.4809 |
| 1662 | HELD_MAL_CC | 0.0153 | 0.0278 | 0.0067 | 0.0006 | 0.0007 | 0.0001 |
| 1714 | CVD_MAL | 0.0716 | 0.0776 | 0.0817 | 0.0388 | 0.0484 | 0.041 |
| 1722 | HELD_FEM_ADR5ULN | 0.0325 | 0.0304 | 0.0429 | 0.1144 | 0.1401 | 0.1146 |
| 1757 | HELD_FEM_EFF | 0.0289 | 0.0296 | 0.0153 | 0.1752 | 0.1926 | 0.1779 |
| 1765 | HELD_ALL_ADR3ULN | 0.0044 | 0.0049 | 0.0024 | 0.0023 | 0.0029 | 0.0012 |
| 1765 | HELD_ALL_ADR3ULN | 0.0044 | 0.0049 | 0.0024 | 0.0023 | 0.0029 | 0.0012 |
| 1765 | HELD_ALL_ADR5ULN | 0.0469 | 0.0457 | 0.0235 | 0.0166 | 0.0163 | 0.0077 |
| 1765 | HELD_ALL_ADR5ULN | 0.0469 | 0.0457 | 0.0235 | 0.0166 | 0.0163 | 0.0077 |
| 1765 | HELD_MAL_ADR3ULN | 0.0428 | 0.0505 | 0.0211 | 0.0131 | 0.0174 | 0.0058 |
| 1765 | HELD_MAL_ADR3ULN | 0.0428 | 0.0505 | 0.0211 | 0.0131 | 0.0174 | 0.0058 |
| 1765 | HELD_MAL_ADR5ULN | 0.0997 | 0.0786 | 0.0255 | 0.0396 | 0.0451 | 0.0069 |
| 1765 | HELD_MAL_ADR5ULN | 0.0997 | 0.0786 | 0.0255 | 0.0396 | 0.0451 | 0.0069 |
| 1765 | HELD_FEM_ADR3ULN | 0.0666 | 0.0733 | 0.0522 | 0.0513 | 0.0579 | 0.0423 |
| 1765 | HELD_FEM_ADR3ULN | 0.0666 | 0.0733 | 0.0522 | 0.0513 | 0.0579 | 0.0423 |
| 1776 | HELD_ALL_CC | 0.0614 | 0.1 | 0.0311 | 0.0082 | 0.0098 | 0.0023 |
| 1776 | HELD_FEM_CC | 0.087 | 0.1676 | 0.0568 | 0.0155 | 0.0273 | 0.0071 |
| 1799 | HELD_FEM_LIP2 | 0.006 | 0.0058 | 0.0061 | 0.2598 | 0.268 | 0.2601 |
| 1799 | HELD_MAL_CC | 0.1419 | 0.1545 | 0.134 | 0.0408 | 0.0604 | 0.0406 |
| 1806 | HELD_FEM_EFF | 0.1946 | 0.236 | 0.128 | 0.047 | 0.0817 | 0.0299 |
| 1837 | HELD_FEM_LIP2 | 0.0049 | 0.0047 | 0.0048 | 0.569 | 0.5843 | 0.5688 |
| 1837 | HELD_ALL_LIP2 | 0.0085 | 0.0085 | 0.0084 | 0.0433 | 0.0445 | 0.0431 |
| 1837 | HELD_ALL_ADR5ULN | 0.0159 | 0.015 | 0.0135 | 0.0245 | 0.0271 | 0.019 |
| 1837 | HELD_MAL_ADR | 0.0544 | 0.0558 | 0.0529 | 0.078 | 0.0897 | 0.0779 |
| 1837 | HELD_MAL_LIP2 | 0.0694 | 0.0696 | 0.0684 | 0.0215 | 0.0237 | 0.0213 |
| 1870 | HELD_ALL_CC | 0.0213 | 0.018 | 0.0195 | 0.0874 | 0.1157 | 0.0854 |
| 1870 | HELD_FEM_CC | 0.0621 | 0.0435 | 0.059 | 0.0937 | 0.1293 | 0.0894 |
| 1882 | CVD_MAL | 0.0296 | 0.028 | 0.0055 | 0.4108 | 0.4529 | 0.4093 |
| 1988 | HELD_ALL_LIP | 0.1287 | 0.1307 | 0.1234 | 0.0385 | 0.0414 | 0.0379 |
| 2000 | CVD_MAL | 0.0237 | 0.0363 | 0.0295 | 0.0014 | 0.0025 | 0.0021 |
| 2000 | CVD_ALL | 0.034 | 0.0425 | 0.035 | 0.0027 | 0.0035 | 0.0029 |
| 2000 | HELD_FEM_CC2 | 0.0705 | 0.0992 | 0.061 | 0.0105 | 0.0145 | 0.0081 |
| 2000 | HELD_MAL_HDL | 0.1671 | 0.489 | 0.1018 | 0.0507 | 0.1177 | 0.0207 |
| 2000 | HELD_FEM_ADR | 0.1624 | 0.2773 | 0.1528 | 0.0482 | 0.0704 | 0.0432 |
| 2000 | HELD_MAL_CC | 0.1597 | 0.2882 | 0.1581 | 0.0467 | 0.063 | 0.0459 |
| 2071 | CVD_ALL | 0.0823 | 0.09 | 0.0741 | 0.0349 | 0.0411 | 0.0339 |
| 2078 | HELD_MAL_LIP | 0.0667 | 0.0395 | 0.0572 | 0.0468 | 0.0583 | 0.0507 |
| 2085 | HELD_FEM_VEFF | 0.0707 | 0.0839 | 0.0347 | 0.019 | 0.0349 | 0.0165 |
| 2095 | CVD_ALL | 0.0917 | 0.1451 | 0.0384 | 0.0935 | 0.1473 | 0.0392 |
| 2119 | HELD_MAL_LIP | 0.0309 | 0.0409 | 0.0248 | 0.1269 | 0.148 | 0.1297 |
| 2119 | HELD_ALL_LIP | 0.0382 | 0.0476 | 0.0373 | 0.133 | 0.1514 | 0.1332 |
| 2119 | HELD_FEM_EFF | 0.057 | 0.0796 | 0.0527 | 0.1279 | 0.1563 | 0.1329 |
| 2141 | HELD_FEM_EFF | 0.021 | 0.0256 | 0.0169 | 0.2401 | 0.3207 | 0.2483 |
| 2141 | HELD_ALL_CC | 0.079 | 0.0695 | 0.0439 | 0.9551 | 1 | 0.9551 |
| 2182 | HELD_FEM_EFF | 0.0038 | 0.0027 | 0.0014 | 0.0039 | 0.0051 | 0.0033 |
| 2234 | HELD_MAL_LIP | 0.0604 | 0.0581 | 0.0195 | 0.0315 | 0.0414 | 0.0289 |
| 2281 | HELD_FEM_VEFF | 0.1098 | 0.1234 | 0.0542 | 0.0501 | 0.0685 | 0.0472 |
| 2298 | CVD_FEM | 0.0241 | 0.0171 | 0.0108 | 0.9341 | 1 | 0.934 |
| 2298 | HELD_MAL_CC2 | 0.1235 | 0.1076 | 0.0833 | 0.053 | 0.0671 | 0.0514 |
| 2341 | HELD_FEM_CC | 0.0284 | 0.0709 | 0.0083 | 0.0336 | 0.0796 | 0.0097 |
| 2357 | HELD_ALL_CC2 | 0.042 | 0.0374 | 0.016 | 0.7724 | 0.8793 | 0.7723 |
| 2357 | HELD_ALL_CC | 0.0452 | 0.0325 | 0.0209 | 0.9622 | 1 | 0.9622 |
| 2357 | HELD_MAL_LIP | 0.0438 | 0.0824 | 0.0385 | 0.077 | 0.1278 | 0.0657 |
| 2357 | HELD_FEM_CC | 0.0772 | 0.0829 | 0.0381 | 0.6486 | 0.7985 | 0.6469 |
| 2366 | CVD_FEM | 0.1125 | 0.1171 | 0.1073 | 0.0234 | 0.0304 | 0.023 |
| 2423 | CVD_FEM | 0.086 | 0.0888 | 0.077 | 0.0185 | 0.0274 | 0.0179 |
| 2708 | CVD_FEM | 0.0719 | 0.1262 | 0.054 | 0.0813 | 0.1384 | 0.0609 |
| 2995 | HELD_FEM_ADR5ULN | 0.0882 | 0.0827 | 0.1088 | 0.0448 | 0.0488 | 0.0503 |
| 2995 | HELD_FEM_UEFF | 0.0943 | 0.0942 | 0.0928 | 0.0516 | 0.0693 | 0.0495 |
| 3360 | HELD_MAL_ADR5ULN | 0.1131 | 0.1691 | 0.0302 | 0.0499 | 0.0819 | 0.0097 |
| 3464 | HELD_ALL_CC | 0.0305 | 0.0331 | 0.0278 | 0.0047 | 0.0056 | 0.0046 |
| 3464 | HELD_FEM_CC | 0.0743 | 0.0777 | 0.0721 | 0.0141 | 0.0184 | 0.0144 |
| 3689 | HELD_FEM_EFF | 0.0488 | 0.0584 | 0.0295 | 0.0226 | 0.0378 | 0.0206 |
| 3975 | HELD_FEM_UEFF | 0.0492 | 0.0474 | 0.0407 | 0.0198 | 0.0237 | 0.0188 |
| 3976 | HELD_FEM_UEFF | 0.059 | 0.0605 | 0.0456 | 0.0262 | 0.0327 | 0.025 |
| 4206 | HELD_FEM_ADR3ULN | 0.1395 | 0.1496 | 0.1372 | 0.0522 | 0.0655 | 0.0529 |
| 4838 | HELD_FEM_VEFF | 0.0581 | 0.0772 | 0.0529 | 0.0343 | 0.0681 | 0.0306 |
| 4838 | HELD_FEM_VEFF | 0.0581 | 0.0772 | 0.0529 | 0.0343 | 0.0681 | 0.0306 |
| 4838 | HELD_FEM_VEFF | 0.0581 | 0.0772 | 0.0529 | 0.0343 | 0.0681 | 0.0306 |
| 4912 | HELD_FEM_EFF | 0.1257 | 0.1748 | 0.0921 | 0.0255 | 0.0361 | 0.0255 |
| 4925 | HELD_MAL_CC | 0.0436 | 0.0623 | 0.0423 | 0.0685 | 0.0895 | 0.0679 |
| 4966 | HELD_MAL_ADR3ULN | 0.0269 | 0.0282 | 0.0298 | 0.1675 | 0.1966 | 0.1669 |
| 5014 | HELD_ALL_ADR5ULN | 0.007 | 0.0104 | 0.0022 | 0.0738 | 0.0869 | 0.0611 |
| 5014 | HELD_FEM_ADR5ULN | 0.0574 | 0.0604 | 0.0276 | 0.2347 | 0.2691 | 0.2164 |
| 5296 | CVD_FEM | 0.0459 | 0.0738 | 0.0438 | 0.0585 | 0.0899 | 0.0558 |
| 5296 | HELD_FEM_EFF | 0.0703 | 0.0489 | 0.0461 | 0.4109 | 0.5177 | 0.4006 |
| 5296 | CVD_ALL | 0.145 | 0.1027 | 0.1148 | 0.0579 | 0.0771 | 0.0523 |
| 5298 | HELD_FEM_EFF | 0.0813 | 0.0465 | 0.0567 | 0.4984 | 0.7366 | 0.49 |
| 5298 | CVD_ALL | 0.107 | 0.1065 | 0.0603 | 0.0348 | 0.0376 | 0.0306 |
| 5298 | CVD_FEM | 0.1629 | 0.1593 | 0.1332 | 0.0511 | 0.0885 | 0.049 |
| 5320 | HELD_FEM_EFF | 0.037 | 0.0397 | 0.029 | 0.016 | 0.0243 | 0.0151 |
| 5361 | CVD_MAL | 0.0947 | 0.1065 | 0.0447 | 0.0519 | 0.0654 | 0.0518 |
| 5457 | HELD_FEM_EFF | 0.1213 | 0.134 | 0.0452 | 0.2429 | 0.3056 | 0.2246 |
| 5704 | HELD_MAL_LIP | 0.0385 | 0.0334 | 0.0406 | 0.054 | 0.0678 | 0.0503 |
| 5704 | CVD_MAL | 0.0701 | 0.0755 | 0.07 | 0.0246 | 0.0281 | 0.0259 |
| 5717 | HELD_FEM_ADR3ULN | 0.0736 | 0.0775 | 0.0739 | 0.0219 | 0.026 | 0.021 |
| 5717 | HELD_ALL_ADR3ULN | 0.1246 | 0.1264 | 0.1214 | 0.0391 | 0.0471 | 0.0389 |
| 5959 | HELD_ALL_CC | 0.0126 | 0.0122 | 0.0098 | 0.0046 | 0.0073 | 0.0044 |
| 5959 | CVD_FEM | 0.019 | 0.0225 | 0.0082 | 0.0089 | 0.0137 | 0.0083 |
| 5959 | HELD_MAL_CC | 0.0525 | 0.0589 | 0.0243 | 0.0536 | 0.0708 | 0.053 |
| 5959 | HELD_MAL_ADR5ULN | 0.038 | 0.0364 | 0.0482 | 0.1839 | 0.2158 | 0.1795 |
| 5959 | HELD_FEM_ADR | 0.054 | 0.0574 | 0.0527 | 0.0465 | 0.0539 | 0.0461 |
| 6162 | HELD_ALL_ADR3ULN | 0.0037 | 0.0034 | 0.0015 | 0.8524 | 0.9082 | 0.8522 |
| 6162 | HELD_ALL_ADR | 0.0033 | 0.003 | 0.0028 | 0.663 | 0.722 | 0.663 |
| 6162 | HELD_ALL_ADR5ULN | 0.0206 | 0.0248 | 0.006 | 0.9797 | 1 | 0.9797 |
| 6162 | HELD_MAL_ADR3ULN | 0.0412 | 0.0352 | 0.0108 | 0.4721 | 0.4836 | 0.468 |
| 6162 | HELD_FEM_ADR5ULN | 0.0274 | 0.0257 | 0.0147 | 0.4282 | 0.5487 | 0.4335 |
| 6162 | HELD_MAL_ADR | 0.0219 | 0.0217 | 0.0188 | 0.5399 | 0.6036 | 0.5399 |
| 6236 | HELD_ALL_ADR5ULN | 0.0477 | 0.0396 | 0.0641 | 0.0131 | 0.016 | 0.0158 |
| 6236 | HELD_MAL_ADR3ULN | 0.0787 | 0.0734 | 0.0762 | 0.0279 | 0.0376 | 0.0305 |
| 6236 | HELD_MAL_ADR5ULN | 0.0932 | 0.0861 | 0.0924 | 0.0297 | 0.0375 | 0.0368 |
| 6236 | HELD_ALL_ADR3ULN | 0.1516 | 0.1516 | 0.1604 | 0.0474 | 0.051 | 0.0497 |
| 6482 | HELD_MAL_HDL | 0.0359 | 0.0402 | 0.0326 | 0.009 | 0.013 | 0.0087 |
| 6482 | HELD_ALL_LIP2 | 0.0383 | 0.0381 | 0.0383 | 0.0486 | 0.0506 | 0.0487 |
| 6482 | HELD_MAL_CC2 | 0.0613 | 0.0667 | 0.0572 | 0.0114 | 0.0142 | 0.0106 |
| 6482 | HELD_MAL_LIP2 | 0.0651 | 0.0662 | 0.065 | 0.0357 | 0.04 | 0.0358 |
| 6498 | CVD_FEM | 0.145 | 0.1987 | 0.0811 | 0.0323 | 0.0389 | 0.0281 |
| 6744 | HELD_ALL_ADR5ULN | 0.0659 | 0.07 | 0.0775 | 0.02 | 0.0273 | 0.0243 |
| 7133 | HELD_MAL_CC | 0.0153 | 0.0278 | 0.0067 | 0.0006 | 0.0007 | 0.0001 |
| 8021 | CVD_FEM | 0.039 | 0.0422 | 0.0304 | 0.8726 | 1 | 0.8726 |
| 8060 | CVD_FEM | 0.044 | 0.0304 | 0.0304 | 0.1299 | 0.1961 | 0.1237 |
| 8060 | HELD_FEM_HDL | 0.0558 | 0.0753 | 0.0549 | 0.0759 | 0.0965 | 0.0753 |
| 8210 | HELD_FEM_EFF | 0.0336 | 0.0396 | 0.0276 | 0.3226 | 0.4454 | 0.3207 |
| 8592 | HELD_FEM_VEFF | 0.0395 | 0.0432 | 0.0388 | 0.8842 | 0.9331 | 0.8842 |
| 8816 | HELD_FEM_EFF | 0.0448 | 0.0448 | 0.0202 | 0.0144 | 0.0199 | 0.0128 |
| 8846 | HELD_ALL_LIP | 0.0628 | 0.0654 | 0.0521 | 0.3798 | 0.3932 | 0.3794 |
| 8943 | HELD_MAL_LIP | 0.1444 | 0.1408 | 0.0649 | 0.0389 | 0.0633 | 0.0319 |
| 9193 | HELD_FEM_LIP | 0.0561 | 0.0723 | 0.0548 | 0.0707 | 0.0889 | 0.0691 |
| 9193 | CVD_FEM | 0.1616 | 0.1289 | 0.1306 | 0.0458 | 0.0687 | 0.0424 |
| 9443 | CVD_MAL | 0.0828 | 0.0869 | 0.0213 | 0.0507 | 0.0634 | 0.0455 |
| 9516 | HELD_MAL_CC | 0.0504 | 0.0583 | 0.046 | 0.029 | 0.043 | 0.0283 |
| 9698 | HELD_MAL_ADR | 0.0106 | 0.0048 | 0.0061 | 0.0001 | 0.0001 | 0.0001 |
| 9698 | HELD_MAL_ADR3ULN | 0.0279 | 0.0274 | 0.0035 | 0.0003 | 0.0002 | 0 |
| 9698 | HELD_FEM_EFF | 0.0538 | 0.0557 | 0.0464 | 0.2251 | 0.2386 | 0.2249 |
| 9698 | HELD_MAL_ADR5ULN | 0.2515 | 0.3809 | 0.097 | 0.0239 | 0.0263 | 0.0032 |
| 9698 | CVD_ALL | 0.2256 | 0.2237 | 0.2119 | 0.0274 | 0.0357 | 0.025 |
| 9849 | HELD_FEM_CC | 0.0302 | 0.0602 | 0.0168 | 0.0327 | 0.063 | 0.0182 |
| 9849 | HELD_MAL_LIP | 0.0315 | 0.0448 | 0.0358 | 0.0376 | 0.0505 | 0.043 |
| 9883 | HELD_FEM_CC | 0.006 | 0.0053 | 0.0046 | 0.6913 | 0.8398 | 0.6915 |
| 9883 | HELD_ALL_CC | 0.0345 | 0.035 | 0.0331 | 0.5629 | 0.6344 | 0.563 |
| 10079 | CVD_ALL | 0.118 | 0.0767 | 0.048 | 0.0611 | 0.0864 | 0.0418 |
| 10079 | CVD_MAL | 0.1491 | 0.2983 | 0.0682 | 0.0413 | 0.054 | 0.0099 |
| 10481 | HELD_FEM_ADR5ULN | 0.0697 | 0.0667 | 0.0774 | 0.0136 | 0.0149 | 0.0135 |
| 10542 | HELD_FEM_UEFF | 0.0374 | 0.0214 | 0.0265 | 0.0981 | 0.1126 | 0.0911 |
| 10542 | HELD_MAL_ADR5ULN | 0.1163 | 0.1946 | 0.0404 | 0.1357 | 0.2186 | 0.046 |
| 10600 | HELD_FEM_EFF | 0.0973 | 0.1483 | 0.0418 | 0.104 | 0.1554 | 0.0445 |
| 10621 | HELD_FEM_CC | 0.0622 | 0.0649 | 0.0451 | 0.373 | 0.4126 | 0.3769 |
| 10745 | HELD_ALL_ADR5ULN | 0.0329 | 0.0356 | 0.0723 | 0.0754 | 0.0953 | 0.0832 |
| 10745 | HELD_FEM_VEFF | 0.0308 | 0.0308 | 0.0302 | 0.3022 | 0.3181 | 0.302 |
| 10747 | HELD_MAL_ADR | 0.006 | 0.0053 | 0.0044 | 0.6116 | 0.64 | 0.6115 |
| 10747 | CVD_ALL | 0.0285 | 0.0292 | 0.027 | 0.1252 | 0.1349 | 0.1253 |
| 10747 | HELD_MAL_ADR3ULN | 0.0401 | 0.0412 | 0.0505 | 0.8735 | 1 | 0.8734 |
| 10771 | HELD_MAL_ADR5ULN | 0.0176 | 0.0191 | 0.0469 | 0.0263 | 0.0458 | 0.0291 |
| 10771 | HELD_FEM_EFF | 0.1837 | 0.1844 | 0.1832 | 0.0527 | 0.0543 | 0.0525 |
| 10870 | HELD_MAL_LIP | 0.0323 | 0.0272 | 0.0156 | 0.8328 | 1 | 0.8332 |
| 10870 | HELD_FEM_LIP | 0.0431 | 0.0412 | 0.0421 | 0.0319 | 0.037 | 0.0317 |
| 10870 | HELD_MAL_CC | 0.1157 | 0.0954 | 0.0779 | 0.0341 | 0.0413 | 0.0285 |
| 10870 | HELD_ALL_CC | 0.1146 | 0.1205 | 0.109 | 0.0272 | 0.0351 | 0.027 |
| 10877 | HELD_ALL_HDL | 0.0907 | 0.1181 | 0.0333 | 0.0266 | 0.0356 | 0.007 |
| 10948 | HELD_FEM_LIP | 0.0134 | 0.0136 | 0.0127 | 0.052 | 0.0588 | 0.0517 |
| 10948 | HELD_ALL_LIP | 0.0209 | 0.0207 | 0.0197 | 0.0356 | 0.0432 | 0.0355 |
| 10948 | HELD_FEM_CC2 | 0.0513 | 0.0521 | 0.0493 | 0.3385 | 0.3602 | 0.3382 |
| 10948 | CVD_MAL | 0.0986 | 0.0986 | 0.103 | 0.0481 | 0.0548 | 0.0475 |
| 11001 | HELD_MAL_ADR5ULN | 0.0438 | 0.0618 | 0.1215 | 0.1034 | 0.1201 | 0.1152 |
| 11073 | HELD_MAL_ADR5ULN | 0.1741 | 0.1866 | 0.1892 | 0.0446 | 0.0632 | 0.0503 |
| 11153 | HELD_FEM_CC | 0.0378 | 0.0459 | 0.038 | 0.064 | 0.0726 | 0.0658 |
| 11210 | HELD_MAL_CC | 0.025 | 0.0616 | 0.0225 | 0.0335 | 0.0756 | 0.0304 |
| 11210 | HELD_ALL_ADR3ULN | 0.0344 | 0.027 | 0.0311 | 0.076 | 0.0917 | 0.0844 |
| 11210 | HELD_ALL_ADR | 0.0536 | 0.038 | 0.0354 | 0.2211 | 0.2468 | 0.2195 |
| 11248 | HELD_FEM_ADR | 0.0125 | 0.0119 | 0.0118 | 0.0368 | 0.0494 | 0.0364 |
| 11248 | HELD_MAL_LIP | 0.0478 | 0.0677 | 0.0404 | 0.0784 | 0.1038 | 0.0644 |
| 11248 | HELD_ALL_CC | 0.0431 | 0.0567 | 0.0425 | 0.0874 | 0.1066 | 0.0887 |
| 11372 | HELD_MAL_LIP | 0.2326 | 0.2665 | 0.2343 | 0.0486 | 0.0753 | 0.0477 |
| 11449 | HELD_FEM_CC | 0.0245 | 0.0119 | 0.0204 | 0.0644 | 0.0971 | 0.0663 |
| 11450 | HELD_FEM_EFF | 0.0922 | 0.0949 | 0.0903 | 0.0362 | 0.0394 | 0.036 |
| 11470 | HELD_MAL_LIP | 0.0807 | 0.1484 | 0.0304 | 0.0882 | 0.1582 | 0.033 |
| 11472 | HELD_MAL_LIP | 0.0763 | 0.1465 | 0.0284 | 0.0836 | 0.1565 | 0.031 |
| 11472 | HELD_FEM_LIP | 0.0576 | 0.0991 | 0.0495 | 0.0617 | 0.1046 | 0.053 |
| 11487 | HELD_MAL_ADR5ULN | 0.0033 | 0.0039 | 0.0004 | 0.0122 | 0.0159 | 0.0012 |
| 11487 | HELD_MAL_ADR3ULN | 0.0156 | 0.021 | 0.0131 | 0.038 | 0.0474 | 0.0295 |
| 11488 | HELD_MAL_ADR5ULN | 0.0117 | 0.0227 | 0.0018 | 0.0076 | 0.0087 | 0.0006 |
| 11488 | HELD_FEM_UEFF | 0.0217 | 0.021 | 0.0091 | 0.0655 | 0.0713 | 0.0672 |
| 11488 | HELD_MAL_ADR3ULN | 0.0239 | 0.0311 | 0.0166 | 0.0898 | 0.127 | 0.0797 |
| 11493 | HELD_MAL_CC | 0.0736 | 0.0542 | 0.0493 | 0.6283 | 0.7502 | 0.6293 |
| 11502 | HELD_MAL_ADR3ULN | 0.0881 | 0.0865 | 0.0363 | 0.0283 | 0.0301 | 0.0225 |
| 11502 | HELD_MAL_ADR5ULN | 0.1706 | 0.154 | 0.1118 | 0.0592 | 0.0659 | 0.0396 |
| 11534 | HELD_ALL_LIP | 0.1034 | 0.2501 | 0.0513 | 0.1046 | 0.2518 | 0.0519 |
| 11537 | CVD_FEM | 0.1061 | 0.1119 | 0.0989 | 0.0221 | 0.0256 | 0.0214 |
| 11537 | HELD_FEM_EFF | 0.1916 | 0.2436 | 0.1166 | 0.0438 | 0.0655 | 0.0324 |
| 11560 | HELD_FEM_EFF | 0.1693 | 0.3529 | 0.1436 | 0.0519 | 0.1212 | 0.0386 |
| 11578 | HELD_FEM_LIP | 0.0201 | 0.0333 | 0.0132 | 0.0226 | 0.0366 | 0.0147 |
| 11578 | CVD_FEM | 0.0435 | 0.0775 | 0.0229 | 0.0459 | 0.0799 | 0.0241 |
| 11594 | HELD_FEM_ADR3ULN | 0.1373 | 0.2125 | 0.0418 | 0.0279 | 0.0331 | 0.0052 |
| 11594 | HELD_ALL_ADR5ULN | 0.1669 | 0.1552 | 0.0434 | 0.0516 | 0.0536 | 0.0092 |
| 11594 | HELD_ALL_CC | 0.0539 | 0.0724 | 0.0479 | 0.0648 | 0.0846 | 0.0574 |
| 11594 | HELD_ALL_ADR | 0.1052 | 0.0878 | 0.1 | 0.0304 | 0.036 | 0.0286 |
| 11594 | HELD_FEM_ADR5ULN | 0.3753 | 0.4458 | 0.1824 | 0.1236 | 0.213 | 0.0409 |
| 11624 | HELD_ALL_CC | 0.0352 | 0.0383 | 0.0111 | 0.3119 | 0.388 | 0.3111 |
| 11624 | HELD_MAL_CC | 0.032 | 0.0313 | 0.0164 | 0.6153 | 0.7739 | 0.6163 |
| 11624 | HELD_FEM_EFF | 0.2292 | 0.244 | 0.1389 | 0.053 | 0.0656 | 0.0407 |
| 11627 | HELD_ALL_CC | 0.0337 | 0.0316 | 0.0088 | 0.0936 | 0.1309 | 0.0921 |
| 11627 | HELD_MAL_CC | 0.0931 | 0.0933 | 0.0528 | 0.352 | 0.4146 | 0.3531 |
| 11627 | HELD_FEM_EFF | 0.1916 | 0.2436 | 0.1166 | 0.0438 | 0.0655 | 0.0324 |
| 11644 | HELD_MAL_ADR5ULN | 0.2097 | 0.2525 | 0.1344 | 0.0676 | 0.1027 | 0.0467 |
| 11650 | HELD_FEM_EFF | 0.0366 | 0.0361 | 0.0363 | 0.1123 | 0.1212 | 0.1122 |
| 11654 | HELD_ALL_ADR5ULN | 0.0052 | 0.0046 | 0.0042 | 0.6623 | 0.7404 | 0.6642 |
| 11654 | HELD_FEM_ADR5ULN | 0.0104 | 0.0096 | 0.006 | 0.7072 | 0.832 | 0.7087 |
| 11654 | HELD_FEM_ADR3ULN | 0.0546 | 0.0592 | 0.0524 | 0.6906 | 0.7512 | 0.6913 |
| 11654 | HELD_ALL_ADR3ULN | 0.052 | 0.0518 | 0.0601 | 0.2706 | 0.2742 | 0.2735 |
| 11655 | HELD_ALL_ADR5ULN | 0.0085 | 0.0074 | 0.0058 | 0.8555 | 0.8723 | 0.8558 |
| 11655 | HELD_FEM_ADR5ULN | 0.0136 | 0.0138 | 0.0053 | 0.7681 | 0.8443 | 0.7672 |
| 11655 | HELD_FEM_ADR3ULN | 0.0489 | 0.048 | 0.0432 | 0.9169 | 1 | 0.9169 |
| 11656 | HELD_MAL_LIP | 0.0321 | 0.0317 | 0.0346 | 0.012 | 0.0141 | 0.0126 |
| 11656 | HELD_FEM_EFF | 0.0782 | 0.0909 | 0.0511 | 0.0442 | 0.0652 | 0.0393 |
| 11656 | HELD_ALL_LIP | 0.0617 | 0.0646 | 0.06 | 0.0295 | 0.0353 | 0.0295 |
| 11825 | HELD_MAL_ADR5ULN | 0.0233 | 0.056 | 0.0499 | 0.0278 | 0.0619 | 0.0612 |
| 11914 | HELD_MAL_ADR5ULN | 0.0186 | 0.0915 | 0.0128 | 0.0001 | 0.0001 | 0 |
| 11914 | HELD_ALL_ADR5ULN | 0.1572 | 0.1781 | 0.1391 | 0.0477 | 0.0533 | 0.0487 |
| 12008 | HELD_FEM_EFF | 0.0222 | 0.0317 | 0.0209 | 0.0249 | 0.0351 | 0.0234 |
| 12008 | HELD_ALL_ADR5ULN | 0.1272 | 0.2155 | 0.0422 | 0.135 | 0.225 | 0.0445 |
| 12097 | HELD_ALL_ADR5ULN | 0.0162 | 0.0277 | 0.0308 | 0.019 | 0.0313 | 0.0367 |
| 12097 | HELD_FEM_ADR3ULN | 0.0342 | 0.0487 | 0.042 | 0.0392 | 0.0543 | 0.0484 |
| 12097 | HELD_MAL_ADR5ULN | 0.04 | 0.0749 | 0.0726 | 0.0462 | 0.081 | 0.0857 |
| 12097 | HELD_ALL_ADR3ULN | 0.0465 | 0.073 | 0.056 | 0.0524 | 0.0805 | 0.0633 |
| 12366 | HELD_FEM_UEFF | 0.0342 | 0.0313 | 0.0069 | 0.0364 | 0.0514 | 0.0338 |
| 12366 | HELD_ALL_ADR5ULN | 0.0464 | 0.0391 | 0.0411 | 0.5197 | 0.5929 | 0.5131 |
| 12619 | HELD_MAL_ADR5ULN | 0.0073 | 0.1235 | 0.0387 | 0.0075 | 0.1235 | 0.0398 |
| 12619 | HELD_ALL_ADR5ULN | 0.0121 | 0.0605 | 0.0414 | 0.0125 | 0.0613 | 0.0427 |
| 13025 | HELD_ALL_ADR5ULN | 0.044 | 0.0399 | 0.0593 | 0.3978 | 0.4443 | 0.4018 |
| 13191 | HELD_FEM_LIP | 0.0157 | 0.0149 | 0.015 | 0.0072 | 0.0088 | 0.0071 |
| 13191 | HELD_MAL_CC | 0.0648 | 0.0601 | 0.0431 | 0.0199 | 0.0396 | 0.0196 |
| 13191 | HELD_ALL_LIP | 0.0634 | 0.0669 | 0.0616 | 0.0211 | 0.0217 | 0.0206 |
| 13937 | HELD_FEM_ADR5ULN | 0.076 | 0.0835 | 0.0789 | 0.0402 | 0.0615 | 0.0462 |
| 900002 | CVD_FEM | 0.1492 | 0.1674 | 0.1456 | 0.0364 | 0.04 | 0.0364 |
| 900013 | CVD_FEM | 0.0212 | 0.022 | 0.0192 | 0.2613 | 0.3039 | 0.2602 |
| 900013 | CVD_ALL | 0.0279 | 0.0289 | 0.0279 | 0.1847 | 0.2004 | 0.1858 |
| 900025 | CVD_MAL | 0.1379 | 0.1533 | 0.1361 | 0.0426 | 0.0452 | 0.0439 |
| 900032 | CVD_FEM | 0.0555 | 0.036 | 0.0317 | 0.2549 | 0.3578 | 0.2418 |
| 900045 | HELD_FEM_EFF | 0.162 | 0.2388 | 0.151 | 0.0411 | 0.0579 | 0.0349 |
| 900065 | CVD_FEM | 0.0222 | 0.0175 | 0.0086 | 0.0066 | 0.0077 | 0.0057 |
| 900065 | CVD_MAL | 0.0549 | 0.0421 | 0.0289 | 0.4512 | 0.5001 | 0.453 |
| 900065 | CVD_ALL | 0.0773 | 0.0753 | 0.0754 | 0.0471 | 0.0505 | 0.0477 |
| 900078 | HELD_ALL_ADR3ULN | 0.0283 | 0.036 | 0.0348 | 0.0335 | 0.0417 | 0.0415 |
| 900078 | HELD_ALL_ADR5ULN | 0.03 | 0.0417 | 0.0487 | 0.0349 | 0.0466 | 0.0574 |
| 900078 | HELD_FEM_ADR3ULN | 0.0342 | 0.0487 | 0.042 | 0.0392 | 0.0543 | 0.0484 |
| 900082 | HELD_FEM_ADR3ULN | 0.0377 | 0.0378 | 0.0364 | 0.1073 | 0.111 | 0.1055 |
| 900082 | HELD_FEM_ADR5ULN | 0.0517 | 0.0587 | 0.0566 | 0.0581 | 0.0837 | 0.0542 |
| 900096 | CVD_ALL | 0.0644 | 0.0622 | 0.0602 | 0.032 | 0.0354 | 0.0294 |
| 900107 | HELD_MAL_ADR5ULN | 0.2371 | 0.2767 | 0.1405 | 0.0665 | 0.1045 | 0.0455 |
| 900115 | HELD_MAL_ADR5ULN | 0.0214 | 0.02 | 0.0409 | 0.0148 | 0.0208 | 0.0158 |
| 900115 | HELD_FEM_EFF | 0.0347 | 0.0338 | 0.0316 | 0.4668 | 0.5083 | 0.4661 |
| 900121 | HELD_MAL_ADR | 0.0303 | 0.0297 | 0.0268 | 0.3005 | 0.3162 | 0.3003 |
| 900173 | CVD_ALL | 0.1397 | 0.146 | 0.1347 | 0.0356 | 0.0569 | 0.0349 |
| 10000002 | HELD_FEM_EFF | 0.0781 | 0.0766 | 0.0305 | 0.0098 | 0.0139 | 0.0067 |
| 10000006 | HELD_FEM_CC | 0.0041 | 0.0018 | 0.0035 | 0.0014 | 0.0024 | 0.0014 |
| 10000006 | HELD_ALL_CC | 0.0127 | 0.0087 | 0.0113 | 0.0023 | 0.0034 | 0.002 |
| 10000014 | HELD_ALL_CC | 0.0156 | 0.0099 | 0.013 | 0.0468 | 0.0612 | 0.046 |
| 10000014 | HELD_FEM_CC | 0.0415 | 0.0248 | 0.0336 | 0.1157 | 0.1943 | 0.1184 |
| 10000025 | HELD_MAL_LIP | 0.1055 | 0.1309 | 0.0337 | 0.1763 | 0.2188 | 0.1719 |

TABLE 6a
Correlation of genotypes of PA SNPs to relative risk
For diagnostic conclusions to be drawn from genotyping a
particular patient we calculated the relative risk
RR1, RR2, RR3 for the three possible
genotypes of each SNP. Given the genotype frequencies as

| | gtype1 | gtype2 | gtype3 |
|---|---|---|---|
| case | N11 | N12 | N13 |
| control | N21 | N22 | N23 |

we calculate

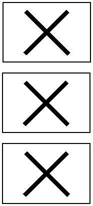

Here, the case and control populations represent any case-control-group pair, or bad(case)-good(control)-group pair, respectively (due to their increased response to statins, ‘high responders’ are treated as a case cohort, whereas ‘low responders’ are treated as the respective control cohort). A value RR1>1, RR2>1, and RR3>1 indicates an increased risk for individuals carrying genotype 1, genotype 2, and genotype 3, respectively. For example, RR1=3 indicates a 3-fold risk of an individual carrying genotype 1 as compared to individuals carrying genotype 2 or 3 (a detailed description of relative risk calculation and statistics can be found in (Biostatistics, L. D. Fisher and G. van Belle, Wiley Interscience 1993)). The baySNP number refers to an internal numbering of the PA SNPs and can be found in the sequence listing null: not defined.

In cases where a relative risk is not given in the table (three times zero or null) the informative genotype can be drawn from the right part of the table where the frequencies of genotypes are given in the cases and control cohorts. For example BaySNP 3360 gave the following results:

| BAYSNP | COMPARISON | GTYPE1 | GTYPE2 | GTYPE3 | RR1 | RR2 | RR3 |
|---|---|---|---|---|---|---|---|
| 3360 | HELD_MAL_ADR5ULN | GG | GT | TT | null | 0 | 0 |

| FQ1_A | FQ2_A | FQ3_A | FQ1_B | FQ2_B | FQ3_B |
|---|---|---|---|---|---|
| 10 | 0 | 0 | 50 | 22 | 1 |

It can be concluded that a GT or TT genotype is only present in the control cohort; these genotypes are somehow protective against ADR. An analoogous proceeding can be used to determine protective alleles if no relative risk is given (table 6b).

| BAYSNP | COMPARISON | GTYPE1 | GTYPE2 | GTYPE3 | RR1 | RR2 |
|---|---|---|---|---|---|---|
| 28 | HELD_FEM_EFF | CC | CT | TT | 0.68 | 0.29 |
| 29 | HELD_ALL_HDL | AA | AG | GG | 0 | 0.90 |
| 29 | HELD_MAL_ADR3ULN | AA | AG | GG | 2.16 | 0.56 |
| 29 | HELD_MAL_ADR5ULN | AA | AG | GG | 3.15 | 0.66 |
| 52 | HELD_FEM_EFF | CC | CG | GG | 1.96 | 1.02 |
| 56 | HELD_FEM_EFF | AA | AG | GG | null | 2.76 |
| 89 | HELD_ALL_CC | AA | AG | null | null | 0 |
| 90 | HELD_FEM_CC | CC | CT | TT | 0.97 | 0.64 |
| 99 | HELD_FEM_LIP | CC | CT | TT | 1.51 | 0.7 |
| 140 | HELD_FEM_EFF | CC | CT | TT | 0 | 0 |
| 152 | HELD_FEM_EFF | AA | AG | GG | 0.42 | 1.27 |
| 214 | HELD_ALL_LIP | AA | AG | GG | 0.92 | 1.18 |
| 214 | HELD_FEM_LIP | AA | AG | GG | 1 | 1.11 |
| 221 | HELD_ALL_CC | CC | CG | GG | 1.36 | 0.56 |
| 221 | HELD_FEM_CC | CC | CG | GG | 1.16 | 0.53 |
| 224 | HELD_FEM_LIP | CC | CT | TT | 0.77 | 1.26 |
| 224 | HELD_MAL_LIP | CC | CT | TT | 2.02 | 1.45 |
| 294 | HELD_ALL_CC | CC | CT | TT | 0.83 | 0.97 |
| 307 | CVD_FEM | CC | CT | TT | 0.34 | 0.8 |
| 307 | HELD_ALL_LIP | CC | CT | TT | null | 1.41 |
| 411 | HELD_ALL_HDL | AA | AT | TT | 1.85 | 0.69 |
| 449 | HELD_MAL_LIP | CC | CG | GG | 0 | 0.42 |
| 466 | CVD_FEM | CC | CT | TT | 0.66 | 0.86 |
| 472 | HELD_FEM_EFF | AA | AG | GG | 0 | 0 |
| 542 | HELD_MAL_CC | AA | AG | GG | 2.58 | 3.07 |
| 542 | HELD_MAL_HDL | AA | AG | GG | 0 | 2.38 |
| 542 | HELD_ALL_ADR | AA | AG | GG | 0 | 1.32 |
| 542 | HELD_FEM_HDL | AA | AG | GG | 0.57 | 0.67 |
| 739 | HELD_ALL_CC | CC | CG | GG | 0.67 | 0.94 |
| 821 | HELD_MAL_LIP2 | AA | AC | CC | 1.4 | 0.96 |
| 821 | HELD_FEM_VEFF | AA | AC | CC | 0 | 0.93 |
| 1005 | HELD_MAL_CC | AA | AG | GG | 2.35 | 0.6 |
| 1055 | HELD_MAL_CC | AA | AT | TT | 0 | 3 |
| 1056 | HELD_FEM_EFF | AA | AG | GG | 1.59 | 0.37 |
| 1085 | HELD_MAL_LIP | AA | AG | GG | 0.37 | 1.31 |
| 1085 | CVD_FEM | AA | AG | GG | 1.51 | 0.88 |
| 1086 | HELD_MAL_LIP | AA | AG | GG | 1.97 | 1 |
| 1092 | HELD_MAL_LIP | CC | CG | GG | 0.94 | 0.4 |
| 1096 | HELD_MAL_CC | GG | GT | TT | null | 2.2 |
| 1096 | CVD_MAL | GG | GT | TT | 1.51 | 0.72 |
| 1101 | HELD_FEM_EFF | CC | CT | TT | null | 0 |
| 1204 | HELD_MAL_LIP | AA | AG | GG | 3.06 | 1.58 |
| 1204 | HELD_ALL_LIP | AA | AG | GG | 1.34 | 1.18 |
| 1504 | HELD_ALL_CC | CC | CT | TT | 0.5 | 1.79 |
| 1504 | HELD_MAL_LIP | CC | CT | TT | 0 | 1.6 |
| 1504 | HELD_MAL_CC | CC | CT | TT | 0.72 | 2.63 |
| 1504 | HELD_FEM_CC | CC | CT | TT | 0.4 | 1.44 |
| 1511 | HELD_FEM_EFF | GG | GT | TT | 0.33 | 3.38 |
| 1524 | HELD_FEM_ADR3ULN | AA | AC | CC | 0 | 1.51 |
| 1556 | HELD_FEM_EFF | CC | CG | GG | null | 3.36 |
| 1561 | CVD_FEM | AA | AC | CC | 1.59 | 0.73 |
| 1582 | HELD_MAL_LIP | CC | CT | TT | 0 | 0.78 |
| 1638 | HELD_FEM_CC | AA | AG | GG | 0.56 | 0.62 |
| 1653 | CVD_MAL | GG | GT | TT | 0.86 | 1.43 |
| 1662 | HELD_MAL_CC | CC | CT | TT | 2.8 | null |
| 1714 | CVD_MAL | AA | AG | GG | 0.48 | 0.98 |
| 1722 | HELD_FEM_ADR5ULN | CC | CT | TT | 2.8 | 0.41 |
| 1757 | HELD_FEM_EFF | AA | AG | GG | 3 | 0.68 |
| 1765 | HELD_ALL_ADR3ULN | AA | AG | GG | 0.67 | 0.36 |
| 1765 | HELD_ALL_ADR3ULN | AA | AG | GG | 0.67 | 0.36 |
| 1765 | HELD_ALL_ADR5ULN | AA | AG | GG | null | 0.31 |
| 1765 | HELD_ALL_ADR5ULN | AA | AG | GG | null | 0.31 |
| 1765 | HELD_MAL_ADR3ULN | AA | AG | GG | 0 | 0.26 |
| 1765 | HELD_MAL_ADR3ULN | AA | AG | GG | 0 | 0.26 |
| 1765 | HELD_MAL_ADR5ULN | AA | AG | GG | 0 | 0 |
| 1765 | HELD_MAL_ADR5ULN | AA | AG | GG | 0 | 0 |
| 1765 | HELD_FEM_ADR3ULN | AA | AG | GG | 1.05 | 0.41 |
| 1765 | HELD_FEM_ADR3ULN | AA | AG | GG | 1.05 | 0.41 |
| 1776 | HELD_ALL_CC | AA | AG | GG | null | null |
| 1776 | HELD_FEM_CC | AA | AG | GG | null | null |
| 1799 | HELD_FEM_LIP2 | CC | CT | TT | 1.04 | 0.82 |
| 1799 | HELD_MAL_CC | CC | CT | TT | 0.45 | 1.46 |
| 1806 | HELD_FEM_EFF | AA | AG | GG | 3.96 | 0.35 |
| 1837 | HELD_FEM_LIP2 | CC | CT | TT | 1.17 | 0.77 |
| 1837 | HELD_ALL_LIP2 | CC | CT | TT | 1.18 | 0.83 |
| 1837 | HELD_ALL_ADR5ULN | CC | CT | TT | 2.82 | 0.34 |
| 1837 | HELD_MAL_ADR | CC | CT | TT | 1.45 | 0.7 |
| 1837 | HELD_MAL_LIP2 | CC | CT | TT | 1.19 | 0.89 |
| 1870 | HELD_ALL_CC | CC | CT | TT | 0.73 | 1.75 |
| 1870 | HELD_FEM_CC | CC | CT | TT | 0.85 | 1.75 |
| 1882 | CVD_MAL | CC | CT | TT | 1.06 | 0.76 |
| 1988 | HELD_ALL_LIP | CC | CT | TT | 1.26 | 0.95 |
| 2000 | CVD_MAL | CC | TT | null | 2.45 | 0.41 |
| 2000 | CVD_ALL | CC | TT | null | 1.98 | 0.51 |
| 2000 | HELD_FEM_CC2 | CC | TT | null | 3.29 | 0.3 |
| 2000 | HELD_MAL_HDL | CC | TT | null | 2.00 | 0.50 |
| 2000 | HELD_FEM_ADR | CC | TT | null | 2.01 | 0.5 |
| 2000 | HELD_MAL_CC | CC | TT | null | 0.51 | 1.98 |
| 2071 | CVD_ALL | AA | AG | GG | 1.4 | 1.09 |
| 2078 | HELD_MAL_LIP | GG | GT | TT | 3.06 | 1.9 |
| 2085 | HELD_FEM_VEFF | GG | GT | TT | 2.5 | 0.79 |
| 2095 | CVD_ALL | AG | GG | null | 1.72 | 0.58 |
| 2119 | HELD_MAL_LIP | AA | AG | null | 0.35 | 2.83 |
| 2119 | HELD_ALL_LIP | AA | AG | null | 0.72 | 1.39 |
| 2119 | HELD_FEM_EFF | AA | AG | null | 0.38 | 2.67 |
| 2141 | HELD_FEM_EFF | AA | AG | GG | 0 | 3.25 |
| 2141 | HELD_ALL_CC | AA | AG | GG | 0 | 1.35 |
| 2182 | HELD_FEM_EFF | AA | AG | GG | 3.71 | 0.65 |
| 2234 | HELD_MAL_LIP | GG | GT | TT | 0 | 0.96 |
| 2281 | HELD_FEM_VEFF | AA | AC | CC | 0 | 1.04 |
| 2298 | CVD_FEM | AA | AC | CC | 2.23 | 0.57 |
| 2298 | HELD_MAL_CC2 | AA | AC | CC | 0 | 0.7 |
| 2341 | HELD_FEM_CC | CC | CT | TT | null | 1.88 |
| 2357 | HELD_ALL_CC2 | AA | AG | GG | 2.03 | 0.76 |
| 2357 | HELD_ALL_CC | AA | AG | GG | 1.98 | 0.62 |
| 2357 | HELD_MAL_LIP | AA | AG | GG | | 0.42 |
| 2357 | HELD_FEM_CC | AA | AG | GG | 1.81 | 0.57 |
| 2366 | CVD_FEM | GG | GT | TT | 1.51 | 1.12 |
| 2423 | CVD_FEM | AA | AG | GG | 1.48 | 1.08 |
| 2708 | CVD_FEM | CC | CT | TT | 3.67 | 0.27 |
| 2995 | HELD_FEM_ADR5ULN | AA | AC | CC | 2.66 | 1.41 |
| 2995 | HELD_FEM_UEFF | AA | AC | CC | 0.67 | 0.68 |
| 3360 | HELD_MAL_ADR5ULN | GG | GT | TT | null | 0 |
| 3464 | HELD_ALL_CC | AA | AG | GG | 0.43 | 0.83 |
| 3464 | HELD_FEM_CC | AA | AG | GG | 0.6 | 0.67 |
| 3689 | HELD_FEM_EFF | CC | CG | GG | 4 | 0.82 |
| 3975 | HELD_FEM_UEFF | AA | AC | CC | 0.37 | 0.83 |
| 3976 | HELD_FEM_UEFF | AA | AG | GG | 0.34 | 0.92 |
| 4206 | HELD_FEM_ADR3ULN | AA | AT | TT | 0.57 | 1.14 |
| 4838 | HELD_FEM_VEFF | AA | AG | GG | 3.27 | 0.35 |
| 4838 | HELD_FEM_VEFF | AA | AG | GG | 3.27 | 0.35 |
| 4838 | HELD_FEM_VEFF | AA | AG | GG | 3.27 | 0.35 |
| 4912 | HELD_FEM_EFF | AA | AG | GG | 2.33 | 0 |
| 4925 | HELD_MAL_CC | AA | AC | CC | 0.45 | 2.2 |
| 4966 | HELD_MAL_ADR3ULN | AA | AG | GG | 1.08 | 0.44 |
| 5014 | HELD_ALL_ADR5ULN | AA | AG | GG | 1.54 | 0.16 |
| 5014 | HELD_FEM_ADR5ULN | AA | AG | GG | 1.64 | 0.15 |
| 5296 | CVD_FEM | AA | AG | GG | null | 1.7 |
| 5296 | HELD_FEM_EFF | AA | AG | GG | 3 | 0.22 |
| 5296 | CVD_ALL | AA | AG | GG | 1.72 | 1.29 |
| 5298 | HELD_FEM_EFF | CC | CT | TT | 3.2 | 0.23 |
| 5298 | CVD_ALL | CC | CT | TT | 1.76 | 1.24 |
| 5298 | CVD_FEM | CC | CT | TT | 2.18 | 1.56 |
| 5320 | HELD_FEM_EFF | AA | AG | GG | 0.23 | 0.88 |
| 5361 | CVD_MAL | AA | AC | CC | 0.77 | 1.54 |
| 5457 | HELD_FEM_EFF | AA | AG | GG | 1.41 | 0 |
| 5704 | HELD_MAL_LIP | CC | CT | TT | 0.7 | 0.45 |
| 5704 | CVD_MAL | CC | CT | TT | 0.65 | 0.87 |
| 5717 | HELD_FEM_ADR3ULN | AA | AG | GG | 1.77 | 0.82 |
| 5717 | HELD_ALL_ADR3ULN | AA | AG | GG | 1.44 | 1.01 |
| 5959 | HELD_ALL_CC | AA | AG | GG | 1.81 | 0.85 |
| 5959 | CVD_FEM | AA | AG | GG | 3.6 | 0.8 |
| 5959 | HELD_MAL_CC | AA | AG | GG | 2.7 | 0.82 |
| 5959 | HELD_MAL_ADR5ULN | AA | AG | GG | 1.16 | 0.22 |
| 5959 | HELD_FEM_ADR | AA | AG | GG | 1.15 | 1.32 |
| 6162 | HELD_ALL_ADR3ULN | CC | CG | GG | 0.15 | 1.78 |
| 6162 | HELD_ALL_ADR | CC | CG | GG | 0.45 | 1.33 |
| 6162 | HELD_ALL_ADR5ULN | CC | CG | GG | 0 | 2.35 |
| 6162 | HELD_MAL_ADR3ULN | CC | CG | GG | 0 | 1.85 |
| 6162 | HELD_FEM_ADR5ULN | CC | CG | GG | 0 | 3.19 |
| 6162 | HELD_MAL_ADR | CC | CG | GG | 0.4 | 1.39 |
| 6236 | HELD_ALL_ADR5ULN | CC | CT | TT | 2.41 | 1.25 |
| 6236 | HELD_MAL_ADR3ULN | CC | CT | TT | 1.74 | 1.63 |
| 6236 | HELD_MAL_ADR5ULN | CC | CT | TT | 2.68 | 2.12 |
| 6236 | HELD_ALL_ADR3ULN | CC | CT | TT | 1.58 | 1.15 |
| 6482 | HELD_MAL_HDL | AA | AG | GG | 0.44 | 1.96 |
| 6482 | HELD_ALL_LIP2 | AA | AG | GG | 0.87 | 1.16 |
| 6482 | HELD_MAL_CC2 | AA | AG | GG | 1.93 | 0.66 |
| 6482 | HELD_MAL_LIP2 | AA | AG | GG | 0.83 | 1.2 |
| 6498 | CVD_FEM | AA | AG | GG | 1.85 | 0.73 |
| 6744 | HELD_ALL_ADR5ULN | CC | CT | TT | 2.27 | 1.54 |
| 7133 | HELD_MAL_CC | CC | CG | GG | 0.36 | null |
| 8021 | CVD_FEM | AA | AG | GG | 0.71 | 1.98 |
| 8060 | CVD_FEM | AA | AG | GG | 2.1 | 0.38 |
| 8060 | HELD_FEM_HDL | AA | AG | GG | 0.47 | 2.13 |
| 8210 | HELD_FEM_EFF | AA | AG | GG | 0.22 | 2.93 |
| 8592 | HELD_FEM_VEFF | CC | CT | TT | 0.7 | 1.32 |
| 8816 | HELD_FEM_EFF | CC | CG | GG | 2.22 | 1.17 |
| 8846 | HELD_ALL_LIP | AA | AG | GG | 1 | 1.18 |
| 8943 | HELD_MAL_LIP | AA | AC | CC | 1.89 | 0.78 |
| 9193 | HELD_FEM_LIP | CC | CG | GG | 1.54 | 0.65 |
| 9193 | CVD_FEM | CC | CG | GG | 0.59 | 1.59 |
| 9443 | CVD_MAL | CC | CT | TT | 1.55 | 1 |
| 9516 | HELD_MAL_CC | AA | AG | GG | 2.56 | 0.52 |
| 9698 | HELD_MAL_ADR | AA | AG | GG | 0.41 | 0 |
| 9698 | HELD_MAL_ADR3ULN | AA | AG | GG | 0 | 0 |
| 9698 | HELD_FEM_EFF | AA | AG | GG | 0.47 | 1.04 |
| 9698 | HELD_MAL_ADR5ULN | AA | AG | GG | 0 | 0 |
| 9698 | CVD_ALL | AA | AG | GG | 1.31 | 1.09 |
| 9849 | HELD_FEM_CC | CC | CT | null | null | 0 |
| 9849 | HELD_MAL_LIP | CC | CT | null | 0.42 | 2.38 |
| 9883 | HELD_FEM_CC | AA | AG | GG | 1.64 | 0.46 |
| 9883 | HELD_ALL_CC | AA | AG | GG | 1.37 | 0.58 |
| 10079 | CVD_ALL | AA | AG | GG | 1.74 | 0 |
| 10079 | CVD_MAL | AA | AG | GG | 1.53 | null |
| 10481 | HELD_FEM_ADR5ULN | AA | AT | TT | 0.4 | 0.85 |
| 10542 | HELD_FEM_UEFF | CC | CT | TT | 2.42 | 0.47 |
| 10542 | HELD MAL_ADR5ULN | CC | CT | TT | null | 0 |
| 10600 | HELD_FEM_EFF | AA | AG | GG | null | 0 |
| 10621 | HELD_FEM_CC | CC | CT | TT | 1.56 | 0.49 |
| 10745 | HELD_ALL_ADR5ULN | AA | AG | GG | 3.09 | 0.86 |
| 10745 | HELD_FEM_VEFF | AA | AG | GG | 0.79 | 1.35 |
| 10747 | HELD_MAL_ADR | CC | CT | TT | 1.71 | 0.62 |
| 10747 | CVD_ALL | CC | CT | TT | 1.75 | 0.73 |
| 10747 | HELD_MAL_ADR3ULN | CC | CT | TT | 2.24 | 0.45 |
| 10771 | HELD_MAL_ADR5ULN | CC | CG | GG | 4.67 | 0.67 |
| 10771 | HELD_FEM_EFF | CC | CG | GG | 1.14 | 1.07 |
| 10870 | HELD_MAL_LIP | AA | AG | GG | 0 | 2.26 |
| 10870 | HELD_FEM_LIP | AA | AG | GG | 0.9 | 0.65 |
| 10870 | HELD_MAL_CC | AA | AG | GG | 0 | 0.52 |
| 10870 | HELD_ALL_CC | AA | AG | GG | 0.45 | 0.83 |
| 10877 | HELD_ALL_HDL | AA | AC | CC | 0.61 | 0.53 |
| 10948 | HELD_FEM_LIP | GG | GT | TT | 0.58 | 1.45 |
| 10948 | HELD_ALL_LIP | GG | GT | TT | 0.62 | 1.35 |
| 10948 | HELD_FEM_CC2 | GG | GT | TT | 0.59 | 1.67 |
| 10948 | CVD_MAL | GG | GT | TT | 0.69 | 1.09 |
| 11001 | HELD_MAL_ADR5ULN | CC | CT | TT | 5.06 | 1.02 |
| 11073 | HELD_MAL_ADR5ULN | CC | CG | GG | 2.71 | 1.32 |
| 11153 | HELD_FEM_CC | CC | CT | TT | 1.76 | 0.57 |
| 11210 | HELD_MAL_CC | CC | CT | TT | 0.4 | 2.5 |
| 11210 | HELD_ALL_ADR3ULN | CC | CT | TT | 0.6 | 1.79 |
| 11210 | HELD_ALL_ADR | CC | CT | TT | 0.8 | 1.32 |
| 11248 | HELD_FEM_ADR | CC | CT | TT | 1.57 | 0.59 |
| 11248 | HELD_MAL_LIP | CC | CT | TT | 2.65 | 0.38 |
| 11248 | HELD_ALL_CC | CC | CT | TT | 1.54 | 0.65 |
| 11372 | HELD_MAL_LIP | AA | AG | GG | 1.8 | 0.83 |
| 11449 | HELD_FEM_CC | CC | CG | GG | 1.73 | 0.41 |
| 11450 | HELD_FEM_EFF | AA | AT | TT | 1.3 | 1.06 |
| 11470 | HELD_MAL_LIP | CC | CT | null | null | 0 |
| 11472 | HELD_MAL_LIP | AA | AT | null | null | 0 |
| 11472 | HELD_FEM_LIP | AA | AT | null | 0.61 | 1.63 |
| 11487 | HELD_MAL_ADR5ULN | AT | TT | null | 0 | null |
| 11487 | HELD_MAL_ADR3ULN | AT | TT | null | 0.4 | 2.5 |
| 11488 | HELD_MAL_ADR5ULN | CC | CG | GG | null | 0 |
| 11488 | HELD_FEM_UEFF | CC | CG | GG | 0.79 | 1.02 |
| 11488 | HELD_MAL_ADR3ULN | CC | CG | GG | 2.48 | 0.3 |
| 11493 | HELD_MAL_CC | AA | AG | GG | 0 | 2.25 |
| 11502 | HELD_MAL_ADR3ULN | CC | CT | TT | 0 | 0.69 |
| 11502 | HELD_MAL_ADR5ULN | CC | CT | TT | 0 | 0.4 |
| 11534 | HELD_ALL_LIP | GG | GT | null | null | 0 |
| 11537 | CVD_FEM | AA | AG | GG | 0.63 | 1.38 |
| 11537 | HELD_FEM_EFF | AA | AG | GG | 2.73 | 0.56 |
| 11560 | HELD_FEM_EFF | AA | AG | GG | 3 | |
| 11578 | HELD_FEM_LIP | CC | CT | null | 4.62 | 0.22 |
| 11578 | CVD_FEM | CC | CT | null | 0.41 | 2.44 |
| 11594 | HELD_FEM_ADR3ULN | CC | CT | TT | 0 | 0 |
| 11594 | HELD_ALL_ADR5ULN | CC | CT | TT | 0 | 0 |
| 11594 | HELD_ALL_CC | CC | CT | TT | null | 1.6 |
| 11594 | HELD_ALL_ADR | CC | CT | TT | 0.66 | 0.58 |
| 11594 | HELD_FEM_ADR5ULN | CC | CT | TT | 0 | 0 |
| 11624 | HELD_ALL_CC | CC | CT | TT | 1 | 0.75 |
| 11624 | HELD_MAL_CC | CC | CT | TT | 1.32 | 0.33 |
| 11624 | HELD_FEM_EFF | CC | CT | TT | 2.5 | 0.63 |
| 11627 | HELD_ALL_CC | CC | CT | TT | 0.86 | 0.86 |
| 11627 | HELD_MAL_CC | CC | CT | TT | 1 | 0.58 |
| 11627 | HELD_FEM_EFF | CC | CT | TT | 2.73 | 0.56 |
| 11644 | HELD_MAL_ADR5ULN | AA | AG | GG | 0 | 0.45 |
| 11650 | HELD_FEM_EFF | AA | AG | GG | 1.07 | 0.8 |
| 11654 | HELD_ALL_ADR5ULN | AA | AG | GG | 2.59 | 0.24 |
| 11654 | HELD_FEM_ADR5ULN | AA | AG | GG | 2.81 | 0.12 |
| 11654 | HELD_FEM_ADR3ULN | AA | AG | GG | 1.81 | 0.48 |
| 11654 | HELD_ALL_ADR3ULN | AA | AG | GG | 1.83 | 0.66 |
| 11655 | HELD_ALL_ADR5ULN | AA | AC | CC | 1.56 | 0.24 |
| 11655 | HELD_FEM_ADR5ULN | AA | AC | CC | 2.03 | 0.11 |
| 11655 | HELD_FEM_ADR3ULN | AA | AC | CC | 1.34 | 0.45 |
| 11656 | HELD_MAL_LIP | CC | CT | TT | 0.53 | 0.96 |
| 11656 | HELD_FEM_EFF | CC | CT | TT | 2.57 | 0.56 |
| 11656 | HELD_ALL_LIP | CC | CT | TT | 0.79 | 1.01 |
| 11825 | HELD_MAL_ADR5ULN | AA | AG | null | 0.25 | 4 |
| 11914 | HELD_MAL_ADR5ULN | AA | AT | TT | 0.11 | 0 |
| 11914 | HELD_ALL_ADR5ULN | AA | AT | TT | 0.45 | 1.43 |
| 12008 | HELD_FEM_EFF | CC | CT | null | 0.72 | 1.38 |
| 12008 | HELD_ALL_ADR5ULN | CC | CT | null | null | 0 |
| 12097 | HELD_ALL_ADR5ULN | AG | GG | null | 2.66 | 0.38 |
| 12097 | HELD_FEM_ADR3ULN | AG | GG | null | 2.05 | 0.49 |
| 12097 | HELD_MAL_ADR5ULN | AG | GG | null | 3.48 | 0.29 |
| 12097 | HELD_ALL_ADR3ULN | AG | GG | null | 1.77 | 0.56 |
| 12366 | HELD_FEM_UEFF | AA | AG | GG | 1.33 | 1.02 |
| 12366 | HELD_ALL_ADR5ULN | AA | AG | GG | 1.82 | 0.34 |
| 12619 | HELD_MAL_ADR5ULN | AG | GG | null | 8.89 | 0.11 |
| 12619 | HELD_ALL_ADR5ULN | AG | GG | null | 4.67 | 0.21 |
| 13025 | HELD_ALL_ADR5ULN | AA | AC | CC | 1.12 | 0.51 |
| 13191 | HELD_FEM_LIP | AA | AG | GG | 0.71 | 0.71 |
| 13191 | HELD_MAL_CC | AA | AG | GG | 2.5 | 1.67 |
| 13191 | HELD_ALL_LIP | AA | AG | GG | 0.65 | 0.81 |
| 13937 | HELD_FEM_ADR5ULN | AA | AC | CC | 0.36 | 1.91 |
| 900002 | CVD_FEM | GG | GT | TT | 1.65 | 1.29 |
| 900013 | CVD_FEM | CC | CG | GG | 1.7 | 0.47 |
| 900013 | CVD_ALL | CC | CG | GG | 1.32 | 0.7 |
| 900025 | CVD_MAL | GG | GT | TT | 0.73 | 0.88 |
| 900032 | CVD_FEM | CC | CT | TT | 2.48 | 0.22 |
| 900045 | HELD_FEM_EFF | CC | CT | TT | 0.42 | 0.48 |
| 900065 | CVD_FEM | AA | AC | CC | 1.91 | 0.7 |
| 900065 | CVD_MAL | AA | AC | CC | 1.29 | 0.72 |
| 900065 | CVD_ALL | AA | AC | CC | 1.36 | 0.77 |
| 900078 | HELD_ALL_ADR3ULN | AA | AG | GG | 0.56 | 1.79 |
| 900078 | HELD_ALL_ADR5ULN | AA | AG | GG | 0.41 | 2.45 |
| 900078 | HELD_FEM_ADR3ULN | AA | AG | GG | 0.49 | 2.05 |
| 900082 | HELD_FEM_ADR3ULN | AA | AG | GG | 1 | 0.49 |
| 900082 | HELD_FEM_ADR5ULN | AA | AG | GG | 0.76 | 0.39 |
| 900096 | CVD_ALL | AA | AG | GG | 0.74 | 1.35 |
| 900107 | HELD_MAL_ADR5ULN | CC | CT | TT | 0 | 0.52 |
| 900115 | HELD_MAL_ADR5ULN | AA | AG | GG | 0.24 | 0.78 |
| 900115 | HELD_FEM_EFF | AA | AG | GG | 1.47 | 0.56 |
| 900121 | HELD_MAL_ADR | GG | GT | TT | 0.46 | 1.42 |
| 900173 | CVD_ALL | GG | GT | TT | 0.5 | 1.35 |
| 10000002 | HELD_FEM_EFF | AA | AG | GG | 2.67 | 0.8 |
| 10000006 | HELD_FEM_CC | AA | AG | GG | 3.35 | 0.26 |
| 10000006 | HELD_ALL_CC | AA | AG | GG | 2.52 | 0.41 |
| 10000014 | HELD_ALL_CC | AA | AC | CC | 2.18 | 0.33 |
| 10000014 | HELD_FEM_CC | AA | AC | CC | 2.17 | 0.34 |
| 10000025 | HELD_MAL_LIP | CC | CT | TT | 1.17 | 1.41 |

| BAYSNP | RR3 | FQ1_A | FQ2_A | FQ3_A | FQ1_B | FQ2_B | FQ3_B |
|---|---|---|---|---|---|---|---|
| 28 | 3.38 | 1 | 2 | 9 | 3 | 12 | 7 |
| 29 | 0.58 | 4 | 4 | 2 | 0 | 7 | 8 |
| 29 | 0.75 | 13 | 7 | 6 | 18 | 32 | 22 |
| 29 | 0.32 | 5 | 3 | 1 | 18 | 32 | 22 |
| 52 | 0.23 | 7 | 10 | 1 | 5 | 17 | 9 |
| 56 | 0.36 | 0 | 5 | 7 | 0 | 2 | 20 |
| 89 | null | 45 | 0 | 0 | 37 | 3 | 0 |
| 90 | 1.82 | 8 | 13 | 10 | 6 | 15 | 1 |
| 99 | 1.16 | 13 | 28 | 41 | 5 | 41 | 34 |
| 140 | null | 0 | 0 | 12 | 1 | 2 | 18 |
| 152 | 2.5 | 3 | 6 | 3 | 12 | 9 | 1 |
| 214 | 0 | 59 | 38 | 0 | 73 | 36 | 4 |
| 214 | 0 | 50 | 31 | 0 | 48 | 26 | 4 |
| 221 | 1.44 | 7 | 12 | 26 | 3 | 21 | 15 |
| 221 | 1.67 | 4 | 9 | 18 | 2 | 14 | 6 |
| 224 | 1.24 | 51 | 8 | 20 | 60 | 5 | 14 |
| 224 | 0.38 | 17 | 1 | 2 | 25 | 1 | 11 |
| 294 | 2 | 16 | 24 | 5 | 18 | 22 | 0 |
| 307 | 1.84 | 2 | 15 | 19 | 9 | 20 | 9 |
| 307 | 0.71 | 0 | 70 | 32 | 0 | 63 | 54 |
| 411 | 0.56 | 7 | 3 | 0 | 5 | 8 | 2 |
| 449 | 2.62 | 0 | 3 | 17 | 1 | 14 | 22 |
| 466 | 1.61 | 6 | 15 | 14 | 12 | 20 | 8 |
| 472 | null | 0 | 0 | 11 | 3 | 6 | 13 |
| 542 | 0.23 | 2 | 8 | 4 | 0 | 2 | 17 |
| 542 | 0.30 | 3 | 8 | 10 | 0 | 3 | 24 |
| 542 | 0.78 | 0 | 53 | 106 | 2 | 33 | 119 |
| 542 | 1.56 | 0 | 2 | 21 | 1 | 8 | 23 |
| 739 | 1.52 | 9 | 21 | 15 | 14 | 20 | 6 |
| 821 | 0.93 | 32 | 116 | 161 | 18 | 138 | 193 |
| 821 | 2.1 | 0 | 4 | 6 | 4 | 6 | 4 |
| 1005 | 0 | 12 | 2 | 0 | 11 | 5 | 2 |
| 1055 | 1 | 0 | 3 | 6 | 4 | 0 | 8 |
| 1056 | 2.04 | 12 | 6 | 6 | 10 | 21 | 2 |
| 1085 | 1.75 | 3 | 11 | 6 | 15 | 16 | 5 |
| 1085 | 0.5 | 20 | 11 | 3 | 16 | 15 | 9 |
| 1086 | 0.44 | 7 | 10 | 3 | 5 | 18 | 13 |
| 1092 | 2.38 | 2 | 5 | 13 | 4 | 21 | 12 |
| 1096 | 0.45 | 0 | 7 | 7 | 0 | 3 | 15 |
| 1096 | 1.22 | 4 | 13 | 52 | 0 | 12 | 21 |
| 1101 | null | 12 | 0 | 0 | 18 | 4 | 0 |
| 1204 | 0.49 | 2 | 8 | 9 | 0 | 9 | 26 |
| 1204 | 0.77 | 12 | 38 | 49 | 8 | 36 | 71 |
| 1504 | 0.78 | 5 | 27 | 12 | 12 | 12 | 15 |
| 1504 | 1.14 | 0 | 12 | 7 | 8 | 17 | 12 |
| 1504 | 0.4 | 2 | 9 | 3 | 4 | 4 | 10 |
| 1504 | 1.13 | 3 | 18 | 9 | 8 | 8 | 5 |
| 1511 | 0 | 3 | 9 | 0 | 14 | 7 | 1 |
| 1524 | 0.89 | 0 | 16 | 22 | 8 | 23 | 51 |
| 1556 | 0.3 | 0 | 7 | 5 | 0 | 3 | 19 |
| 1561 | 0.41 | 23 | 12 | 1 | 17 | 19 | 4 |
| 1582 | 1.89 | 0 | 5 | 15 | 5 | 12 | 20 |
| 1638 | 1.73 | 1 | 8 | 22 | 2 | 11 | 9 |
| 1653 | 0.71 | 15 | 40 | 14 | 10 | 10 | 13 |
| 1662 | 0.36 | 4 | 0 | 10 | 0 | 0 | 18 |
| 1714 | 1.23 | 3 | 26 | 37 | 6 | 14 | 14 |
| 1722 | 0.93 | 8 | 5 | 5 | 14 | 43 | 24 |
| 1757 | 0.88 | 4 | 7 | 9 | 0 | 16 | 16 |
| 1765 | 2.71 | 1 | 7 | 55 | 4 | 48 | 97 |
| 1765 | 2.71 | 1 | 7 | 55 | 4 | 48 | 97 |
| 1765 | 3.64 | 0 | 3 | 24 | 4 | 48 | 97 |
| 1765 | 3.64 | 0 | 3 | 24 | 4 | 48 | 97 |
| 1765 | 4.23 | 0 | 2 | 24 | 2 | 21 | 47 |
| 1765 | 4.23 | 0 | 2 | 24 | 2 | 21 | 47 |
| 1765 | null | 0 | 0 | 10 | 2 | 21 | 47 |
| 1765 | null | 0 | 0 | 10 | 2 | 21 | 47 |
| 1765 | 2.23 | 1 | 5 | 31 | 2 | 27 | 50 |
| 1765 | 2.23 | 1 | 5 | 31 | 2 | 27 | 50 |
| 1776 | 0 | 45 | 0 | 0 | 37 | 0 | 3 |
| 1776 | 0 | 31 | 0 | 0 | 20 | 0 | 2 |
| 1799 | 1.4 | 123 | 119 | 49 | 145 | 178 | 33 |
| 1799 | 1.91 | 4 | 7 | 3 | 11 | 6 | 1 |
| 1806 | 0 | 11 | 1 | 0 | 14 | 6 | 2 |
| 1837 | 1.32 | 164 | 108 | 32 | 166 | 167 | 22 |
| 1837 | 1.04 | 334 | 223 | 50 | 322 | 308 | 52 |
| 1837 | 0.86 | 20 | 6 | 2 | 66 | 76 | 13 |
| 1837 | 0.96 | 37 | 33 | 7 | 21 | 44 | 7 |
| 1837 | 0.77 | 170 | 115 | 18 | 156 | 141 | 30 |
| 1870 | 0.61 | 2 | 25 | 18 | 3 | 10 | 26 |
| 1870 | 0.58 | 1 | 20 | 10 | 1 | 7 | 14 |
| 1882 | 1.59 | 21 | 37 | 11 | 9 | 25 | 0 |
| 1988 | 0.64 | 52 | 39 | 9 | 48 | 48 | 20 |
| 2000 | null | 68 | 2 | 0 | 29 | 5 | 0 |
| 2000 | null | 101 | 4 | 0 | 65 | 9 | 0 |
| 2000 | null | 45 | 1 | 0 | 37 | 5 | 0 |
| 2000 | 0 | 20 | 0 | 0 | 20 | 2 | 0 |
| 2000 | null | 77 | 2 | 0 | 76 | 6 | 0 |
| 2000 | null | 11 | 3 | 0 | 18 | 1 | 0 |
| 2071 | 0.79 | 14 | 52 | 36 | 4 | 34 | 36 |
| 2078 | 0.45 | 1 | 11 | 6 | 0 | 13 | 22 |
| 2085 | 0 | 6 | 4 | 0 | 3 | 7 | 4 |
| 2095 | null | 4 | 101 | 0 | 0 | 73 | 0 |
| 2119 | null | 3 | 17 | 0 | 16 | 21 | 0 |
| 2119 | null | 29 | 73 | 0 | 49 | 68 | 0 |
| 2119 | null | 3 | 9 | 0 | 13 | 9 | 0 |
| 2141 | 0.42 | 0 | 6 | 6 | 2 | 2 | 18 |
| 2141 | 0.87 | 0 | 17 | 28 | 3 | 9 | 27 |
| 2182 | 0 | 6 | 6 | 0 | 1 | 14 | 6 |
| 2234 | 1.75 | 0 | 10 | 10 | 7 | 18 | 10 |
| 2281 | 2.13 | 0 | 5 | 4 | 4 | 7 | 2 |
| 2298 | 1.31 | 4 | 10 | 21 | 0 | 20 | 18 |
| 2298 | 1.65 | 0 | 8 | 21 | 2 | 12 | 14 |
| 2341 | 0.53 | 0 | 6 | 25 | 0 | 0 | 22 |
| 2357 | 1.1 | 5 | 18 | 51 | 0 | 25 | 46 |
| 2357 | 1.21 | 4 | 8 | 33 | 0 | 14 | 26 |
| 2357 | 2.4 | 0 | 4 | 16 | 0 | 17 | 19 |
| 2357 | 1.13 | 4 | 4 | 23 | 0 | 7 | 15 |
| 2366 | 0.55 | 12 | 14 | 7 | 8 | 15 | 17 |
| 2423 | 0.45 | 16 | 13 | 4 | 12 | 14 | 13 |
| 2708 | null | 28 | 1 | 0 | 33 | 7 | 0 |
| 2995 | 0.45 | 3 | 10 | 5 | 4 | 37 | 41 |
| 2995 | 1.57 | 2 | 20 | 32 | 5 | 40 | 30 |
| 3360 | 0 | 10 | 0 | 0 | 50 | 22 | 1 |
| 3464 | 1.61 | 3 | 15 | 27 | 9 | 17 | 14 |
| 3464 | 1.74 | 3 | 7 | 21 | 5 | 9 | 8 |
| 3689 | 0 | 3 | 3 | 0 | 1 | 8 | 5 |
| 3975 | 1.5 | 2 | 24 | 30 | 10 | 38 | 27 |
| 3976 | 1.41 | 2 | 24 | 30 | 11 | 35 | 29 |
| 4206 | 1.61 | 8 | 20 | 9 | 31 | 41 | 11 |
| 4838 | 0.56 | 7 | 2 | 1 | 3 | 8 | 3 |
| 4838 | 0.56 | 7 | 2 | 1 | 3 | 8 | 3 |
| 4838 | 0.56 | 7 | 2 | 1 | 3 | 8 | 3 |
| 4912 | 0.56 | 7 | 0 | 5 | 5 | 2 | 13 |
| 4925 | null | 7 | 7 | 0 | 15 | 3 | 0 |
| 4966 | 2.26 | 7 | 8 | 11 | 18 | 41 | 13 |
| 5014 | 3.07 | 3 | 2 | 23 | 10 | 57 | 85 |
| 5014 | 2.73 | 2 | 1 | 15 | 5 | 27 | 49 |
| 5296 | 0.59 | 0 | 10 | 26 | 0 | 4 | 36 |
| 5296 | 2.39 | 1 | 1 | 10 | 0 | 9 | 13 |
| 5296 | 0.76 | 1 | 25 | 78 | 0 | 10 | 64 |
| 5298 | 2.25 | 1 | 1 | 9 | 0 | 9 | 13 |
| 5298 | 0.76 | 3 | 22 | 76 | 0 | 10 | 64 |
| 5298 | 0.61 | 1 | 8 | 26 | 0 | 4 | 36 |
| 5320 | 2.18 | 1 | 10 | 8 | 9 | 19 | 5 |
| 5361 | 1.16 | 24 | 5 | 35 | 18 | 0 | 14 |
| 5457 | 3.52 | 1 | 0 | 11 | 1 | 6 | 14 |
| 5704 | 2.44 | 1 | 8 | 11 | 3 | 26 | 8 |
| 5704 | 1.32 | 5 | 30 | 33 | 6 | 18 | 9 |
| 5717 | 0.55 | 17 | 16 | 5 | 21 | 41 | 21 |
| 5717 | 0.64 | 21 | 32 | 12 | 34 | 76 | 46 |
| 5959 | 0.59 | 16 | 20 | 7 | 4 | 21 | 13 |
| 5959 | 0.27 | 4 | 4 | 1 | 0 | 7 | 6 |
| 5959 | 0.57 | 4 | 7 | 3 | 0 | 10 | 7 |
| 5959 | 4.03 | 2 | 2 | 5 | 13 | 41 | 13 |
| 5959 | 0.62 | 15 | 41 | 16 | 11 | 29 | 28 |
| 6162 | 0.77 | 1 | 35 | 28 | 19 | 52 | 80 |
| 6162 | 0.9 | 6 | 76 | 74 | 19 | 52 | 80 |
| 6162 | 0.66 | 0 | 16 | 11 | 19 | 52 | 80 |
| 6162 | 0.87 | 0 | 13 | 13 | 11 | 21 | 39 |
| 6162 | 0.43 | 0 | 13 | 5 | 8 | 31 | 41 |
| 6162 | 0.91 | 3 | 34 | 37 | 11 | 21 | 39 |
| 6236 | 0.49 | 6 | 12 | 9 | 13 | 58 | 81 |
| 6236 | 0.47 | 4 | 15 | 8 | 5 | 28 | 39 |
| 6236 | 0.25 | 2 | 6 | 2 | 5 | 28 | 39 |
| 6236 | 0.71 | 10 | 27 | 26 | 13 | 58 | 81 |
| 6482 | 1.79 | 5 | 8 | 4 | 15 | 4 | 2 |
| 6482 | 1 | 340 | 238 | 41 | 436 | 226 | 47 |
| 6482 | 0.47 | 18 | 7 | 2 | 10 | 12 | 6 |
| 6482 | 1.08 | 173 | 115 | 21 | 220 | 99 | 20 |
| 6498 | 0 | 28 | 4 | 0 | 25 | 7 | 3 |
| 6744 | 0.47 | 4 | 13 | 9 | 9 | 56 | 84 |
| 7133 | 2.8 | 10 | 0 | 4 | 18 | 0 | 0 |
| 8021 | 0.26 | 8 | 19 | 1 | 15 | 14 | 7 |
| 8060 | 2.18 | 31 | 3 | 1 | 28 | 12 | 0 |
| 8060 | 0 | 11 | 7 | 0 | 20 | 3 | 0 |
| 8210 | 0.81 | 1 | 7 | 4 | 9 | 4 | 9 |
| 8592 | 0.86 | 15 | 92 | 43 | 25 | 68 | 50 |
| 8816 | 0.36 | 4 | 7 | 2 | 0 | 5 | 6 |
| 8846 | 0.4 | 57 | 47 | 3 | 62 | 42 | 12 |
| 8943 | 0 | 15 | 5 | 0 | 20 | 12 | 5 |
| 9193 | null | 72 | 11 | 0 | 60 | 20 | 0 |
| 9193 | 2.14 | 28 | 7 | 1 | 37 | 3 | 0 |
| 9443 | 0.85 | 9 | 25 | 35 | 0 | 12 | 21 |
| 9516 | 0.67 | 7 | 3 | 4 | 2 | 8 | 8 |
| 9698 | 2.78 | 4 | 0 | 70 | 14 | 2 | 56 |
| 9698 | | 0 | 0 | 27 | 14 | 2 | 56 |
| 9698 | 1.04 | 5 | 95 | 194 | 16 | 91 | 191 |
| 9698 | null | 0 | 0 | 10 | 14 | 2 | 56 |
| 9698 | 0.8 | 17 | 12 | 73 | 6 | 7 | 59 |
| 9849 | null | 31 | 0 | 0 | 18 | 3 | 0 |
| 9849 | null | 15 | 5 | 0 | 35 | 2 | 0 |
| 9883 | 1.55 | 7 | 9 | 15 | 1 | 16 | 5 |
| 9883 | 1.42 | 9 | 15 | 21 | 4 | 24 | 11 |
| 10079 | 0.72 | 4 | 0 | 99 | 0 | 1 | 72 |
| 10079 | 0.65 | 4 | 0 | 64 | 0 | 0 | 34 |
| 10481 | 2.53 | 3 | 6 | 8 | 32 | 33 | 18 |
| 10542 | 1.86 | 1 | 6 | 47 | 0 | 21 | 54 |
| 10542 | null | 0 | 0 | 10 | 0 | 14 | 55 |
| 10600 | null | 0 | 0 | 21 | 0 | 4 | 29 |
| 10621 | 1.71 | 24 | 4 | 2 | 12 | 8 | 0 |
| 10745 | 0.72 | 5 | 10 | 12 | 7 | 61 | 80 |
| 10745 | 0.8 | 11 | 68 | 74 | 16 | 45 | 89 |
| 10747 | 1.29 | 14 | 46 | 16 | 3 | 58 | 9 |
| 10747 | 0.95 | 15 | 24 | 23 | 6 | 39 | 29 |
| 10747 | 1.77 | 4 | 16 | 7 | 3 | 58 | 9 |
| 10771 | 0.42 | 4 | 4 | 2 | 6 | 36 | 28 |
| 10771 | 0.86 | 52 | 118 | 114 | 40 | 105 | 131 |
| 10870 | 0.64 | 0 | 11 | 9 | 5 | 9 | 23 |
| 10870 | 1.5 | 7 | 18 | 57 | 8 | 30 | 39 |
| 10870 | 2.51 | 0 | 3 | 11 | 2 | 8 | 8 |
| 10870 | 1.47 | 2 | 13 | 30 | 6 | 15 | 19 |
| 10877 | 2.00 | 0 | 0 | 9 | 1 | 5 | 9 |
| 10948 | 1.04 | 16 | 51 | 17 | 31 | 33 | 15 |
| 10948 | 1.1 | 22 | 60 | 22 | 44 | 50 | 21 |
| 10948 | 0.83 | 9 | 28 | 7 | 17 | 16 | 9 |
| 10948 | 1.23 | 12 | 39 | 18 | 12 | 17 | 5 |
| 11001 | 0.51 | 2 | 5 | 3 | 2 | 37 | 36 |
| 11073 | 0.33 | 3 | 4 | 2 | 9 | 25 | 34 |
| 11153 | null | 24 | 7 | 0 | 11 | 11 | 0 |
| 11210 | null | 9 | 5 | 0 | 18 | 1 | 0 |
| 11210 | 0 | 47 | 16 | 0 | 125 | 17 | 2 |
| 11210 | 0 | 122 | 31 | 0 | 125 | 17 | 2 |
| 11248 | 1.08 | 56 | 19 | 6 | 38 | 36 | 5 |
| 11248 | null | 15 | 3 | 0 | 19 | 15 | 0 |
| 11248 | null | 27 | 14 | 0 | 13 | 18 | 0 |
| 11372 | 0.6 | 10 | 5 | 5 | 10 | 11 | 15 |
| 11449 | 2.05 | 1 | 4 | 26 | 0 | 10 | 12 |
| 11450 | 0.87 | 28 | 114 | 147 | 16 | 107 | 167 |
| 11470 | null | 20 | 0 | 0 | 31 | 5 | 0 |
| 11472 | null | 20 | 0 | 0 | 30 | 5 | 0 |
| 11472 | null | 75 | 8 | 0 | 78 | 2 | 0 |
| 11487 | null | 0 | 10 | 0 | 34 | 35 | 0 |
| 11487 | null | 6 | 21 | 0 | 34 | 35 | 0 |
| 11488 | 0 | 10 | 0 | 0 | 35 | 32 | 3 |
| 11488 | 2.57 | 29 | 20 | 5 | 49 | 28 | 0 |
| 11488 | 1.52 | 20 | 4 | 2 | 35 | 32 | 3 |
| 11493 | 0.61 | 0 | 6 | 8 | 2 | 2 | 14 |
| 11502 | 1.94 | 0 | 8 | 19 | 7 | 30 | 36 |
| 11502 | 3.55 | 0 | 2 | 8 | 7 | 30 | 36 |
| 11534 | null | 102 | 0 | 0 | 114 | 3 | 0 |
| 11537 | 1.75 | 20 | 12 | 4 | 30 | 8 | 1 |
| 11537 | 0 | 10 | 2 | 0 | 12 | 7 | 3 |
| 11560 | 0.33 | 1 | 0 | 11 | 0 | 0 | 22 |
| 11578 | null | 60 | 1 | 0 | 57 | 8 | 0 |
| 11578 | null | 27 | 3 | 0 | 39 | 0 | 0 |
| 11594 | null | 0 | 0 | 37 | 2 | 6 | 72 |
| 11594 | null | 0 | 0 | 27 | 2 | 16 | 133 |
| 11594 | 0.62 | 0 | 10 | 35 | 0 | 3 | 38 |
| 11594 | 1.71 | 1 | 7 | 147 | 2 | 16 | 133 |
| 11594 | null | 0 | 0 | 18 | 2 | 6 | 72 |
| 11624 | 2.11 | 21 | 15 | 6 | 20 | 20 | 0 |
| 11624 | 2.8 | 8 | 2 | 3 | 9 | 9 | 0 |
| 11624 | 0 | 10 | 2 | 0 | 12 | 6 | 3 |
| 11627 | 2.05 | 20 | 18 | 7 | 21 | 19 | 0 |
| 11627 | 2.64 | 7 | 4 | 3 | 9 | 9 | 0 |
| 11627 | 0 | 10 | 2 | 0 | 12 | 7 | 3 |
| 11644 | 3.26 | 0 | 2 | 8 | 7 | 26 | 35 |
| 11650 | 1.21 | 26 | 105 | 160 | 23 | 135 | 132 |
| 11654 | 1.48 | 7 | 3 | 15 | 14 | 56 | 66 |
| 11654 | 1.65 | 5 | 1 | 9 | 8 | 31 | 32 |
| 11654 | 1.25 | 8 | 7 | 17 | 8 | 31 | 32 |
| 11654 | 1.02 | 12 | 15 | 26 | 14 | 56 | 66 |
| 11655 | 2.3 | 16 | 3 | 7 | 72 | 59 | 17 |
| 11655 | 2.11 | 11 | 1 | 5 | 35 | 34 | 11 |
| 11655 | 1.64 | 19 | 7 | 9 | 35 | 34 | 11 |
| 11656 | 2.57 | 6 | 8 | 6 | 19 | 15 | 2 |
| 11656 | 0 | 7 | 5 | 0 | 5 | 14 | 3 |
| 11656 | 1.5 | 35 | 49 | 18 | 51 | 54 | 9 |
| 11825 | null | 6 | 3 | 0 | 58 | 5 | 0 |
| 11914 | 9.83 | 1 | 0 | 8 | 41 | 1 | 27 |
| 11914 | 1.48 | 6 | 12 | 9 | 63 | 52 | 36 |
| 12008 | null | 251 | 27 | 0 | 264 | 13 | 0 |
| 12008 | null | 24 | 0 | 0 | 122 | 12 | 0 |
| 12097 | null | 6 | 22 | 0 | 11 | 144 | 0 |
| 12097 | null | 7 | 31 | 0 | 5 | 78 | 0 |
| 12097 | null | 3 | 7 | 0 | 6 | 66 | 0 |
| 12097 | null | 10 | 53 | 0 | 11 | 144 | 0 |
| 12366 | 0 | 32 | 18 | 0 | 39 | 26 | 9 |
| 12366 | 2.26 | 18 | 4 | 3 | 85 | 59 | 7 |
| 12619 | null | 1 | 9 | 0 | 0 | 71 | 0 |
| 12619 | null | 2 | 25 | 0 | 1 | 150 | 0 |
| 13025 | 2.38 | 13 | 8 | 7 | 65 | 71 | 15 |
| 13191 | 1.55 | 6 | 30 | 47 | 10 | 42 | 27 |
| 13191 | 0.43 | 2 | 7 | 5 | 0 | 5 | 13 |
| 13191 | 1.38 | 6 | 39 | 56 | 13 | 55 | 46 |
| 13937 | 2.53 | 4 | 11 | 2 | 42 | 38 | 3 |
| 900002 | 0.64 | 5 | 13 | 16 | 2 | 11 | 27 |
| 900013 | 1.34 | 20 | 9 | 6 | 13 | 23 | 4 |
| 900013 | 1.16 | 58 | 34 | 12 | 29 | 39 | 6 |
| 900025 | 1.3 | 7 | 27 | 32 | 7 | 17 | 10 |
| 900032 | 2.54 | 23 | 1 | 1 | 28 | 9 | 0 |
| 900045 | 2.67 | 1 | 2 | 9 | 5 | 8 | 9 |
| 900065 | 0 | 22 | 10 | 0 | 16 | 18 | 5 |
| 900065 | 1.53 | 25 | 30 | 4 | 7 | 22 | 0 |
| 900065 | 0.77 | 47 | 40 | 4 | 23 | 40 | 5 |
| 900078 | null | 52 | 12 | 0 | 142 | 13 | 0 |
| 900078 | null | 21 | 6 | 0 | 142 | 13 | 0 |
| 900078 | null | 31 | 7 | 0 | 78 | 5 | 0 |
| 900082 | 1.9 | 8 | 9 | 18 | 17 | 36 | 21 |
| 900082 | 2.76 | 3 | 4 | 10 | 17 | 36 | 21 |
| 900096 | 1.15 | 60 | 37 | 4 | 55 | 15 | 2 |
| 900107 | 3.06 | 0 | 2 | 8 | 9 | 25 | 39 |
| 900115 | 4.6 | 1 | 4 | 4 | 27 | 37 | 8 |
| 900115 | 1.8 | 22 | 14 | 4 | 17 | 28 | 1 |
| 900121 | 0.95 | 5 | 37 | 24 | 15 | 26 | 26 |
| 900173 | 1.38 | 5 | 7 | 11 | 11 | 4 | 7 |
| 10000002 | 0 | 9 | 3 | 0 | 9 | 7 | 6 |
| 10000006 | 0.56 | 28 | 2 | 1 | 11 | 9 | 2 |
| 10000006 | 0.45 | 39 | 4 | 1 | 23 | 12 | 3 |
| 10000014 | 1.26 | 40 | 3 | 2 | 26 | 12 | 1 |
| 10000014 | 1.73 | 28 | 2 | 1 | 15 | 7 | 0 |
| 10000025 | 0 | 9 | 11 | 0 | 14 | 15 | 7 |

TABLE 6b
Correlation of PA SNP alleles to relative risk
For diagnostic conclusions to be drawn from genotyping a
particular patient we calculated the relative risks
RR1, and RR2 for the two possible
alleles of each SNP. Given the allele frequencies as

| | allele1 | allele2 |
|---|---|---|
| case | N11 | N12 |
| control | N21 | N22 |

we calculate

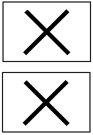

Here, the case and control populations represent any case-control-group pair, or bad(case)-good(control)-group pair, respectively (due to their increased response to statins, ‘high responders’ are treated as a case cohort, whereas ‘low responders’ are treated as the respective control cohort). A value RR1>1, and RR2>1 indicates an increased risk for individuals carrying allele 1, and allele2, respectively. For example, RR1=3 indicates a 3-fold risk of an individual carrying allele 1 as compared to individuals not carrying allele 1 (a detailed description of relative risk calculation and statistics can be found in (Biostatistics, L. D. Fisher and G. van Belle, Wiley Interscience 1993)). The baySNP number refers to an internal numbering of the PA SNPs and can be found in the sequence listing null: not defined.

| | | | | | | SIZE | FREQ1 | FREQ2 | SIZE | FREQ1 | FREQ2 |
|---|---|---|---|---|---|---|---|---|---|---|---|
| BAYSNP | ALLELE1 | ALLELE2 | COMPARISON | RR1 | RR2 | A | A | A | B | B | B |
| 28 | C | T | HELD_FEM_EFF | 0.42 | 2.39 | 12 | 4 | 20 | 22 | 18 | 26 |
| 29 | A | G | HELD_ALL_HDL | 2.01 | 0.5 | 10 | 12 | 8 | 15 | 7 | 23 |
| 29 | A | G | HELD_MAL_ADR3ULN | 1.63 | 0.61 | 26 | 33 | 19 | 72 | 68 | 76 |
| 29 | A | G | HELD_MAL_ADR5ULN | 2.6 | 0.38 | 9 | 13 | 5 | 72 | 68 | 76 |
| 52 | C | G | HELD_FEM_EFF | 1.84 | 0.54 | 18 | 24 | 12 | 31 | 27 | 35 |
| 56 | A | G | HELD_FEM_EFF | 2.29 | 0.44 | 12 | 5 | 19 | 22 | 2 | 42 |
| 89 | A | G | HELD_ALL_CC | null | 0 | 45 | 90 | 0 | 40 | 77 | 3 |
| 90 | C | T | HELD_FEM_CC | 0.78 | 1.27 | 31 | 29 | 33 | 22 | 27 | 17 |
| 99 | C | T | HELD_FEM_LIP | 1.02 | 0.98 | 82 | 54 | 110 | 80 | 51 | 109 |
| 140 | C | T | HELD_FEM_EFF | null | 0 | 12 | 24 | 0 | 21 | 4 | 38 |
| 152 | A | G | HELD_FEM_EFF | 0.51 | 1.96 | 12 | 12 | 12 | 22 | 33 | 11 |
| 214 | A | G | HELD_ALL_LIP | 1 | 1 | 97 | 156 | 38 | 113 | 182 | 44 |
| 214 | A | G | HELD_FEM_LIP | 1.09 | 0.92 | 81 | 131 | 31 | 78 | 122 | 34 |
| 221 | C | G | HELD_ALL_CC | 0.88 | 1.13 | 45 | 26 | 64 | 39 | 27 | 51 |
| 221 | C | G | HELD_FEM_CC | 0.77 | 1.3 | 31 | 17 | 45 | 22 | 18 | 26 |
| 224 | C | T | HELD_FEM_LIP | 0.79 | 1.27 | 79 | 110 | 48 | 79 | 125 | 33 |
| 224 | C | T | HELD_MAL_LIP | 2.28 | 0.44 | 20 | 35 | 5 | 37 | 51 | 23 |
| 294 | C | T | HELD_ALL_CC | 0.81 | 1.24 | 45 | 56 | 34 | 40 | 58 | 22 |
| 307 | C | T | CVD_FEM | 0.57 | 1.75 | 36 | 19 | 53 | 38 | 38 | 38 |
| 307 | C | T | HELD_ALL_LIP | 1.2 | 0.83 | 102 | 70 | 134 | 117 | 63 | 171 |
| 411 | A | T | HELD_ALL_HDL | 1.56 | 0.64 | 10 | 17 | 3 | 15 | 18 | 12 |
| 449 | C | G | HELD_MAL_LIP | 0.41 | 2.47 | 20 | 3 | 37 | 37 | 16 | 58 |
| 466 | C | T | CVD_FEM | 0.7 | 1.43 | 35 | 27 | 43 | 40 | 44 | 36 |
| 472 | A | G | HELD_FEM_EFF | null | 0 | 11 | 22 | 0 | 22 | 12 | 32 |
| 542 | A | G | HELD_MAL_CC | 2.79 | 0.36 | 14 | 12 | 16 | 19 | 2 | 36 |
| 542 | A | G | HELD_MAL_HDL | 3.66 | 0.27 | 21 | 14 | 28 | 27 | 3 | 51 |
| 542 | A | G | HELD_ALL_ADR | 1.19 | 0.84 | 159 | 53 | 265 | 154 | 37 | 271 |
| 542 | A | G | HELD_FEM_HDL | 0.66 | 1.51 | 23 | 2 | 44 | 32 | 10 | 54 |
| 739 | C | G | HELD_ALL_CC | 0.73 | 1.37 | 45 | 39 | 51 | 40 | 48 | 32 |
| 821 | A | C | HELD_MAL_LIP2 | 1.12 | 0.9 | 309 | 180 | 438 | 349 | 174 | 524 |
| 821 | A | C | HELD_FEM_VEFF | 0.42 | 2.4 | 10 | 4 | 16 | 14 | 14 | 14 |
| 1005 | A | G | HELD_MAL_CC | 2.7 | 0.37 | 14 | 26 | 2 | 18 | 27 | 9 |
| 1055 | A | T | HELD_MAL_CC | 0.56 | 1.77 | 9 | 3 | 15 | 12 | 8 | 16 |
| 1056 | A | G | HELD_FEM_EFF | 1.01 | 0.99 | 24 | 30 | 18 | 33 | 41 | 25 |
| 1085 | A | G | HELD_MAL_LIP | 0.57 | 1.74 | 20 | 17 | 23 | 36 | 46 | 26 |
| 1085 | A | G | CVD_FEM | 1.53 | 0.65 | 34 | 51 | 17 | 40 | 47 | 33 |
| 1086 | A | G | HELD_MAL_LIP | 1.73 | 0.58 | 20 | 24 | 16 | 36 | 28 | 44 |
| 1092 | C | G | HELD_MAL_LIP | 0.58 | 1.72 | 20 | 9 | 31 | 37 | 29 | 45 |
| 1096 | G | T | HELD_MAL_CC | 1.8 | 0.56 | 14 | 7 | 21 | 18 | 3 | 33 |
| 1096 | G | T | CVD_MAL | 0.93 | 1.08 | 69 | 21 | 117 | 33 | 12 | 54 |
| 1101 | C | T | HELD_FEM_EFF | null | 0 | 12 | 24 | 0 | 22 | 40 | 4 |
| 1204 | A | G | HELD_MAL_LIP | 1.91 | 0.52 | 19 | 12 | 26 | 35 | 9 | 61 |
| 1204 | A | G | HELD_ALL_LIP | 1.26 | 0.8 | 99 | 62 | 136 | 115 | 52 | 178 |
| 1504 | C | T | HELD_ALL_CC | 0.92 | 1.08 | 44 | 37 | 51 | 39 | 36 | 42 |
| 1504 | C | T | HELD_MAL_LIP | 0.69 | 1.46 | 19 | 12 | 26 | 37 | 33 | 41 |
| 1504 | C | T | HELD_MAL_CC | 1.35 | 0.74 | 14 | 13 | 15 | 18 | 12 | 24 |
| 1504 | C | T | HELD_FEM_CC | 0.75 | 1.33 | 30 | 24 | 36 | 21 | 24 | 18 |
| 1511 | G | T | HELD_FEM_EFF | 0.6 | 1.67 | 12 | 15 | 9 | 22 | 35 | 9 |
| 1524 | A | C | HELD_FEM_ADR3ULN | 0.9 | 1.11 | 38 | 16 | 60 | 82 | 39 | 125 |
| 1556 | C | G | HELD_FEM_EFF | 2.39 | 0.42 | 12 | 7 | 17 | 22 | 3 | 41 |
| 1561 | A | C | CVD_FEM | 1.53 | 0.65 | 36 | 58 | 14 | 40 | 53 | 27 |
| 1582 | C | T | HELD_MAL_LIP | 0.46 | 2.17 | 20 | 5 | 35 | 37 | 22 | 52 |
| 1638 | A | G | HELD_FEM_CC | 0.62 | 1.6 | 31 | 10 | 52 | 22 | 15 | 29 |
| 1653 | G | T | CVD_MAL | 1.07 | 0.93 | 69 | 70 | 68 | 33 | 30 | 36 |
| 1662 | C | T | HELD_MAL_CC | 0.18 | 5.5 | 14 | 8 | 20 | 18 | 36 | 0 |
| 1714 | A | G | CVD_MAL | 0.78 | 1.28 | 66 | 32 | 100 | 34 | 26 | 42 |
| 1722 | C | T | HELD_FEM_ADR5ULN | 1.61 | 0.62 | 18 | 21 | 15 | 81 | 71 | 91 |
| 1757 | A | G | HELD_FEM_EFF | 1.41 | 0.71 | 20 | 15 | 25 | 32 | 16 | 48 |
| 1765 | A | G | HELD_ALL_ADR3ULN | 0.42 | 2.35 | 63 | 9 | 117 | 149 | 56 | 242 |
| 1765 | A | G | HELD_ALL_ADR3ULN | 0.42 | 2.35 | 63 | 9 | 117 | 149 | 56 | 242 |
| 1765 | A | G | HELD_ALL_ADR5ULN | 0.29 | 3.42 | 27 | 3 | 51 | 149 | 56 | 242 |
| 1765 | A | G | HELD_ALL_ADR5ULN | 0.29 | 3.42 | 27 | 3 | 51 | 149 | 56 | 242 |
| 1765 | A | G | HELD_MAL_ADR3ULN | 0.24 | 4.09 | 26 | 2 | 50 | 70 | 25 | 115 |
| 1765 | A | G | HELD_MAL_ADR3ULN | 0.24 | 4.09 | 26 | 2 | 50 | 70 | 25 | 115 |
| 1765 | A | G | HELD_MAL_ADR5ULN | null | 0 | 10 | 20 | 0 | 70 | 25 | 115 |
| 1765 | A | G | HELD_MAL_ADR5ULN | null | 0 | 10 | 20 | 0 | 70 | 25 | 115 |
| 1765 | A | G | HELD_FEM_ADR3ULN | 0.53 | 1.87 | 37 | 7 | 67 | 79 | 31 | 127 |
| 1765 | A | G | HELD_FEM_ADR3ULN | 0.53 | 1.87 | 37 | 7 | 67 | 79 | 31 | 127 |
| 1776 | A | G | HELD_ALL_CC | null | 0 | 45 | 90 | 0 | 40 | 74 | 6 |
| 1776 | A | G | HELD_FEM_CC | null | 0 | 31 | 62 | 0 | 22 | 40 | 4 |
| 1799 | C | T | HELD_FEM_LIP2 | 0.93 | 1.07 | 291 | 365 | 217 | 356 | 468 | 244 |
| 1799 | C | T | HELD_MAL_CC | 0.56 | 1.77 | 14 | 15 | 13 | 18 | 28 | 8 |
| 1806 | A | G | HELD_FEM_EFF | 4.44 | 0.23 | 12 | 23 | 1 | 22 | 34 | 10 |
| 1837 | C | T | HELD_FEM_LIP2 | 1.04 | 0.96 | 304 | 436 | 172 | 355 | 499 | 211 |
| 1837 | C | T | HELD_ALL_LIP2 | 1.1 | 0.91 | 607 | 891 | 323 | 682 | 952 | 412 |
| 1837 | C | T | HELD_ALL_ADR5ULN | 2.03 | 0.49 | 28 | 46 | 10 | 155 | 208 | 102 |
| 1837 | C | T | HELD_MAL_ADR | 1.24 | 0.81 | 77 | 107 | 47 | 72 | 86 | 58 |
| 1837 | C | T | HELD_MAL_LIP2 | 1.17 | 0.86 | 303 | 455 | 151 | 327 | 453 | 201 |
| 1870 | C | T | HELD_ALL_CC | 1.3 | 0.77 | 45 | 29 | 61 | 39 | 16 | 62 |
| 1870 | C | T | HELD_FEM_CC | 1.33 | 0.75 | 31 | 22 | 40 | 22 | 9 | 35 |
| 1882 | C | T | CVD_MAL | 0.92 | 1.08 | 69 | 79 | 59 | 34 | 43 | 25 |
| 1988 | C | T | HELD_ALL_LIP | 1.27 | 0.79 | 100 | 143 | 57 | 116 | 144 | 88 |
| 2000 | C | T | CVD_MAL | 2.45 | 0.41 | 70 | 136 | 4 | 34 | 58 | 10 |
| 2000 | C | T | CVD_ALL | 1.98 | 0.51 | 105 | 202 | 8 | 74 | 130 | 18 |
| 2000 | C | T | HELD_FEM_CC2 | 3.29 | 0.3 | 46 | 90 | 2 | 42 | 74 | 10 |
| 2000 | C | T | HELD_MAL_HDL | 2 | 0.5 | 20 | 40 | 0 | 22 | 40 | 4 |
| 2000 | C | T | HELD_FEM_ADR | 2.01 | 0.5 | 79 | 154 | 4 | 82 | 152 | 12 |
| 2000 | C | T | HELD_MAL_CC | 0.51 | 1.98 | 14 | 22 | 6 | 19 | 36 | 2 |
| 2071 | A | G | CVD_ALL | 1.22 | 0.82 | 102 | 80 | 124 | 74 | 42 | 106 |
| 2078 | G | T | HELD_MAL_LIP | 1.74 | 0.58 | 18 | 13 | 23 | 35 | 13 | 57 |
| 2085 | G | T | HELD_FEM_VEFF | 2.62 | 0.38 | 10 | 16 | 4 | 14 | 13 | 15 |
| 2095 | A | G | CVD_ALL | 0.03 | 37.5 | 105 | 4 | 206 | 73 | 146 | 0 |
| 2119 | A | G | HELD_MAL_LIP | 0.68 | 1.48 | 20 | 23 | 17 | 37 | 53 | 21 |
| 2119 | A | G | HELD_ALL_LIP | 0.85 | 1.17 | 102 | 131 | 73 | 117 | 166 | 68 |
| 2119 | A | G | HELD_FEM_EFF | 0.6 | 1.67 | 12 | 15 | 9 | 22 | 35 | 9 |
| 2141 | A | G | HELD_FEM_EFF | 1.56 | 0.64 | 12 | 6 | 18 | 22 | 6 | 38 |
| 2141 | A | G | HELD_ALL_CC | 0.99 | 1.01 | 45 | 17 | 73 | 39 | 15 | 63 |
| 2182 | A | G | HELD_FEM_EFF | 2.82 | 0.35 | 12 | 18 | 6 | 21 | 16 | 26 |
| 2234 | G | T | HELD_MAL_LIP | 0.54 | 1.85 | 20 | 10 | 30 | 35 | 32 | 38 |
| 2281 | A | C | HELD_FEM_VEFF | 0.46 | 2.17 | 9 | 5 | 13 | 13 | 15 | 11 |
| 2298 | A | C | CVD_FEM | 0.98 | 1.02 | 35 | 18 | 52 | 38 | 20 | 56 |
| 2298 | A | C | HELD_MAL_CC2 | 0.6 | 1.67 | 29 | 8 | 50 | 28 | 16 | 40 |
| 2341 | C | T | HELD_FEM_CC | 0.12 | 8.33 | 31 | 6 | 56 | 22 | 44 | 0 |
| 2357 | A | G | HELD_ALL_CC2 | 1.04 | 0.96 | 74 | 28 | 120 | 71 | 25 | 117 |
| 2357 | A | G | HELD_ALL_CC | 1.01 | 0.99 | 45 | 16 | 74 | 40 | 14 | 66 |
| 2357 | A | G | HELD_MAL_LIP | 0.48 | 2.08 | 20 | 4 | 36 | 36 | 17 | 55 |
| 2357 | A | G | HELD_FEM_CC | 1.1 | 0.91 | 31 | 12 | 50 | 22 | 7 | 37 |
| 2366 | G | T | CVD_FEM | 1.51 | 0.66 | 33 | 38 | 28 | 40 | 31 | 49 |
| 2423 | A | G | CVD_FEM | 1.57 | 0.63 | 33 | 45 | 21 | 39 | 38 | 40 |
| 2708 | C | T | CVD_FEM | 3.51 | 0.29 | 29 | 57 | 1 | 40 | 73 | 7 |
| 2995 | A | C | HELD_FEM_ADR5ULN | 1.82 | 0.55 | 18 | 16 | 20 | 82 | 45 | 119 |
| 2995 | A | C | HELD_FEM_UEFF | 0.71 | 1.41 | 54 | 24 | 84 | 75 | 50 | 100 |
| 3360 | G | T | HELD_MAL_ADR5ULN | null | 0 | 10 | 20 | 0 | 73 | 122 | 24 |
| 3464 | A | G | HELD_ALL_CC | 0.62 | 1.61 | 45 | 21 | 69 | 40 | 35 | 45 |
| 3464 | A | G | HELD_FEM_CC | 0.61 | 1.63 | 31 | 13 | 49 | 22 | 19 | 25 |
| 3689 | C | G | HELD_FEM_EFF | 3.32 | 0.3 | 6 | 9 | 3 | 14 | 10 | 18 |
| 3975 | A | C | HELD_FEM_UEFF | 0.68 | 1.47 | 56 | 28 | 84 | 75 | 58 | 92 |
| 3976 | A | G | HELD_FEM_UEFF | 0.69 | 1.44 | 56 | 28 | 84 | 75 | 57 | 93 |
| 4206 | A | T | HELD_FEM_ADR3ULN | 0.69 | 1.45 | 37 | 36 | 38 | 83 | 103 | 63 |
| 4838 | A | G | HELD_FEM_VEFF | 2.4 | 0.42 | 10 | 16 | 4 | 14 | 14 | 14 |
| 4838 | A | G | HELD_FEM_VEFF | 2.4 | 0.42 | 10 | 16 | 4 | 14 | 14 | 14 |
| 4838 | A | G | HELD_FEM_VEFF | 2.4 | 0.42 | 10 | 16 | 4 | 14 | 14 | 14 |
| 4912 | A | G | HELD_FEM_EFF | 2.05 | 0.49 | 12 | 14 | 10 | 20 | 12 | 28 |
| 4925 | A | C | HELD_MAL_CC | 0.56 | 1.8 | 14 | 21 | 7 | 18 | 33 | 3 |
| 4966 | A | G | HELD_MAL_ADR3ULN | 0.72 | 1.39 | 26 | 22 | 30 | 72 | 77 | 67 |
| 5014 | A | G | HELD_ALL_ADR5ULN | 0.54 | 1.85 | 28 | 8 | 48 | 152 | 77 | 227 |
| 5014 | A | G | HELD_FEM_ADR5ULN | 0.6 | 1.67 | 18 | 5 | 31 | 81 | 37 | 125 |
| 5296 | A | G | CVD_FEM | 1.59 | 0.63 | 36 | 10 | 62 | 40 | 4 | 76 |
| 5296 | A | G | HELD_FEM_EFF | 0.67 | 1.5 | 12 | 3 | 21 | 22 | 9 | 35 |
| 5296 | A | G | CVD_ALL | 1.29 | 0.78 | 104 | 27 | 181 | 74 | 10 | 138 |
| 5298 | C | T | HELD_FEM_EFF | 0.71 | 1.41 | 11 | 3 | 19 | 22 | 9 | 35 |
| 5298 | C | T | CVD_ALL | 1.32 | 0.76 | 101 | 28 | 174 | 74 | 10 | 138 |
| 5298 | C | T | CVD_FEM | 1.62 | 0.62 | 35 | 10 | 60 | 40 | 4 | 76 |
| 5320 | A | G | HELD_FEM_EFF | 0.52 | 1.93 | 19 | 12 | 26 | 33 | 37 | 29 |
| 5361 | A | C | CVD_MAL | 0.82 | 1.22 | 64 | 53 | 75 | 32 | 36 | 28 |
| 5457 | A | G | HELD_FEM_EFF | 0.51 | 1.96 | 12 | 2 | 22 | 21 | 8 | 34 |
| 5704 | C | T | HELD_MAL_LIP | 0.57 | 1.75 | 20 | 10 | 30 | 37 | 32 | 42 |
| 5704 | C | T | CVD_MAL | 0.79 | 1.27 | 68 | 40 | 96 | 33 | 30 | 36 |
| 5717 | A | G | HELD_FEM_ADR3ULN | 1.58 | 0.63 | 38 | 50 | 26 | 83 | 83 | 83 |
| 5717 | A | G | HELD_ALL_ADR3ULN | 1.36 | 0.74 | 65 | 74 | 56 | 156 | 144 | 168 |
| 5959 | A | G | HELD_ALL_CC | 1.53 | 0.65 | 43 | 52 | 34 | 38 | 29 | 47 |
| 5959 | A | G | CVD_FEM | 2.63 | 0.38 | 9 | 12 | 6 | 13 | 7 | 19 |
| 5959 | A | G | HELD_MAL_CC | 1.71 | 0.59 | 14 | 15 | 13 | 17 | 10 | 24 |
| 5959 | A | G | HELD_MAL_ADR5ULN | 0.54 | 1.85 | 9 | 6 | 12 | 67 | 67 | 67 |
| 5959 | A | G | HELD_FEM_ADR | 1.26 | 0.79 | 72 | 71 | 73 | 68 | 51 | 85 |
| 6162 | C | G | HELD_ALL_ADR3ULN | 0.97 | 1.03 | 64 | 37 | 91 | 151 | 90 | 212 |
| 6162 | C | G | HELD_ALL_ADR | 0.96 | 1.04 | 156 | 88 | 224 | 151 | 90 | 212 |
| 6162 | C | G | HELD_ALL_ADR5ULN | 0.99 | 1.01 | 27 | 16 | 38 | 151 | 90 | 212 |
| 6162 | C | G | HELD_MAL_ADR3ULN | 0.82 | 1.22 | 26 | 13 | 39 | 71 | 43 | 99 |
| 6162 | C | G | HELD_FEM_ADR5ULN | 1.28 | 0.78 | 18 | 13 | 23 | 80 | 47 | 113 |
| 6162 | C | G | HELD_MAL_ADR | 0.92 | 1.08 | 74 | 40 | 108 | 71 | 43 | 99 |
| 6236 | C | T | HELD_ALL_ADR5ULN | 1.85 | 0.54 | 27 | 24 | 30 | 152 | 84 | 220 |
| 6236 | C | T | HELD_MAL_ADR3ULN | 1.67 | 0.6 | 27 | 23 | 31 | 72 | 38 | 106 |
| 6236 | C | T | HELD_MAL_ADR5ULN | 2.42 | 0.41 | 10 | 10 | 10 | 72 | 38 | 106 |
| 6236 | C | T | HELD_ALL_ADR3ULN | 1.36 | 0.74 | 63 | 47 | 79 | 152 | 84 | 220 |
| 6482 | A | G | HELD_MAL_HDL | 0.51 | 1.96 | 17 | 18 | 16 | 21 | 34 | 8 |
| 6482 | A | G | HELD_ALL_LIP2 | 0.91 | 1.1 | 619 | 918 | 320 | 709 | 1098 | 320 |
| 6482 | A | G | HELD_MAL_CC2 | 1.82 | 0.55 | 27 | 43 | 11 | 28 | 32 | 24 |
| 6482 | A | G | HELD_MAL_LIP2 | 0.87 | 1.15 | 309 | 461 | 157 | 339 | 539 | 139 |
| 6498 | A | G | CVD_FEM | 2.18 | 0.46 | 32 | 60 | 4 | 35 | 57 | 13 |
| 6744 | C | T | HELD_ALL_ADR5ULN | 1.82 | 0.55 | 26 | 21 | 31 | 149 | 74 | 224 |
| 7133 | C | G | HELD_MAL_CC | 0.36 | 2.8 | 14 | 20 | 8 | 18 | 36 | 0 |
| 8021 | A | G | CVD_FEM | 1.03 | 0.97 | 28 | 35 | 21 | 36 | 44 | 28 |
| 8060 | A | G | CVD_FEM | 1.66 | 0.6 | 35 | 65 | 5 | 40 | 68 | 12 |
| 8060 | A | G | HELD_FEM_HDL | 0.5 | 1.99 | 18 | 29 | 7 | 23 | 43 | 3 |
| 8210 | A | G | HELD_FEM_EFF | 0.72 | 1.4 | 12 | 9 | 15 | 22 | 22 | 22 |
| 8592 | C | T | HELD_FEM_VEFF | 0.99 | 1.01 | 150 | 122 | 178 | 143 | 118 | 168 |
| 8816 | C | G | HELD_FEM_EFF | 1.91 | 0.52 | 13 | 15 | 11 | 11 | 5 | 17 |
| 8846 | A | G | HELD_ALL_LIP | 1.11 | 0.9 | 107 | 161 | 53 | 116 | 166 | 66 |
| 8943 | A | C | HELD_MAL_LIP | 2.17 | 0.46 | 20 | 35 | 5 | 37 | 52 | 22 |
| 9193 | C | G | HELD_FEM_LIP | 1.48 | 0.68 | 83 | 155 | 11 | 80 | 140 | 20 |
| 9193 | C | G | CVD_FEM | 0.6 | 1.67 | 36 | 63 | 9 | 40 | 77 | 3 |
| 9443 | C | T | CVD_MAL | 1.23 | 0.82 | 69 | 43 | 95 | 33 | 12 | 54 |
| 9516 | A | G | HELD_MAL_CC | 1.87 | 0.54 | 14 | 17 | 11 | 18 | 12 | 24 |
| 9698 | A | G | HELD_MAL_ADR | 0.38 | 2.62 | 74 | 8 | 140 | 72 | 30 | 114 |
| 9698 | A | G | HELD_MAL_ADR3ULN | null | 0 | 27 | 54 | 0 | 72 | 30 | 114 |
| 9698 | A | G | HELD_FEM_EFF | 0.91 | 1.1 | 294 | 105 | 483 | 298 | 123 | 473 |
| 9698 | A | G | HELD_MAL_ADR5ULN | | 0 | 10 | 20 | 0 | 72 | 30 | 114 |
| 9698 | A | G | CVD_ALL | 1.27 | 0.79 | 102 | 46 | 158 | 72 | 19 | 125 |
| 9849 | C | T | HELD_FEM_CC | null | 0 | 31 | 62 | 0 | 21 | 39 | 3 |
| 9849 | C | T | HELD_MAL_LIP | 0.46 | 2.18 | 20 | 35 | 5 | 37 | 72 | 2 |
| 9883 | A | G | HELD_FEM_CC | 0.93 | 1.07 | 31 | 23 | 39 | 22 | 18 | 26 |
| 9883 | A | G | HELD_ALL_CC | 0.92 | 1.09 | 45 | 33 | 57 | 39 | 32 | 46 |
| 10079 | A | G | CVD_ALL | 1.54 | 0.65 | 103 | 8 | 198 | 73 | 1 | 145 |
| 10079 | A | G | CVD_MAL | 0.11 | 9.5 | 68 | 8 | 128 | 34 | 68 | 0 |
| 10481 | A | T | HELD_FEM_ADR5ULN | 0.46 | 2.2 | 17 | 12 | 22 | 83 | 97 | 69 |
| 10542 | C | T | HELD_FEM_UEFF | 0.63 | 1.58 | 54 | 8 | 100 | 75 | 21 | 129 |
| 10542 | C | T | HELD_MAL_ADR5ULN | null | 0 | 10 | 20 | 0 | 69 | 14 | 124 |
| 10600 | A | G | HELD_FEM_EFF | null | 0 | 21 | 42 | 0 | 33 | 4 | 62 |
| 10621 | C | T | HELD_FEM_CC | 1.24 | 0.81 | 30 | 52 | 8 | 20 | 32 | 8 |
| 10745 | A | G | HELD_ALL_ADR5ULN | 1.58 | 0.63 | 27 | 20 | 34 | 148 | 75 | 221 |
| 10745 | A | G | HELD_FEM_VEFF | 1.1 | 0.91 | 153 | 90 | 216 | 150 | 77 | 223 |
| 10747 | C | T | HELD_MAL_ADR | 1.06 | 0.94 | 76 | 74 | 78 | 70 | 64 | 76 |
| 10747 | C | T | CVD_ALL | 1.23 | 0.82 | 62 | 54 | 70 | 74 | 51 | 97 |
| 10747 | C | T | HELD_MAL_ADR3ULN | 0.96 | 1.04 | 27 | 24 | 30 | 70 | 64 | 76 |
| 10771 | C | G | HELD_MAL_ADR5ULN | 2.5 | 0.4 | 10 | 12 | 8 | 70 | 48 | 92 |
| 10771 | C | G | HELD_FEM_EFF | 1.12 | 0.89 | 284 | 222 | 346 | 276 | 185 | 367 |
| 10870 | A | G | HELD_MAL_LIP | 1.06 | 0.94 | 20 | 11 | 29 | 37 | 19 | 55 |
| 10870 | A | G | HELD_FEM_LIP | 0.75 | 1.34 | 82 | 32 | 132 | 77 | 46 | 108 |
| 10870 | A | G | HELD_MAL_CC | 0.39 | 2.55 | 14 | 3 | 25 | 18 | 12 | 24 |
| 10870 | A | G | HELD_ALL_CC | 0.67 | 1.5 | 45 | 17 | 73 | 40 | 27 | 53 |
| 10877 | A | C | HELD_ALL_HDL | 3.57 | 0.28 | 9 | 18 | 0 | 15 | 7 | 23 |
| 10948 | G | T | HELD_FEM_LIP | 0.81 | 1.23 | 84 | 83 | 85 | 79 | 95 | 63 |
| 10948 | G | T | HELD_ALL_LIP | 0.81 | 1.23 | 104 | 104 | 104 | 115 | 138 | 92 |
| 10948 | G | T | HELD_FEM_CC2 | 0.87 | 1.15 | 44 | 46 | 42 | 42 | 50 | 34 |
| 10948 | G | T | CVD_MAL | 0.82 | 1.21 | 69 | 63 | 75 | 34 | 41 | 27 |
| 11001 | C | T | HELD_MAL_ADR5ULN | 1.96 | 0.51 | 10 | 9 | 11 | 75 | 41 | 109 |
| 11073 | C | G | HELD_MAL_ADR5ULN | 2.38 | 0.42 | 9 | 10 | 8 | 68 | 43 | 93 |
| 11153 | C | T | HELD_FEM_CC | 1.61 | 0.62 | 31 | 55 | 7 | 22 | 33 | 11 |
| 11210 | C | T | HELD_MAL_CC | 0.46 | 2.17 | 14 | 23 | 5 | 19 | 37 | 1 |
| 11210 | C | T | HELD_ALL_ADR3ULN | 0.67 | 1.48 | 63 | 110 | 16 | 144 | 267 | 21 |
| 11210 | C | T | HELD_ALL_ADR | 0.85 | 1.17 | 153 | 275 | 31 | 144 | 267 | 21 |
| 11248 | C | T | HELD_FEM_ADR | 1.34 | 0.75 | 81 | 131 | 31 | 79 | 112 | 46 |
| 11248 | C | T | HELD_MAL_LIP | 2.3 | 0.43 | 18 | 33 | 3 | 34 | 53 | 15 |
| 11248 | C | T | HELD_ALL_CC | 1.39 | 0.72 | 41 | 68 | 14 | 31 | 44 | 18 |
| 11372 | A | G | HELD_MAL_LIP | 1.67 | 0.6 | 20 | 25 | 15 | 36 | 31 | 41 |
| 11449 | C | G | HELD_FEM_CC | 0.6 | 1.66 | 31 | 6 | 56 | 22 | 10 | 34 |
| 11450 | A | T | HELD_FEM_EFF | 1.14 | 0.87 | 289 | 170 | 408 | 290 | 139 | 441 |
| 11470 | C | T | HELD_MAL_LIP | null | 0 | 20 | 40 | 0 | 36 | 67 | 5 |
| 11472 | A | T | HELD_MAL_LIP | null | 0 | 20 | 40 | 0 | 35 | 65 | 5 |
| 11472 | A | T | HELD_FEM_LIP | 0.63 | 1.6 | 83 | 158 | 8 | 80 | 158 | 2 |
| 11487 | A | T | HELD_MAL_ADR5ULN | null | 0 | 10 | 20 | 0 | 69 | 34 | 104 |
| 11487 | A | T | HELD_MAL_ADR3ULN | 0.48 | 2.11 | 27 | 6 | 48 | 69 | 34 | 104 |
| 11488 | C | G | HELD_MAL_ADR5ULN | null | 0 | 10 | 20 | 0 | 70 | 102 | 38 |
| 11488 | C | G | HELD_FEM_UEFF | 0.74 | 1.35 | 54 | 78 | 30 | 77 | 126 | 28 |
| 11488 | C | G | HELD_MAL_ADR3ULN | 1.73 | 0.58 | 26 | 44 | 8 | 70 | 102 | 38 |
| 11493 | A | G | HELD_MAL_CC | 1.18 | 0.85 | 14 | 6 | 22 | 18 | 6 | 30 |
| 11502 | C | T | HELD_MAL_ADR3ULN | 0.49 | 2.02 | 27 | 8 | 46 | 73 | 44 | 102 |
| 11502 | C | T | HELD_MAL_ADR5ULN | 0.29 | 3.45 | 10 | 2 | 18 | 73 | 44 | 102 |
| 11534 | G | T | HELD_ALL_LIP | null | 0 | 102 | 204 | 0 | 117 | 231 | 3 |
| 11537 | A | G | CVD_FEM | 0.65 | 1.54 | 36 | 52 | 20 | 39 | 68 | 10 |
| 11537 | A | G | HELD_FEM_EFF | 3.11 | 0.32 | 12 | 22 | 2 | 22 | 31 | 13 |
| 11560 | A | G | HELD_FEM_EFF | 0.04 | 23 | 12 | 2 | 22 | 22 | 44 | 0 |
| 11578 | C | T | HELD_FEM_LIP | 4.48 | 0.22 | 61 | 121 | 1 | 65 | 122 | 8 |
| 11578 | C | T | CVD_FEM | 0.42 | 2.37 | 30 | 57 | 3 | 39 | 78 | 0 |
| 11594 | C | T | HELD_FEM_ADR3ULN | null | 0 | 37 | 74 | 0 | 80 | 10 | 150 |
| 11594 | C | T | HELD_ALL_ADR5ULN | null | 0 | 27 | 54 | 0 | 151 | 20 | 282 |
| 11594 | C | T | HELD_ALL_CC | 1.53 | 0.65 | 45 | 10 | 80 | 41 | 3 | 79 |
| 11594 | C | T | HELD_ALL_ADR | 0.6 | 1.66 | 155 | 9 | 301 | 151 | 20 | 282 |
| 11594 | C | T | HELD_FEM_ADR5ULN | null | 0 | 18 | 36 | 0 | 80 | 10 | 150 |
| 11624 | C | T | HELD_ALL_CC | 0.85 | 1.18 | 42 | 57 | 27 | 40 | 60 | 20 |
| 11624 | C | T | HELD_MAL_CC | 0.85 | 1.18 | 13 | 18 | 8 | 18 | 27 | 9 |
| 11624 | C | T | HELD_FEM_EFF | 2.96 | 0.34 | 12 | 22 | 2 | 21 | 30 | 12 |
| 11627 | C | T | HELD_ALL_CC | 0.78 | 1.29 | 45 | 58 | 32 | 40 | 61 | 19 |
| 11627 | C | T | HELD_MAL_CC | 0.76 | 1.32 | 14 | 18 | 10 | 18 | 27 | 9 |
| 11627 | C | T | HELD_FEM_EFF | 3.11 | 0.32 | 12 | 22 | 2 | 22 | 31 | 13 |
| 11644 | A | G | HELD_MAL_ADR5ULN | 0.3 | 3.32 | 10 | 2 | 18 | 68 | 40 | 96 |
| 11650 | A | G | HELD_FEM_EFF | 0.9 | 1.11 | 291 | 157 | 425 | 290 | 181 | 399 |
| 11654 | A | G | HELD_ALL_ADR5ULN | 1.13 | 0.89 | 25 | 17 | 33 | 136 | 84 | 188 |
| 11654 | A | G | HELD_FEM_ADR5ULN | 1.14 | 0.88 | 15 | 11 | 19 | 71 | 47 | 95 |
| 11654 | A | G | HELD_FEM_ADR3ULN | 1.09 | 0.92 | 32 | 23 | 41 | 71 | 47 | 95 |
| 11654 | A | G | HELD_ALL_ADR3ULN | 1.21 | 0.83 | 53 | 39 | 67 | 136 | 84 | 188 |
| 11655 | A | C | HELD_ALL_ADR5ULN | 0.95 | 1.05 | 26 | 35 | 17 | 148 | 203 | 93 |
| 11655 | A | C | HELD_FEM_ADR5ULN | 1.1 | 0.91 | 17 | 23 | 11 | 80 | 104 | 56 |
| 11655 | A | C | HELD_FEM_ADR3ULN | 0.98 | 1.02 | 35 | 45 | 25 | 80 | 104 | 56 |
| 11656 | C | T | HELD_MAL_LIP | 0.53 | 1.87 | 20 | 20 | 20 | 36 | 53 | 19 |
| 11656 | C | T | HELD_FEM_EFF | 2.21 | 0.45 | 12 | 19 | 5 | 22 | 24 | 20 |
| 11656 | C | T | HELD_ALL_LIP | 0.8 | 1.25 | 102 | 119 | 85 | 114 | 156 | 72 |
| 11825 | A | G | HELD_MAL_ADR5ULN | 0.29 | 3.4 | 9 | 15 | 3 | 63 | 121 | 5 |
| 11914 | A | T | HELD_MAL_ADR5ULN | 0.1 | 9.58 | 9 | 2 | 16 | 69 | 83 | 55 |
| 11914 | A | T | HELD_ALL_ADR5ULN | 0.61 | 1.64 | 27 | 24 | 30 | 151 | 178 | 124 |
| 12008 | C | T | HELD_FEM_EFF | 0.73 | 1.37 | 278 | 529 | 27 | 277 | 541 | 13 |
| 12008 | C | T | HELD_ALL_ADR5ULN | null | 0 | 24 | 48 | 0 | 134 | 256 | 12 |
| 12097 | A | G | HELD_ALL_ADR5ULN | 2.46 | 0.41 | 28 | 6 | 50 | 155 | 11 | 299 |
| 12097 | A | G | HELD_FEM_ADR3ULN | 1.94 | 0.51 | 38 | 7 | 69 | 83 | 5 | 161 |
| 12097 | A | G | HELD_MAL_ADR5ULN | 3.04 | 0.33 | 10 | 3 | 17 | 72 | 6 | 138 |
| 12097 | A | G | HELD_ALL_ADR3ULN | 1.7 | 0.59 | 63 | 10 | 116 | 155 | 11 | 299 |
| 12366 | A | G | HELD_FEM_UEFF | 1.52 | 0.66 | 50 | 82 | 18 | 74 | 104 | 44 |
| 12366 | A | G | HELD_ALL_ADR5ULN | 1.23 | 0.81 | 25 | 40 | 10 | 151 | 229 | 73 |
| 12619 | A | G | HELD_MAL_ADR5ULN | 0.01 | 143 | 10 | 1 | 19 | 71 | 142 | 0 |
| 12619 | A | G | HELD_ALL_ADR5ULN | 4.53 | 0.22 | 27 | 2 | 52 | 151 | 1 | 301 |
| 13025 | A | C | HELD_ALL_ADR5ULN | 0.81 | 1.24 | 28 | 34 | 22 | 151 | 201 | 101 |
| 13191 | A | G | HELD_FEM_LIP | 0.72 | 1.4 | 83 | 42 | 124 | 79 | 62 | 96 |
| 13191 | A | G | HELD_MAL_CC | 1.94 | 0.52 | 14 | 11 | 17 | 18 | 5 | 31 |
| 13191 | A | G | HELD_ALL_LIP | 0.76 | 1.31 | 101 | 51 | 151 | 114 | 81 | 147 |
| 13937 | A | C | HELD_FEM_ADR5ULN | 0.53 | 1.89 | 17 | 19 | 15 | 83 | 122 | 44 |
| 900002 | G | T | CVD_FEM | 1.48 | 0.68 | 34 | 23 | 45 | 40 | 15 | 65 |
| 900013 | C | G | CVD_FEM | 1.24 | 0.81 | 35 | 49 | 21 | 40 | 49 | 31 |
| 900013 | C | G | CVD_ALL | 1.14 | 0.88 | 104 | 150 | 58 | 74 | 97 | 51 |
| 900025 | G | T | CVD_MAL | 0.8 | 1.25 | 66 | 41 | 91 | 34 | 31 | 37 |
| 900032 | C | T | CVD_FEM | 1.68 | 0.6 | 25 | 47 | 3 | 37 | 65 | 9 |
| 900045 | C | T | HELD_FEM_EFF | 0.42 | 2.39 | 12 | 4 | 20 | 22 | 18 | 26 |
| 900065 | A | C | CVD_FEM | 1.97 | 0.51 | 32 | 54 | 10 | 39 | 50 | 28 |
| 900065 | A | C | CVD_MAL | 1.09 | 0.92 | 59 | 80 | 38 | 29 | 36 | 22 |
| 900065 | A | C | CVD_ALL | 1.24 | 0.8 | 91 | 134 | 48 | 68 | 86 | 50 |
| 900078 | A | G | HELD_ALL_ADR3ULN | 0.59 | 1.71 | 64 | 116 | 12 | 155 | 297 | 13 |
| 900078 | A | G | HELD_ALL_ADR5ULN | 0.44 | 2.27 | 27 | 48 | 6 | 155 | 297 | 13 |
| 900078 | A | G | HELD_FEM_ADR3ULN | 0.51 | 1.94 | 38 | 69 | 7 | 83 | 161 | 5 |
| 900082 | A | G | HELD_FEM_ADR3ULN | 0.72 | 1.39 | 35 | 25 | 45 | 74 | 70 | 78 |
| 900082 | A | G | HELD_FEM_ADR5ULN | 0.53 | 1.88 | 17 | 10 | 24 | 74 | 70 | 78 |
| 900096 | A | G | CVD_ALL | 0.79 | 1.26 | 101 | 157 | 45 | 72 | 125 | 19 |
| 900107 | C | T | HELD_MAL_ADR5ULN | 0.3 | 3.35 | 10 | 2 | 18 | 73 | 43 | 103 |
| 900115 | A | G | HELD_MAL_ADR5ULN | 0.34 | 2.98 | 9 | 6 | 12 | 72 | 91 | 53 |
| 900115 | A | G | HELD_FEM_EFF | 1.14 | 0.88 | 40 | 58 | 22 | 46 | 62 | 30 |
| 900121 | G | T | HELD_MAL_ADR | 0.88 | 1.14 | 66 | 47 | 85 | 67 | 56 | 78 |
| 900173 | G | T | CVD_ALL | 0.64 | 1.56 | 23 | 17 | 29 | 22 | 26 | 18 |
| 10000002 | A | G | HELD_FEM_EFF | 3.35 | 0.3 | 12 | 21 | 3 | 22 | 25 | 19 |
| 10000006 | A | G | HELD_FEM_CC | 2.77 | 0.36 | 31 | 58 | 4 | 22 | 31 | 13 |
| 10000006 | A | G | HELD_ALL_CC | 2.34 | 0.43 | 44 | 82 | 6 | 38 | 58 | 18 |
| 10000014 | A | C | HELD_ALL_CC | 1.69 | 0.59 | 45 | 83 | 7 | 39 | 64 | 14 |
| 10000014 | A | C | HELD_FEM_CC | 1.68 | 0.6 | 31 | 58 | 4 | 22 | 37 | 7 |
| 10000025 | C | T | HELD_MAL_LIP | 1.46 | 0.68 | 20 | 29 | 11 | 36 | 43 | 29 |

1-16. (canceled)

17. A method of calculating a patient's relative risk (RR) for adverse drug reactions (ADRs) from statin therapy by genotyping a single nucleotide polymorphism (SNP) in DNA of the patient, wherein for three possible genotypes of each SNP, the relative risk associate with each genotype is calculated as follows:
$$\begin{matrix}RR 1 = \frac{N 11}{N 21} / \frac{N 12 + N 13}{N 22 + N 23} \\ RR 2 = \frac{N 12}{N 22} / \frac{N 11 + N 13}{N 21 + N 23} \\ RR 3 = \frac{N 13}{N 23} / \frac{N 11 + N 12}{N 21 + N 22}\end{matrix}$$
wherein:
RR1 represents the relative risk for genotype 1;
RR2 represents the relative risk for genotype 2;
RR3 represents the relative risk for genotype 3;
N11 represents genotype 1, N12 represents genotype 2, and N13 represents genotype 3 for a population of patients that are being tested for ADRs from statin therapy;
N21 represents genotype 1, N22 represents genotype 2, and N23 represents genotype 3 for a population of patients that are known not to be at risk for ADRs from statin therapy;
a value of RR1>1 indicates an increased risk for ADRs from statin therapy for individuals carrying genotype 1;
a value of RR2>1 indicates an increased risk for ADRs from statin therapy for individuals carrying genotype 2; and
a value of RR3>1 indicates an increased risk for ADRs from statin therapy for individuals carrying genotype 3.

18. The method of claim 17, wherein genotype 1, genotype 2, and genotype 3 represent a single nucleotide polymorphism (SNP).

19. The method of claim 18, wherein the SNP is a C to T SNP.

20. The method of claim 19, wherein genotype 1, genotype 2, and genotype 3 are CC, TT, and CT.

21. The method of claim 18, wherein the SNP is an A to G SNP.

22. The method of claim 21, wherein genotype 1, genotype 2, and genotype 3 are AA, AG, and GG.

23. The method of claim 18, wherein the SNP is a C to G SNP.

24. The method of claim 23, wherein genotype 1, genotype 2, and genotype 3 are CC, CG, and GG.

25. The method of claim 18, wherein the SNP is an A to T SNP.

26. The method of claim 25, wherein genotype 1, genotype 2, and genotype 3 are AA, AT, and TT.

27. The method of claim 18, wherein the SNP is a G to T SNP.

28. The method of claim 27, wherein genotype 1, genotype 2, and genotype 3 are GG, GT, and TT.

29. The method of claim 18, wherein the SNP is an A to C SNP.

30. The method of claim 29, wherein genotype 1, genotype 2, and genotype 3 are AA, AC, and CC.

31. A method of calculating a patient's relative risk (RR) for adverse drug reactions (ADRs) from statin therapy by determining allele frequency in a single nucleotide polymorphism (SNP) in DNA of the patient, wherein for two possible alleles of each SNP, the relative risk associate with each allele is calculated as follows:
$$\begin{matrix}RR 1 = \frac{N 11}{N 21} / \frac{N 12}{N 22} \\ RR 2 = \frac{N 12}{N 22} / \frac{N 11}{N 21}\end{matrix}$$
wherein:
RR1 represents the relative risk for allele 1;
RR2 represents the relative risk for allele 2;
N11 represents allele 1 and N 12 represents allele 2 for a population of patients that are being tested for ADRs from statin therapy;
N21 represents allele 1 and N22 represents allele 2 for a population of patients that are known not to be at risk for ADRs from statin therapy;
a value of RR1>1 indicates an increased risk for ADRs from statin therapy for individuals carrying allele 1; and
a value of RR2>1 indicates an increased risk for ADRs from statin therapy for individuals carrying allele 2.

32. The method of claim 31, wherein allele 1 and allele 2 are independently selected from A, C, T, and G.

33. The method of claim 32, wherein allele 1 and allele 2 are C and T, respectively.

34. The method of claim 32, wherein allele 1 and allele 2 are A and G, respectively.

35. The method of claim 32, wherein allele 1 and allele 2 are A and T, respectively.

36. The method of claim 32, wherein allele 1 and allele 2 are C and G, respectively.

37. The method of claim 32, wherein allele 1 and allele 2 are A and C, respectively.

38. The method of claim 32, wherein allele 1 and allele 2 are G and T, respectively.

39. The method of claims 17 and 31, wherein patients with RR1<1, RR2<1, or RR3<1 should receive low doses of statins or switch to alternative therapies to avoid ADRs.

40. A method of calculating a patient's relative risk (RR) for being a high responder to statin therapy by genotyping a single nucleotide polymorphism (SNP) in DNA of the patient, wherein for three possible genotypes of each SNP, the relative risk associate with each genotype is calculated as follows:
$$\begin{matrix}RR 1 = \frac{N 11}{N 21} / \frac{N 12 + N 13}{N 22 + N 23} \\ RR 2 = \frac{N 12}{N 22} / \frac{N 11 + N 13}{N 21 + N 23} \\ RR 3 = \frac{N 13}{N 23} / \frac{N 11 + N 12}{N 21 + N 22}\end{matrix}$$
wherein:
RR1 represents the relative risk for genotype 1;
RR2 represents the relative risk for genotype 2;
RR3 represents the relative risk for genotype 3;
N11 represents genotype 1, N12 represents genotype 2, and N13 represents genotype 3 for a population of patients that are being tested for high response to statin therapy;
N21 represents genotype 1, N22 represents genotype 2, and N23 represents genotype 3 for a population of patients that are low responders statin therapy;
a value of RR1>1 indicates an increased risk for being a high responder to statin therapy for individuals carrying genotype 1;
a value of RR2>1 indicates an increased risk for being a high responder to statin therapy for individuals carrying genotype 2; and
a value of RR>1 indicates an increased risk for being a high responder to statin therapy individuals carrying genotype 3.

41. The method of claim 40, wherein genotype 1, genotype 2, and genotype 3 represent a single nucleotide polymorphism (SNP).

42. The method of claim 41, wherein the SNP is a C to T SNP.

43. The method of claim 42, wherein genotype 1, genotype 2, and genotype 3 are CC, TT, and CT.

44. The method of claim 41, wherein the SNP is an A to G SNP.

45. The method of claim 44, wherein genotype 1, genotype 2, and genotype 3 are AA, AG, and GG.

46. The method of claim 41, wherein the SNP is a C to G SNP.

47. The method of claim 46, wherein genotype 1, genotype 2, and genotype 3 are CC, CG, and GG.

48. The method of claim 41, wherein the SNP is an A to T SNP.

49. The method of claim 48, wherein genotype 1, genotype 2, and genotype 3 are AA, AT, and TT.

50. The method of claim 41, wherein the SNP is a G to T SNP.

51. The method of claim 50, wherein genotype 1, genotype 2, and genotype 3 are GG, GT, and TT.

52. The method of claim 41, wherein the SNP is an A to C SNP.

53. The method of claim 52, wherein genotype 1, genotype 2, and genotype 3 are AA, AC, and CC.

54. A method of calculating a patient's relative risk (RR) for being a high responder to statin therapy by determining allele frequency in a single nucleotide polymorphism (SNP) in DNA of the patient, wherein for two possible alleles of each SNP, the relative risk associate with each allele is calculated as follows:
$$\begin{matrix}RR 1 = \frac{N 11}{N 21} / \frac{N 12}{N 22} \\ RR 2 = \frac{N 12}{N 22} / \frac{N 11}{N 21}\end{matrix}$$
wherein:
RR1 represents the relative risk for allele 1;
RR2 represents the relative risk for allele 2;
N11 represents allele 1 and N 12 represents allele 2 for a population of patients that are being tested for high response to statin therapy;
N21 represents allele 1 and N22 represents allele 2 for a population of patients that are known to be low responders to statin therapy;
a value of RR1>1 indicates an increased risk for being a high responder to statin therapy for individuals carrying allele 1; and
a value of RR2>1 indicates an increased risk for being a high responder to statin therapy for individuals carrying allele 2.

55. The method of claim 54, wherein allele 1 and allele 2 are independently selected from A, C, T, and G.

56. The method of claim 55, wherein allele 1 and allele 2 are C and T, respectively.

57. The method of claim 55, wherein allele 1 and allele 2 are A and G, respectively.

58. The method of claim 55, wherein allele 1 and allele 2 are A and T, respectively.

59. The method of claim 55, wherein allele 1 and allele 2 are C and G, respectively.

60. The method of claim 55, wherein allele 1 and allele 2 are A and C, respectively.

61. The method of claim 55, wherein allele 1 and allele 2 are G and T, respectively.

62. The method of claims 31 and 54, wherein patients with RR1<1, RR2<1, or RR3<1 should receive low doses of statins in order to avoid adverse drug reactions.

63. A method of calculating a patient's relative risk (RR) for cardiovascular disease (CVD) by genotyping a single nucleotide polymorphism (SNP) in DNA of the patient, wherein for three possible genotypes of each SNP, the relative risk associate with each genotype is calculated as follows:
$$\begin{matrix}RR 1 = \frac{N 11}{N 21} / \frac{N 12 + N 13}{N 22 + N 23} \\ RR 2 = \frac{N 12}{N 22} / \frac{N 11 + N 13}{N 21 + N 23} \\ RR 3 = \frac{N 13}{N 23} / \frac{N 11 + N 12}{N 21 + N 22}\end{matrix}$$
wherein:
RR1 represents the relative risk for genotype 1;
RR2 represents the relative risk for genotype 2;
RR3 represents the relative risk for genotype 3;
N11 represents genotype 1, N12 represents genotype 2, and N13 represents genotype 3 for a population of patients that are being tested for CVD;
N21 represents genotype 1, N22 represents genotype 2, and N23 represents genotype 3 for a population of patients that are known not to be at risk for CVD;
a value of RR1>1 indicates an increased risk for CVD for individuals carrying genotype 1;
a value of RR2>1 indicates an increased risk for CVD for individuals carrying genotype 2; and
a value of RR3>1 indicates an increased risk for CVD for individuals carrying genotype 3.

64. The method of claim 63, wherein genotype 1, genotype 2, and genotype 3 represent a single nucleotide polymorphism (SNP).

65. The method of claim 64, wherein the SNP is a C to T SNP.

66. The method of claim 65, wherein genotype 1, genotype 2, and genotype 3 are CC, TT, and CT.

67. The method of claim 64, wherein the SNP is an A to G SNP.

68. The method of claim 67, wherein genotype 1, genotype 2, and genotype 3 are AA, AG, and GG.

69. The method of claim 64, wherein the SNP is a C to G SNP.

70. The method of claim 69, wherein genotype 1, genotype 2, and genotype 3 are CC, CG, and GG.

71. The method of claim 64, wherein the SNP is an A to T SNP.

72. The method of claim 71, wherein genotype 1, genotype 2, and genotype 3 are AA, AT, and TT.

73. The method of claim 64, wherein the SNP is a G to T SNP.

74. The method of claim 73, wherein genotype 1, genotype 2, and genotype 3 are GG, GT, and TT.

75. The method of claim 64, wherein the SNP is an A to C SNP.

76. The method of claim 75, wherein genotype 1, genotype 2, and genotype 3 are AA, AC, and CC.

77. A method of calculating a patient's relative risk (RR) for cardiovascular disease (CVD) by determining allele frequency in a single nucleotide polymorphism (SNP) in DNA of the patient, wherein for two possible alleles of each SNP, the relative risk associate with each allele is calculated as follows:
$$\begin{matrix}RR 1 = \frac{N 11}{N 21} / \frac{N 12}{N 22} \\ RR 2 = \frac{N 12}{N 22} / \frac{N 11}{N 21}\end{matrix}$$
wherein:
RR1 represents the relative risk for allele 1;
RR2 represents the relative risk for allele 2;
N11 represents allele 1 and N 12 represents allele 2 for a population of patients that are being tested for CVD;
N21 represents allele 1 and N22 represents allele 2 for a population of patients that are known not to be at risk for CVD;
a value of RR1>1 indicates an increased risk for CVD for individuals carrying allele 1; and
a value of RR2>1 indicates an increased risk for CVD for individuals carrying allele 2.

78. The method of claim 77, wherein allele 1 and allele 2 are independently selected from A, C, T, and G.

79. The method of claim 78, wherein allele 1 and allele 2 are C and T, respectively.

80. The method of claim 78, wherein allele 1 and allele 2 are A and G, respectively.

81. The method of claim 78, wherein allele 1 and allele 2 are A and T, respectively.

82. The method of claim 78, wherein allele 1 and allele 2 are C and G, respectively.

83. The method of claim 78, wherein allele 1 and allele 2 are A and C, respectively.

84. The method of claim 78, wherein allele 1 and allele 2 are G and T, respectively.

85. The method of claim 19, wherein the C to T SNP is genotyped using oligonucleotide primers of SEQ ID NOs: 157-160 (baySNP 1722); SEQ ID NOs: 181-184 (baySNP 1837); SEQ ID NOs: 197-200 (baySNP 2000); SEQ ID NOs: 321-324 (baySNP 6236); SEQ ID NOs: 325-328 (baySNP 6744); SEQ ID NOs: 365-368 (baySNP 10542); SEQ ID NOs: 397-400 (baySNP 1001); SEQ ID NOs: 401-404 (baySNP 11001)SEQ ID NOs: 413-416 (baySNP 11210); SEQ ID NOs: 417-420 (baySNP 11248); SEQ ID NOs: 453-456 (baySNP 11502); SEQ ID NOs: 469-42 (baySNP 11594); and SEQ ID NOs: 533-536 (baySNP 900107).

86. The method of claim 21, wherein the A to G SNP is genotyped using oligonucleotide primers selected from the group consisting of SEQ ID NOs: 5-8 (baySNP 29); SEQ ID NOs: 73-76 (baySNP 542): SEQ ID NOs: 165-168 (baySNP 1765): SEQ ID NOs: 285-288 (baySNP 4966): SEQ ID NOs: 290-292 (baySNP 5014); SEQ ID NOs: 309-312 (baySNP 5717); SEQ ID NOs: 313-316 (baySNP 5959); SEQ ID NOs: 353-356 (baySNP 9698); SEQ ID NOs: 377-380 (baySNP 10745); SEQ ID NOs: 485-488 (baySNP 11654); SEQ ID NOs: 497-500 (baySNP 11825); SEQ ID NOs: 505-508 (baySNP 12097); SEQ ID NOs: 509-512 (baySNP 12366); SEQ ID NOs: 513-516 (baySNP 12619); and SEQ ID NOs: 529-532 (baySNP 900078).

87. The method of claim 23, wherein the C to G SNP is genotyped using oligonucleotide primers selected from the group consisting of SEQ ID NOs: 317-320 (baySNP 6162); SEQ ID NOs: 381-384 (baySNP 10771); SEQ ID NOs: 405-408 (baySNP 11073); and SEQ ID NOs: 445-448 (baySNP 11488).

88. The method of claim 25, wherein the A to T SNP is genotyped using oligonucleotide primers selected from the group consisting of SEQ ID NOs: 273-276 (baySNP 4206); SEQ ID NOs: 362-364 (baySNP 10481); SEQ ID NOs: 441-444 (baySNP 11487); and SEQ ID NOs: 501-504 (baySNP 11914).

89. The method of claim 27, wherein the G to T SNP is genotyped using oligonucleotide primers of SEQ ID NOs: 257-260 (baySNP 3360).

90. The method of claim 29, wherein the A to C SNP is genotyped using oligonucleotide primers selected from the group consisting of SEQ ID NOs: 129-132 (baySNP 1524); SEQ ID NOs: 253-256 (baySNP 2995) SEQ ID NOs: 489-492 (baySNP 11655); and SEQ ID NOs: 517-520 (baySNP 13025).

91. The method of claim 42, wherein the C to T SNP is genotyped using oligonucleotide primers selected from the group consisting of SEQ ID NOs: 1-4 (baySNP 28); SEQ ID NOs: 29-32 (baySNP 140); SEQ ID NOs: 113-116 (baySNP 1101); SEQ ID NOs: 297-300 (baySNP 5298); SEQ ID NOs: 365-268 (baySNP 10542); SEQ ID NOs: 473-476 (baySNP 11624); SEQ ID NOs: 477-480 (baySNP 11627); SEQ ID NOs: 493-496 (baySNP 11656); and SEQ ID NOs: 525-528 (baySNP 900045).

92. The method of claim 44, wherein the A to G SNP is genotyped using oligonucleotide primer selected form the group consisting of SEQ ID NOs: 33-36 (baySNP 152); SEQ ID NOs: 69-72 (baySNP 472); SEQ ID NOs: 93-96 (baySNP 1056); SEQ ID NOs: 161-164 (baySNP 1757); SEQ ID NOs: 177-180 (baySNP 1806); SEQ ID NOs: 217-220 (baySNP 2119); SEQ ID NOs: 221-224 (baySNP 2141); SEQ ID NOs: (baySNP 3976269-272); SEQ ID NOs: 277-280 (baySNP 4912); SEQ ID NOs: 293-296 (baySNP 5296); SEQ ID NOs: 301-304 (baySNP 5457); SEQ ID NOs: 333-336 (baySNP 8210); SEQ ID NOs: 369-372 (baySNP 10600); SEQ ID NOs: 377-380 (baySNP 10745); SEQ ID NOs: 461-464 (baySNP 11537); SEQ ID NOs: 465-468 (baySNP 11560); SEQ ID NOs: 481-484 (baySNP 11650); SEQ ID NOs: 509-512 (baySNP 12366); and SEQ ID NOs: 537-540 (baySNP 10000002).

93. The method of claim 46, wherein the C to G SNP is genotyped using oligonucletoide primers selected from the group consisting of SEQ ID NOs: 9-12 (baySNP 52); SEQ ID NOs: 13-16 (baySNP 56); SEQ ID NOs: 133-136 (baySNP 1556); SEQ ID NOs: 381-384 (baySNP 10771); SEQ ID NOs: 445-448 (baySNP 11488).

94. The method of claim 48, wherein the A to T SN P is genotyped using oligonucleotide primers of SEQ ID NOs: 429-432 (baySNP 11450).

95. The method of claim 50, wherein the G to T SNP is genotyped using oligonucleotide primers selected from SEQ ID NOs: 125-128 (baySNP 1511) and SEQ ID NOs: 209-212 (baySNP 2085).

96. The method of claim 52, wherein the A to C SNP is genotyped using oligonucleotide primers selected from the group consisting of SEQ ID NOs: 81-84 (baySNP 821); SEQ ID NOs: 233-236 (baySNP 2281); SEQ ID NOs: 253-256 (baySNP 2995); and SEQ ID NOs: 265-268 (baySNP 3975).

97. The method of claim 65, wherein the C to T SNP is genotyped using oligonucleotide primers selected from the group consisting of SEQ ID NOs: 21-24 (baySNP 90); SEQ ID NOs: 25-28 (baySNP 99); SEQ ID NOs: 45-48 (baySNP 224); SEQ ID NOs: 49-52 (baySNP 294); SEQ ID NOs: 53-56 (baySNP 307); SEQ ID NOs: 65-68 (baySNP 466); SEQ ID NOs: 121-124 (baySNP 1504); SEQ ID NOs: 141-144 (baySNP 1582); SEQ ID NOs: 149-152 (baySNP 1662); SEQ ID NOs: 173-176 (baySNP 1799); SEQ ID NOs: 181-184 (baySNP 1837); SEQ ID NOs: 185-188 (baySNP 1870); SEQ ID NOs: 189-192 (baySNP 1882); SEQ ID NOs: 193-196 (baySNP 1988); SEQ ID NOs: 197-200 (baySNP 2000); SEQ ID NOs: 241-244 (baySNP 2341); SEQ ID NOs: 297-300 (baySNP 5298); SEQ ID NOs: 305-308 (baySNP 5704); SEQ ID NOs: 373-376 (baySNP 10621); SEQ ID NOs: 409-412 (baySNP 11153); SEQ ID NOs: 413-415 (baySNP 11210); SEQ ID NOs: 417-420 (baySNP 11248); SEQ ID NOs: 433-436 (baySNP 11470); SEQ ID NOs: 473-476 (baySNP 11624); SEQ ID NOs: 477-480 (baySNP 11627); SEQ ID NOs: 493-396 (baySNP 11656); and SEQ ID NOs: 549-552 (baySNP 10000025).

98. The method of claim 67, wherein the A to G SNP is genotyped using oligonucleotide primers selected from the group consisting of SEQ ID NOs: 5-8 (baySNP 29); SEQ ID NOs: 17-20 (baySNP 89); SEQ ID NOs: 37-40 (baySNP 214); SEQ ID NOs: 73-76 (baySNP 542); SEQ ID NOs: 85-88 (baySNP 1005); SEQ ID NOs: 97-100 (baySNP 1085); SEQ ID NOs: 101-104 (baySNP 1086); SEQ ID NOs: 117-120 (baySNP 1204); SEQ ID NOs: 144-148 (baySNP 1638); SEQ ID NOs: 153-156 (baySNP 1714); SEQ ID NOs: 169-172 (baySNP 1776); SEQ ID NOs: 201-204 (baySNP 2071) SEQ ID NOs: 213-216 (baySNP 2095); SEQ ID NOs: 217-220 (baySNP 2119); SEQ ID NOs: 221-224 (baySNP 2141); SEQ ID NOs: 245-248 (baySNP 2357); SEQ ID NOs: 261-264 (baySNP 3464); SEQ ID NOs: 293-296 (baySNP 5296); SEQ ID NOs: 313-316 (baySNP 5959); SEQ ID NOs: 349-352 (baySNP 9516); SEQ ID NOs: 353-356 (baySNP 9698); SEQ ID NOs: 357-360 (baySNP 9883); SEQ ID NOs: 385-388 (baySNP 10870); SEQ ID NOs: 421-424 (baySNP 11372); SEQ ID NOs: 449-452 (baySNP 11493); SEQ ID NOs: 461-464 (baySNP 11537); and SEQ ID NOs: 541-544 (baySNP 10000006).

99. The method of claim 69, wherein the C to G SNP is genotyped with oligonucleotide primers selected from the group consisting of SEQ ID NOs: 41-44 (baySNP 221); SEQ ID NOs: 61-64 (baySNP 449); SEQ ID NOs: 77-80 (baySNP 739); SEQ ID NOs: 105-108 (baySNP 1092); SEQ ID NOs: 329-332 (baySNP 7133); SEQ ID NOs: 345-348 (baySNP 9193); and SEQ ID NOs: 425-428 (baySNP 11449).

100. The method of claim 71, wherein the A to T SNP is genotyped with oligonucleotide primers selected from the group consisting of SEQ ID NOs: 57-60 (baySNP 411); SEQ ID NOs: 93-96 (baySNP 1055); and SEQ ID NOs: 436-440 (baySNP 11472).

101. The method of claim 73, wherein the G to T SNP is genotyped with oligonucleotide primers selected from the group consisting of SEQ ID NOs: 109-112 (baySNP 1096); SEQ ID NOs: 205-208 (baySNP 2078); SEQ ID NOs: 229-232 (baySNP 2234); SEQ ID NOs: 249-252 (baySNP 2366); SEQ ID NOs: 393-396 (baySNP 10948); and SEQ ID NOs: 457-460 (baySNP 11534).

102. The method of claim 75, wherein the A to C SNP is genotyped with oligonucleotide primers selected from the group consisting of SEQ ID NOs: 81-84 (baySNP 821); SEQ ID NOs: 137-140 (baySNP 1561); SEQ ID NOs: 237-240 (baySNP 2298); SEQ ID NOs: 281-284 (baySNP 4925); SEQ ID NOs: 341-344 (baySNP 8943); SEQ ID NOs: 389-392 (baySNP 10877); and SEQ ID NOs: 545-548 (baySNP 10000014).

103. The method of claims 19 and 42, wherein the C to T SNP is used to concurrently determine the patient's risk for ADRs from statin therapy and the patient's risk of being a high responder to statin therapy, wherein the C to T SNP is genotyped using oligonucleotide primers of SEQ ID NOs: 365-368 (baySNP 10542).

104. The method of claims 19 and 65, wherein the C to T SNP is used to concurrently determine the patient's risk for ADRs from statin therapy and the patient's risk for CVD, wherein the C to T SNP is genotyped with oligonucleotide primers selected from SEQ ID NOs: 181-184 (baySNP 1837); SEQ ID NOs: 197-200 (baySNP 2000); SEQ ID NOs: 417-420 (baySNP 11248); and SEQ ID NOs: 469-472 (baySNP 11594).

105. The method of claim 42 and 65, wherein the C to T SNP is used to concurrently determine the patient's risk for being a high responder to statin therapy and the patient's risk for CVD, wherein the C to T SNP is genotyped with oligonucleotide primers selected from SEQ ID NOs: 297-300 (baySNP 5298); SEQ ID NOs: 473-476 (baySNP 11624); SEQ ID NOs: 477-480 (baySNP 11627); and SEQ ID NOs:493-496 (baySNP 11656).

106. The method of claims 21 and 44, wherein the A to G SNP is used to concurrently determine the patient's risk for ADRs from statin therapy and the patient's risk of being a high responder to statin therapy, wherein the A to G SNP is genotyped with oligonucleotide primers selected from SEQ ID NOs: 353-356 (baySNP 9698); SEQ ID NOs: 377-380 (baySNP 10745); and SEQ ID NOs: 509-512 (baySNP 12366).

107. The method of claims 21 and 67, wherein the A to G SNP is used to concurrently determine the patient's risk for ADRs from statin therapy and for the patient's risk for CVD, wherein the A to G SNP is genotyped with oligonucleotide primers selected from SEQ ID NOs: 5-8 (baySNP 29) and SEQ ID NOs: 313-316 (baySNP 5959).

108. The method of claims 44 and 67, wherein the A to G SNP is used to concurrently determine the patient's risk for being a high responder to statin therapy and the patient's risk for CVD, wherein the A to G SNP is genotyped with oligonucleotide primers selected from SEQ ID NOs: 217-220 (baySNP 2119); SEQ ID NOs: 221-224 (baySNP 2141); SEQ ID NOs: 293-296 (baySNP 5296); and SEQ ID NOs: 461-464 (baySNP 11537).

109. The method of claims 52 and 75, wherein the A to C SNP is used to concurrently determine the patient's risk for being a high responder to statin therapy and for CVD, wherein the A to C SNP is genotyped using oligonucleotide primers of SEQ ID NOs: 81-84 (baySNP 821).

110. The method of claims 23 and 46, wherein the C to G SNP is used concurrently determine the patient's risk for ADRs from statin therapy and the patient's risk of being a high responder to statin therapy, wherein the C to G SNP is genotyped using oligonucleotide primers of SEQ ID NOs: 445-448 (baySNP 11488).
